(19) United States
(12) Patent Application Publication
(10) Pub. No.: US 2021/0223531 A1
(43) Pub. Date: Jul. 22, 2021

(54) OPTICAL SYSTEM FOR FLUORESCENCE IMAGING

(71) Applicant: Element Biosciences, Inc., San Diego, CA (US)

(72) Inventors: Minghao Guo, San Diego, CA (US); Michael Previte, San Diego, CA (US); Steven Xiangling Chen, San Diego, CA (US); Chunhong Zhou, San Diego, CA (US)

(21) Appl. No.: 17/151,020

(22) Filed: Jan. 15, 2021

Related U.S. Application Data
(60) Provisional application No. 62/962,723, filed on Jan. 17, 2020.
(60) Provisional application No. 63/037,544, filed on Jun. 10, 2020.
(60) Provisional application No. 63/039,384, filed on Jun. 15, 2020.
(60) Provisional application No. 63/136,592, filed on Jan. 12, 2021.

(51) Int. Cl. G02B 21/16; B01L 3/00; G01N 21/64; G02B 21/06; G02B 21/02

(52) U.S. Cl. CPC G02B 21/16; B01L 3/502; G01N 21/6428; G01N 2021/6439; G02B 21/06; G02B 21/02; G01N 21/6458

(57) ABSTRACT

Optical systems for DNA sequencing and other assays are described. Microscope designs may include a light source configured to emit an excitation beam and an objective lens disposed to receive the excitation beam, direct the excitation beam to a specimen, and receive emission light emitted by the specimen in response to the excitation beam. A plurality of detection channels includes optics configured to receive at least a portion of the emission light. A first dichroic filter can be disposed to reflect the excitation beam into the objective lens and to transmit the emission light, and a second dichroic filter can be disposed to receive the transmitted emission light, transmit a first portion of the transmitted emission light to a first channel of the plurality of channels, and reflect a second portion of the transmitted emission light to a second channel of the plurality of channels. Imaging or detection performance may further be improved by a reduced angle of incidence between the emission light and some or all of the dichroic filters, and/or by linearly polarizing the excitation beam such that the excitation beam is s-polarized with respect to the first dichroic filter.

### CROSS-REFERENCE TO RELATED APPLICATIONS

This application claims the benefit of U.S. Provisional Application Ser. No. 62/962,723, filed Jan. 17, 2020, titled “HIGH PERFORMANCE FLUORESCENCE IMAGING MODULE FOR GENOMIC TESTING ASSAY,” U.S. Provisional Application Ser. No. 63/037,544, filed Jun. 10, 2020, titled “MULTI-CHANNEL FLUORESCENCE MICROSCOPE,” U.S. Provisional Application Ser. No. 63/039,384, filed Jun. 15, 2020, titled “MULTI-CHANNEL FLUORESCENCE MICROSCOPE,” and U.S. Provisional Application Ser. No. 63/136,592, filed Jan. 12, 2021, titled “OPTICAL SYSTEM FOR FLUORESCENCE IMAGING,” all of which are hereby incorporated herein by reference in their entirety.

### BACKGROUND

### Field

The present disclosure relates to optical systems configured to detect fluorescence such as fluorescence microscopes, and more particularly to optical systems such as multi-channel fluorescence microscopes for DNA sequencing or analyzing other assays by detecting fluorescence.

### Description of the Related Art

DNA sequencing and other analyte analysis can be performed using fluorescence microscopy. One or more excitation beams may induce fluorescence, for example, of one or more fluorescent dyes associated with a sample or specimen that is detected with a sensor. To quickly analyze numerous reactions, for example, in a multiplexed process, an imaging system such as an optical microscope images different sample sites disposed across a substrate or support structure (e.g., a flow cell, microfluidic chip, capillary tube, etc.). In many cases, for example, a sample substrate or support structure includes numerous sample sites disposed across the substrate or support structure where sample binds. The sample sites are spaced apart by small distances such that an optical microscope that forms an optical image of the plurality of sample sites on an optical detector array may be used to capture images of the numerous sites where sample binds to detect fluorescence.

In some cases, different dyes are employed that produce fluorescence at different wavelengths or bands. To detect the different wavelengths or bands individually, fluorescence microscopes have multiple channels, different channels configured to detect the different wavelengths or bands, respectively. For example, dichroic filters or beamsplitters may be used to direct fluorescence emission of different wavelengths or bands to different respective channels for detection.

Multi-channel fluorescence microscopes having a large field-of-view (FOV) generally provide for relatively high throughput DNA sequencing or analyte analysis. The increased FOV enables more sample sites on the substrate or support to be interrogated simultaneously. However, many existing multi-channel large FOV fluorescence microscope designs require large dichroic filters to split light propagating into the different detection channels. The large filter size may make it more difficult to provide a flat filter surface, potentially introducing wavefront error. In addition, a large FOV may reduce the effective sharpness of the edge of the spectral filter. Transmission and reflection of the spectral filters is angle dependent. Thus, rays of the same wavelength incident on the filter at different angles will have different transmissivity and reflectivity diluting the sharpness of any transition from a spectrally transmissive region to a spectrally reflective region of the filter. Conventional fluorescence microscopy may also include other limitations.

In typical fluorescence-based genomic testing assays, e.g., genotyping or nucleic acid sequencing (using either real time, cyclic, or stepwise reaction schemes), dye molecules that are attached to nucleic acid molecules tethered on a substrate are excited using an excitation light source, a fluorescence photon signal is generated in one or more spatially-localized positions on the substrate, and the fluorescence is subsequently imaged through an optical system onto an image sensor. An analysis process is then used to analyze the images, find the positions of labeled molecules (or clonally amplified clusters of molecules) on the substrate, and quantify the fluorescence photon signal in terms of wavelength and spatial coordinates, which may then be correlated with the degree to which a specific chemical reaction, e.g., a hybridization event or base addition event, occurred in the specified locations on the substrate. Imaging-based methods provide large scale parallelism and multiplexing capabilities, which help to drive down the cost and accessibility of such technologies. However, detection errors that arise from, for example, overly dense packing of labeled molecules (or clonally-amplified clusters of molecules) within a small region of the substrate surface, or due to low contrast-to-noise ratio (CNR) in the image, may lead to errors in attributing the fluorescence signal to the correct molecules (or clonally amplified clusters of molecules). Thus, there may be a need for fluorescence imaging methods and systems that provide increased optical resolution and/or improved image quality for genomics applications that lead to corresponding improvements in genomic testing accuracy.

Flow-cell devices are widely used in chemistry and biotechnology applications. Particularly in next-generation sequencing (NGS) systems, such devices are used to immobilize template nucleic acid molecules derived from biological samples and then introduce a repetitive flow of sequencing-by-synthesis reagents to attach labeled nucleotides to specific positions in the template sequences. A series of label signals are detected and decoded to reveal the nucleotide sequences of the template molecules, e.g., immobilized and/or amplified nucleic acid template molecules attached to an internal surface of the flow cell.

Typical NGS flow cells are multi-layer structures fabricated from planar surface substrates and other flow cell components (see, for example, U.S. Patent Application Publication No. 2018/0178215 A1), which are then bonded through mechanical, chemical, or laser bonding techniques to form fluid flow channels. Such flow cells may involve costly multi-step, precision fabrication techniques to achieve the required design specifications. On the other hand, inexpensive and off-the-shelf, single lumen (flow channel) capillaries are available in a variety of sizes and shapes but may not be suited for ease of handling and compatibility with the repetitive switching between reagents that are employed for applications such as NGS.

### SUMMARY

Various innovative fluorescence microscope, microscope, and other optical system designs such as optical imaging systems as well as innovative support structures such as flow cells, microfluidic chips and capillary tube that may potentially provide improved performance are disclosed herein.

The systems, methods, and devices described herein each have several aspects, no single one of which is solely responsible for its desirable attributes. Without limiting the scope of this disclosure, several non-limiting features will now be discussed briefly. The following paragraphs describe various example implementations of the devices, systems, and methods described herein.

### Part I

1. An optical system comprising:
a light source configured to emit an excitation beam;
an objective lens disposed to receive the excitation beam, direct the excitation beam to a specimen, and receive emission light in response to the excitation beam;
a plurality of channels comprising optics configured to receive at least a portion of the emission light;
a first dichroic filter disposed to reflect the excitation beam into the objective lens and to transmit the emission light; and
a second dichroic filter disposed to receive the transmitted emission light, transmit a first portion of the transmitted emission light to a first channel of the plurality of channels, and reflect a second portion of the transmitted emission light to a second channel of the plurality of channels.

2. The optical system of Example 1, wherein the second dichroic filter is disposed to receive the transmitted emission light such that a central beam axis of the transmitted emission light has an angle of incidence of between 25 degrees and 35 degrees.

3. The optical system of Example 1 or 2, wherein the second dichroic filter is disposed to receive the transmitted emission light such that the central beam axis of the transmitted emission light has an angle of incidence of between 27.5 degrees and 32.5 degrees.

4. The optical system of any one of Examples 1-3, wherein the second dichroic filter is disposed to receive the transmitted emission light such that the central beam axis of the transmitted emission light has an angle of incidence of 30 degrees.

5. The optical system of any one of Examples 1-4, wherein the objective lens is configured such that the transmitted emission light is incident upon the second dichroic filter at angles of incidence within 5 degrees of an angle of incidence of a central beam axis of the transmitted emission light.

6. The optical system of any one of Examples 1-5, wherein the objective lens is configured such that the transmitted emission light is incident upon the second dichroic filter at angles of incidence within 2.5 degrees of the angle of incidence of the central beam axis of the transmitted emission light.

7. The optical system of any one of Examples 1-6, wherein the objective lens has a focal length of between 30 mm and 40 mm.

8. The optical system of any one of Examples 1-7, wherein the objective lens has a focal length of between 35 mm and 37 mm.

9. The optical system of any one of Examples 1-8, wherein the second dichroic filter has a transmission edge with a spectral span that varies less than 15 nm across a full field of view of the transmitted emission light.

10. The optical system of any one of Examples 1-9, wherein the second dichroic filter has a transmission edge with a spectral span that varies less than 8 nm across a full field of view of the transmitted emission light.

11. The optical system of any one of Examples 1-10, wherein the light source is a laser source.

12. The optical system of Example 11, wherein the laser source generates a linearly polarized excitation beam.

13. The optical system of Example 11 or 12, wherein the excitation beam is s-polarized with respect to the first dichroic filter.

14. The optical system of any one of Examples 1-13, further comprising a third dichroic filter disposed to receive one of the first portion or the second portion of the transmitted emission light and to reflect a portion of the received one of the first portion or second portion of the transmitted emission light to a third channel of the plurality of channels.

15. The optical system of Example 14, wherein the third dichroic filter is disposed to receive the one of the first portion or the second portion of the transmitted emission light at an angle of incidence of between 25 degrees and 35 degrees.

16. The optical system of Example 14 or 15, wherein the third dichroic filter is disposed to receive the one of the first portion or the second portion of the transmitted emission light at an angle of incidence of between 27.5 degrees and 32.5 degrees.

17. The optical system of any one of Examples 14-16, further comprising a fourth dichroic filter disposed to receive the other of the first portion or the second portion of the transmitted emission light and to reflect a portion of the received other of the first portion or the second portion of the transmitted emission light to a fourth channel of the plurality of channels.

18. The optical system of any one of Examples 14-17, wherein the fourth dichroic filter is disposed to receive the other of the first portion or the second portion of the transmitted emission light at an angle of incidence of between 25 degrees and 35 degrees.

19. The optical system of any one of Examples 14-18, wherein the fourth dichroic filter is disposed to receive the other of the first portion or the second portion of the transmitted emission light at an angle of incidence of between 27.5 degrees and 32.5 degrees.

20. The optical system of any one of Examples 1-19, wherein each channel of the plurality of channels comprises a tube lens.

21. The optical system of Example 20, wherein each channel of the plurality of channels comprises a photodetector, the tube lens disposed to direct a respective portion of the emission light onto the photodetector.

22. The optical system of any one of Examples 1-21, wherein the multi-channel optical system is configured to receive the specimen in a microscopy flow cell.

23. An optical system comprising:
a light source configured to emit an excitation beam;
an objective lens disposed to receive the excitation beam, direct the excitation beam to a specimen, and receive emission light emitted in response to the excitation beam;
a plurality of channels comprising optics configured to receive at least a portion of the emission light; and
a dichroic filter disposed to receive the emission light such that a central beam axis of the emission light has an angle of incidence of less than 45 degrees, transmit a first portion of the emission light to a first channel of the plurality of channels, and reflect a second portion of the emission light to a second channel of the plurality of channels.

24. The optical system of Example 23, wherein the dichroic filter is disposed such that the central beam axis of the emission light has an angle of incidence between 25 degrees and 35 degrees.

25. The optical system of Example 23 or 24, wherein the dichroic filter is disposed such that the central beam axis of the emission light has an angle of incidence between 25 degrees and 35 degrees.

26. The optical system of any one of Examples 23-25, wherein the dichroic filter is disposed such that the central beam axis of the emission light has an angle of incidence of 30 degrees.

27. The optical system of any one of Examples 23-26, wherein the objective lens is configured such that the emission light received by the objective lens is incident upon the dichroic filter at angles of incidence within 5 degrees of the angle of incidence of the central beam axis.

28. The optical system of any one of Examples 23-27, wherein the objective lens is configured such that the emission light received by the objective lens is incident upon the dichroic filter at angles of incidence within 2.5 degrees of the angle of incidence of the central beam axis.

29. The optical system of any one of Examples 23-28, wherein the objective lens has a focal length of between 30 mm and 40 mm.

30. The optical system of any one of Examples 23-29, wherein the objective lens has a focal length of between 35 mm and 37 mm.

31. The optical system of any one of Examples 23-30, wherein the dichroic filter has a transmission edge with a spectral span that varies less than 15 nm across a full field of view of the emission light.

32. The optical system of any one of Examples 23-31, wherein the dichroic filter has a transmission edge with a spectral span that varies less than 8 nm across a full field of view of the emission light.

33. The optical system of any one of Examples 23-32, wherein the light source is a laser source.

34. The optical system of Example 33, wherein the laser source generates a linearly polarized excitation beam.

35. The optical system of Example 33 or 34, wherein the excitation beam is s-polarized with respect to a second dichroic filter disposed to reflect the excitation beam into the objective lens and to transmit the emission light to the dichroic filter.

36. The optical system of any one of Examples 23-35, further comprising a third dichroic filter disposed to receive one of the first portion or the second portion of the emission light and to reflect a portion of the received one of the first portion or second portion of the emission light to a third channel of the plurality of channels.

37. The optical system of Example 36, wherein the third dichroic filter is disposed to receive the one of the first portion or the second portion of the emission light at an angle of incidence of between 25 degrees and 35 degrees.

38. The optical system of Example 36 or 37, wherein the third dichroic filter is disposed to receive the one of the first portion or the second portion of the emission light at an angle of incidence of between 27.5 degrees and 32.5 degrees.

39. The optical system of any one of Examples 36-38, further comprising a fourth dichroic filter disposed to receive the other of the first portion or the second portion of the emission light and to reflect a portion of the received other of the first portion or the second portion of the emission light to a fourth channel of the plurality of channels.

40. The optical system of Example 39, wherein the fourth dichroic filter is disposed to receive the other of the first portion or the second portion of the emission light at an angle of incidence of between 25 degrees and 35 degrees.

41. The optical system of Example 39 or 40, wherein the fourth dichroic filter is disposed to receive the other of the first portion or the second portion of the emission light at an angle of incidence of between 27.5 degrees and 32.5 degrees.

42. The optical system of any one of Examples 23-41, wherein each channel of the plurality of channels comprises a tube lens.

43. The optical system of Example 42, wherein each channel of the plurality of channels comprises a photodetector, the tube lens disposed to direct a respective portion of the emission light onto the photodetector.

44. The optical system of any one of Examples 23-43, wherein the optical system is configured to receive the specimen in a microscopy flow cell.

45. An optical system comprising:
a light source configured to emit an excitation beam;
an objective lens disposed to receive the excitation beam, direct the excitation beam to a specimen, and receive emission light in response to the excitation beam;
at least one channel comprising optics configured to receive at least a portion of the emission light; and
a dichroic filter disposed to reflect the excitation beam into the objective lens and to transmit the emission light to the at least one channel, wherein the excitation beam is s-polarized with respect to the dichroic filter.

46. The optical system of Example 45, wherein the at least one channel comprises a plurality of channels each comprising optics configured to receive at least a portion of the emission light transmitted by the dichroic filter, the optical system further comprising a second dichroic filter disposed to receive the emission light such that a central beam axis of the emission light has an angle of incidence of less than 45 degrees, reflect a first portion of the emission light to a first channel of the plurality of channels, and transmit a second portion of the emission light to a second channel of the plurality of channels.

47. The optical system of Example 46, wherein the second dichroic filter is disposed to receive the transmitted emission light such that a central beam axis of the transmitted emission light has an angle of incidence of between 25 degrees and 35 degrees.

48. The optical system of Example 46 or 47, wherein the second dichroic filter is disposed to receive the transmitted emission light such that the central beam axis of the transmitted emission light has an angle of incidence of between 27.5 degrees and 32.5 degrees.

49. The optical system of any one of Examples 46-48, wherein the second dichroic filter is disposed to receive the transmitted emission light such that the central beam axis of the transmitted emission light has an angle of incidence of 30 degrees.

50. The optical system of any one of Examples 46-49, wherein the objective lens is configured such that the transmitted emission light is incident upon the second dichroic filter at angles of incidence within 5 degrees of an angle of incidence of a central beam axis of the transmitted emission light.

51. The optical system of any one of Examples 46-50, wherein the objective lens is configured such that the transmitted emission light is incident upon the second dichroic filter at angles of incidence within 2.5 degrees of the angle of incidence of the central beam axis of the transmitted emission light.

52. The optical system of any one of Examples 46-51, wherein the second dichroic filter has a transmission edge with a spectral span that varies less than 15 nm across a full field of view of the transmitted emission light.

53. The optical system of any one of Examples 46-52, wherein the second dichroic filter has a transmission edge with a spectral span that varies less than 8 nm across a full field of view of the transmitted emission light.

54. The optical system of any one of Examples 46-53, further comprising a third dichroic filter disposed to receive one of the first portion or the second portion of the transmitted emission light and to reflect a portion of the received one of the first portion or second portion of the transmitted emission light to a third channel of the plurality of channels.

55. The optical system of Example 54, wherein the third dichroic filter is disposed to receive the one of the first portion or the second portion of the transmitted emission light at an angle of incidence of between 25 degrees and 35 degrees.

56. The optical system of Example 54 or 55, wherein the third dichroic filter is disposed to receive the one of the first portion or the second portion of the transmitted emission light at an angle of incidence of between 27.5 degrees and 32.5 degrees.

57. The optical system of any one of Examples 54-56, further comprising a fourth dichroic filter disposed to receive the other of the first portion or the second portion of the transmitted emission light and to reflect a portion of the received other of the first portion or the second portion of the transmitted emission light to a fourth channel of the plurality of channels.

58. The optical system of Example 57, wherein the fourth dichroic filter is disposed to receive the other of the first portion or the second portion of the transmitted emission light at an angle of incidence of between 25 degrees and 35 degrees.

59. The optical system of Example 57 or 58, wherein the fourth dichroic filter is disposed to receive the other of the first portion or the second portion of the transmitted emission light at an angle of incidence of between 27.5 degrees and 32.5 degrees.

60. The optical system of any one of Examples 45-59, wherein the objective lens has a focal length of between 30 mm and 40 mm.

61. The multi-channel optical system of any one of Examples 45-60, wherein the objective lens has a focal length of between 35 mm and 37 mm.

62. The optical system of any one of Examples 45-61, wherein the light source is a laser source.

63. The optical system of any one of Examples 45-62, wherein the channel comprises a tube lens.

64. The optical system of Example 63, wherein the channel comprises a photodetector, the tube lens disposed to direct a respective portion of the emission light onto the photodetector.

65. The optical system of any one of Examples 45-64, wherein the optical system is configured to receive a specimen in a microscopy flow cell.

66. The optical system of any one of Examples 1-65, wherein the objective lens has a numerical aperture of less than 0.6.

67. The optical system of any one of Examples 1-66, wherein the optical system is capable of simultaneous imaging of two or more surfaces separated by 0.075 mm or more.

68. The optical system of any one of Examples 1-67, wherein the optical system has a field of view greater than 1.5 mm.

69. The optical system of any one of Examples 1-68, wherein the optical system does not require additional optical compensation for multi-surface imaging.

70. The optical system of any one of Examples 1-69, further comprising one or more tube lenses.

71. The optical system of any one of Examples 1-70, further comprising a flow cell with two or more imaging surfaces at different distances from the objective lens.

72. The optical system of Example 71, wherein the two or more imaging surfaces comprise a hydrophilic coating.

73. The optical system of Example 71 or 72, wherein the two or more imaging surfaces yield a contrast-to-noise ratio greater than 20.

74. The optical system of any one of Examples 1-73, wherein the optical system is configured for high throughput assays.

75. A method of detecting features on one or more surfaces using the optical system of any one of Examples 1-74, comprising imaging the one or more surfaces using a combination of optical elements including the objective lens.

76. The method of Example 75, wherein the combination of optical elements is capable of simultaneous imaging of two or more surfaces separated by 0.075 mm or more.

77. The method of Example 75 or 76, wherein the combination of optical elements has a field of view greater than 1.5 mm.

78. The method of any one of Examples 75-77, wherein the combination of optical elements does not require additional optical compensation for multi-surface imaging.

79. The method of any one of Examples 75-78, wherein the one or more objective lenses has a numerical aperture of less than 0.6.

80. The method of Example 75, wherein the combination of optical elements further comprises a tube lens.

81. A method of sequencing one or more nucleic acids using the optical system of any one of Examples 1-74, comprising detecting features on one or more surfaces.

82. The method of Example 81, further comprising imaging the one or more surfaces using a combination of optical elements comprising including the objective lens.

83. The method of Example 81 or 82, wherein the combination of optical elements further comprises a tube lens.

84. The method of any one of Examples 81-83, wherein the combination of optical elements is capable of simultaneous imaging of two or more surfaces separated by 0.075 mm or more.

85. The method of any one of Examples 81-84, wherein the combination of optical elements has a field of view greater than 1.5 mm.

86. The method of any one of Examples 81-85, wherein the combination of optical elements does not require additional optical compensation for multi-surface imaging.

87. The optical system or method of any one of Examples 1-86, wherein the one or more surfaces comprise one or more surfaces of a flow cell modified such that both surfaces yield a contrast-to-noise ratio of greater than 20 for a single sequencing cycle.

88. The optical system or method of any one of Examples 1-87, wherein the one or more surfaces comprise one or more surfaces of a flow cell such that both surfaces yield a contrast-to-noise ratio greater than 20 for 5 consecutive sequencing cycles.

89. The optical system or method of any one of Examples 1-88, wherein the one or more surfaces comprise one or more surfaces of a flow cell such that both surfaces yield a contrast-to-noise ratio of greater than 20 for 10 consecutive sequencing cycles.

90. The optical system or method of any one of Examples 1-89, wherein the flow cell is coated with a hydrophilic coating.

91. The optical system or method of any one of Examples 1-90, wherein the flow cell comprises a hydrophilic substrate comprising labeled nucleic acid colonies.

92. The optical system or method of any one of Examples 1-91, further comprising a flow cell with labeled nucleic acid colonies have a density of at least 10000/mm².

93. The optical system or method of any one of Examples 1-92, wherein an image of the surface shows a contrast to noise ratio of at least 20.

94. The optical system or method of any one of Examples 1-93, wherein the surface comprises nucleic acid colonies comprising 1, 2, 3, or 4 distinct detectable labels.

95. The optical system or method of any one of Examples 1-94, further comprising imaging channels to detect 1, 2, 3, or 4 distinct labels.

96. A method of sequencing a nucleic acid comprising carrying out the sequencing by binding or sequencing by synthesis reaction on one or more surfaces using the optical system of any one of Examples 1-74 or the method of any one of Examples 75-95

97. The method of Example 96, further comprising detecting a bound or incorporated base.

98. A method of determining a genotype of a sample comprising a nucleic acid molecule, comprising preparing said nucleic acid molecule for sequencing, and then sequencing said nucleic acid molecule using the optical system of any one of Examples 1-74 or the method of any one of Examples 75-95.

99. The optical system or method of any one of the preceding Examples, wherein a flow cell or sample chamber or sample support structure comprises one, two, three, four, five, or six imaging surfaces.

100. The optical system or method of any one of the preceding Examples, wherein the system or method does not require additional compensation in order to achieve imaging of both imaging surfaces of a dual-surface flow cell or sample support structure.

101. The optical system or method of any one of the preceding Examples, wherein the system or method does not require additional compensation in order to achieve imaging of multiple surfaces of a multiple-surface flow cell or sample chamber or sample support structure.

102. The optical system or method of any one of the preceding Examples, further comprising polarization optics configured to polarize the excitation beam.

103. The optical system or method of any one of the preceding Examples, further comprising polarization optics configured to linearly polarize the excitation beam.

104. The optical system or method of any one of the preceding Examples, further comprising polarization optics configured to orient the polarization of the excitation beam such that the excitation beam is s-polarized.

105. The optical system or method of any one of the preceding Examples, wherein the light source is configured to output polarized light.

106. The optical system or method of any one of the preceding Examples, wherein the light source is configured to output linearly polarized light.

107. The optical system or method of any one of the preceding Examples, wherein the light source is oriented such that the excitation beam is s-polarized.

108. An optical system of any one of the Examples above, wherein said optical system has a field-of-view of at least 1.0 mm wide with less than 0.1 waves of aberration over at least 80% of the field-of-view.

109. An optical system of any one of the Examples above, wherein said optical system has a field-of-view of at least 1.0 mm wide with less than 0.1 waves of aberration over at least 90% of the field-of-view.

110. An optical system of any one of the Examples above, wherein said n has a field-of-view of at least 2.0 mm wide with less than 0.09 waves of aberration over at least 80% of the field-of-view.

111. An optical system of any one of the Examples above, wherein said optical system has a field-of-view of at least 2.0 mm wide with less than 0.09 waves of aberration over at least 90% of the field-of-view.

112. An optical system of any one of the Examples above, wherein said optical system has a field-of-view of at least 3.0 mm wide with less than 0.09 waves of aberration over at least 80% of the field-of-view.

113. An optical system of any one of the Examples above, wherein said optical system has a field-of-view of at least 3.0 mm wide with less than 0.09 waves of aberration over at least 90% of the field-of-view.

114. An optical system of any one of the Examples above, wherein said optical system has a field-of-view of at least 3.2 mm wide with less than 0.09 waves of aberration over at least 80% of the field-of-view.

115. An optical system of any one of the Examples above, wherein said optical system has a field-of-view of at least 3.2 mm wide with less than 0.09 waves of aberration over at least 90% of the field-of-view.

116. An optical system of any one of the Examples above, wherein said optical system has a work distance of in a range from 2 to 6 mm.

117. An optical system of any one of the Examples above, wherein said optical system has a work distance of in a range from 2.5 to 5.5 mm.

118. An optical system of any one of the Examples above, wherein said optical system has a work distance of in a range from 3 to 5 mm.

119. An optical system of any one of the Examples above, wherein said optical system has a work distance of at least 3 mm.

120. The optical system of any one of the Examples above, wherein said optical system is capable of capturing with said photodetector array an image of fluorescence emitting sample sites on first and second surfaces on a sample support structure.

121. The optical system of Example 120, wherein said first surface is between said objective lens and said second surface, said first and second surfaces separated from each other by at least 0.075 mm.

122. The optical system of any of one the Examples 120 or 121, wherein said optical system has less than 0.1 waves of aberration over at least 80% of the field of view for both said first and second surfaces.

123. The optical system of any one of the Examples 120-122, wherein said optical system has less than 0.1 waves of aberration over at least 90% of the field of view for both said first and second surfaces.

124. The optical system of any one of the Examples 110-122, wherein said optical system provide diffraction limited imaging of for both said first and second surfaces.

125. An optical system of any one of the Examples above, wherein the optical system has a field-of-view of at least 1.0 mm wide and is diffraction limited.

126. An optical system of any one of the Examples above, wherein said optical system has a field-of-view of at least 1.0 mm wide.

127. An optical system of any one of the Examples above, wherein said optical system has a field-of-view of at least 2.0 mm wide.

128. An optical system of any one of the Examples above, wherein said optical system has a field-of-view of at least 3.0 mm wide.

129. An optical system of any one of the Examples above, wherein said optical system has a field-of-view of at least 3.2 mm wide.

130. The optical system of any one of the Examples above, wherein said optical system is capable of capturing with said photodetector array an image of fluorescence emitting sample sites on first and second surfaces on a sample support structure.

131. The optical system of any one of the Examples above, wherein said optical system provides diffraction limited imaging of for first and second surfaces on a sample support structure.

132. The optical system of any one of the Examples above, wherein said optical system provides diffraction limited imaging of first and second surfaces on a sample support structure separated by 0.075 mm.

133. The optical system of any one of the Examples above, wherein said optical system provides diffraction limited imaging of first and second surfaces on a sample support structure separated by 0.075 mm along a direction parallel to the optical axis of the objective lens.

134. The optical system of any one of the Examples 16-18, wherein said light source is configured to produce an excitation beam, said light source having an optical output power of at least 0.8 W.

135. The optical system of any of the Examples above, wherein said light source has an optical output power of at least 1 W.

136. The optical system of any of the Examples above, wherein said light source comprises a laser.

137. The optical system of any of the Examples above, wherein said light source comprises a laser diode.

138. The optical system of any of the Examples above, wherein said light source comprises a visible color light source.

139. The optical system of any of the Examples above, wherein said light source comprises a green or red light source.

140. The optical system of any of the Examples above, comprising a plurality of light sources.

141. The optical system of any of the Examples above, wherein said light source comprises at least first and second light sources each having an optical output power of at least 0.8 W.

142. The optical system of any of the Examples above, wherein said light source comprises at least first and second light sources each having an optical output power of at least 1 W.

143. The optical system of any of the Examples above, wherein said light source comprises at least first and second laser light sources.

144. The optical system of any of the Examples above, wherein said light source comprises at least first and second light sources comprising laser diodes.

145. The optical system of any of the Examples above, wherein said light source comprises at least first and second visible color light sources.

146. The optical system of any of the Examples above, wherein said light source comprises at least a first green light source and a second red light source.

147. The optical system of any of the Examples above, wherein said optical system is configured to image first and second surfaces at the same time and optical aberration is less for imaging said first and second surfaces than elsewhere in a region from 1 to 10 mm from said objective lens.

148. The optical system of any of the Examples above, wherein said first surface is between said objective lens and said second surface, said first separated from each other by at least 0.075 mm.

149. The optical system of any of the Examples above, wherein said optical system has less than 0.1 waves of aberration over at least 80% of the field of view for both said first and second surfaces.

150. The optical system of any of the Examples above, wherein said optical system has less than 0.1 waves of aberration over at least 90% of the field of view for both said first and second surfaces.

151. The optical system of any of the Examples above, wherein said optical system provide diffraction limited imaging of for both said first and second surfaces.

152. The optical system of any of the Examples above, wherein optical aberration of said objective lens is less for imaging said first and second surfaces than elsewhere in a region from 1 to 10 mm from said objective lens.

153. The optical system of any of the Examples above, wherein optical aberration of said tube lens is less for imaging said first and second surfaces than elsewhere in a region from 1 to 10 mm from said objective lens.

154. The optical system of any of the Examples above, wherein said objective lens is configured such that said optical system has a magnification of less than 10 (10×).

155. The optical system of any of the Examples above, wherein said objective lens is configured such that said optical system has a magnification of 8 (8×) or less.

156. The optical system of any of the Examples above, wherein said objective lens is configured such that said optical system has a magnification of 6 (6×) or less.

157. The optical system of any of the Examples above, wherein said objective lens is configured such that said optical system has a magnification of 5.5 (5.5×) or less.

158. The optical system of any of the Examples above, wherein said objective lens is configured such that said optical system has a magnification of 5 (5×) or less.

159. The optical system of any of the Examples above, wherein said at least one detection channel is configured to satisfy the Nyquist theorem for diffraction limited imaging.

160. The optical system of any of the Examples above, wherein said photodetector array comprises a plurality of pixels having a dimension that satisfies the Nyquist theorem for diffraction limited imaging.

161. The optical system of any of the Examples above, wherein said photodetector array comprises a plurality of pixels having a pixel size or pitch of less than 5 mm.

162. The optical system of any of the Examples above, wherein said photodetector array comprises a plurality of pixels having a pixel size or pitch of less than 4 mm.

163. The optical system of any of the Examples above, wherein said photodetector array comprises a plurality of pixels having a pixel size or pitch of 3 mm or less.

164. The optical system of any of the Examples above, wherein said photodetector array comprises a plurality of pixels having a pixel size or pitch 2.5 mm or less.

165. The optical system of any of the Examples above, wherein said photodetector array comprises a plurality of pixels having a pixel size or pitch in the range from 5 mm to 1 mm.

166. The optical system of any of the Examples above, wherein said photodetector array comprises a plurality of pixels having a pixel size or pitch in the range from 4 mm to 2 mm.

167. The optical system of any of the Examples above, wherein said photodetector array comprises a plurality of pixels having a pixel size or pitch in the range from 3 mm to 2 mm.

168. The optical system of any of the Examples above, wherein said optical system has a field of view of at least 1 mm wide.

169. The optical system of any of the Examples above, wherein said optical system has a field of view of at least 2 mm wide.

170. The optical system of any of the Examples above, wherein said optical system has a field of view of at least 3 mm wide.

171. The optical system of any of the Examples above, wherein said optical system has working distance in the range from 1 to 10 mm.

172. The optical system of any of the Examples above, wherein said optical system has working distance in the range from 2.5 to 5.5 mm.

173. The optical system of any of the Examples above, wherein said optical system has working distance in the range from 3 to 5 mm.

174. The optical system of any of the Examples above, wherein said photodetector array has an active area having a diagonal of 10 mm or more.

175. The optical system of any of the Examples above, wherein said photodetector array has an active area having a diagonal of 13 mm or more.

176. The optical system of any of the Examples above, wherein said photodetector array has an active area having a diagonal of 14 mm or more.

177. The optical system of any of the Examples above, wherein said photodetector array has an active area having a diagonal of 15 mm or more.

178. The optical system of any of the Examples above, wherein said photodetector array has an active area having a diagonal in a range from 10 mm to 20 mm.

179. The optical system of any of the Examples above, wherein said photodetector array has an active area having a diagonal in a range from 12 mm to 18 mm.

180. The optical system of any of the Examples above, wherein said photodetector array has an active area having a diagonal in a range from 13 mm to 17 mm.

181. The optical system of any of the Examples above, wherein said photodetector array has an active area having a diagonal in a range from 14 mm to 17 mm.

182. The optical system of any of the Examples above, wherein said photodetector array has an active area having a diagonal in a range from 15 mm to 16 mm.

183. The optical system of any of the Examples above, further comprising a translation stage such that a sample support structure can be translated with respect to the objective lens, said optical system configured to capture multiple images such that the multiple images can be assembled to provide a view of the sample support structure that is larger than the field of view of the objective lens.

184. The optical system of any of the Examples above, further comprising a translation stage such that a sample support structure can be translated with respect to the objective lens, said optical system configured to capture multiple images such that the multiple images can be tiled to provide a view of the sample support structure that is larger than the field of view of the objective lens.

185. The optical system of any of the Examples above, further comprising a translation stage such that a sample support structure can be translated with respect to the objective lens and electronics configured to cause multiple images to be captured by the photodetector array and to assemble said multiple images to provide a view of the sample support structure that is larger than the field of view of the objective lens.

186. The optical system of any of the Examples above, wherein said optical system is capable of capturing with said photodetector array an image of fluorescence emitting sample sites on first and second surfaces on said sample support structure.

187. The optical system of any of the Examples above, wherein said optical system is configured to correct aberrations introduced by a layer on a sample support structure through which first and second surfaces of the sample support structure are imaged at the same time.

188. The optical system of any of the Examples above, wherein said optics in said detection channel is configured to correct aberrations introduced by a layer that is part of a sample support structure through which a sample supported by the sample support structure is imaged.

189. The optical system of any of the Examples above, wherein said optics comprises a tube lens configured to correct aberrations introduced by a layer that is part of a sample support structure through which a sample supported by the sample support structure is imaged.

190. The optical system of any of the Examples above, wherein said objective lens is configured to correct aberrations introduced by a layer that is part of a sample support structure through which a sample is supported by the sample support structure is imaged.

191. The optical system of any of the Examples above, wherein said optics in said detection channel is configured to correct aberrations introduced by a layer that is part of a sample support structure through which first and second surfaces on the sample support structure is imaged.

192. The optical system of any of the Examples above, wherein said optics comprises a tube lens configured to correct aberrations introduced by a layer that is part of a sample support structure through which first and second surfaces on the sample support structure is imaged.

193. The optical system of any of the Examples above, wherein said objective lens is configured to correct aberrations introduced by a layer that is part of a sample support structure through which first and second surfaces on the sample support structure is imaged.

194. The optical system of any of the Examples 187-193, wherein said layer is between 0.1 mm to 1.5 mm thick.

195. The optical system of any of the Examples 187-193, wherein said layer has a thickness in the range from 0.15 mm to 1.3 mm thick.

196. The optical system of any of the Examples 187-193, wherein said layer has a thickness in the range from 0.5 mm to 1.3 mm thick.

197. The optical system of the Examples 187-193, wherein said layer has a thickness in the range from 0.75 mm to 1.25 mm thick.

198. The optical system of any of the Examples 187-197, wherein said layer comprises glass.

199. The optical system of any of the Examples 187-197, wherein said layer comprises a glass plate.

200. The optical system of any of the Examples 187-197, wherein said layer comprises a cover slip.

201. The optical system of any of the Examples 187-197, wherein said layer comprises quartz.

202. The optical system of any of the Examples 187-197, wherein said layer comprises plastic.

203. The optical system of any of the Examples above, wherein
said optical system is capable of capturing with said photodetector array an images of fluorescence emitting sample sites on first and second surfaces on a sample support structure; and
said optical system is configured to capture an image of said first and second surface at the same time.

204. The optical system of any of the Examples above, wherein said one or more objective lenses has a numerical aperture of less than 0.6.

205. The optical system of any of the Examples above, having a depth of field of at least 0.075 mm.

206. The optical system of any of the Examples, wherein said optical system is capable of capturing with said photodetector array an image of emission emitting sample sites on first and second surfaces on said sample support structure, said first surface between said objective lens and said second surface, said first separated from each other by at least 0.075 mm.

207. The optical system of any of the Examples above, wherein said optical system is capable of capturing with said photodetector array an image of emission emitting sample sites on first and second planar surfaces on said sample support structure, said first separated from each other by at least 0.075 mm along a direction normal to said first and second planar surfaces.

208. The optical system of any of the Examples above, wherein said optical system is capable of capturing with said photodetector array an image of emission emitting sample sites on first and second surfaces on said sample support structure, said disposed objective above both said first and said second surfaces, said first surface disposed above said second surface by at least 0.075 mm.

209. The optical system of any of the Examples above, wherein said optical system is capable of capturing with said photodetector array an image of emission emitting sample sites on first and second surfaces on said sample support structure, wherein said objective lens has an optical axis and said first and second surfaces are separated from each other by at least 0.075 mm along the direction of said optical axis.

210. The optical system of any of the Examples above, wherein the optical system has a field of view greater than 1.5 mm.

211. The optical system of any of the Examples above, further comprising a sample support structure configured to produce a contrast-to-noise ratio greater than 20.

212. The optical system of any of the Examples above, further comprising a sample support structure having at least one surface comprise a hydrophilic coating.

213. The optical system of any of the Examples above, wherein the optical system is configured for high throughput assays.

214. The optical system of any of the Examples above, wherein the objective lens is disposed to receive the excitation beam, direct the excitation beam to the sample support structure.

215. The optical system of any of the Examples above, further comprising a dichroic filter disposed to reflect the excitation beam into the objective lens and to transmit the emission light to the at least one detection channel.

216. The optical system of any one of the Examples above, wherein no optical element enters an optical path between the sample support structure and a photodetector array in said at least one detection channels in order to form an image of fluorescing sample sites on said a first surface of said sample support structure onto the photodetector array and exits said optical path to form an image of fluorescing sample sites on said second surface of said sample support structure onto the photodetector array.

217. The optical system of any one of the Examples above, wherein no optical compensation is used to form an image of fluorescing sample sites on a first surface of said sample support structure onto the photodetector array that is not identical to optical compensation used to form an image of fluorescing sample sites on a second surface of said sample support structure onto the photodetector array.

218. The optical system of any one of the Examples above, wherein no optical element in an optical path between the sample support structure and a photodetector array in said at least one detection channels is adjusted differently to form an image of fluorescing sample sites on a first surface of said sample support structure onto the photodetector array than to form an image of fluorescing sample sites on a second surface of said sample support structure onto the photodetector array.

219. The optical system of any one of the Examples above, wherein no optical element in an optical path between the sample support structure and a photodetector array in said at least one detection channels is moved a different amount or a different direction to form an image of fluorescing sample sites on said a first surface of said sample support structure onto the photodetector array than to form an image of fluorescing sample sites on a second surface of said sample support structure onto the photodetector array.

220. The optical system of any of Examples 216 to 219, wherein first and second surfaces on said sample support structure are separated from each other by at least 0.075 mm.

221. The optical system of any of Examples 216 to 220, wherein first surface is between said objective lens and said second surface.

222. The optical system of any of Examples 216 to 220, wherein said first and second surfaces are planar surfaces and said first is separated from each other along a direction normal to said first and second planar surfaces.

223. The optical system of any of Examples 216 to 220, wherein said first and second surfaces are planar surfaces and said first is separated from each other by at least 0.075 mm along a direction normal to said first and second planar surfaces.

224. The optical system of any of Examples 216 to 220, wherein said objective is disposed above both said first and said second surfaces and said first surface is disposed above said second surface.

225. The optical system of any of Examples 216 to 219, wherein said objective is disposed above both said first and said second surfaces and said first surface is disposed above said second surface by at least 0.075 mm.

226. The optical system of any of Examples 216 to 219, wherein said objective lens has an optical axis and said first and second surfaces are separated from each other along the direction of said optical axis.

227. The optical system of any of Examples 216 to 219, wherein said objective lens has an optical axis and said first and second surfaces are separated from each other by at least 0.075 mm along the direction of said optical axis.

228. The optical system of any one of the Examples 216-227, wherein said first and second surfaces are both configured to provide a contrast-to-noise ratio of greater than 20 for a single sequencing cycle.

229. The optical system of any one of the Examples 216-227, wherein said first and second surfaces are both configured to provide a contrast-to-noise ratio greater than 20 for 5 consecutive sequencing cycles.

230. The optical system of any one of the Examples 216-227, wherein said first and second surfaces are both configured to provide a contrast-to-noise ratio of greater than 20 for 10 consecutive sequencing cycles.

231. The optical system of any one of the Examples 216-227, wherein said first and second surfaces both comprise hydrophilic surfaces comprising labeled nucleic acid colonies at a density of at least 10000/mm², and are configured to provide a contrast-to-noise ratio of at least 20.

232. The optical system of any one of the Examples above, wherein said objective lens has a numerical aperture less than 0.55.

233. The optical system of any one of the Examples above, wherein said objective lens has a numerical aperture less than 0.5.

234. The optical system of any one of the Examples above, wherein said objective lens has a numerical aperture less than 0.45.

235. The optical system of any one of the Examples above, wherein said objective lens has a numerical aperture less than 0.4.

236. The optical system of any one of the Examples above, further comprising one or more tube lenses.

237. The optical system of any one of the Examples above, further comprising one or more tube lenses in said at least one detection channel.

238. The optical system of any one of the Examples above, wherein said objective lens and said at least one detection channel provide a field of at least 1 mm.

239. The optical system of any one of the Examples above, wherein said objective lens and said at least one detection channel provide a field of view greater than 1.5 mm.

240. The optical system of any one of the Examples above, wherein a sample support structure is configured to provide a contrast-to-noise ratio of greater than 20 for a single sequencing cycle.

241. The optical system of any one of the Examples above, wherein a sample support structure is configured to provide a contrast-to-noise ratio greater than 20 for 5 consecutive sequencing cycles.

242. The optical system of any one of the Examples above, wherein a sample support structure is configured to provide a contrast-to-noise ratio of greater than 20 for 10 consecutive sequencing cycles.

243. The optical system of any one of the Examples above, wherein said sample support structure comprises a hydrophilic substrate comprising labeled nucleic acid colonies at a density of at least 10000/mm², and is configured to provide a contrast-to-noise ratio of at least 20.

244. The optical system of any one of the Examples above, wherein a sample support structure comprises fluorescing sample sites comprising nucleic acid colonies comprising 1, 2, 3, or 4 distinct detectable labels.

245. A optical system of any one of the Examples above, wherein said at least one detecting channel comprise imaging channels to detect 1, 2, 3, or 4 distinct labels.

246. A method of sequencing a nucleic acid comprising binding or sequencing by synthesis reaction on one or more surfaces of a sample support structure, and detecting a bound or incorporated base using the optical system of any of the Examples above.

247. A method of determining a genotype of a sample comprising a nucleic acid molecule, comprising preparing said nucleic acid molecule for sequencing, and then sequencing said nucleic acid molecule using the optical system of any of the Examples above.

248. An optical system of any one of the Examples above, wherein the sample support structure comprises one, two, three, four, five, or six imaging surfaces comprising fluorescing sample sites.

249. An optical system of any one of the Examples above, wherein the sample support structure comprises a flow cell.

250. An optical system of any one of the Examples above, wherein the sample support structure comprises a sample chamber.

251. An optical system of any one of the Examples above, further comprising said sample support structure.

252. The optical system of any of the Examples above, wherein said objective lens has a numerical aperture in the range between 0.5 to 0.4.

253. The optical system of any of the Examples above, wherein said optical system has an optical resolution in a range from 500 to 1000 nm.

254. The optical system of any of the Examples above, having an optical resolution in a range from 600 to 900 nm.

255. The optical system of any of the Examples above, wherein said optical system has an optical resolution in a range from 650 to 850 nm.

256. The optical system of any of the Examples above, further comprising said sample support structure.

257. The optical system of any of the Examples above, further comprising said sample support structure having said first and second surfaces.

258. The optical system of any of the Examples above, further comprising the sample support structure, said sample support structure comprising a flow cell.

259. The optical system of any of the Examples above, further comprising the sample support structure comprising a flow cell having a flow channel and said first and second surfaces comprise interior surfaces of said flow cell configured to be in contact with a sample flowing through said flow cell.

260. The optical system of any of the Examples above, configured for DNA sequencing.

261. The optical system of any of the Examples above, comprising four channels configured to capture images at four different spectral regions.

262. The optical system of any of the Examples above, comprising electronics configured to process images captured by a plurality of optical channels to obtain information from the fluorescing sample sites.

263. The optical system of any of the Examples above, comprising electronics configured to process images captured by a plurality of optical channels to obtain information from the fluorescing sample sites based on their locations.

264. The optical system of any of the Examples above, wherein said optical system comprises a sequencer.

265. The optical system of any of the Examples above, wherein said optical system comprises a nucleic acid sequencing apparatus.

266. The optical system of any of the Examples above, further comprising a fluid flow controller configured to control the flow of fluid through a flow cell or sample support structure.

267. The optical system of any of the Examples above, further comprising conduits configured to flow fluid through a flow cell or sample support structure.

268. The optical system of any of the Examples above, further comprising tubing that provides fluid communication with a fluid flow control system to provide fluid to a flow cell or sample support structure.

269. The optical system of any of the Examples above, further comprising a fluid flow controller configured to provide programmable control of fluid flow velocity, volumetric fluid flow rate, the timing of reagent or buffer introduction, or any combination thereof.

270. The optical system of any of the Examples above, further comprising a temperature controller configured to control the temperature of a flow cell or sample support structure.

271. The optical system of any of the Examples above, further comprising a reagent reservoir configured to provide reagent to the flow cell or sample support structure.

272. The optical system of any of the Examples above, further comprising a replaceable reagent configured to provide reagent to the flow cell or sample support structure.

273. The optical system of any of the Examples above, further comprising motorized translation stage configured to move the flow cell or sample support structure.

274. The optical system of any of the Examples above, further comprising motor controllers to control movement of a translation stage configured to move the flow cell or sample support structure.

275. The optical system of any of the Examples above, wherein said optical system comprises a nucleic sequencing apparatus.

### Part II

1. An optical system comprising:
one or more objective lenses having a numerical aperture of less than 0.6;
wherein the optical system is capable of simultaneous imaging of two or more surfaces separated by 0.075 mm or more, said two or more surfaces located at different distances from the objective;
wherein the optical system has a field of view of at least 1 mm; and
wherein the optical system does not require additional optical compensation for imaging said two or more surfaces.

2. The optical system of Example 1, further comprising one or more tube lenses.

3. An optical system comprising:
one or more objective lenses having a numerical aperture of less than 0.6; and
a flow cell with two or more imaging surfaces, the imaging surfaces comprising a hydrophilic coating, said imaging surfaces producing a contrast to noise ratio greater than 20;
wherein the optical system is capable of simultaneous imaging of two or more surfaces separated by 0.075 mm or more;
wherein the optical system has a field of view of at least 1 mm; and
wherein the optical system does not require additional optical compensation for imaging said two or more surfaces.

4. The optical system of Example 3, further comprising one or more tube lenses.

5. An optical system comprising:
one or more objective lenses having a numerical aperture of less than 0.6;
wherein the optical system is capable of simultaneous imaging of two or more surfaces separated by 0.075 mm or more;
wherein the optical system has a field of view of at least 1 mm;
wherein the optical system does not require additional optical compensation imaging said two or more surfaces; and
wherein the optical system is configured for high throughput assays.

6. The optical system of Example 5, further comprising one or more tube lenses.

7. A method of detecting features on two or more surfaces, the method comprising:
imaging the two or more surfaces using a combination of optical elements comprising an objective lens having a numerical aperture of less than 0.6;
wherein the combination of optical elements is capable of simultaneous imaging of two or more surfaces separated by 0.075 mm or more;
wherein the combination of optical elements has a field of view of at least 1 mm; and
wherein the combination of optical elements does not require additional optical compensation for imaging the two or more surfaces.

8. The method of Example 7, wherein the combination of optical elements further comprises one or more tube lenses.

9. A method of sequencing one or more nucleic acids, the method comprising:
detecting features on two or more surfaces by imaging the two or more surfaces using a combination of optical elements comprising an objective lens;
wherein the combination of optical elements is capable of simultaneous imaging of two or more surfaces separated by 0.075 mm or more;
wherein the combination of optical elements has a field of view of at least 1 mm; and
wherein the combination of optical elements does not require additional optical compensation for imaging the two or more surfaces.

10. The method of Example 9, wherein the combination of optical elements further comprises one or more tube lenses.

11. The optical system or method of any one of Examples 1-10, wherein the two or more surfaces comprise two or more surfaces of a flow cell configured such that both surfaces yield a contrast to noise ratio of greater than 20 for a single sequencing cycle.

12. The optical system or method of any one of Examples 1-11, wherein the two or more surfaces comprise two or more surfaces of a flow cell configured such that both surfaces yield a contrast to noise ratio greater than 20 for 5 consecutive sequencing cycles.

13. The optical system or method of any one of Examples 1-12, wherein the two or more surfaces comprise two or more surfaces of a flow cell configured such that both surfaces yield a contrast to noise ratio of greater than 20 for 10 consecutive sequencing cycles.

14. The optical system or method of any one of Examples 1-13, wherein the flow cell is coated with a hydrophilic coating.

15. The optical system or method of any one of Examples 1-14, wherein the flow cell comprises a hydrophilic substrate comprising labeled nucleic acid colonies having a density of at least 10K/mm², wherein an image of one of the two or more surfaces shows a contrast to noise ratio of at least 20.

16. The optical system or method of any one of Examples 1-15, wherein at least one of the two or more surfaces comprises nucleic acid colonies comprising 1, 2, 3, or 4 distinct detectable labels.

17. The optical system or method of any one of Examples 1-16, further comprising imaging channels to detect 1, 2, 3, or 4 distinct labels.

18. A method of sequencing a nucleic acid comprising carrying out a sequencing by binding or sequencing by synthesis reaction on at least one of the two or more surfaces, and detecting a bound or incorporated base using the optical system or method of any one of Examples 1-17.

19. A method of determining a genotype of a sample comprising a nucleic acid molecule, the method comprising preparing the nucleic acid molecule for sequencing, and then sequencing the nucleic acid molecule using the optical system or method of any one of Examples 1-17.

20. The optical system or method of any one of Examples 1-19, further comprising a flow cell or sample chamber comprising one, two, three, four, five, or six imaging surfaces.

21. The optical system or method of any one of Examples 1-20, wherein the optical system or method does not require additional compensation in order to achieve imaging of both surfaces of a dual-surface flow cell or sample chamber having two surfaces at different distances from the objective lens having sample sites configured to bind with sample.

22. The optical system or method of any one of Examples 1-21, wherein the optical system or method does not require additional compensation in order to achieve imaging of multiple surfaces of a multiple-surface flow cell or sample chamber at different distances from the microscope objective.

23. The optical system or method of any one of Examples 1-22, wherein the optical system or method does not require movement of one or more optical element into or out of the path of fluorescent emission in order to achieve imaging of multiple surfaces of a multiple-surface flow cell or sample chamber at different distances from the microscope objective.

24. The optical system or method of any one of Examples 1-23, wherein no optical element enters or leaves the light path between the flow cell or sample chamber and a photodetector array that captures images of fluorescent emission from sample sites the two or more surfaces at different distances from the objective lens in order to form an image of said fluorescent emission from said the onto the photodetector array.

25. The optical system or method of any one of Examples 1-24, wherein an objective lens has a numerical aperture less than 0.6.

26. The optical system or method of any one of Examples 1-24, wherein an objective lens has a numerical aperture less than 0.55.

27. The optical system or method of any one of Examples 1-24, wherein an objective lens has a numerical aperture less than 0.5.

28. The optical system or method of any one of Examples 1-24, wherein an objective lens has a numerical aperture less than 0.45.

29. The optical system or method of any one of Examples 1-24, wherein an objective lens has a numerical aperture less than 0.4.

30. The optical system of any of the Examples above, wherein said optical system comprises a sequencer.

31. The optical system of any of the Examples above, wherein said optical system comprises a nucleic acid sequencing apparatus.

32. The optical system of any of the Examples above, further comprising a fluid flow controller configured to control the flow of fluid through a flow cell or sample support structure.

33. The optical system of any of the Examples above, further comprising conduits configured to flow fluid through a flow cell or sample support structure.

34. The optical system of any of the Examples above, further comprising tubing that provides fluid communication with a fluid flow control system to provide fluid to a flow cell or sample support structure.

35. The optical system of any of the Examples above, further comprising a fluid flow controller configured to provide programmable control of fluid flow velocity, volumetric fluid flow rate, the timing of reagent or buffer introduction, or any combination thereof.

36. The optical system of any of the Examples above, further comprising a temperature controller configured to control the temperature of a flow cell or sample support structure.

37. The optical system of any of the Examples above, further comprising a reagent reservoir configured to provide reagent to the flow cell or sample support structure.

38. The optical system of any of the Examples above, further comprising a replaceable reagent configured to provide reagent to the flow cell or sample support structure.

39. The optical system of any of the Examples above, further comprising motorized translation stage configured to move the flow cell or sample support structure.

40. The optical system of any of the Examples above, further comprising motor controllers to control movement of a translation stage configured to move the flow cell or sample support structure.

41. The optical system of any of the Examples above, wherein said optical system comprises a nucleic sequencing apparatus.

### Part III

1. An optical system comprising one or more objective lenses and optionally one or more tube lenses, wherein said system is capable of simultaneous imaging of two or more surfaces separated by 0.075 mm or more; has a field of view of at least 1 mm; and does not require additional optical compensation for multi-surface imaging; and wherein said one or more objective lenses has a numerical aperture of less than 0.6.

2. An optical system comprising one or more objective lenses and optionally one or more tube lenses, wherein said system is capable of simultaneous imaging of two or more surfaces separated by 0.075 mm or more; has a field of view of at least 1 mm; and does not require additional optical compensation for multi-surface imaging; and wherein said one or more objective lenses has a numerical aperture of less than 0.6; further comprising a flow cell with two or more image surfaces, said surfaces comprising a hydrophilic coating and contrast to noise ratio greater than 20.

3. An optical system comprising one or more objective lenses and optionally one or more tube lenses, wherein said system is capable of simultaneous imaging of two or more surfaces separated by 0.075 mm or more; has a field of view of at least 1 mm; and does not require additional optical compensation for multi-surface imaging; and wherein said one or more objective lenses has a numerical aperture of less than 0.6; wherein said system is configured for high throughput assays.

4. A method of detecting features on one or more surfaces, comprising imaging said surface using a combination of optical elements comprising one or more objective lenses and optionally one or more tube lenses, wherein said combination of optical elements is capable of simultaneous imaging of two or more surfaces separated by 0.075 mm or more; has a field of view of at least 1 mm; and does not require additional optical compensation for multi-surface imaging; and wherein said one or more objective lenses has a numerical aperture of less than 0.6.

5. A method of sequencing one or more nucleic acids, comprising detecting features on one or more surfaces, comprising imaging said surface using a combination of optical elements comprising one or more objective lenses and optionally one or more tube lenses, wherein said combination of optical elements is capable of simultaneous imaging of two or more surfaces separated by 0.075 mm or more; has a field of view of at least 1 mm; and does not require additional optical compensation for multi-surface imaging.

6. A system of any of Examples 1-3 or a method of any of Examples 4-5 wherein said one or more surfaces comprise one or more surfaces of a flow cell modified such that both surfaces yield a CNR of >20 for a single sequencing cycle.

7. A system or method of any of Examples 1-6 wherein said surfaces comprise one or more surfaces of a flow cell such that both surfaces yield a CNR greater than 20 for 5 consecutive sequencing cycles.

8. A system or method of any of Examples 1-7 wherein said surfaces comprise one or more surfaces of a flow cell such that both surfaces yield a CNR of >20 for 10 consecutive sequencing cycles.

9. A system or method of any of Examples 1-8 wherein said flow cell is coated with a hydrophilic substrate.

10. A system or method of any of Examples 1-9 wherein said flow cell comprises a hydrophilic substrate comprising labeled nucleic acid colonies at a density of at least 10K/mm2, wherein an image of the surface shows a contrast to noise ratio of at least 20.

11. A system or method of any of Examples 1-10 wherein said surface comprises nucleic acid colonies comprising 1, 2, 3, or 4 distinct detectable labels.

12. A system or method of any of Examples 1-11 further comprising imaging channels to detect 1, 2, 3, or 4 distinct labels.

13. A method of sequencing a nucleic acid comprising carrying out a sequencing by binding or sequencing by synthesis reaction on one or more surfaces, and detecting a bound or incorporated base using a system or method of any of Examples 1-12.

14. A method of determining a genotype of a sample comprising a nucleic acid molecule, comprising preparing said nucleic acid molecule for sequencing, and then sequencing said nucleic acid molecule using a system or method of any of Examples 1-13.

15. A system or method of any of Examples 1-14 wherein a flow cell or sample chamber comprises one, two, three, four, five, or six imaging surfaces.

16. A system or method of any of Examples 1-15 wherein said system or method does not require additional compensation in order to achieve imaging of both surfaces of a dual-surface flow cell or sample chamber.

17. A system or method of any of Examples 1-16 wherein said system or method does not require additional compensation in order to achieve imaging of multiple surfaces of a multiple-surface flow cell or sample chamber.

18. A system or method of any of Examples 1-17 wherein said system or method does not require movement of any part into or out of the light path in order to achieve imaging of multiple surfaces of a multiple-surface flow cell or sample chamber.

19. A system or method of any of Examples 1-18 wherein no object enters or leaves the light path upstream of the sample chamber during the operation of the system or method.

20. A system or method of any of Examples 1-19 wherein an objective lens has a numerical aperture less than 0.6.

21. The optical system of any of the Examples above, wherein said optical system comprises a sequencer.

22. The optical system of any of the Examples above, wherein said optical system comprises a nucleic acid sequencing apparatus.

23. The optical system of any of the Examples above, further comprising a fluid flow controller configured to control the flow of fluid through a flow cell or sample support structure.

24. The optical system of any of the Examples above, further comprising conduits configured to flow fluid through a flow cell or sample support structure.

25. The optical system of any of the Examples above, further comprising tubing that provides fluid communication with a fluid flow control system to provide fluid to a flow cell or sample support structure.

26. The optical system of any of the Examples above, further comprising a fluid flow controller configured to provide programmable control of fluid flow velocity, volumetric fluid flow rate, the timing of reagent or buffer introduction, or any combination thereof.

27. The optical system of any of the Examples above, further comprising a temperature controller configured to control the temperature of a flow cell or sample support structure.

28. The optical system of any of the Examples above, further comprising a reagent reservoir configured to provide reagent to the flow cell or sample support structure.

29. The optical system of any of the Examples above, further comprising a replaceable reagent configured to provide reagent to the flow cell or sample support structure.

30. The optical system of any of the Examples above, further comprising motorized translation stage configured to move the flow cell or sample support structure.

31. The optical system of any of the Examples above, further comprising motor controllers to control movement of a translation stage configured to move the flow cell or sample support structure.

32. The optical system of any of the Examples above, wherein said optical system comprises a nucleic sequencing apparatus.

### Part IV

1. An optical system comprising:
a light source configured to produce an excitation beam;
an objective lens configured to receive emission light from a sample on a support structure in response to the excitation beam;
at least one detection channel comprising optics and a photodetector array configured to receive at least a portion of the emission light and capture an image of at least one fluorescing sample site on said sample support structure; and
wherein said one or more objective lenses has a numerical aperture of less than 0.6.

2. The optical system of Example 1, having a depth of field of at least 0.075 mm.

3. The optical system of any of Examples 1 or 2, wherein said optical system is capable of capturing with said photodetector array an image of emission emitting sample sites on first and second surfaces on said sample support structure, said first surface between said objective lens and said second surface, said first separated from each other by at least 0.075 mm.

4. The optical system of any of Examples 1 or 2, wherein said optical system is capable of capturing with said photodetector array an image of emission emitting sample sites on first and second planar surfaces on said sample support structure, said first separated from each other by at least 0.075 mm along a direction normal to said first and second planar surfaces.

5. The optical system of any of Examples 1 or 2, wherein said optical system is capable of capturing with said photodetector array an image of emission emitting sample sites on first and second surfaces on said sample support structure, said disposed objective above both said first and said second surfaces, said first surface disposed above said second surface by at least 0.075 mm.

6. The optical system of any of Examples 1 or 2, wherein said optical system is capable of capturing with said photodetector array an image of emission emitting sample sites on first and second surfaces on said sample support structure, wherein said objective lens has an optical axis and said first and second surfaces are separated from each other by at least 0.075 mm along the direction of said optical axis.

7. The optical system of any of the Examples above, wherein the optical system has a field of view of at least 1 mm.

8. The optical system of any of the Examples above, further comprising a sample support structure configured to produce a contrast-to-noise ratio greater than 20.

9. The optical system of any of the Examples above, further comprising a sample support structure having at least one surface comprise a hydrophilic coating.

10. The optical system of any of the Examples above, wherein the optical system is configured for high throughput assays.

11. The optical system of any of the Examples above, wherein the objective lens is disposed to receive the excitation beam, direct the excitation beam to the sample support structure.

12. The optical system of any of the Examples above, further comprising a dichroic filter disposed to reflect the excitation beam into the objective lens and to transmit the emission light to the at least one detection channel.

13. The optical system of any one of the Examples above, wherein no optical element enters an optical path between the sample support structure and a photodetector array in said at least one detection channels in order to form an image of fluorescing sample sites on said a first surface of said sample support structure onto the photodetector array and exits said optical path to form an image of fluorescing sample sites on said second surface of said sample support structure onto the photodetector array.

14. The optical system of any one of the Examples above, wherein no optical compensation is used to form an image of fluorescing sample sites on a first surface of said sample support structure onto the photodetector array that is not identical to optical compensation used to form an image of fluorescing sample sites on a second surface of said sample support structure onto the photodetector array.

15. The optical system of any one of the Examples above, wherein no optical element in an optical path between the sample support structure and a photodetector array in said at least one detection channels is adjusted differently to form an image of fluorescing sample sites on a first surface of said sample support structure onto the photodetector array than to form an image of fluorescing sample sites on a second surface of said sample support structure onto the photodetector array.

16. The optical system of any one of the Examples above, wherein no optical element in an optical path between the sample support structure and a photodetector array in said at least one detection channels is moved a different amount or a different direction to form an image of fluorescing sample sites on said a first surface of said sample support structure onto the photodetector array than to form an image of fluorescing sample sites on a second surface of said sample support structure onto the photodetector array.

17. The optical system of any of Examples 13 to 16, wherein first and second surfaces on said sample support structure are separated from each other by at least 0.075 mm.

18. The optical system of any of Examples 13 to 16, wherein first surface is between said objective lens and said second surface.

19. The optical system of any of Examples 13 to 17, wherein said first and second surfaces are planar surfaces and said first is separated from each other along a direction normal to said first and second planar surfaces.

20. The optical system of any of Examples 13 to 17, wherein said first and second surfaces are planar surfaces and said first is separated from each other by at least 0.075 mm along a direction normal to said first and second planar surfaces.

21. The optical system of any of Examples 13 to 17, wherein said objective is disposed above both said first and said second surfaces and said first surface is disposed above said second surface.

22. The optical system of any of Examples 13 to 17, wherein said objective is disposed above both said first and said second surfaces and said first surface is disposed above said second surface by at least 0.075 mm.

23. The optical system of any of Examples 13 to 17, wherein said objective lens has an optical axis and said first and second surfaces are separated from each other along the direction of said optical axis.

24. The optical system of any of Examples 13 to 17, wherein said objective lens has an optical axis and said first and second surfaces are separated from each other by at least 0.075 mm along the direction of said optical axis.

25. The optical system of any one of the Examples 13-24, wherein said first and second surfaces are both configured to provide a contrast-to-noise ratio of greater than 20 for a single sequencing cycle.

26. The optical system of any one of the Examples 13-24, wherein said first and second surfaces are both configured to provide a contrast-to-noise ratio greater than 20 for 5 consecutive sequencing cycles.

27. The optical system of any one of the Examples 13-24, wherein said first and second surfaces are both configured to provide a contrast-to-noise ratio of greater than 20 for 10 consecutive sequencing cycles.

28. The optical system of any one of the Examples 13-24, wherein said first and second surfaces both comprise hydrophilic surfaces comprising labeled nucleic acid colonies at a density of at least 10000/mm², and are configured to provide a contrast-to-noise ratio of at least 20.

29. The optical system of any one of the Examples above, wherein said objective lens has a numerical aperture less than 0.6.

30. The optical system of any one of the Examples above, wherein said objective lens has a numerical aperture less than 0.55.

31. The optical system of any one of the Examples above, wherein said objective lens has a numerical aperture less than 0.5.

32. The optical system of any one of the Examples above, wherein said objective lens has a numerical aperture less than 0.45.

33. The optical system of any one of the Examples above, wherein said objective lens has a numerical aperture less than 0.4.

34. The optical system of any one of the Examples above, further comprising one or more tube lenses.

35. The optical system of any one of the Examples above, further comprising one or more tube lenses in said at least one detection channel.

36. The optical system of any one of the Examples above, wherein objective lens and said at least one detection channel provide a field of view of at least 1 mm.

37. The optical system of any one of the Examples above, wherein said sample support structure is configured to provide a contrast-to-noise ratio of greater than 20 for a single sequencing cycle.

38. The optical system of any one of the Examples above, wherein said sample support structure is configured to provide a contrast-to-noise ratio greater than 20 for 5 consecutive sequencing cycles.

39. The optical system of any one of the Examples above, wherein said sample support structure is configured to provide a contrast-to-noise ratio of greater than 20 for 10 consecutive sequencing cycles.

40. The optical system of any one of the Examples above, wherein said sample support structure comprises a hydrophilic substrate comprising labeled nucleic acid colonies at a density of at least 10000/mm², and is configured to provide a contrast-to-noise ratio of at least 20.

41. The optical system of any one of the Examples above, wherein said fluorescing sample sites comprise nucleic acid colonies comprising 1, 2, 3, or 4 distinct detectable labels.

42. An optical system of any one of the Examples above, wherein said at least one detecting channel comprise imaging channels to detect 1, 2, 3, or 4 distinct labels.

43. A method of sequencing a nucleic acid comprising binding or sequencing by synthesis reaction on one or more surfaces of said sample support structure, and detecting a bound or incorporated base using the optical system of any of the Examples above.

44. A method of determining a genotype of a sample comprising a nucleic acid molecule, comprising preparing said nucleic acid molecule for sequencing, and then sequencing said nucleic acid molecule using the optical system of any of the Examples above.

45. An optical system of any one of the Examples above, wherein the sample support structure comprises one, two, three, four, five, or six imaging surfaces comprising fluorescing sample sites.

46. An optical system of any one of the Examples above, wherein the sample support structure comprises a flow cell.

47. An optical system of any one of the Examples above, wherein the sample support structure comprises a sample chamber.

48. An optical system of any one of the Examples above, further comprising said sample support structure.

49. An optical system of any one of the Examples above, wherein said optical system has a field-of-view of at least 1.0 mm wide with less than 0.1 waves of aberration over at least 80% of the field-of-view.

50. An optical system of any one of the Examples above, wherein said optical system has a field-of-view of at least 1.0 mm wide with less than 0.1 waves of aberration over at least 90% of the field-of-view.

51. An optical system of any one of the Examples above, wherein said optical system has a field-of-view of at least 2.0 mm wide with less than 0.09 waves of aberration over at least 80% of the field-of-view.

52. An optical system of any one of the Examples above, wherein said optical system has a field-of-view of at least 2.0 mm wide with less than 0.09 waves of aberration over at least 90% of the field-of-view.

53. An optical system of any one of the Examples above, wherein said optical system has a field-of-view of at least 3.0 mm wide with less than 0.09 waves of aberration over at least 80% of the field-of-view.

54. An optical system of any one of the Examples above, wherein said optical system has a field-of-view of at least 3.0 mm wide with less than 0.09 waves of aberration over at least 90% of the field-of-view.

55. An optical system of any one of the Examples above, wherein said optical system has a field-of-view of at least 3.2 mm wide with less than 0.09 waves of aberration over at least 80% of the field-of-view.

56. An optical system of any one of the Examples above, wherein said optical system has a field-of-view of at least 3.2 mm wide with less than 0.09 waves of aberration over at least 90% of the field-of-view.

57. An optical system of any one of the Examples above, wherein said optical system has a work distance of in a range from 2 to 6 mm.

58. An optical system of any one of the Examples above, wherein said optical system has a work distance of in a range from 2.5 to 5.5 mm.

59. An optical system of any one of the Examples above, wherein said optical system has a work distance of in a range from 3 to 5 mm.

60. An optical system of any one of the Examples above, wherein said optical system has a work distance of at least 3 mm.

61. The optical system of any one of the Examples above, wherein said optical system is capable of capturing with said photodetector array an image of fluorescence emitting sample sites on first and second surfaces on said sample support structure.

62. The optical system of any one of the Examples 118, wherein said first surface is between said objective lens and said second surface, said first and second surfaces separated from each other by at least 0.075 mm.

63. The optical system of any of one the Examples 118 or 119, wherein said optical system has less than 0.1 waves of aberration over at least 80% of the field of view for both said first and second surfaces.

64. The optical system of any one of the Examples 118-120, wherein said optical system has less than 0.1 waves of aberration over at least 90% of the field of view for both said first and second surfaces.

65. The optical system of any one of the Examples 118-120, wherein said optical system provide diffraction limited imaging of for both said first and second surfaces.

66. An optical system of any one of the Examples above, wherein the optical system has a field-of-view of at least 1.0 mm wide and is diffraction limited.

67. An optical system of any one of the Examples above, wherein said optical system has a field-of-view of at least 1.0 mm wide.

68. An optical system of any one of the Examples above, wherein said optical system has a field-of-view of at least 2.0 mm wide.

69. An optical system of any one of the Examples above, wherein said optical system has a field-of-view of at least 3.0 mm wide.

70. An optical system of any one of the Examples above, wherein optical system has a field-of-view of at least 3.2 mm wide.

71. The optical system of any one of the Examples above, wherein said optical system is capable of capturing with said photodetector array an image of fluorescence emitting sample sites on first and second surfaces on said sample support structure.

72. The optical system of any one of the Examples above, wherein said optical system provides diffraction limited imaging of for first and second surfaces on said sample support structure.

73. The optical system of any one of the Examples above, wherein said optical system provides diffraction limited imaging of first and second surfaces on said sample support structure separated by 0.075 mm.

74. The optical system of any one of the Examples above, wherein said optical system provides diffraction limited imaging of first and second surfaces on said sample support structure separated by 0.075 mm along a direction parallel to the optical axis of the objective lens.

75. The optical system of any one of the Examples 16-18, wherein said at least one light source is configured to produce an excitation beam, said light source having an optical output power of at least 0.8 W.

76. The optical system of any of the Examples above, wherein said light source has an optical output power of at least 1 W.

77. The optical system of any of the Examples above, wherein said at least one light source comprises a laser.

78. The optical system of any of the Examples above, wherein said at least one light source comprises a laser diode.

79. The optical system of any of the Examples above, wherein said at least one light source comprises a visible color light source.

80. The optical system of any of the Examples above, wherein said at least one light source comprises a green or red light source.

81. The optical system of any of the Examples above, comprising a plurality of light sources.

82. The optical system of any of the Examples above, wherein said at least one light source comprises at least first and second light sources each having an optical output power of at least 0.8 W.

83. The optical system of any of the Examples above, wherein said at least one light source comprises at least first and second light sources each having an optical output power of at least 1 W.

84. The optical system of any of the Examples above, wherein said at least one light source comprises at least first and second laser light sources.

85. The optical system of any of the Examples above, wherein said at least one light source comprises at least first and second light sources comprising laser diodes.

86. The optical system of any of the Examples above, wherein said at least one light source comprises at least first and second visible color light sources.

87. The optical system of any of the Examples above, wherein said at least one light source comprises at least a first green light source and a second red light source.

88. The optical system of any of the Examples above, wherein said optical system is configured to image said first and second surfaces at the same time and optical aberration is less for imaging said first and second surfaces than elsewhere in a region from 1 to 10 mm from said objective lens.

89. The optical system of any of the Examples above, wherein said first surface is between said objective lens and said second surface, said first separated from each other by at least 0.075 mm.

90. The optical system of any of the Examples above, wherein said optical system has less than 0.1 waves of aberration over at least 80% of the field of view for both said first and second surfaces.

91. The optical system of any of the Examples above, wherein said optical system has less than 0.1 waves of aberration over at least 90% of the field of view for both said first and second surfaces.

92. The optical system of any of the Examples above, wherein said optical system provide diffraction limited imaging of for both said first and second surfaces.

93. The optical system of any of the Examples above, wherein optical aberration of said objective lens is less for imaging said first and second surfaces than elsewhere in a region from 1 to 10 mm from said objective lens.

94. The optical system of any of the Examples above, wherein optical aberration of said tube lens is less for imaging said first and second surfaces than elsewhere in a region from 1 to 10 mm from said objective lens.

95. The optical system of any of the Examples above, wherein said objective lens is configured such that said optical system has a magnification of less than 10 (10×).

96. The optical system of any of the Examples above, wherein said objective lens is configured such that said optical system has a magnification of 8 (8×) or less.

97. The optical system of any of the Examples above, wherein said objective lens is configured such that said optical system has a magnification of 6 (6×) or less.

98. The optical system of any of the Examples above, wherein said objective lens is configured such that said optical system has a magnification of 5.5 (5.5×) or less.

99. The optical system of any of the Examples above, wherein said objective lens is configured such that said optical system has a magnification of 5 (5×) or less.

100. The optical system of any of the Examples above, wherein said at least one detection channel is configured to satisfy the Nyquist theorem for diffraction limited imaging.

101. The optical system of any of the Examples above, wherein said photodetector array comprises a plurality of pixels having a dimension that satisfies the Nyquist theorem for diffraction limited imaging.

102. The optical system of any of the Examples above, wherein said photodetector array comprises a plurality of pixels having a pixel size or pitch of less than 5 mm.

103. The optical system of any of the Examples above, wherein said photodetector array comprises a plurality of pixels having a pixel size or pitch of less than 4 mm.

104. The optical system of any of the Examples above, wherein said photodetector array comprises a plurality of pixels having a pixel size or pitch of 3 mm or less.

105. The optical system of any of the Examples above, wherein said photodetector array comprises a plurality of pixels having a pixel size or pitch 2.5 mm or less.

106. The optical system of any of the Examples above, wherein said photodetector array comprises a plurality of pixels having a pixel size or pitch in the range from 5 mm to 1 mm.

107. The optical system of any of the Examples above, wherein said photodetector array comprises a plurality of pixels having a pixel size or pitch in the range from 4 mm to 2 mm.

108. The optical system of any of the Examples above, wherein said photodetector array comprises a plurality of pixels having a pixel size or pitch in the range from 3 mm to 2 mm.

109. The optical system of any of the Examples above, wherein said optical system has a field of view of at least 1 mm wide.

110. The optical system of any of the Examples above, wherein said optical system has a field of view of at least 2 mm wide.

111. The optical system of any of the Examples above, wherein said optical system has a field of view of at least 3 mm wide.

112. The optical system of any of the Examples above, wherein said optical system has working distance in the range from 1 to 10 mm.

113. The optical system of any of the Examples above, wherein said optical system has working distance in the range from 2.5 to 5.5 mm.

114. The optical system of any of the Examples above, wherein optical system has working distance in the range from 3 to 5 mm.

115. The optical system of any of the Examples above, wherein said photodetector array has an active area having a diagonal of 10 mm or more.

116. The optical system of any of the Examples above, wherein said photodetector array has an active area having a diagonal of 13 mm or more.

117. The optical system of any of the Examples above, wherein said photodetector array has an active area having a diagonal of 14 mm or more.

118. The optical system of any of the Examples above, wherein said photodetector array has an active area having a diagonal of 15 mm or more.

119. The optical system of any of the Examples above, wherein said photodetector array has an active area having a diagonal in a range from 10 mm to 20 mm.

120. The optical system of any of the Examples above, wherein said photodetector array has an active area having a diagonal in a range from 12 mm to 18 mm.

121. The optical system of any of the Examples above, wherein said photodetector array has an active area having a diagonal in a range from 13 mm to 17 mm.

122. The optical system of any of the Examples above, wherein said photodetector array has an active area having a diagonal in a range from 14 mm to 17 mm.

123. The optical system of any of the Examples above, wherein said photodetector array has an active area having a diagonal in a range from 15 mm to 16 mm.

124. The optical system of any of the Examples above, further comprising a translation stage such that the sample support structure can be translated with respect to the objective lens, said optical system configured to capture multiple images such that the multiple images can be assembled to provide a view of the sample support structure that is larger than the field of view of the objective lens.

125. The optical system of any of the Examples above, further comprising a translation stage such that the sample support structure can be translated with respect to the objective lens, said optical system configured to capture multiple images such that the multiple images can be tiled to provide a view of the sample support structure that is larger than the field of view of the objective lens.

126. The optical system of any of the Examples above, further comprising a translation stage such that the sample support structure can be translated with respect to the objective lens and electronics configured to cause multiple images to be captured by the photodetector array and to assemble said multiple images to provide a view of the sample support structure that is larger than the field of view of the objective lens.

127. The optical system of any of the Examples above, wherein said optical system is capable of capturing with said photodetector array an image of fluorescence emitting sample sites on first and second surfaces on said sample support structure.

128. The optical system of any of the Examples above, wherein said optical system is configured to correct aberrations introduced by a layer through which said first and second surfaces of said sample support structure are imaged at the same time.

129. The optical system of any of the Examples above, wherein said optics in said detection channel is configured to correct aberrations introduced by a layer through which said sample on said sample support structure is imaged.

130. The optical system of any of the Examples above, wherein said optics comprises a tube lens configured to correct aberrations introduced by a layer through which said sample on said sample support structure is imaged.

131. The optical system of any of the Examples above, wherein said objective lens is configured to correct aberrations introduced by a layer through which said sample on said sample support structure is imaged.

132. The optical system of any of the Examples above, wherein said optics in said detection channel is configured to correct aberrations introduced by a layer through which said first and second surfaces on said sample support structure is imaged.

133. The optical system of any of the Examples above, wherein said optics comprises a tube lens configured to correct aberrations introduced by a layer through which said first and second surfaces on said sample support structure is imaged.

134. The optical system of any of the Examples above, wherein said objective lens is configured to correct aberrations introduced by a layer through which said first and second surfaces on said sample support structure is imaged.

135. The optical system of any of the Examples above, wherein said layer is between 0.1 mm to 1.5 mm thick.

136. The optical system of any of the Examples above, wherein said layer has a thickness in the range from 0.15 mm to 1.3 mm thick.

137. The optical system of any of the Examples above, wherein said layer has a thickness in the range from 0.5 mm to 1.3 mm thick.

138. The optical system of the Examples above, wherein said layer has a thickness in the range from 0.75 mm to 1.25 mm thick.

139. The optical system of any of the Examples above, wherein said layer comprises glass.

140. The optical system of any of the Examples above, wherein said layer comprises a glass plate.

141. The optical system of any of the Examples above, wherein said layer comprises a cover slip.

142. The optical system of any of the Examples above, wherein said layer comprises quartz.

143. The optical system of any of the Examples above, wherein said layer comprises plastic.

144. The optical system of any of the Examples above, wherein
said optical system is capable of capturing with said photodetector array an image of fluorescence emitting sample sites on first and second surfaces on said sample support structure; and
said optical system is configured to image capture an image of said first and second surface at the same time.

145. The optical system of any of the Examples above, wherein said objective lens has a numerical aperture in the range between 0.5 to 0.4.

146. The optical system of any of the Examples above, wherein said optical system has an optical resolution in a range from 500 to 1000 nm.

147. The optical system of any of the Examples above, having an optical resolution in a range from 600 to 900 nm.

148. The optical system of any of the Examples above, wherein said optical system has an optical resolution in a range from 650 to 850 nm.

149. The optical system of any of the Examples above, further comprising said sample support structure.

150. The optical system of any of the Examples above, further comprising said sample support structure having said first and second surfaces.

151. The optical system of any of the Examples above, further comprising the sample support structure, said sample support structure comprising a flow cell.

152. The optical system of any of the Examples above, further comprising the sample support structure comprising a flow cell having a flow channel and said first and second surfaces comprise interior surfaces of said flow cell configured to be in contact with a sample flowing through said flow cell.

153. The optical system of any of the Examples above, configured for DNA sequencing.

154. The optical system of any of the Examples above, comprising four channels configured to capture images at four different spectral regions.

155. The optical system of any of the Examples above, comprising electronics configured to process images captured by a plurality of optical channels to obtain information from the fluorescing sample sites.

156. The optical system of any of the Examples above, comprising electronics configured to process images captured by a plurality of optical channels to obtain information from the fluorescing sample sites based on their locations.

157. The optical system of any of the Examples above, wherein said optical system comprises a sequencer.

158. The optical system of any of the Examples above, wherein said optical system comprises a nucleic acid sequencing apparatus.

159. The optical system of any of the Examples above, further comprising a fluid flow controller configured to control the flow of fluid through a flow cell or sample support structure.

160. The optical system of any of the Examples above, further comprising conduits configured to flow fluid through a flow cell or sample support structure.

161. The optical system of any of the Examples above, further comprising tubing that provides fluid communication with a fluid flow control system to provide fluid to a flow cell or sample support structure.

162. The optical system of any of the Examples above, further comprising a fluid flow controller configured to provide programmable control of fluid flow velocity, volumetric fluid flow rate, the timing of reagent or buffer introduction, or any combination thereof.

163. The optical system of any of the Examples above, further comprising a temperature controller configured to control the temperature of a flow cell or sample support structure.

164. The optical system of any of the Examples above, further comprising a reagent reservoir configured to provide reagent to the flow cell or sample support structure.

165. The optical system of any of the Examples above, further comprising a replaceable reagent configured to provide reagent to the flow cell or sample support structure.

166. The optical system of any of the Examples above, further comprising motorized translation stage configured to move the flow cell or sample support structure.

167. The optical system of any of the Examples above, further comprising motor controllers to control movement of a translation stage configured to move the flow cell or sample support structure.

168. The optical system of any of the Examples above, wherein said optical system comprises a nucleic sequencing apparatus.

### Part V

1. An optical system comprising:
a light source configured to produce an excitation beam;
an objective lens configured to receive emission light from a sample on a sample support structure in response to the excitation beam; and
at least one detection channel comprising optics and a photodetector array configured to receive at least a portion of the emission light and capture an image of at least one fluorescing sample site on said sample support structure,
wherein no optical element enters an optical path between the sample support structure and the photodetector array in said at least one detection channel in order to form an image of fluorescing sample sites on a first surface of said sample support structure onto the photodetector array and exits said optical path to form an image of fluorescing sample sites on a second surface of said sample support structure onto the photodetector array.

2. An optical system comprising:
a light source configured to produce an excitation beam;
an objective lens configured to receive emission light from a sample on a sample support structure in response to the excitation beam; and
at least one detection channel comprising optics and a photodetector array configured to receive at least a portion of the emission light and capture an image of at least one fluorescing sample site on said sample support structure,
wherein no optical compensation is used to form an image of fluorescing sample sites on a first surface of said sample support structure onto the photodetector array that is not identical to optical compensation used to form an image of fluorescing sample sites on a second surface of said sample support structure onto the photodetector array.

3. An optical system comprising:
a light source configured to produce an excitation beam;
an objective lens configured to receive emission light from a sample on a sample support structure in response to the excitation beam; and
at least one detection channel comprising optics and a photodetector array configured to receive at least a portion of the emission light and capture an image of at least one fluorescing sample site on said sample support structure,
wherein no optical element in an optical path between the sample support structure and a photodetector array in said at least one detection channel is adjusted differently to form an image of fluorescing sample sites on a first surface of said sample support structure onto the photodetector array than to form an image of fluorescing sample sites on a second surface of said sample support structure onto the photodetector array.

4. An optical system comprising:
a light source configured to produce an excitation beam;
an objective lens configured to receive emission light from a sample on a sample support structure in response to the excitation beam; and
at least one detection channel comprising optics and a photodetector array configured to receive at least a portion of the emission light and capture an image of at least one fluorescing sample site on said sample support structure,
wherein no optical element in an optical path between the sample support structure and a photodetector array in said at least one detection channels is moved a different amount or a different direction to form an image of fluorescing sample sites on said a first surface of said sample support structure onto the photodetector array than to form an image of fluorescing sample sites on a second surface of said sample support structure onto the photodetector array.

5. The optical system any of the Examples above, having a depth of field of at least 0.075 mm.

6. The optical system of any of the Examples above, wherein the optical system has a field of view of at least 1 mm.

7. The optical system of any of the Examples above, further comprising a sample support structure having a surface configured to produce a contrast-to-noise ratio greater than 20.

8. The optical system of any of the Examples above, further comprising a sample support structure having at least one surface comprise a hydrophilic coating.

9. The optical system of any of the Examples above, wherein the optical system is configured for high throughput assays.

10. The optical system of any of the Examples above, wherein the objective lens is disposed to receive the excitation beam, direct the excitation beam to the sample support structure.

11. The optical system of any of the Examples above, further comprising a dichroic filter disposed to reflect the excitation beam into the objective lens and to transmit the emission light to the at least one detection channel.

12. The optical system of any of the Examples above, wherein first and second surfaces on said sample support structure are separated from each other by at least 0.075 mm.

13. The optical system of any of the Examples above, wherein first surface is between said objective lens and said second surface.

14. The optical system of any of the Examples above, wherein said first and second surfaces are planar surfaces and said first is separated from each other along a direction normal to said first and second planar surfaces.

15. The optical system of any of the Examples above, wherein said first and second surfaces are planar surfaces and said first is separated from each other by at least 0.075 mm along a direction normal to said first and second planar surfaces.

16. The optical system of any of the Examples above, wherein said objective is disposed above both said first and said second surfaces and said first surface is disposed above said second surface.

17. The optical system of any of the Examples above, wherein said objective is disposed above both said first and said second surfaces and said first surface is disposed above said second surface by at least 0.075 mm.

18. The optical system of any of the Examples above, wherein said objective lens has an optical axis and said first and second surfaces are separated from each other along the direction of said optical axis.

19. The optical system of any of the Examples above, wherein said objective lens has an optical axis and said first and second surfaces are separated from each other by at least 0.075 mm along the direction of said optical axis.

20. The optical system of any one of the Examples above, wherein said first and second surfaces are both configured to provide a contrast-to-noise ratio of greater than 20 for a single sequencing cycle.

21. The optical system of any one of the Examples above, wherein said first and second surfaces are both configured to provide a contrast-to-noise ratio greater than 20 for 5 consecutive sequencing cycles.

22. The optical system of any one of the Examples above, wherein said first and second surfaces are both configured to provide a contrast-to-noise ratio of greater than 20 for 10 consecutive sequencing cycles.

23. The optical system of any one of the Examples above, wherein said first and second surfaces both comprise hydrophilic surfaces comprising labeled nucleic acid colonies at a density of at least 10000/mm² and are configured to provide a contrast-to-noise ratio of at least 20.

24. The optical system of any one of the Examples above, wherein said objective lens has a numerical aperture less than 0.6.

25. The optical system of any one of the Examples above, wherein said objective lens has a numerical aperture less than 0.55.

26. The optical system of any one of the Examples above, wherein said objective lens has a numerical aperture less than 0.5.

27. The optical system of any one of the Examples above, wherein said objective lens has a numerical aperture less than 0.45.

28. The optical system of any one of the Examples above, wherein said objective lens has a numerical aperture less than 0.4.

29. The optical system of any one of the Examples above, further comprising one or more tube lenses.

30. The optical system of any one of the Examples above, further comprising one or more tube lenses in said at least one detection channel.

31. The optical system of any one of the Examples above, wherein said objective lens and said at least one detection channel provide a field of view of at least 1 mm.

32. The optical system of any one of the Examples above, wherein said sample support structure is configured to provide a contrast-to-noise ratio of greater than 20 for a single sequencing cycle.

33. The optical system of any one of the Examples above, wherein said sample support structure is configured to provide a contrast-to-noise ratio greater than 20 for 5 consecutive sequencing cycles.

34. The optical system of any one of the Examples above, wherein said sample support structure is configured to provide a contrast-to-noise ratio of greater than 20 for 10 consecutive sequencing cycles.

35. The optical system of any one of the Examples above, wherein said sample support structure comprises a hydrophilic substrate comprising labeled nucleic acid colonies at a density of at least 10000/mm² and is configured to provide a contrast-to-noise ratio of at least 20.

36. The optical system of any one of the Examples above, wherein said first and second surfaces comprise nucleic acid colonies comprising 1, 2, 3, or 4 distinct detectable labels.

37. An optical system of any one of the Examples above, wherein said at least one detecting channel comprise imaging channels to detect 1, 2, 3, or 4 distinct labels.

38. A method of sequencing a nucleic acid comprising binding or sequencing by synthesis reaction on one or more surfaces of said sample support structure, and detecting a bound or incorporated base using the optical system of any of the Examples above.

39. A method of determining a genotype of a sample comprising a nucleic acid molecule, comprising preparing said nucleic acid molecule for sequencing, and then sequencing said nucleic acid molecule using the optical system of any of the Examples above.

40. An optical system of any one of the Examples above, wherein the sample support structure comprises one, two, three, four, five, or six imaging surfaces comprising fluorescing sample sites.

41. An optical system of any one of the Examples above, wherein the sample support structure comprises a flow cell.

42. An optical system of any one of the Examples above, wherein the sample support structure comprises a sample chamber.

43. An optical system of any one of the Examples above, further comprising said sample support structure.

44. An optical system of any one of the Examples above, wherein said optical system has a field-of-view of at least 1.0 mm wide with less than 0.1 waves of aberration over at least 80% of the field-of-view.

45. An optical system of any one of the Examples above, wherein said optical system has a field-of-view of at least 1.0 mm wide with less than 0.1 waves of aberration over at least 90% of the field-of-view.

46. An optical system of any one of the Examples above, wherein said optical system has a field-of-view of at least 2.0 mm wide with less than 0.09 waves of aberration over at least 80% of the field-of-view.

47. An optical system of any one of the Examples above, wherein said optical system has a field-of-view of at least 2.0 mm wide with less than 0.09 waves of aberration over at least 90% of the field-of-view.

48. An optical system of any one of the Examples above, wherein said optical system has a field-of-view of at least 3.0 mm wide with less than 0.09 waves of aberration over at least 80% of the field-of-view.

49. An optical system of any one of the Examples above, wherein said optical system has a field-of-view of at least 3.0 mm wide with less than 0.09 waves of aberration over at least 90% of the field-of-view.

50. An optical system of any one of the Examples above, wherein said optical system has a field-of-view of at least 3.2 mm wide with less than 0.09 waves of aberration over at least 80% of the field-of-view.

51. An optical system of any one of the Examples above, wherein said optical system has a field-of-view of at least 3.2 mm wide with less than 0.09 waves of aberration over at least 90% of the field-of-view.

52. An optical system of any one of the Examples above, wherein said optical system has a work distance of in a range from 2 to 6 mm.

53. An optical system of any one of the Examples above, wherein said optical system has a work distance of in a range from 2.5 to 5.5 mm.

54. An optical system of any one of the Examples above, wherein said optical system has a work distance of in a range from 3 to 5 mm.

55. An optical system of any one of the Examples above, wherein said optical system has a work distance of at least 3 mm.

56. The optical system of any one of the Examples above, wherein said optical system is capable of capturing with said photodetector array an image of fluorescence emitting sample sites on first and second surfaces on said sample support structure.

57. The optical system of any one of the Examples 118, wherein said first surface is between said objective lens and said second surface, said first and second surfaces separated from each other by at least 0.075 mm.

58. The optical system of any of one the Examples 118 or 119, wherein said optical system has less than 0.1 waves of aberration over at least 80% of the field of view for both said first and second surfaces.

59. The optical system of any one of the Examples 118-120, wherein said optical system has less than 0.1 waves of aberration over at least 90% of the field of view for both said first and second surfaces.

60. The optical system of any one of the Examples 118-120, wherein said optical system provide diffraction limited imaging of for both said first and second surfaces.

61. An optical system of any one of the Examples above, wherein the optical system has a field-of-view of at least 1.0 mm wide and is diffraction limited.

62. An optical system of any one of the Examples above, wherein said optical system has a field-of-view of at least 1.0 mm wide.

63. An optical system of any one of the Examples above, wherein said optical system has a field-of-view of at least 2.0 mm wide.

64. An optical system of any one of the Examples above, wherein said optical system has a field-of-view of at least 3.0 mm wide.

65. An optical system of any one of the Examples above, wherein optical system has a field-of-view of at least 3.2 mm wide.

66. The optical system of any one of the Examples above, wherein said optical system is capable of capturing with said photodetector array an image of fluorescence emitting sample sites on first and second surfaces on said sample support structure.

67. The optical system of any one of the Examples above, wherein said optical system provides diffraction limited imaging of for first and second surfaces on said sample support structure.

68. The optical system of any one of the Examples above, wherein said optical system provides diffraction limited imaging of first and second surfaces on said sample support structure separated by 0.075 mm.

69. The optical system of any one of the Examples above, wherein said optical system provides diffraction limited imaging of first and second surfaces on said sample support structure separated by 0.075 mm along a direction parallel to the optical axis of the objective lens.

70. The optical system of any one of the Examples 16-18, wherein said at least one light source is configured to produce an excitation beam, said light source having an optical output power of at least 0.8 W.

71. The optical system of any of the Examples above, wherein said light source has an optical output power of at least 1 W.

72. The optical system of any of the Examples above, wherein said at least one light source comprises a laser.

73. The optical system of any of the Examples above, wherein said at least one light source comprises a laser diode.

74. The optical system of any of the Examples above, wherein said at least one light source comprises a visible color light source.

75. The optical system of any of the Examples above, wherein said at least one light source comprises a green or red light source.

76. The optical system of any of the Examples above, comprising a plurality of light sources.

77. The optical system of any of the Examples above, wherein said at least one light source comprises at least first and second light sources each having an optical output power of at least 0.8 W.

78. The optical system of any of the Examples above, wherein said at least one light source comprises at least first and second light sources each having an optical output power of at least 1 W.

79. The optical system of any of the Examples above, wherein said at least one light source comprises at least first and second laser light sources.

80. The optical system of any of the Examples above, wherein said at least one light source comprises at least first and second light sources comprising laser diodes.

81. The optical system of any of the Examples above, wherein said at least one light source comprises at least first and second visible color light sources.

82. The optical system of any of the Examples above, wherein said at least one light source comprises at least a first green light source and a second red light source.

83. The optical system of any of the Examples above, wherein said optical system is configured to image said first and second surfaces at the same time and optical aberration is less for imaging said first and second surfaces than elsewhere in a region from 1 to 10 mm from said objective lens.

84. The optical system of any of the Examples above, wherein said first surface is between said objective lens and said second surface, said first separated from each other by at least 0.075 mm.

85. The optical system of any of the Examples above, wherein said optical system has less than 0.1 waves of aberration over at least 80% of the field of view for both said first and second surfaces.

86. The optical system of any of the Examples above, wherein said optical system has less than 0.1 waves of aberration over at least 90% of the field of view for both said first and second surfaces.

87. The optical system of any of the Examples above, wherein said optical system provide diffraction limited imaging of for both said first and second surfaces.

88. The optical system of any of the Examples above, wherein optical aberration of said objective lens is less for imaging said first and second surfaces than elsewhere in a region from 1 to 10 mm from said objective lens.

89. The optical system of any of the Examples above, wherein optical aberration of said tube lens is less for imaging said first and second surfaces than elsewhere in a region from 1 to 10 mm from said objective lens.

90. The optical system of any of the Examples above, wherein said objective lens is configured such that said optical system has a magnification of less than 10 (10×).

91. The optical system of any of the Examples above, wherein said objective lens is configured such that said optical system has a magnification of 8 (8×) or less.

92. The optical system of any of the Examples above, wherein said objective lens is configured such that said optical system has a magnification of 6 (6×) or less.

93. The optical system of any of the Examples above, wherein said objective lens is configured such that said optical system has a magnification of 5.5 (5.5×) or less.

94. The optical system of any of the Examples above, wherein said objective lens is configured such that said optical system has a magnification of 5 (5×) or less.

95. The optical system of any of the Examples above, wherein said at least one detection channel is configured to satisfy the Nyquist theorem for diffraction limited imaging.

96. The optical system of any of the Examples above, wherein said photodetector array comprises a plurality of pixels having a dimension that satisfies the Nyquist theorem for diffraction limited imaging.

97. The optical system of any of the Examples above, wherein said photodetector array comprises a plurality of pixels having a pixel size or pitch of less than 5 mm.

98. The optical system of any of the Examples above, wherein said photodetector array comprises a plurality of pixels having a pixel size or pitch of less than 4 mm.

99. The optical system of any of the Examples above, wherein said photodetector array comprises a plurality of pixels having a pixel size or pitch of 3 mm or less.

100. The optical system of any of the Examples above, wherein said photodetector array comprises a plurality of pixels having a pixel size or pitch 2.5 mm or less.

101. The optical system of any of the Examples above, wherein said photodetector array comprises a plurality of pixels having a pixel size or pitch in the range from 5 mm to 1 mm.

102. The optical system of any of the Examples above, wherein said photodetector array comprises a plurality of pixels having a pixel size or pitch in the range from 4 mm to 2 mm.

103. The optical system of any of the Examples above, wherein said photodetector array comprises a plurality of pixels having a pixel size or pitch in the range from 3 mm to 2 mm.

104. The optical system of any of the Examples above, wherein said optical system has a field of view of at least 1 mm wide.

105. The optical system of any of the Examples above, wherein said optical system has a field of view of at least 2 mm wide.

106. The optical system of any of the Examples above, wherein said optical system has a field of view of at least 3 mm wide.

107. The optical system of any of the Examples above, wherein said optical system has working distance in the range from 1 to 10 mm.

108. The optical system of any of the Examples above, wherein said optical system has working distance in the range from 2.5 to 5.5 mm.

109. The optical system of any of the Examples above, wherein optical system has working distance in the range from 3 to 5 mm.

110. The optical system of any of the Examples above, wherein said photodetector array has an active area having a diagonal of 10 mm or more.

111. The optical system of any of the Examples above, wherein said photodetector array has an active area having a diagonal of 13 mm or more.

112. The optical system of any of the Examples above, wherein said photodetector array has an active area having a diagonal of 14 mm or more.

113. The optical system of any of the Examples above, wherein said photodetector array has an active area having a diagonal of 15 mm or more.

114. The optical system of any of the Examples above, wherein said photodetector array has an active area having a diagonal in a range from 10 mm to 20 mm.

115. The optical system of any of the Examples above, wherein said photodetector array has an active area having a diagonal in a range from 12 mm to 18 mm.

116. The optical system of any of the Examples above, wherein said photodetector array has an active area having a diagonal in a range from 13 mm to 17 mm.

117. The optical system of any of the Examples above, wherein said photodetector array has an active area having a diagonal in a range from 14 mm to 17 mm.

118. The optical system of any of the Examples above, wherein said photodetector array has an active area having a diagonal in a range from 15 mm to 16 mm.

119. The optical system of any of the Examples above, further comprising a translation stage such that the sample support structure can be translated with respect to the objective lens, said optical system configured to capture multiple images such that the multiple images can be assembled to provide a view of the sample support structure that is larger than the field of view of the objective lens.

120. The optical system of any of the Examples above, further comprising a translation stage such that the sample support structure can be translated with respect to the objective lens, said optical system configured to capture multiple images such that the multiple images can be tiled to provide a view of the sample support structure that is larger than the field of view of the objective lens.

121. The optical system of any of the Examples above, further comprising a translation stage such that the sample support structure can be translated with respect to the objective lens and electronics configured to cause multiple images to be captured by the photodetector array and to assemble said multiple images to provide a view of the sample support structure that is larger than the field of view of the objective lens.

122. The optical system of any of the Examples above, wherein said optical system is capable of capturing with said photodetector array an image of fluorescence emitting sample sites on first and second surfaces on said sample support structure.

123. The optical system of any of the Examples above, wherein said optical system is configured to correct aberrations introduced by a layer through which said first and second surfaces of said sample support structure are imaged at the same time.

124. The optical system of any of the Examples above, wherein said optics in said detection channel is configured to correct aberrations introduced by a layer through which said sample on said sample support structure is imaged.

125. The optical system of any of the Examples above, wherein said optics comprises a tube lens configured to correct aberrations introduced by a layer through which said sample on said sample support structure is imaged.

126. The optical system of any of the Examples above, wherein said objective lens is configured to correct aberrations introduced by a layer through which said sample on said sample support structure is imaged.

127. The optical system of any of the Examples above, wherein said optics in said detection channel is configured to correct aberrations introduced by a layer through which said first and second surfaces on said sample support structure is imaged.

128. The optical system of any of the Examples above, wherein said optics comprises a tube lens configured to correct aberrations introduced by a layer through which said first and second surfaces on said sample support structure is imaged.

129. The optical system of any of the Examples above, wherein said objective lens is configured to correct aberrations introduced by a layer through which said first and second surfaces on said sample support structure is imaged.

130. The optical system of any of the Examples above, wherein said layer is between 0.1 mm to 1.5 mm thick.

131. The optical system of any of the Examples above, wherein said layer has a thickness in the range from 0.15 mm to 1.3 mm thick.

132. The optical system of any of the Examples above, wherein said layer has a thickness in the range from 0.5 mm to 1.3 mm thick.

133. The optical system of the Examples above, wherein said layer has a thickness in the range from 0.75 mm to 1.25 mm thick.

134. The optical system of any of the Examples above, wherein said layer comprises glass.

135. The optical system of any of the Examples above, wherein said layer comprises a glass plate.

136. The optical system of any of the Examples above, wherein said layer comprises a cover slip.

137. The optical system of any of the Examples above, wherein said layer comprises quartz.

138. The optical system of any of the Examples above, wherein said layer comprises plastic.

139. The optical system of any of the Examples above, wherein said optical system is capable of capturing with said photodetector images of fluorescence emitting sample sites on first and second surfaces on said sample support structure; and said optical system is configured to image capture an image of said first and second surface at the same time.

140. The optical system of any of the Examples above, wherein said objective lens has a numerical aperture in the range between 0.5 to 0.4.

141. The optical system of any of the Examples above, wherein optical system has an optical resolution in a range from 500 to 1000 nm.

142. The optical system of any of the Examples above, having an optical resolution in a range from 600 to 900 nm.

143. The optical system of any of the Examples above, wherein optical system has an optical resolution in a range from 650 to 850 nm.

144. The optical system of any of the Examples above, further comprising said sample support structure.

145. The optical system of any of the Examples above, further comprising said sample support structure having said first and second surfaces.

146. The optical system of any of the Examples above, further comprising the sample support structure, said sample support structure comprising a flow cell.

147. The optical system of any of the Examples above, further comprising the sample support structure comprising a flow cell having a flow channel and said first and second surfaces comprise interior surfaces of said flow cell configured to be in contact with a sample flowing through said flow cell.

148. The optical system of any of the Examples above, configured for DNA sequencing.

149. The optical system of any of the Examples above, comprising four channels configured to capture images at four different spectral regions.

150. The optical system of any of the Examples above, comprising electronics configured to process images captured by a plurality of optical channels to obtain information from the fluorescing sample sites.

151. The optical system of any of the Examples above, comprising electronics configured to process images captured by a plurality of optical channels to obtain information from the fluorescing sample sites based on their locations.

152. The optical system of any of the Examples above, wherein said optical system comprises a sequencer.

153. The optical system of any of the Examples above, wherein said optical system comprises a nucleic acid sequencing apparatus.

154. The optical system of any of the Examples above, further comprising a fluid flow controller configured to control the flow of fluid through a flow cell or sample support structure.

155. The optical system of any of the Examples above, further comprising conduits configured to flow fluid through a flow cell or sample support structure.

156. The optical system of any of the Examples above, further comprising tubing that provides fluid communication with a fluid flow control system to provide fluid to a flow cell or sample support structure.

157. The optical system of any of the Examples above, further comprising a fluid flow controller configured to provide programmable control of fluid flow velocity, volumetric fluid flow rate, the timing of reagent or buffer introduction, or any combination thereof.

158. The optical system of any of the Examples above, further comprising a temperature controller configured to control the temperature of a flow cell or sample support structure.

159. The optical system of any of the Examples above, further comprising a reagent reservoir configured to provide reagent to the flow cell or sample support structure.

160. The optical system of any of the Examples above, further comprising a replaceable reagent configured to provide reagent to the flow cell or sample support structure.

161. The optical system of any of the Examples above, further comprising motorized translation stage configured to move the flow cell or sample support structure.

162. The optical system of any of the Examples above, further comprising motor controllers to control movement of a translation stage configured to move the flow cell or sample support structure.

163. The optical system of any of the Examples above, wherein said optical system comprises a nucleic sequencing apparatus.

### Part VI

1. An optical system comprising:
at least one light source configured to produce an excitation beam;
an objective lens configured to receive emission light from a sample on a support structure in response to the excitation beam;
at least one detection channel comprising optics and a photodetector array configured to receive at least a portion of the emission light and capture an image of at least one fluorescing sample site on said sample support structure; and
wherein said optical system has a field-of-view of at least 1.0 mm wide with less than 0.1 waves of aberration over at least 80% of the field-of-view.

2. An optical system of any one of the Examples above, wherein said optical system has a field-of-view of at least 1.0 mm wide with less than 0.1 waves of aberration over at least 90% of the field-of-view.

3. An optical system of any one of the Examples above, wherein said optical system has a field-of-view of at least 2.0 mm wide with less than 0.09 waves of aberration over at least 80% of the field-of-view.

4. An optical system of any one of the Examples above, wherein said optical system has a field-of-view of at least 2.0 mm wide with less than 0.09 waves of aberration over at least 90% of the field-of-view.

5. An optical system of any one of the Examples above, wherein optical system has a field-of-view of at least 3.0 mm wide with less than 0.09 waves of aberration over at least 80% of the field-of-view.

6. An optical system of any one of the Examples above, wherein said optical system has a field-of-view of at least 3.0 mm wide with less than 0.09 waves of aberration over at least 90% of the field-of-view.

7. An optical system of any one of the Examples above, wherein said optical system has a field-of-view of at least 3.2 mm wide with less than 0.09 waves of aberration over at least 80% of the field-of-view.

8. An optical system of any one of the Examples above, wherein said optical system has a field-of-view of at least 3.2 mm wide with less than 0.09 waves of aberration over at least 90% of the field-of-view.

9. An optical system of any one of the Examples above, wherein said optical system has a work distance of in a range from 2 to 6 mm.

10. An optical system of any one of the Examples above, wherein said optical system has a work distance of in a range from 2.5 to 5.5 mm.

11. An optical system of any one of the Examples above, wherein said optical system has a work distance of in a range from 3 to 5 mm.

12. An optical system of any one of the Examples above, wherein said optical system has a work distance of at least 3 mm.

13. An optical system of any one of the Examples above, wherein said objective lens has a numerical aperture of less than 0.6.

14. An optical system of any one of the Examples above, wherein said objective lens has a numerical aperture of 0.55 or less.

15. An optical system of any of the Examples above, wherein said objective lens has a numerical aperture of 0.5 or less.

16. The optical system of any one of the Examples above, wherein said optical system is capable of capturing with said photodetector array an image of fluorescence emitting sample sites on first and second surfaces on said sample support structure.

17. The optical system of any one of the Examples 16, wherein said first surface is between said objective lens and said second surface, said first and second surfaces separated from each other by at least 0.075 mm.

18. The optical system of any of one the Examples 16 or 17, wherein said optical system has less than 0.1 waves of aberration over at least 80% of the field of view for both said first and second surfaces.

19. The optical system of any one of the Examples 16-18, wherein said optical system has less than 0.1 waves of aberration over at least 90% of the field of view for both said first and second surfaces.

20. The optical system of any one of the Examples 16-18, wherein said optical system provide diffraction limited imaging of for both said first and second surfaces.

21. An optical system comprising:
at least one light source configured to produce an excitation beam;
an objective lens configured to receive emission light from a sample on a support structure in response to the excitation beam; and
at least one detection channel comprising optics and a photodetector array configured to receive at least a portion of the emission light and capture an image of at least one fluorescing sample site on said sample support structure,
wherein the optical system has a field-of-view of at least 1.0 mm wide and is diffraction limited.

22. An optical system of any one of the Examples above, wherein said optical system has a field-of-view of at least 2.0 mm wide.

23. An optical system of any one of the Examples above, wherein said optical system has a field-of-view of at least 2.5 mm wide.

24. An optical system of any one of the Examples above, wherein said optical system has a field-of-view of at least 3.0 mm wide.

25. An optical system of any one of the Examples above, wherein optical system has a field-of-view of at least 3.2 mm wide.

26. An optical system of any one of the Examples above, wherein said optical system has a work distance of in a range from 2 to 6 mm.

27. An optical system of any one of the Examples above, wherein said optical system has a work distance of in a range from 2.5 to 5.5 mm.

28. An optical system of any one of the Examples above, wherein said optical system has a work distance of in a range from 3 to 5 mm.

29. An optical system of any one of the Examples above, wherein said optical system has a work distance of at least 3 mm.

30. An optical system of any one of the Examples above, wherein said objective lens has a numerical aperture of less than 0.6.

31. An optical system of any one of the Examples above, wherein said objective lens has a numerical aperture of 0.55 or less.

32. An optical system of any one of the Examples above, wherein said objective lens has a numerical aperture of 0.5 or less.

33. The optical system of any one of the Examples above, wherein said optical system is capable of capturing with said photodetector array an image of fluorescence emitting sample sites on first and second surfaces on said sample support structure.

34. The optical system of any one of the Examples above, wherein said optical system provides diffraction limited imaging of first and second surfaces on said sample support structure.

35. The optical system of any one of the Examples above, wherein said optical system provides diffraction limited imaging of first and second surfaces on said sample support structure separated by 0.075 mm.

36. The optical system of any one of the Examples above, wherein said optical system provides diffraction limited imaging of first and second surfaces on said sample support structure separated by 0.075 mm along a direction parallel to the optical axis of the objective lens.

37. An optical system comprising:
at least one light source configured to produce an excitation beam, said light source having an optical output power of at least 0.8 W;
an objective lens configured to receive emission light from a sample on a support structure in response to the excitation beam;
a plurality of detection channels comprising optics and photodetector arrays configured to receive at least a portion of the emission light and capture an image of at least one fluorescing sample site on said sample support structure.

38. The optical system of any of the Examples above, wherein said light source has an optical output power of at least 1 W.

39. The optical system of any of the Examples above, wherein said at least one light source comprises a laser.

40. The optical system of any of the Examples above, wherein said at least one light source comprises a laser diode.

41. The optical system of any of the Examples above, wherein said at least one light source comprises a visible color light source.

42. The optical system of any of the Examples above, wherein said at least one light source comprises a green or red light source.

43. The optical system of any of the Examples above, comprising a plurality of light sources.

44. The optical system of any of the Examples above, wherein said at least one light source comprises at least first and second light sources each having an optical output power of at least 0.8 W.

45. The optical system of any of the Examples above, wherein said at least one light source comprises at least first and second light sources each having an optical output power of at least 1 W.

46. The optical system of any of the Examples above, wherein said at least one light source comprises at least first and second laser light sources.

47. The optical system of any of the Examples above, wherein said at least one light source comprises at least first and second light sources comprising laser diodes.

48. The optical system of any of the Examples above, wherein said at least one light source comprises at least first and second visible color light sources.

49. The optical system of any of the Examples above, wherein said at least one light source comprises at least a first green light source and a second red light source.

50. An optical system comprising:
a light source configured to produce an excitation beam;
an objective lens configured to receive emission light from a sample on a support structure in response to the excitation beam; and
at least one detection channel comprising optics and a photodetector array configured to receive at least a portion of the emission light and capture an image of at least one fluorescing sample site on said sample support structure,
wherein said optical system is capable of capturing with said photodetector array images of fluorescence emitting sample sites on first and second surfaces on said sample support structure; and
wherein said optical system is configured to image said first and second surfaces at the same time and optical aberration is less for imaging said first and second surfaces than elsewhere in a region from 1 to 10 mm from said objective lens.

51. The optical system of any of the Examples above, wherein said first surface is between said objective lens and said second surface, said first separated from each other by at least 0.075 mm.

52. The optical system of any of the Examples above, wherein said optical system has less than 0.1 waves of aberration over at least 80% of the field of view for both said first and second surfaces.

53. The optical system of any of the Examples above, wherein said optical system has less than 0.1 waves of aberration over at least 90% of the field of view for both said first and second surfaces.

54. The optical system of any of the Examples above, wherein said optical system provide diffraction limited imaging of for both said first and second surfaces.

55. The optical system of any of the Examples above, wherein optical aberration of said objective lens is less for imaging said first and second surfaces than elsewhere in a region from 1 to 10 mm from said objective lens.

56. The optical system of any of the Examples above, wherein optical aberration of said tube lens is less for imaging said first and second surfaces than elsewhere in a region from 1 to 10 mm from said objective lens.

57. An optical system comprising:
at least one light source configured to produce an excitation beam;
an objective lens configured to receive emission light from a sample on a support structure in response to the excitation beam; and
a plurality of detection channels comprising optics and photodetector arrays configured to receive at least a portion of the emission light and capture an image of at least one fluorescing sample site on said sample support structure,
wherein said objective lens is configured such that said optical system has a magnification of less than 10 (10×).

58. The optical system of any of the Examples above, wherein said objective lens is configured such that said optical system has a magnification of 8 (8×) or less.

59. The optical system of any of the Examples above, wherein said objective lens is configured such that said optical system has a magnification of 6 (6×) or less.

60. The optical system of any of the Examples above, wherein said objective lens is configured such that said optical system has a magnification of 5.5 (5.5×) or less.

61. The optical system of any of the Examples above, wherein said objective lens is configured such that said optical system has a magnification of 5 (5×) or less.

62. The optical system of any of the Examples above, wherein said one or more objective lens has a numerical aperture of 0.5 or less.

63. The optical system of any of the Examples above, wherein said one or more objective lens has a numerical aperture of 0.6 or less.

64. The optical system of any of the Examples above, wherein said at least one detection channel is configured to satisfy the Nyquist theorem for diffraction limited imaging.

65. The optical system of any of the Examples above, wherein said photodetector array comprises a plurality of pixels having a dimension that satisfies the Nyquist theorem for diffraction limited imaging.

66. The optical system of any of the Examples above, wherein said photodetector array comprises a plurality of pixels having a pixel size or pitch of less than 5 mm.

67. The optical system of any of the Examples above, wherein said photodetector array comprises a plurality of pixels having a pixel size or pitch of less than 4 mm.

68. The optical system of any of the Examples above, wherein said photodetector array comprises a plurality of pixels having a pixel size or pitch of 3 mm or less.

69. The optical system of any of the Examples above, wherein said photodetector array comprises a plurality of pixels having a pixel size or pitch 2.5 mm or less.

70. The optical system of any of the Examples above, wherein said photodetector array comprises a plurality of pixels having a pixel size or pitch in the range from 5 mm to 1 mm.

71. The optical system of any of the Examples above, wherein said photodetector array comprises a plurality of pixels having a pixel size or pitch in the range from 4 mm to 2 mm.

72. The optical system of any of the Examples above, wherein said photodetector array comprises a plurality of pixels having a pixel size or pitch in the range from 3 mm to 2 mm.

73. The optical system of any of the Examples above, wherein said optical system has a field of view of at least 1 mm wide.

74. The optical system of any of the Examples above, wherein said optical system has a field of view of at least 2 mm wide.

75. The optical system of any of the Examples above, wherein said optical system has a field of view of at least 3 mm wide.

76. The optical system of any of the Examples above, wherein said optical system has working distance in the range from 1 to 10 mm.

77. The optical system of any of the Examples above, wherein said optical system has working distance in the range from 2.5 to 5.5 mm.

78. The optical system of any of the Examples above, wherein optical system has working distance in the range from 3 to 5 mm.

79. The optical system of any of the Examples above, wherein said photodetector array has an active area having a diagonal of 10 mm or more.

80. The optical system of any of the Examples above, wherein said photodetector array has an active area having a diagonal of 13 mm or more.

81. The optical system of any of the Examples above, wherein said photodetector array has an active area having a diagonal of 14 mm or more.

82. The optical system of any of the Examples above, wherein said photodetector array has an active area having a diagonal of 15 mm or more.

83. The optical system of any of the Examples above, wherein said photodetector array has an active area having a diagonal in a range from 10 mm to 20 mm.

84. The optical system of any of the Examples above, wherein said photodetector array has an active area having a diagonal in a range from 12 mm to 18 mm.

85. The optical system of any of the Examples above, wherein said photodetector array has an active area having a diagonal in a range from 13 mm to 17 mm.

86. The optical system of any of the Examples above, wherein said photodetector array has an active area having a diagonal in a range from 14 mm to 17 mm.

87. The optical system of any of the Examples above, wherein said photodetector array has an active area having a diagonal in a range from 15 mm to 16 mm.

88. The optical system of any of the Examples above, further comprising a translation stage such that the sample support structure can be translated with respect to the objective lens, said optical system configured to capture multiple images such that the multiple images can be assembled to provide a view of the sample support structure that is larger than the field of view of the objective lens.

89. The optical system of any of the Examples above, further comprising a translation stage such that the sample support structure can be translated with respect to the objective lens, said optical system configured to capture multiple images such that the multiple images can be tiled to provide a view of the sample support structure that is larger than the field of view of the objective lens.

90. The optical system of any of the Examples above, further comprising a translation stage such that the sample support structure can be translated with respect to the objective lens and electronics configured to cause multiple images to be captured by the photodetector array and to assemble said multiple images to provide a view of the sample support structure that is larger than the field of view of the objective lens.

91. The optical system of any of the Examples above, wherein said optical system is capable of capturing with said photodetector array an image of fluorescence emitting sample sites on first and second surfaces on said sample support structure.

92. An optical system comprising:
a light source configured to produce an excitation beam;
an objective lens configured to receive emission light from a sample on a support structure in response to the excitation beam; and
at least one detection channel comprising optics and a photodetector array configured to receive at least a portion of the emission light and capture an image of at least one fluorescing sample site on said sample support structure,
wherein said optical system is capable of capturing with said photodetector array images of fluorescence emitting sample sites on first and second surfaces on said sample support structure; and
wherein said optical system is configured to correct aberrations introduced by a layer through which said first and second surfaces of said sample support structure are imaged at the same time.

93. The optical system of any of the Examples above, wherein said optics in said detection channel is configured to correct aberrations introduced by a layer through which said sample on said sample support structure is imaged.

94. The optical system of any of the Examples above, wherein said optics comprises a tube lens configured to correct aberrations introduced by a layer through which said sample on said sample support structure is imaged.

95. The optical system of any of the Examples above, wherein said objective lens is configured to correct aberrations introduced by a layer through which said sample on said sample support structure is imaged.

96. The optical system of any of the Examples above, wherein said optics in said detection channel is configured to correct aberrations introduced by a layer through which said first and second surfaces on said sample support structure is imaged.

97. The optical system of any of the Examples above, wherein said optics comprises a tube lens configured to correct aberrations introduced by a layer through which said first and second surfaces on said sample support structure is imaged.

98. The optical system of any of the Examples above, wherein said objective lens is configured to correct aberrations introduced by a layer through which said first and second surfaces on said sample support structure is imaged.

99. The optical system of any of the Examples above, wherein said layer is between 0.1 mm to 1.5 mm thick.

100. The optical system of any of the Examples above, wherein said layer has a thickness in the range from 0.15 mm to 1.3 mm thick.

101. The optical system of any of the Examples above, wherein said layer has a thickness in the range from 0.5 mm to 1.3 mm thick.

102. The optical system of the Examples above, wherein said layer has a thickness in the range from 0.75 mm to 1.25 mm thick.

103. The optical system of any of the Examples above, wherein said layer comprises glass.

104. The optical system of any of the Examples above, wherein said layer comprises a glass plate.

105. The optical system of any of the Examples above, wherein said layer comprises a cover slip.

106. An optical system comprising:
at least one light source configured to produce an excitation beam;
an objective lens configured to receive emission light from a sample on a support structure in response to the excitation beam; and
at least one detection channel comprising optics and a photodetector array configured to receive at least a portion of the emission light and capture an image of at least one fluorescing sample site on said sample support structure,
wherein said optical system is capable of capturing with said photodetector array images of fluorescence emitting sample sites on first and second surfaces on said sample support structure, said optical system configured to form an image of said first and second surfaces on said photodetector array at the same time.

107. An optical system of any one of the Examples above, wherein said optical system has a field-of-view of at least 1.0 mm wide with less than 0.1 waves of aberration over at least 80% of the field-of-view.

108. An optical system of any one of the Examples above, wherein said optical system has a field-of-view of at least 1.0 mm wide with less than 0.1 waves of aberration over at least 90% of the field-of-view.

109. An optical system of any one of the Examples above, wherein said optical system has a field-of-view of at least 2.0 mm wide with less than 0.09 waves of aberration over at least 80% of the field-of-view.

110. An optical system of any one of the Examples above, wherein said optical system has a field-of-view of at least 2.0 mm wide with less than 0.09 waves of aberration over at least 90% of the field-of-view.

111. An optical system of any one of the Examples above, wherein optical system has a field-of-view of at least 3.0 mm wide with less than 0.09 waves of aberration over at least 80% of the field-of-view.

112. An optical system of any one of the Examples above, wherein said optical system has a field-of-view of at least 3.0 mm wide with less than 0.09 waves of aberration over at least 90% of the field-of-view.

113. An optical system of any one of the Examples above, wherein said optical system has a field-of-view of at least 3.2 mm wide with less than 0.09 waves of aberration over at least 80% of the field-of-view.

114. An optical system of any one of the Examples above, wherein said optical system has a field-of-view of at least 3.2 mm wide with less than 0.09 waves of aberration over at least 90% of the field-of-view.

115. An optical system of any one of the Examples above, wherein said optical system has a work distance of in a range from 2 to 6 mm.

116. An optical system of any one of the Examples above, wherein said optical system has a work distance of in a range from 2.5 to 5.5 mm.

117. An optical system of any one of the Examples above, wherein said optical system has a work distance of in a range from 3 to 5 mm.

118. An optical system of any one of the Examples above, wherein said optical system has a work distance of at least 3 mm.

119. The optical system of any one of the Examples above, wherein said optical system is capable of capturing with said photodetector array an image of fluorescence emitting sample sites on first and second surfaces on said sample support structure.

120. The optical system of any one of the Examples 118, wherein said first surface is between said objective lens and said second surface, said first and second surfaces separated from each other by at least 0.075 mm.

121. The optical system of any of one the Examples 118 or 119, wherein said optical system has less than 0.1 waves of aberration over at least 80% of the field of view for both said first and second surfaces.

122. The optical system of any one of the Examples 118-120, wherein said optical system has less than 0.1 waves of aberration over at least 90% of the field of view for both said first and second surfaces.

123. The optical system of any one of the Examples 118-120, wherein said optical system provide diffraction limited imaging of for both said first and second surfaces.

124. An optical system of any one of the Examples above, wherein the optical system has a field-of-view of at least 1.0 mm wide and is diffraction limited.

125. An optical system of any one of the Examples above, wherein said optical system has a field-of-view of at least 2.0 mm wide.

126. An optical system of any one of the Examples above, wherein said optical system has a field-of-view of at least 2.5 mm wide.

127. An optical system of any one of the Examples above, wherein said optical system has a field-of-view of at least 3.0 mm wide.

128. An optical system of any one of the Examples above, wherein optical system has a field-of-view of at least 3.2 mm wide.

129. The optical system of any one of the Examples above, wherein said optical system is capable of capturing with said photodetector array an image of fluorescence emitting sample sites on first and second surfaces on said sample support structure.

130. The optical system of any one of the Examples above, wherein said optical system provides diffraction limited imaging of for first and second surfaces on said sample support structure.

131. The optical system of any one of the Examples above, wherein said optical system provides diffraction limited imaging of first and second surfaces on said sample support structure separated by 0.075 mm.

132. The optical system of any one of the Examples above, wherein said optical system provides diffraction limited imaging of first and second surfaces on said sample support structure separated by 0.075 mm along a direction parallel to the optical axis of the objective lens.

133. The optical system of any one of the Examples 16-18, wherein said at least one light source is configured to produce an excitation beam, said light source having an optical output power of at least 0.8 W.

134. The optical system of any of the Examples above, wherein said light source has an optical output power of at least 1 W.

135. The optical system of any of the Examples above, wherein said at least one light source comprises a laser.

136. The optical system of any of the Examples above, wherein said at least one light source comprises a laser diode.

137. The optical system of any of the Examples above, wherein said at least one light source comprises a visible color light source.

138. The optical system of any of the Examples above, wherein said at least one light source comprises a green or red light source.

139. The optical system of any of the Examples above, comprising a plurality of light sources.

140. The optical system of any of the Examples above, wherein said at least one light source comprises at least first and second light sources each having an optical output power of at least 0.8 W.

141. The optical system of any of the Examples above, wherein said at least one light source comprises at least first and second light sources each having an optical output power of at least 1 W.

142. The optical system of any of the Examples above, wherein said at least one light source comprises at least first and second laser light sources.

143. The optical system of any of the Examples above, wherein said at least one light source comprises at least first and second light sources comprising laser diodes.

144. The optical system of any of the Examples above, wherein said at least one light source comprises at least first and second visible color light sources.

145. The optical system of any of the Examples above, wherein said at least one light source comprises at least a first green light source and a second red light source.

146. The optical system of any of the Examples above, wherein said optical system is configured to image said first and second surfaces at the same time and optical aberration is less for imaging said first and second surfaces than elsewhere in a region from 1 to 10 mm from said objective lens.

147. The optical system of any of the Examples above, wherein said first surface is between said objective lens and said second surface, said first separated from each other by at least 0.075 mm.

148. The optical system of any of the Examples above, wherein said optical system has less than 0.1 waves of aberration over at least 80% of the field of view for both said first and second surfaces.

149. The optical system of any of the Examples above, wherein said optical system has less than 0.1 waves of aberration over at least 90% of the field of view for both said first and second surfaces.

150. The optical system of any of the Examples above, wherein said optical system provide diffraction limited imaging of for both said first and second surfaces.

151. The optical system of any of the Examples above, wherein optical aberration of said objective lens is less for imaging said first and second surfaces than elsewhere in a region from 1 to 10 mm from said objective lens.

152. The optical system of any of the Examples above, wherein optical aberration of said tube lens is less for imaging said first and second surfaces than elsewhere in a region from 1 to 10 mm from said objective lens.

153. The optical system of any of the Examples above, wherein said objective lens is configured such that said optical system has a magnification of less than 10 (10×).

154. The optical system of any of the Examples above, wherein said objective lens is configured such that said optical system has a magnification of 8 (8×) or less.

155. The optical system of any of the Examples above, wherein said objective lens is configured such that said optical system has a magnification of 6 (6×) or less.

156. The optical system of any of the Examples above, wherein said objective lens is configured such that said optical system has a magnification of 5.5 (5.5×) or less.

157. The optical system of any of the Examples above, wherein said objective lens is configured such that said optical system has a magnification of 5 (5×) or less.

158. The optical system of any of the Examples above, wherein said at least one detection channel is configured to satisfy the Nyquist theorem for diffraction limited imaging.

159. The optical system of any of the Examples above, wherein said photodetector array comprises a plurality of pixels having a dimension that satisfies the Nyquist theorem for diffraction limited imaging.

160. The optical system of any of the Examples above, wherein said photodetector array comprises a plurality of pixels having a pixel size or pitch of less than 5 mm.

161. The optical system of any of the Examples above, wherein said photodetector array comprises a plurality of pixels having a pixel size or pitch of less than 4 mm.

162. The optical system of any of the Examples above, wherein said photodetector array comprises a plurality of pixels having a pixel size or pitch of 3 mm or less.

163. The optical system of any of the Examples above, wherein said photodetector array comprises a plurality of pixels having a pixel size or pitch 2.5 mm or less.

164. The optical system of any of the Examples above, wherein said photodetector array comprises a plurality of pixels having a pixel size or pitch in the range from 5 mm to 1 mm.

165. The optical system of any of the Examples above, wherein said photodetector array comprises a plurality of pixels having a pixel size or pitch in the range from 4 mm to 2 mm.

166. The optical system of any of the Examples above, wherein said photodetector array comprises a plurality of pixels having a pixel size or pitch in the range from 3 mm to 2 mm.

167. The optical system of any of the Examples above, wherein said optical system has a field of view of at least 1 mm wide.

168. The optical system of any of the Examples above, wherein said optical system has a field of view of at least 2 mm wide.

169. The optical system of any of the Examples above, wherein said optical system has a field of view of at least 3 mm wide.

170. The optical system of any of the Examples above, wherein said optical system has working distance in the range from 1 to 10 mm.

171. The optical system of any of the Examples above, wherein said optical system has working distance in the range from 2.5 to 5.5 mm.

172. The optical system of any of the Examples above, wherein optical system has working distance in the range from 3 to 5 mm.

173. The optical system of any of the Examples above, wherein said photodetector array has an active area having a diagonal of 10 mm or more.

174. The optical system of any of the Examples above, wherein said photodetector array has an active area having a diagonal of 13 mm or more.

175. The optical system of any of the Examples above, wherein said photodetector array has an active area having a diagonal of 14 mm or more.

176. The optical system of any of the Examples above, wherein said photodetector array has an active area having a diagonal of 15 mm or more.

177. The optical system of any of the Examples above, wherein said photodetector array has an active area having a diagonal in a range from 10 mm to 20 mm.

178. The optical system of any of the Examples above, wherein said photodetector array has an active area having a diagonal in a range from 12 mm to 18 mm.

179. The optical system of any of the Examples above, wherein said photodetector array has an active area having a diagonal in a range from 13 mm to 17 mm.

180. The optical system of any of the Examples above, wherein said photodetector array has an active area having a diagonal in a range from 14 mm to 17 mm.

181. The optical system of any of the Examples above, wherein said photodetector array has an active area having a diagonal in a range from 15 mm to 16 mm.

182. The optical system of any of the Examples above, further comprising a translation stage such that the sample support structure can be translated with respect to the objective lens, said optical system configured to capture multiple images such that the multiple images can be assembled to provide a view of the sample support structure that is larger than the field of view of the objective lens.

183. The optical system of any of the Examples above, further comprising a translation stage such that the sample support structure can be translated with respect to the objective lens, said optical system configured to capture multiple images such that the multiple images can be tiled to provide a view of the sample support structure that is larger than the field of view of the objective lens.

184. The optical system of any of the Examples above, further comprising a translation stage such that the sample support structure can be translated with respect to the objective lens and electronics configured to cause multiple images to be captured by the photodetector array and to assemble said multiple images to provide a view of the sample support structure that is larger than the field of view of the objective lens.

185. The optical system of any of the Examples above, wherein said optical system is capable of capturing with said photodetector array an image of fluorescence emitting sample sites on first and second surfaces on said sample support structure.

186. The optical system of any of the Examples above, wherein said optical system is configured to correct aberrations introduced by a layer through which said first and second surfaces of said sample support structure are imaged at the same time.

187. The optical system of any of the Examples above, wherein said optics in said detection channel is configured to correct aberrations introduced by a layer through which said sample on said sample support structure is imaged.

188. The optical system of any of the Examples above, wherein said optics comprises a tube lens configured to correct aberrations introduced by a layer through which said sample on said sample support structure is imaged.

189. The optical system of any of the Examples above, wherein said objective lens is configured to correct aberrations introduced by a layer through which said sample on said sample support structure is imaged.

190. The optical system of any of the Examples above, wherein said optics in said detection channel is configured to correct aberrations introduced by a layer through which said first and second surfaces on said sample support structure is imaged.

191. The optical system of any of the Examples above, wherein said optics comprises a tube lens configured to correct aberrations introduced by a layer through which said first and second surfaces on said sample support structure is imaged.

192. The optical system of any of the Examples above, wherein said objective lens is configured to correct aberrations introduced by a layer through which said first and second surfaces on said sample support structure is imaged.

193. The optical system of any of the Examples above, wherein said layer is between 0.1 mm to 1.5 mm thick.

194. The optical system of any of the Examples above, wherein said layer has a thickness in the range from 0.15 mm to 1.3 mm thick.

195. The optical system of any of the Examples above, wherein said layer has a thickness in the range from 0.5 mm to 1.3 mm thick.

196. The optical system of the Examples above, wherein said layer has a thickness in the range from 0.75 mm to 1.25 mm thick.

197. The optical system of any of the Examples above, wherein said layer comprises glass.

198. The optical system of any of the Examples above, wherein said layer comprises a glass plate.

199. The optical system of any of the Examples above, wherein said layer comprises a cover slip.

200. The optical system of any of the Examples above, wherein said layer comprises quartz.

201. The optical system of any of the Examples above, wherein said layer comprises plastic.

202. The optical system of any of the Examples above, wherein
said optical system is capable of capturing with said photodetector array images of fluorescence emitting sample sites on first and second surfaces on said sample support structure; and
said optical system is configured to image capture an image of said first and second surface at the same time.

203. The optical system of any of the Examples above, wherein said one or more objective lenses has a numerical aperture of less than 0.6.

204. The optical system of any of the Examples above, having a depth of field of at least 0.075 mm.

205. The optical system of any of the Examples, wherein said optical system is capable of capturing with said photodetector array an image of emission emitting sample sites on first and second surfaces on said sample support structure, said first surface between said objective lens and said second surface, said first separated from each other by at least 0.075 mm.

206. The optical system of any of the Examples above, wherein said optical system is capable of capturing with said photodetector array an image of emission emitting sample sites on first and second planar surfaces on said sample support structure, said first separated from each other by at least 0.075 mm along a direction normal to said first and second planar surfaces.

207. The optical system of any of the Examples above, wherein said optical system is capable of capturing with said photodetector array an image of emission emitting sample sites on first and second surfaces on said sample support structure, said disposed objective above both said first and said second surfaces, said first surface disposed above said second surface by at least 0.075 mm.

208. The optical system of any of the Examples above, wherein said optical system is capable of capturing with said photodetector array an image of emission emitting sample sites on first and second surfaces on said sample support structure, wherein said objective lens has an optical axis and said first and second surfaces are separated from each other by at least 0.075 mm along the direction of said optical axis.

209. The optical system of any of the Examples above, wherein the optical system has a field of view of at least 1 mm.

210. The optical system of any of the Examples above, further comprising a sample support structure configured to produce a contrast-to-noise ratio greater than 20.

211. The optical system of any of the Examples above, further comprising a sample support structure having at least one surface comprise a hydrophilic coating.

212. The optical system of any of the Examples above, wherein the optical system is configured for high throughput assays.

213. The optical system of any of the Examples above, wherein the objective lens is disposed to receive the excitation beam, direct the excitation beam to the sample support structure.

214. The optical system of any of the Examples above, further comprising a dichroic filter disposed to reflect the excitation beam into the objective lens and to transmit the emission light to the at least one detection channel.

215. The optical system of any one of the Examples above, wherein no optical element enters an optical path between the sample support structure and a photodetector array in said at least one detection channels in order to form an image of fluorescing sample sites on said a first surface of said sample support structure onto the photodetector array and exits said optical path to form an image of fluorescing sample sites on said second surface of said sample support structure onto the photodetector array.

216. The optical system of any one of the Examples above, wherein no optical compensation is used to form an image of fluorescing sample sites on a first surface of said sample support structure onto the photodetector array that is not identical to optical compensation used to form an image of fluorescing sample sites on a second surface of said sample support structure onto the photodetector array.

217. The optical system of any one of the Examples above, wherein no optical element in an optical path between the sample support structure and a photodetector array in said at least one detection channels is adjusted differently to form an image of fluorescing sample sites on a first surface of said sample support structure onto the photodetector array than to form an image of fluorescing sample sites on a second surface of said sample support structure onto the photodetector array.

218. The optical system of any one of the Examples above, wherein no optical element in an optical path between the sample support structure and a photodetector array in said at least one detection channels is moved a different amount or a different direction to form an image of fluorescing sample sites on said a first surface of said sample support structure onto the photodetector array than to form an image of fluorescing sample sites on a second surface of said sample support structure onto the photodetector array.

219. The optical system of any of Examples 213 to 216, wherein first and second surfaces on said sample support structure are separated from each other by at least 0.075 mm.

220. The optical system of any of Examples 213 to 216, wherein first surface is between said objective lens and said second surface.

221. The optical system of any of Examples 213 to 217, wherein said first and second surfaces are planar surfaces and said first is separated from each other along a direction normal to said first and second planar surfaces.

222. The optical system of any of Examples 213 to 217, wherein said first and second surfaces are planar surfaces and said first is separated from each other by at least 0.075 mm along a direction normal to said first and second planar surfaces.

223. The optical system of any of Examples 213 to 217, wherein said objective is disposed above both said first and said second surfaces and said first surface is disposed above said second surface.

224. The optical system of any of Examples 213 to 217, wherein said objective is disposed above both said first and said second surfaces and said first surface is disposed above said second surface by at least 0.075 mm.

225. The optical system of any of Examples 213 to 217, wherein said objective lens has an optical axis and said first and second surfaces are separated from each other along the direction of said optical axis.

226. The optical system of any of Examples 213 to 217, wherein said objective lens has an optical axis and said first and second surfaces are separated from each other by at least 0.075 mm along the direction of said optical axis.

227. The optical system of any one of the Examples 213-224, wherein said first and second surfaces are both configured to provide a contrast-to-noise ratio of greater than 20 for a single sequencing cycle.

228. The optical system of any one of the Examples 213-224, wherein said first and second surfaces are both configured to provide a contrast-to-noise ratio greater than 20 for 5 consecutive sequencing cycles.

229. The optical system of any one of the Examples 213-224, wherein said first and second surfaces are both configured to provide a contrast-to-noise ratio of greater than 20 for 10 consecutive sequencing cycles.

230. The optical system of any one of the Examples 213-224, wherein said first and second surfaces both comprise hydrophilic surfaces comprising labeled nucleic acid colonies at a density of at least 10000/mm², and are configured to provide a contrast-to-noise ratio of at least 20.

231. The optical system of any one of the Examples above, wherein said objective lens has a numerical aperture less than 0.55.

232. The optical system of any one of the Examples above, wherein said objective lens has a numerical aperture less than 0.5.

233. The optical system of any one of the Examples above, wherein said objective lens has a numerical aperture less than 0.45.

234. The optical system of any one of the Examples above, wherein said objective lens has a numerical aperture less than 0.4.

235. The optical system of any one of the Examples above, further comprising one or more tube lenses.

236. The optical system of any one of the Examples above, further comprising one or more tube lenses in said at least one detection channel.

237. The optical system of any one of the Examples above, wherein objective lens and said at least one detection channel provide a field of view of at least 1 mm.

238. The optical system of any one of the Examples above, wherein said sample support structure is configured to provide a contrast-to-noise ratio of greater than 20 for a single sequencing cycle.

239. The optical system of any one of the Examples above, wherein said sample support structure is configured to provide a contrast-to-noise ratio greater than 20 for 5 consecutive sequencing cycles.

240. The optical system of any one of the Examples above, wherein said sample support structure is configured to provide a contrast-to-noise ratio of greater than 20 for 10 consecutive sequencing cycles.

241. The optical system of any one of the Examples above, wherein said sample support structure comprises a hydrophilic substrate comprising labeled nucleic acid colonies at a density of at least 10000/mm², and is configured to provide a contrast-to-noise ratio of at least 20.

242. The optical system of any one of the Examples above, wherein said fluorescing sample sites comprise nucleic acid colonies comprising 1, 2, 3, or 4 distinct detectable labels.

243. An optical system of any one of the Examples above, wherein said at least one detecting channel comprise imaging channels to detect 1, 2, 3, or 4 distinct labels.

244. A method of sequencing a nucleic acid comprising binding or sequencing by synthesis reaction on one or more surfaces of said sample support structure, and detecting a bound or incorporated base using the optical system of any of the Examples above.

245. A method of determining a genotype of a sample comprising a nucleic acid molecule, comprising preparing said nucleic acid molecule for sequencing, and then sequencing said nucleic acid molecule using the optical system of any of the Examples above.

246. An optical system of any one of the Examples above, wherein the sample support structure comprises one, two, three, four, five, or six imaging surfaces comprising fluorescing sample sites.

247. An optical system of any one of the Examples above, wherein the sample support structure comprises a flow cell.

248. An optical system of any one of the Examples above, wherein the sample support structure comprises a sample chamber.

249. An optical system of any one of the Examples above, further comprising said sample support structure.

250. The optical system of any of the Examples above, wherein said objective lens has a numerical aperture in the range between 0.5 to 0.4.

251. The optical system of any of the Examples above, wherein optical system has an optical resolution in a range from 500 to 1000 nm.

252. The optical system of any of the Examples above, having an optical resolution in a range from 600 to 900 nm.

253. The optical system of any of the Examples above, wherein optical system has an optical resolution in a range from 650 to 850 nm.

254. The optical system of any of the Examples above, further comprising said sample support structure.

255. The optical system of any of the Examples above, further comprising said sample support structure having said first and second surfaces.

256. The optical system of any of the Examples above, further comprising the sample support structure, said sample support structure comprising a flow cell.

257. The optical system of any of the Examples above, further comprising the sample support structure comprising a flow cell having a flow channel and said first and second surfaces comprise interior surfaces of said flow cell configured to be in contact with a sample flowing through said flow cell.

258. The optical system of any of the Examples above, configured for DNA sequencing.

259. The optical system of any of the Examples above, comprising four channels configured to capture images at four different spectral regions.

260. The optical system of any of the Examples above, comprising electronics configured to process images captured by a plurality of optical channels to obtain information from the fluorescing sample sites.

261. The optical system of any of the Examples above, comprising electronics configured to process images captured by a plurality of optical channels to obtain information from the fluorescing sample sites based on their locations.

262. The optical system of any of the Examples above, wherein said optical system comprises a sequencer.

263. The optical system of any of the Examples above, wherein said optical system comprises a nucleic acid sequencing apparatus.

264. The optical system of any of the Examples above, further comprising a fluid flow controller configured to control the flow of fluid through a flow cell or sample support structure.

265. The optical system of any of the Examples above, further comprising conduits configured to flow fluid through a flow cell or sample support structure.

266. The optical system of any of the Examples above, further comprising tubing that provides fluid communication with a fluid flow control system to provide fluid to a flow cell or sample support structure.

267. The optical system of any of the Examples above, further comprising a fluid flow controller configured to provide programmable control of fluid flow velocity, volumetric fluid flow rate, the timing of reagent or buffer introduction, or any combination thereof.

268. The optical system of any of the Examples above, further comprising a temperature controller configured to control the temperature of a flow cell or sample support structure.

269. The optical system of any of the Examples above, further comprising a reagent reservoir configured to provide reagent to the flow cell or sample support structure.

270. The optical system of any of the Examples above, further comprising a replaceable reagent configured to provide reagent to the flow cell or sample support structure.

271. The optical system of any of the Examples above, further comprising motorized translation stage configured to move the flow cell or sample support structure.

272. The optical system of any of the Examples above, further comprising motor controllers to control movement of a translation stage configured to move the flow cell or sample support structure.

273. The optical system of any of the Examples above, wherein said optical system comprises a nucleic sequencing apparatus.

### Part VII

1. An optical system comprising:
a) at least one light source configured to provide excitation light within one or more specified wavelength ranges;
b) an objective lens configured to collect fluorescence arising from within a specified field-of-view of a sample plane upon exposure of the sample plane to the excitation light, wherein a numerical aperture of the objective lens is at least 0.3, wherein a working distance of the objective lens is at least 700 μm, and wherein the field-of-view has an area of at least 1 mm²; and
c) at least one image sensor, wherein the fluorescence collected by the objective lens is imaged onto the image sensor, and wherein a pixel dimension for the image sensor is chosen such that a spatial sampling frequency for the optical system is at least twice an optical resolution of the optical system.
   2. The optical system of Example 1, wherein the numerical aperture is at least 0.75.
   3. The optical system of Example 1, wherein the numerical aperture is at least 1.0.
   4. The optical system of any one of Examples 1 to 3, wherein the working distance is at least 850 μm.
   5. The optical system of any one of Examples 1 to 3, wherein the working distance is at least 1,000 μm.
   6. The optical system of any one of Examples 1 to 5, wherein the field-of-view has an area of at least 2.5 mm².
   7. The optical system of any one of Examples 1 to 5, wherein the field-of-view has an area of at least 3 mm².
   8. The optical system of any one of Examples 1 to 5, wherein the spatial sampling frequency is at least 2.5 times the optical resolution of the optical system.
   9. The optical system of any one of Examples 1 to 5, wherein the spatial sampling frequency is at least 3 times the optical resolution of the optical system.
   10. The optical system of any one of Examples 1 to 9, further comprising an X-Y-Z translation stage such that the system is configured to acquire a series of two or more fluorescence images in an automated fashion, wherein each image of the series is acquired for a different field-of-view.
   11. The optical system of Examples 10, wherein a position of the sample plane is simultaneously adjusted in an X direction, a Y direction, and a Z direction to match the position of an objective lens focal plane in between acquiring images for different fields-of-view.
   12. The optical system of Examples 11, wherein the time required for the simultaneous adjustments in the X direction, Y direction, and Z direction is less than 0.4 seconds.
   13. The optical system of any one of Examples 10 to 12, further comprising an autofocus mechanism configured to adjust the focal plane position prior to acquiring an image of a different field-of-view if an error signal indicates that a difference in the position of the focal plane and the sample plane in the Z direction is greater than a specified error threshold.
   14. The optical system of Example 13, wherein the specified error threshold is 100 nm.
   15. The optical system of Example 13, wherein the specified error threshold is 50 nm.
   16. The optical system of any one of Examples 1 to 15, wherein the system comprises three or more image sensors, and wherein the system is configured to image fluorescence in each of three or more wavelength ranges onto a different image sensor.
   17. The optical system of Example 16, wherein a difference in the position of a focal plane for each of the three or more image sensors and the sample plane is less than 100 nm.
   18. The optical system of Example 16, wherein a difference in the position of a focal plane for each of the three or more image sensors and the sample plane is less than 50 nm.
   19. The optical system of any one of Examples 10 to 18, wherein the total time required to reposition the sample plane, adjust focus if necessary, and acquire an image is less than 0.4 seconds per field-of-view.
   20. The optical system of any one of Examples 10 to 18, wherein the total time required to reposition the sample plane, adjust focus if necessary, and acquire an image is less than 0.3 seconds per field-of-view.
   21. A optical system for dual-side imaging of a flow cell comprising:
a) an objective lens configured to collect fluorescence arising from within a specified field-of-view of a sample plane within the flow cell;
b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm and a gap between an upper interior surface and a lower interior surface of at least 50 μm;
wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.
   22. The optical system of Example 21, wherein the objective lens is a commercially-available microscope objective.
   23. The optical system of Example 22, wherein the commercially-available microscope objective has a numerical aperture of at least 0.3.
   24. The optical system of any one of Examples 21 to 23, wherein the objective lens has a working distance of at least 700 μm.
   25. The optical system of any one of Examples 21 to 24, wherein the objective lens is corrected to compensate for a cover slip thickness (or flow cell wall thickness) of 0.17 mm.
   26. The optical system of any one of Examples 21 to 25, further comprising an electro-optical phase plate positioned adjacent to the objective lens and between the objective lens and the tube lens, wherein the electro-optical phase plate provides correction for optical aberrations caused by a fluid filling the gap between the upper interior surface and the lower interior surface of the flow cell.
   27. The optical system of any one of Examples 21 to 26, wherein the at least one tube lens is a compound lens comprising three or more optical components.
   28. The optical system of any one of Examples 21 to 27, wherein the at least one tube lens is a compound lens comprising four optical components.
   29. The optical system of Example 28, wherein the four optical components comprise, in order, a first asymmetric convex-convex lens, a second convex-plano lens, a third asymmetric concave-concave lens, and a fourth asymmetric convex-concave lens.
   30. The optical system of any one of Examples 21 to 29, wherein the at least one tube lens is configured to correct an imaging performance metric for a combination of the objective lens, the at least one tube lens, and the at least one image sensor when imaging an interior surface of a flow cell having a wall thickness of at least 1 mm.
   31. The optical system of any one of Examples 21 to 30, wherein the at least one tube lens is configured to correct an imaging performance metric for a combination of the objective lens, the at least one tube lens, and the at least one image sensor when imaging an interior surface of a flow cell having a gap of at least 100 μm.
   32. The optical system of any one of Examples 21 to 31, wherein the at least one tube lens is configured to correct an imaging performance metric for a combination of the objective lens, the at least one tube lens, and the at least one image sensor when imaging an interior surface of a flow cell having a gap of at least 200 μm.
   33. The optical system of any one of Examples 21 to 32, wherein the system comprises a single objective lens, two tube lenses, and two image sensors, and each of the two tube lenses is designed to provide optimal imaging performance at a different fluorescence wavelength.
   34. The optical system of any one of Examples 21 to 32, wherein the system comprises a single objective lens, three tube lenses, and three image sensors, and each of the three tube lenses is designed to provide optimal imaging performance at a different fluorescence wavelength.
   35. The optical system of any one of Examples 21 to 32, wherein the system comprises a single objective lens, four tube lenses, and four image sensors, and each of the four tube lenses is designed to provide optimal imaging performance at a different fluorescence wavelength.
   36. The optical system of any one of Examples 21 to 35, wherein the design of the objective lens or the at least one tube lens is configured to optimize the modulation transfer function in the mid to high spatial frequency range.
   37. The optical system of any one of Examples 21 to 36, wherein the imaging performance metric comprises at least one of a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof.
   38. The optical system of any one of Examples 21 to 37, wherein the difference in the imaging performance metric for imaging the upper interior surface and the lower interior surface of the flow cell is less than 10%.
   39. The optical system of any one of Examples 21 to 38, wherein the difference in imaging performance metric for imaging the upper interior surface and the lower interior surface of the flow cell is less than 5%.
   40. The optical system of any one of Examples 21 to 39, wherein the use of the at least one tube lens provides for an at least equivalent or better improvement in the imaging performance metric for dual-side imaging compared to that for a conventional system comprising an objective lens, a motion-actuated compensator, and an image sensor.
   41. The optical system of any one of Examples 21 to 40, wherein the use of the at least one tube lens provides for an at least 10% improvement in the imaging performance metric for dual-side imaging compared to that for a conventional system comprising an objective lens, a motion-actuated compensator, and an image sensor.
   42. An illumination system for use in imaging-based solid-phase genotyping and sequencing applications, the illumination system comprising:
a) a light source; and
b) a liquid light-guide configured to collect light emitted by the light source and deliver it to a specified field-of-illumination on a support surface comprising tethered biological macromolecules.
   43. The illumination system of Example 42, further comprising a condenser lens.
   44. The illumination system of Example 42 or Example 43, wherein the specified field-of-illumination has an area of at least 2 mm².
   45. The illumination system of any one of Examples 42 to 44, wherein the light delivered to the specified field-of-illumination is of uniform intensity across a specified field-of-view for an imaging system used to acquire images of the support surface.
   46. The illumination system of Example 45, wherein the specified field-of-view has an area of at least 1 mm².
   47. The illumination system of Example 45, wherein the light delivered to the specified field-of-illumination is of uniform intensity across the specified field-of-view when a coefficient of variation (CV) for light intensity is less than 10%.
   48. The illumination system of Example 45, wherein the light delivered to the specified field-of-illumination is of uniform intensity across the specified field-of-view when a coefficient of variation (CV) for light intensity is less than 5%.
   49. The illumination system of any one of Examples 42 to 48, wherein the light delivered to the specified field-of-illumination has a speckle contrast value of less than 0.1.
   50. The illumination system of any one of Examples 42 to 49, wherein the light delivered to the specified field-of-illumination has a speckle contrast value of less than 0.05.
   274. An optical system comprising:
a light source configured to emit an excitation beam;
an objective lens disposed to receive the excitation beam, direct the excitation beam to a specimen, and receive emission light in response to the excitation beam; and
a plurality of channels comprising optics configured to receive at least a portion of the emission light,
wherein different channels comprise different lens elements such that said different channels are configured for different wavelengths.
   52. The optical system of Examples 51, wherein lens elements in the different channels have different shapes, materials, thicknesses, spacings or any combination thereof such that the different channels are configured for different respective wavelengths.
   53. The optical system of Examples 51 or 52, wherein lens elements in the different channels have different shapes, materials, thicknesses, spacings or any combination thereof such that the different channels are configured so as to reduce aberration for different respective wavelengths of the respective channels.
   54. The optical system of any of Examples 51-53, wherein lens elements in the different channels are optimized for the different wavelengths of the respective different channels.
   55. The optical system of any of Examples 51-54, wherein said lens elements are included in at least one tube lens.
   56. The optical system of Example 55, wherein the at least one tube lens comprises four lens elements.
   57. The optical system of Example 56, wherein the four lens elements comprise a first asymmetric convex-convex lens, a second convex-plano lens, a third asymmetric concave-concave lens, and a fourth asymmetric convex-concave lens.
   58. The optical system of Example 56, wherein the four lens elements comprise, in order, a first asymmetric convex-convex lens, a second convex-plano lens, a third asymmetric concave-concave lens, and a fourth asymmetric convex-concave lens.
   59. An optical system comprising:
a) at least one light source configured to provide excitation light within one or more specified wavelength ranges;
b) an objective lens configured to collect fluorescence arising from within a specified field-of-view of a sample plane upon exposure of the sample plane to the excitation light; and
c) at least one image sensor, wherein the fluorescence collected by the objective lens is imaged onto the image sensor, and wherein a pixel dimension for the image sensor is chosen such that a spatial sampling frequency for the optical system is at least twice an optical resolution of the optical system.
   60. An optical system comprising:
a) at least one light source configured to provide excitation light within one or more specified wavelength ranges;
b) an objective lens configured to collect fluorescence arising from within a specified field-of-view of a sample plane upon exposure of the sample plane to the excitation light, wherein the field-of-view has an area of at least 1 mm²; and
c) at least one image sensor, wherein the fluorescence collected by the objective lens is imaged onto the image sensor.
   61. The optical system of Example 60, wherein a numerical aperture of the objective lens is at least 0.3.
   62. The optical system of Example 60 or 61, wherein a working distance of the objective lens is at least 700 μm.
   63. An optical system comprising:
a) at least one light source configured to provide excitation light within one or more specified wavelength ranges;
b) an objective lens configured to collect fluorescence arising from within a specified field-of-view of a sample plane upon exposure of the sample plane to the excitation light, wherein a numerical aperture of the objective lens is at least 0.3; and
c) at least one image sensor, wherein the fluorescence collected by the objective lens is imaged onto the image sensor.
   64. The optical system of Example 63, wherein the field-of-view has an area of at least 1 mm².
   65. The optical system of Example 63 or 64, wherein a working distance of the objective lens is at least 700 μm.
   66. An optical system comprising:
a) at least one light source configured to provide excitation light within one or more specified wavelength ranges;
b) an objective lens configured to collect fluorescence arising from within a specified field-of-view of a sample plane upon exposure of the sample plane to the excitation light, wherein a working distance of the objective lens is at least 700 μm; and
c) at least one image sensor, wherein the fluorescence collected by the objective lens is imaged onto the image sensor.
   67. The optical system of Example 66, wherein the field-of-view has an area of at least 1 mm².
   68. The optical system of Example 66 or 67, a numerical aperture of the objective lens is at least 0.3.
   69. An optical system comprising:
a) at least one light source configured to provide excitation light within one or more specified wavelength ranges;
b) an objective lens configured to collect fluorescence arising from within a specified field-of-view of a sample plane upon exposure of the sample plane to the excitation light; and
c) at least one image sensor, wherein the fluorescence collected by the objective lens is imaged onto the image sensor; and
d) an X-Y-Z translation stage such that the system is configured to acquire a series of two or more fluorescence images in an automated fashion,
wherein a position of the sample plane is simultaneously adjusted in an X direction, a Y direction, and a Z direction to match the position of an objective lens focal plane in between acquiring images for different fields-of-view.
   70. The optical system of Example 69, wherein the time required for the simultaneous adjustments in the X direction, Y direction, and Z direction is less than 0.4 seconds.
   71. The optical system of Example 69 or 70, further comprising an autofocus mechanism configured to adjust the focal plane position prior to acquiring an image of a different field-of-view if an error signal indicates that a difference in the position of the focal plane and the sample plane in the Z direction is greater than a specified error threshold.
   72. The optical system of Example 71, wherein the specified error threshold is 100 nm.
   73. The optical system of Example 71, wherein the specified error threshold is 50 nm.
   74. An optical system comprising:
a) at least one light source configured to provide excitation light within one or more specified wavelength ranges;
b) an objective lens configured to collect fluorescence arising from within a specified field-of-view of a sample plane upon exposure of the sample plane to the excitation light; and
c) at least one image sensor, wherein the fluorescence collected by the objective lens is imaged onto the image sensor; and
d) an autofocus mechanism configured to adjust the focal plane position prior to acquiring an image of a different field-of-view if an error signal indicates that a difference in the position of the focal plane and the sample plane in the Z direction is greater than a specified error threshold,
   75. The optical system of Example 74, wherein the specified error threshold is 100 nm.
   76. The optical system of Example 74, wherein the specified error threshold is 50 nm.
   77. The optical system of any of Examples 74-76, further comprising an X-Y-Z translation stage such that the system is configured to acquire a series of two or more fluorescence images in an automated fashion.
   78. An optical system comprising:
a light source configured to emit an excitation beam;
an objective lens disposed to receive the excitation beam, direct the excitation beam to a specimen, and receive emission light in response to the excitation beam; and
a plurality of channels comprising optics configured to receive at least a portion of the emission light,
wherein as least one of the channels comprises a tube lens comprising four lens elements.
   79. The optical system of Example 78, wherein the four lens elements comprise a first asymmetric convex-convex lens, a second convex-plano lens, a third asymmetric concave-concave lens, and a fourth asymmetric convex-concave lens.
   80. The optical system of Example 78, wherein the four lens elements comprise, in order, a first asymmetric convex-convex lens, a second convex-plano lens, a third asymmetric concave-concave lens, and a fourth asymmetric convex-concave lens.
   81. The optical system of any of the Examples above, wherein said optical system comprises a sequencer.
   82. The optical system of any of the Examples above, wherein said optical system comprises a nucleic acid sequencing apparatus.
   83. The optical system of any of the Examples above, further comprising a fluid flow controller configured to control the flow of fluid through a flow cell or sample support structure.
   84. The optical system of any of the Examples above, further comprising conduits configured to flow fluid through a flow cell or sample support structure.
   85. The optical system of any of the Examples above, further comprising tubing that provides fluid communication with a fluid flow control system to provide fluid to a flow cell or sample support structure.
   86. The optical system of any of the Examples above, further comprising a fluid flow controller configured to provide programmable control of fluid flow velocity, volumetric fluid flow rate, the timing of reagent or buffer introduction, or any combination thereof.
   87. The optical system of any of the Examples above, further comprising a temperature controller configured to control the temperature of a flow cell or sample support structure.
   88. The optical system of any of the Examples above, further comprising a reagent reservoir configured to provide reagent to the flow cell or sample support structure.
   89. The optical system of any of the Examples above, further comprising a replaceable reagent configured to provide reagent to the flow cell or sample support structure.
   90. The optical system of any of the Examples above, further comprising motorized translation stage configured to move the flow cell or sample support structure.
   91. The optical system of any of the Examples above, further comprising motor controllers to control movement of a translation stage configured to move the flow cell or sample support structure.
   92. The optical system of any of the Examples above, wherein said optical system comprises a nucleic sequencing apparatus.

Disclosed herein are optical systems comprising: a) at least one light source configured to provide excitation light within one or more specified wavelength ranges; b) an objective lens configured to collect fluorescence arising from within a specified field-of-view of a sample plane upon exposure of the sample plane to the excitation light, wherein a numerical aperture of the objective lens is at least 0.3, wherein a working distance of the objective lens is at least 700 μm, and wherein the field-of-view has an area of at least 1 mm²; and c) at least one image sensor, wherein the fluorescence collected by the objective lens is imaged onto the image sensor, and wherein a pixel dimension for the image sensor is chosen such that a spatial sampling frequency for the optical system is at least twice an optical resolution of the optical system.

In some embodiments, the numerical aperture is at least 0.75. In some embodiments, the numerical aperture is at least 1.0. In some embodiments, the working distance is at least 850 μm. In some embodiments, the working distance is at least 1,000 μm. In some embodiments, the field-of-view has an area of at least 2.5 mm². In some embodiments, the field-of-view has an area of at least 3 mm². In some embodiments, the spatial sampling frequency is at least 2.5 times the optical resolution of the optical system. In some embodiments, the spatial sampling frequency is at least 3 times the optical resolution of the optical system. In some embodiments, the system further comprises an X-Y-Z translation stage such that the system is configured to acquire a series of two or more fluorescence images in an automated fashion, wherein each image of the series is acquired for a different field-of-view. In some embodiments, a position of the sample plane is simultaneously adjusted in an X direction, a Y direction, and a Z direction to match the position of an objective lens focal plane in between acquiring images for different fields-of-view. In some embodiments, the time required for the simultaneous adjustments in the X direction, Y direction, and Z direction is less than 0.4 seconds. In some embodiments, the system further comprises an autofocus mechanism configured to adjust the focal plane position prior to acquiring an image of a different field-of-view if an error signal indicates that a difference in the position of the focal plane and the sample plane in the Z direction is greater than a specified error threshold. In some embodiments, the specified error threshold is 100 nm. In some embodiments, the specified error threshold is 50 nm. In some embodiments, the system comprises three or more image sensors, and wherein the system is configured to image fluorescence in each of three or more wavelength ranges onto a different image sensor. In some embodiments, a difference in the position of a focal plane for each of the three or more image sensors and the sample plane is less than 100 nm. In some embodiments, a difference in the position of a focal plane for each of the three or more image sensors and the sample plane is less than 50 nm. In some embodiments, the total time required to reposition the sample plane, adjust focus if necessary, and acquire an image is less than 0.4 seconds per field-of-view. In some embodiments, the total time required to reposition the sample plane, adjust focus if necessary, and acquire an image is less than 0.3 seconds per field-of-view.

Also discloser herein are optical systems for dual-side imaging of a flow cell comprising: a) an objective lens configured to collect fluorescence arising from within a specified field-of-view of a sample plane within the flow cell; b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm and a gap between an upper interior surface and a lower interior surface of at least 50 μm; wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.

In some embodiments, the objective lens is a commercially-available microscope objective. In some embodiments, the commercially-available microscope objective has a numerical aperture of at least 0.3. In some embodiments, the objective lens has a working distance of at least 700 μm. In some embodiments, the objective lens is corrected to compensate for a cover slip thickness (or flow cell wall thickness) of 0.17 mm. In some embodiments, the optical system further comprising an electro-optical phase plate positioned adjacent to the objective lens and between the objective lens and the tube lens, wherein the electro-optical phase plate provides correction for optical aberrations caused by a fluid filling the gap between the upper interior surface and the lower interior surface of the flow cell. In some embodiments, the at least one tube lens is a compound lens comprising three or more optical components. In some embodiments, the at least one tube lens is a compound lens comprising four optical components. In some embodiments, the four optical components comprise, in order, a first asymmetric convex-convex lens, a second convex-plano lens, a third asymmetric concave-concave lens, and a fourth asymmetric convex-concave lens. In some embodiments, the at least one tube lens is configured to correct an imaging performance metric for a combination of the objective lens, the at least one tube lens, and the at least one image sensor when imaging an interior surface of a flow cell having a wall thickness of at least 1 mm. In some embodiments, the at least one tube lens is configured to correct an imaging performance metric for a combination of the objective lens, the at least one tube lens, and the at least one image sensor when imaging an interior surface of a flow cell having a gap of at least 100 μm. In some embodiments, the at least one tube lens is configured to correct an imaging performance metric for a combination of the objective lens, the at least one tube lens, and the at least one image sensor when imaging an interior surface of a flow cell having a gap of at least 200 μm. In some embodiments, the system comprises a single objective lens, two tube lenses, and two image sensors, and each of the two tube lenses is designed to provide optimal imaging performance at a different fluorescence wavelength. In some embodiments, the system comprises a single objective lens, three tube lenses, and three image sensors, and each of the three tube lenses is designed to provide optimal imaging performance at a different fluorescence wavelength. In some embodiments, the system comprises a single objective lens, four tube lenses, and four image sensors, and each of the four tube lenses is designed to provide optimal imaging performance at a different fluorescence wavelength. In some embodiments, the design of the objective lens or the at least one tube lens is configured to improve or optimize the modulation transfer function in the mid to high spatial frequency range. In some embodiments, the imaging performance metric comprises a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof. In some embodiments, the difference in the imaging performance metric for imaging the upper interior surface and the lower interior surface of the flow cell is less than 10%. In some embodiments, the difference in imaging performance metric for imaging the upper interior surface and the lower interior surface of the flow cell is less than 5%. In some embodiments, the use of the at least one tube lens provides for an at least equivalent or better improvement in the imaging performance metric for dual-side imaging compared to that for a conventional system comprising an objective lens, a motion-actuated compensator, and an image sensor. In some embodiments, the use of the at least one tube lens provides for an at least 10% improvement in the imaging performance metric for dual-side imaging compared to that for a conventional system comprising an objective lens, a motion-actuated compensator, and an image sensor.

Disclosed herein are illumination systems for use in imaging-based solid-phase genotyping and sequencing applications, the illumination system comprising: a) a light source; and b) a liquid light-guide configured to collect light emitted by the light source and deliver it to a specified field-of-illumination on a support surface comprising tethered biological macromolecules.

In some embodiments, the illumination system further comprises a condenser lens. In some embodiments, the specified field-of-illumination has an area of at least 2 mm². In some embodiments, the light delivered to the specified field-of-illumination is of uniform intensity across a specified field-of-view for an imaging system used to acquire images of the support surface. In some embodiments, the specified field-of-view has an area of at least 1 mm². In some embodiments, the light delivered to the specified field-of-illumination is of uniform intensity across the specified field-of-view when a coefficient of variation (CV) for light intensity is less than 10%. In some embodiments, the light delivered to the specified field-of-illumination is of uniform intensity across the specified field-of-view when a coefficient of variation (CV) for light intensity is less than 5%. In some embodiments, the light delivered to the specified field-of-illumination has a speckle contrast value of less than 0.1. In some embodiments, the light delivered to the specified field-of-illumination has a speckle contrast value of less than 0.05.

### Part VIII

1. A flow cell device, comprising:
(a) a first reservoir housing a first solution and having an inlet end and an outlet end, wherein the first agent flows from the inlet end to the outlet end in the first reservoir;
(b) a second reservoir housing a second solution and having an inlet end and an outlet end, wherein the second agent flows from the inlet end to the outlet end in the second reservoir;
(c) a central region having an inlet end fluidically coupled to the outlet end of the first reservoir and the outlet end of the second reservoir through at least one valve;
wherein the volume of the first solution flowing from the outlet of the first reservoir to the inlet of the central region is less than the volume of the second solution flowing from the outlet of the second reservoir to the inlet of the central region.

2. The device of Example 1, wherein the first solution is different from the second solution.

3. The device of Example 1, wherein the second solution comprises at least one reagent common to a plurality of reactions occurring in the central region.

4. The device of Example 1, wherein the second solution comprises at least one reagent selected from the list consisting of a solvent, a polymerase, and a dNTP.

5. The device of Example 1, wherein the second solution comprise low cost reagents.

6. The device of Example 1 wherein the first reservoir is fluidically coupled to the central region through a first valve and the second reservoir is fluidically coupled to the central region through a second valve.

7. The device of Example 1, wherein the valve is a diaphragm valve.

8. The device of Example 1, wherein the first solution comprises a reagent and the second solution comprises a reagent and the reagent in the first solution is more expensive than the reagent in the second solution.

9. The device of Example 1, wherein the first solution comprises a reaction-specific reagent and the second solution comprises nonspecific reagent common to all reaction occurring in the central region, and wherein the reaction specific reagent is more expensive than the nonspecific reagent.

10. The device of Example 1, wherein the first reservoir is positioned in close proximity to the inlet of the central region to reduce dead volume for delivery of the first solutions.

11. The device of Example 1, wherein the first reservoir is places closer to the inlet of the central region than the second reservoir.

12. The device of Example 1, wherein the reaction-specific reagent is configured in close proximity to the second diaphragm valve so as to reduce dead volume relative to delivery of the plurality of nonspecific reagents from the plurality of reservoirs to the first diaphragm valve.

13. The device of Example 1, wherein the central region comprises a capillary tube.

14. The device of Example 13, wherein the capillary tube is an off-shelf product.

15. The device of Example 13, wherein the capillary tube is removable from the device.

16. The device of Example 13, wherein the capillary tube comprises an oligonucleotide population directed to sequence a eukaryotic genome.

17. The device of Example 1, wherein the central region comprises a microfluidic chip.

18. The device of Example 17, wherein the microfluidic chip comprises a single etched layer.

19. The device of Example 17, wherein the microfluidic chip comprises at least one chip channel.

20. The device of Example 19, wherein the channel has an average depth in the range of 50 to 300 μm.

21. The device of Example 19, wherein the channel has an average length in the range of 1 to 200 mm.

22. The device of Example 19, wherein the channel has an average width in the range of 0.1 to 30 mm.

23. The device of Example 19, wherein the channel is formed by laser irradiation.

24. The device of Example 17, wherein the microfluidic chip comprises one etched layer.

25. The device of Example 17, wherein the microfluidic chip comprises one non-etched layer, and wherein the etched layer is bond with the non-etched layer.

26. The device of Example 17, wherein the microfluidic chip comprises two non-etched layers, and wherein the etched layer is positioned between the two non-etched layers.

27. The device of Example 17, wherein the microfluidic chip comprises at least two bonded layers.

28. The device of Example 17, wherein the microfluidic chip comprises quartz.

29. The device of Example 17, wherein the microfluidic chip comprises borosilicate glass.

30. The device of Example 19, wherein the chip channel comprises an oligonucleotide population directed to sequence a prokaryotic genome.

31. The device of Example 19, wherein the chip channel comprises an oligonucleotide population directed to sequence a transcriptome.

32. The device of Example 19, wherein the chip channel is formed by laser irradiation.

33. The device of Example 19, wherein the chip channel has an open top.

34. The device of Example 19, wherein the chip channel is positioned between a top layer and a bottom layer.

35. The device of Example 19, wherein the chip channel is positioned adjacent to a top layer.

36. The device of Example 1, wherein the central region comprises a window that allows at least a part of the central region to be illuminated and imaged.

37. The device of Example 13, wherein the capillary tube comprises a window that allows at least a part of the capillary tube to be illuminated and imaged.

38. The device of Example 19, wherein the etched channel comprises a window that allows at least a part of the chip channel to be illuminated and imaged.

39. The device of Example 1, wherein the central region comprises a surface having at least one oligonucleotide tethered thereto.

40. The device of Example 39, wherein the surface is an interior surface of channel or capillary tube.

41. The device of Example 39 or 40, wherein the surface is a locally planar surface.

42. The device of Example 39, wherein the oligonucleotide is directly tethered to the surface.

43. The device of Example 39, wherein the oligonucleotide is tethered to the surface through an intermediate molecule.

44. The device of Example 39, wherein the oligonucleotide exhibits a segment that specifically hybridizes to a eukaryotic genomic nucleic acid segment.

45. The device of Example 39, wherein the oligonucleotide exhibits a segment that specifically hybridizes to a prokaryotic genomic nucleic acid segment.

46. The device of Example 39, wherein the oligonucleotide exhibits a segment that specifically hybridizes to a viral nucleic acid segment.

47. The device of Example 39, wherein the oligonucleotide exhibits a segment that specifically hybridizes to a transcriptome nucleic acid segment

48. The device of Example 1, wherein the central region comprises an interior volume suitable for sequencing a eukaryotic genome.

49. The device of Example 1, wherein the central region comprises an interior volume suitable for sequencing a prokaryotic genome.

50. The device of Example 1, wherein the central region comprises an interior volume suitable for sequencing a transcriptome.

51. The device of Example 1, comprises a temperature modulator thermally coupled to the central region.

52. The device of Example 1, wherein the temperature modulator comprises a heat block.

53. The device of Example 1, wherein the temperature modulator comprises a vent.

54. The device of Example 1, wherein the temperature modulator comprises a course for air flow.

55. The device of Example 1, wherein the temperature modulator comprises a fan.

56. A flow cell device comprising:
(d) a framework;
(e) a plurality of reservoirs harboring reagents common to a plurality of reactions compatible with the flow cell;
(f) a single reservoir harboring a reaction-specific reagent;
(g) a removable capillary having 1) a first diaphragm valve gating intake of a plurality of nonspecific reagents from the plurality of reservoirs, and 2) a second diaphragm valve gating intake of a single reagent from a source reservoir in close proximity to the second diaphragm valve.

57. The flow cell device of Example 56, wherein the framework comprises a thermal modulator.

58. The flow cell device of Example 57, wherein the thermal modulator comprises a heat block.

59. The flow cell device of Example 57, wherein the thermal modulator comprises a vent.

60. The flow cell device of Example 57, wherein the thermal modulator comprises a course for air flow.

61. The flow cell device of Example 57, wherein the thermal modulator comprises a fan.

62. The capillary flow cell device of Example 56, wherein the framework comprises a light detection access region.

63. The flow cell device of Example 62, wherein the light detection access region allows exposure of the removable capillary to an excitation spectrum.

64. The flow cell device of Example 62, wherein the light detection access region allows detection of an emission spectrum arising from the removable capillary.

65. The flow cell device of Example 56, wherein the reagents common to a plurality of reactions comprise at least one reagent selected from the list consisting of a solvent, a polymerase, and a dNTP.

66. The flow cell device of Example 56, wherein the reagents common to a plurality of reactions comprise low cost reagents.

67. The flow cell device of Example 56, wherein the reagents common to a plurality of reactions are directed to the first diaphragm valve through a first channel that is longer than a second channel connecting the second diaphragm valve to the single reservoir.

68. The flow cell device of Example 56, wherein the reaction-specific reagent is more expensive than any one nonspecific reagent.

69. The flow cell device of Example 56, wherein the reaction-specific reagent is more expensive than all nonspecific reagents.

70. The flow cell device of Example 56, wherein the reaction-specific reagent is configured in close proximity to the second diaphragm valve so as to reduce dead volume relative to delivery of the plurality of nonspecific reagents from the plurality of reservoirs to the first diaphragm valve.

71. The flow cell device of Example 56, wherein the capillary comprises a locally planar surface.

72. The flow cell device of Example 71, wherein the locally planar surface is at least partially transparent to an excitation wavelength.

73. The flow cell device of Example 71, wherein the locally planar surface is at least partially transparent to an emission wavelength.

74. The flow cell device of Example 71, wherein the locally planar surface comprises an oligonucleotide tethered thereto.

75. The flow cell device of Example 74, wherein the oligonucleotide is directly tethered to the surface.

76. The flow cell device of Example 74, wherein the oligonucleotide is tethered to the surface through an intermediate molecule.

77. The flow cell device of Example 74, wherein the oligonucleotide exhibits a segment that specifically hybridizes to a eukaryotic genomic nucleic acid segment.

78. The flow cell device of Example 74, wherein the oligonucleotide exhibits a segment that specifically hybridizes to a prokaryotic genomic nucleic acid segment.

79. The flow cell device of Example 74, wherein the oligonucleotide exhibits a segment that specifically hybridizes to a viral nucleic acid segment.

80. The flow cell device of Example 74, wherein the oligonucleotide exhibits a segment that specifically hybridizes to a transcriptome nucleic acid segment

81. The flow cell device of Example 56, wherein the capillary comprises an interior volume suitable for sequencing a eukaryotic genome.

82. The flow cell device of Example 56, wherein the capillary comprises an interior volume suitable for sequencing a prokaryotic genome.

83. The flow cell device of Example 56, wherein the capillary comprises an interior volume suitable for sequencing a transcriptome.

84. The flow cell device of Example 56, wherein the capillary comprises a tube.

85. The flow cell device of Example 84, wherein the tube is an off-shelf product.

86. The capillary flow cell device of Example 85, wherein the tube is manufactured to match specifications of the framework.

87. The flow cell device of Example 85, wherein the tube comprises an oligonucleotide population directed to sequence a eukaryotic genome.

88. The flow cell device of Example 56, wherein the device comprises a microfluidic chip.

89. The flow cell device of Example 88, wherein the microfluidic chip comprises a single etched layer.

90. The flow cell device of Example 88, wherein the microfluidic chip comprises at least one chip channel.

91. The flow cell device of Example 88, wherein the microfluidic chip comprises one etched layer.

92. The flow cell device of Example 91, wherein the microfluidic chip comprises one non-etched layer

93. The flow cell device of Example 91, wherein the microfluidic chip comprises two non-etched layers.

94. The flow cell device of Example 91, wherein the microfluidic chip comprises at least two bonded layers.

95. The flow cell device of Example 88, wherein the microfluidic chip comprises quartz.

96. The flow cell device of Example 88, wherein the microfluidic chip comprises borosilicate glass.

97. The flow cell device of Example 90, wherein the chip channel comprises an oligonucleotide population directed to sequence a prokaryotic genome.

98. The flow cell device of Example 90, wherein the chip channel comprises an oligonucleotide population directed to sequence a transcriptome.

99. A flow cell device comprising:
a) one or more capillaries, wherein the one or more capillaries are replaceable;
b) two or more fluidic adaptors attached to the one or more capillaries and configured to mate with tubing that provides fluid communication between each of the one or more capillaries and a fluid control system that is external to the flow cell device; and
c) optionally, a cartridge configured to mate with the one or more capillaries such that the one or more capillaries are held in a fixed orientation relative to the cartridge, and wherein the two or more fluidic adaptors are integrated with the cartridge.

100. The flow cell device of Example 99, wherein at least a portion of the one or more capillaries are optically transparent.

101. The flow cell device of Example 99 or 100, wherein the one or more capillaries are fabricated from glass, fused-silica, acrylic, polycarbonate, cyclic olefin copolymer (COC), cyclic olefin polymer (COP), or any combination thereof.

102. The flow cell device of any one of Examples 99 to 101, wherein the one or more capillaries have a circular, square, or rectangular cross-section.

103. The flow cell device of any one of Examples 99 to 102, wherein the largest internal cross-sectional dimension of a capillary lumen is between about 10 μm to about 1 mm.

104. The flow cell device of any one of Examples 99 to 103, wherein the largest internal cross-sectional dimension of a capillary lumen is less than about 500 μm.

105. The flow cell device of any one of Examples 99 to 104, wherein the two or more fluidic adaptors are fabricated from polydimethylsiloxane (PDMS; elastomer), polymethylmethacrylate (PMMA), polycarbonate (PC), polypropylene (PP), polyethylene (PE), high density polyethylene (HDPE), polyethyleneimine (PEI), polyimide, cyclic olefin polymers (COP), cyclic olefin copolymers (COC), polyethylene terephthalate (PET), epoxy resin, or any combination thereof.

106. The flow cell device of any one of Examples 99 to 105, wherein the cartridge is fabricated from polymethylmethacrylate (PMMA), polycarbonate (PC), polypropylene (PP), polyethylene (PE), high density polyethylene (HDPE), polyethyleneimine (PEI), polyimide, cyclic olefin polymers (COP), cyclic olefin copolymers (COC), polyethylene terephthalate (PET), epoxy resin, or any combination thereof.

107. The flow cell device of any one of Examples 99 to 106, wherein the cartridge further comprises one or more miniature valves, miniature pumps, temperature control components, or any combination thereof.

108. The flow cell device of any one of Examples 99 to 107, wherein a capillary lumen of the one or more capillaries comprises a low nonspecific binding coating.

109. The flow cell device of Example 108, wherein the low nonspecific binding coating further comprises covalently-tethered oligonucleotide primers.

110. The flow cell device of Example 109, wherein the covalently-tethered oligonucleotides are tethered at a surface density of about 1000 per μm².

111. The flow cell device of any one of Examples 108 to 110, wherein a surface property of low nonspecific binding coating is adjusted to provide optimal performance of a solid-phase nucleic acid amplification method performed within the one or more capillaries.

112. The flow cell device of any one of Examples 108 to 110, wherein the flow cell device comprises two or more capillaries, and wherein the low nonspecific binding coating of the two or more capillaries is the same.

113. The flow cell device of any one of Examples 108 to 112, wherein the flow cell device comprises two or more capillaries, and wherein the low nonspecific binding coating of one or more capillaries is different from that of the other capillaries.

114. The flow cell device of any one of Examples 108 to 113, wherein the flow cell device comprises an interior surface that is passivated.

115. The flow cell device of Example 114, wherein the interior surface comprises:
a) a substrate;
b) at least one hydrophilic polymer coating layer;
c) a plurality of oligonucleotide molecules attached to at least one hydrophilic polymer coating layer; and
d) at least one discrete region of the surface that comprises a plurality of clonally-amplified, sample nucleic acid molecules that have been annealed to the plurality of attached oligonucleotide molecules,
wherein a fluorescence image of the surface exhibits a contrast-to-noise ratio (CNR) of at least 20.

116. The flow cell device of Example 115, wherein the hydrophilic polymer coating layer has a water contact angle of less than 50 degrees.

117. The flow cell device of any one of Examples 114-116, wherein the substrate is glass or plastic.

118. A system comprising:
a) one or more of the flow cell devices of any one of Examples 99-113;
b) a fluid flow controller; and
c) optionally, a temperature controller or an imaging apparatus.

119. The system of Example 118, wherein the fluid flow controller comprises one or more pumps, valves, mixing manifolds, reagent reservoirs, waste reservoirs, or any combination thereof.

120. The system of Example 118 or 119, wherein the fluid flow controller is configured to provide programmable control of fluid flow velocity, volumetric fluid flow rate, the timing of reagent or buffer introduction, or any combination thereof.

121. The system of any one of Examples 118 to 120, wherein the temperature controller comprises a metal plate positioned so that it makes contact with the one or more capillaries, and a peltier or resistive heater.

122. The system of Example 121, wherein the metal plate is integrated into the cartridge.

123. The system of any one of Examples 118 to 122, wherein the temperature controller comprises one or more air delivery devices configured to direct a stream of heated or cooled air such that it makes contact with the one or more capillaries.

124. The system of any one of Examples 121 to 123, wherein the temperature controller further comprises one or more temperature sensors.

125. The system of Example 124, wherein the one or more temperature sensors are integrated into the cartridge.

126. The system of any one of Examples 118 to 125, wherein the temperature controller allows the temperature of the one or more capillaries to be held at a fixed temperature.

127. The system of any one of Examples 118 to 126, wherein the temperature controller allows the temperature of the one or more capillaries to be cycled between at least two set temperatures in a programmable manner.

128. The system of any one of Examples 118 to 127, wherein the imaging apparatus comprises a microscope equipped with a CCD or CMOS camera.

129. The system of any one of Examples 118 to 128, wherein the imaging apparatus comprises one or more light sources, one or more lenses, one or more mirrors, one or more prisms, one or more bandpass filters, one or more long-pass filters, one or more short-pass filters, one or more dichroic reflectors, one or more apertures, and one or more image sensors, or any combination thereof.

130. The system of any one of Examples 118 to 129, wherein the imaging apparatus is configured to acquire bright-field images, dark-field images, fluorescence images, two-photon fluorescence images, or any combination thereof.

131. The system of any one of Examples 118 to 130, wherein the imaging apparatus is configured to acquire video images.

132. A flow cell device comprising a one-piece or unitary flow cell construction.

133. The flow cell device of Example 132, wherein the one-piece or unitary flow cell construction comprises a glass or polymer capillary.

134. The flow cell device of Example 132 or 133, wherein in a surface of a fluid channel within the device comprises a low nonspecific binding coating.

135. A method of sequencing a nucleic acid sample and a second nucleic acid sample, comprising:
a) delivering a plurality of oligonucleotides to an interior surface of an at least partially transparent chamber;
b) delivering a first nucleic acid sample to the interior surface;
c) delivering a plurality of nonspecific reagents through a first channel to the interior surface;
d) delivering a specific reagent through a second channel to the interior surface, wherein the second channel has a lower volume than the first channel;
e) visualizing a sequencing reaction on the interior surface of the at least partially transparent chamber; and
f) replacing the at least partially transparent chamber prior to a second sequencing reaction.

136. The method of Examples 135, comprising flowing an air current past an exterior surface of the at least partially transparent surface.

137. The method of Example 135, comprising selecting the plurality of oligonucleotides to sequence a eukaryotic genome.

138. The method of Example 137, comprising selecting a prefabricated tube as the at least partially transparent chamber.

139. The method of Example 135, comprising selecting the plurality of oligonucleotides to sequence a prokaryotic genome.

140. The method of Example 135, comprising selecting the plurality of oligonucleotides to sequence a transcriptome.

141. The method of Example 139, comprising selecting a capillary tube as the at least partially transparent chamber.

142. The method of Example 140, comprising selecting a microfluidic chip as the at least partially transparent chamber.

143. A method of making a microfluidic chip in a flow cell device of claim 1, comprising:
providing a surface; and
etching the surface to form at least one channel.

144. The method of Example 143, wherein the etching is performed using laser radiation.

145. The method of Example 143, wherein the channel has an average depth of 50 to 300 μm.

146. The method of Example 143, wherein the channel has an average width of 0.1 to 30 mm.

147. The method of Example 143, wherein the channel has an average length in the range of 1 to 200 mm.

148. The method of Example 143, further comprising bonding a first layer to the etched surface.

149. The method of Example 143, further comprising bonding a second layer to the etched surface, wherein the etched surface is positioned between the first layer and the second layer.

150. A method of reducing a reagent used in a sequencing reaction, comprising:
(a) providing a first reagent in a first reservoir;
(h) providing a second reagent in a first second reservoir, wherein each of the first reservoir and the second reservoir are fluidically coupled to a central region, and wherein the central region comprises a surface for the sequencing reaction; and
(i) sequentially introducing the first reagent and the second reagent into a central region of the flow cell device, wherein the volume of the first reagent flowing from the first reservoir to the inlet of the central region is less than the volume of the second reagent flowing from the second reservoir to the central region.

151. A method of increasing the efficient use of a regent in a sequencing reaction, comprising:
(a) providing a first reagent in a first reservoir;
(b) providing a second reagent in a first second reservoir, wherein each of the first reservoir and the second reservoir are fluidically coupled to a central region, and wherein the central region comprises a surface for the sequencing reaction; and
(c) maintaining the volume of the first reagent flowing from the first reservoir to the inlet of the central region to be less than the volume of the second reagent flowing from the second reservoir to the central region.

152. The method of Example 150 or 151, wherein the first reagent is more expensive than the second agent.

153. The system of any of the Examples above, wherein said optical system comprises a sequencer.

154. The system of any of the Examples above, wherein said optical system comprises a nucleic acid sequencing apparatus.

155. The system of any of the Examples above, further comprising a fluid flow controller configured to control the flow of fluid through a flow cell or sample support structure.

156. The system of any of the Examples above, further comprising conduits configured to flow fluid through a flow cell or sample support structure.

157. The system of any of the Examples above, further comprising tubing that provides fluid communication with a fluid flow control system to provide fluid to a flow cell or sample support structure.

158. The system of any of the Examples above, further comprising a fluid flow controller configured to provide programmable control of fluid flow velocity, volumetric fluid flow rate, the timing of reagent or buffer introduction, or any combination thereof.

159. The system of any of the Examples above, further comprising a temperature controller configured to control the temperature of a flow cell or sample support structure.

160. The system of any of the Examples above, further comprising a reagent reservoir configured to provide reagent to the flow cell or sample support structure.

161. The system of any of the Examples above, further comprising a replaceable reagent configured to provide reagent to the flow cell or sample support structure.

162. The system of any of the Examples above, further comprising motorized translation stage configured to move the flow cell or sample support structure.

163. The system of any of the Examples above, further comprising motor controllers to control movement of a translation stage configured to move the flow cell or sample support structure.

164. The system of any of the Examples above, wherein said optical system comprises a nucleic sequencing apparatus.

153. The method of Example 150 or 151, wherein the first reagent is selected from the group consisting of a polymerase, a nucleotide, and a nucleotide analog.

Described herein are novel flow cell devices and systems for sequencing nucleic acids. The devices and systems described herein can achieve a more efficient use of the reagents help reduce the cost and time of the DNA sequencing process. The devices and systems can utilize a commercially-available, off-the-shelf capillaries or a micro or nano scale fluidic chip with a selected pattern of channels. The flow cell devices and systems described herein are suitable for rapid DNA sequencing and can help achieve more efficient use of expensive reagents and reduce the amount of time required for sample pre-treatment and replication compared to other DNA sequencing techniques. The result is a much faster and cost-effective sequencing method.

Some embodiments relate to A flow cell device, comprising: a first reservoir housing a first solution and having an inlet end and an outlet end, wherein the first agent flows from the inlet end to the outlet end in the first reservoir; a second reservoir housing a second solution and having an inlet end and an outlet end, wherein the second agent flows from the inlet end to the outlet end in the second reservoir; a central region having an inlet end fluidically coupled to the outlet end of the first reservoir and the outlet end of the second reservoir through at least one valve; wherein the volume of the first solution flowing from the outlet of the first reservoir to the inlet of the central region is less than the volume of the second solution flowing from the outlet of the second reservoir to the inlet of the central region

Some embodiments relate to A flow cell device comprising: a framework; a plurality of reservoirs harboring reagents common to a plurality of reactions compatible with the flow cell; a single reservoir harboring a reaction-specific reagent; a removable capillary having 1) a first diaphragm valve gating intake of a plurality of nonspecific reagents from the plurality of reservoirs, and 2) a second diaphragm valve gating intake of a single reagent from a source reservoir in close proximity to the second diaphragm valve.

Some embodiments relate to A flow cell device comprising: a) one or more capillaries, wherein the one or more capillaries are replaceable; b) two or more fluidic adaptors attached to the one or more capillaries and configured to mate with tubing that provides fluid communication between each of the one or more capillaries and a fluid control system that is external to the flow cell device; and c) optionally, a cartridge configured to mate with the one or more capillaries such that the one or more capillaries are held in a fixed orientation relative to the cartridge, and wherein the two or more fluidic adaptors are integrated with the cartridge.

Some embodiments relate to a method of sequencing a nucleic acid sample and a second nucleic acid sample, comprising: delivering a plurality of oligonucleotides to an interior surface of an at least partially transparent chamber; delivering a first nucleic acid sample to the interior surface; delivering a plurality of nonspecific reagents through a first channel to the interior surface; delivering a specific reagent through a second channel to the interior surface, wherein the second channel has a lower volume than the first channel; visualizing a sequencing reaction on the interior surface of the at least partially transparent chamber; and replacing the at least partially transparent chamber prior to a second sequencing reaction.

Some embodiments relate to a method of reducing a reagent used in a sequencing reaction, comprising: providing a first reagent in a first reservoir; providing a second reagent in a first second reservoir, wherein each of the first reservoir and the second reservoir are fluidically coupled to a central region, and wherein the central region comprises a surface for the sequencing reaction; and sequentially introducing the first reagent and the second reagent into a central region of the flow cell device, wherein the volume of the first reagent flowing from the first reservoir to the inlet of the central region is less than the volume of the second reagent flowing from the second reservoir to the central region.

Some embodiments relate to a method of increasing the efficient use of a regent in a sequencing reaction, comprising: providing a first reagent in a first reservoir; providing a second reagent in a first second reservoir, wherein each of the first reservoir and the second reservoir are fluidically coupled to a central region, and wherein the central region comprises a surface for the sequencing reaction; and maintaining the volume of the first reagent flowing from the first reservoir to the inlet of the central region to be less than the volume of the second reagent flowing from the second reservoir to the central region.

Although a wide range of features are discussed herein with respect to optical systems, any of the features and methods describe herein may be applied to other types of optical systems such as other types of optical imaging systems including without limitation bright-field, dark-field imaging and may also apply to luminescence or phosphorescence imaging. Accordingly, any of the examples provided above or elsewhere in this application, even if the example recites an optical system, fluorescence, emission, fluorescence emission, fluorescing sample sites, etc., such example may apply instead to other types of optical systems or optical imaging systems which do not necessarily include an optical system, fluorescence, emission, fluorescence emission, fluorescing sample sites, etc.

### Part IX

1. A method for fluorescence miscroscopy, the method comprising:
emitting an excitation beam from a light source of an optical system;
reflecting the excitation beam, by a first dichroic filter, into an objective lens of the optical system;
receiving the excitation beam at the objective lens;
directing the excitation beam, by the objective lens, to a specimen;
receiving, by the objective lens, emission light in response to the excitation beam;
transmitting the emission light by the first dichroic filter;
receiving the transmitted emission light at a second dichroic filter of the optical system;
transmitting, by the second dichroic filter, a first portion of the transmitted emission light to a first channel of a plurality of channels of the optical system;
reflecting, by the second dichroic filter, a second portion of the transmitted emission light to a second channel of the plurality of channels; and
receiving at least a portion of the emission light at optics of the plurality of channels.

2. A method for fluorescence microscopy, the method comprising:
emitting an excitation beam from a light source of an optical system;
receiving the excitation beam at an objective lens of the optical system;
directing the excitation beam, by the objective lens, to a specimen;
receiving, by the objective lens, emission light in response to the excitation beam;
receiving the emission light at a dichroic filter of the optical system disposed such that a central beam axis of the emission light has an angle of incidence of less than 45 degrees;
transmitting, by the dichroic filter, a first portion of the emission light to a first channel of a plurality of channels of the optical system;
reflecting, by the dichroic filter, a second portion of the emission light to a second channel of the plurality of channels; and
receiving at least a portion of the emission light at optics of the plurality of channels.

3. A method for fluorescence microscopy, the method comprising:
emitting an excitation beam from a light source of an optical system;
reflecting the excitation beam, by a dichroic filter, into an objective lens of the optical system, wherein the excitation beam is s-polarized with respect to the dichroic filter;
receiving the excitation beam at the objective lens;
directing the excitation beam, by the objective lens, to a specimen;
receiving, by the objective lens, emission light in response to the excitation beam;
transmitting the emission light by the dichroic filter to at least one channel of the optical system; and
receiving at least a portion of the emission light at optics of the at least one channel.

4. A method for fluorescence microscopy, the method comprising:
producing an excitation beam by a light source of an optical system;
receiving, at an objective lens of the optical system having a numerical aperture of less than 0.6, emission light from a sample on a support structure in response to the excitation beam;
receiving at least a portion of the emission light by at least one detection channel comprising optics and a photodetector array; and
capturing an image of at least one fluorescing sample said on said sample support structure.

5. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure;
wherein no optical element enters an optical path between the sample support structure and the photodetector array in said at least one detection channel in order to form an in-focus image of fluorescing sample sites on a first surface of said sample support structure onto the photodetector array and exits said optical path to form an in-focus image of fluorescing sample sites on a second surface of said sample support structure onto the photodetector array.

6. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure;
wherein no optical compensation is used to form an in-focus image of fluorescing sample sites on a first surface of said sample support structure onto the photodetector array that is not identical to optical compensation used to form an in-focus image of fluorescing sample sites on a second surface of said sample support structure onto the photodetector array.

7. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure;
wherein no optical element in an optical path between the sample support structure and a photodetector array in said at least one detection channel is adjusted differently to form an in-focus image of fluorescing sample sites on a first surface of said sample support structure onto the photodetector array than to form an in-focus image of fluorescing sample sites on a second surface of said sample support structure onto the photodetector array.

8. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure;
wherein no optical element in an optical path between the sample support structure and a photodetector array in said at least one detection channels is moved a different amount or a different direction to form an in-focus image of fluorescing sample sites on said a first surface of said sample support structure onto the photodetector array than to form an in-focus image of fluorescing sample sites on a second surface of said sample support structure onto the photodetector array.

9. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure;
wherein said optical system has a field-of-view of at least 1.0 mm wide with less than 0.1 waves of aberration over at least 80% of the field-of-view.

10. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure;
wherein the optical system has a field-of-view of at least 1.0 mm wide and is diffraction limited.

11. A method for fluorescence microscopy, the method comprising:
producing an excitation beam by a light source of an optical system, said light source having an optical output power of at least 0.8 W;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure.

12. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure;
wherein said optical system is capable of capturing with said photodetector array in-focus images of fluorescence emitting sample sites on first and second surfaces on said sample support structure; and wherein said optical system is configured to image said first and second surfaces at the same time and optical aberration is less for imaging said first and second surfaces than elsewhere in a region from 1 to 10 mm from said objective lens.

13. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by a plurality of detection channels comprising optics and photodetector arrays; and
capturing an image of at least one fluorescing sample said on said sample support structure;
wherein said objective lens is configured such that said optical system has a magnification of less than 10 (10×).

14. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure;
wherein said optical system is capable of capturing with said photodetector array in-focus images of fluorescence emitting sample sites on first and second surfaces on said sample support structure; and
wherein said optical system is configured to correct aberrations introduced by a layer through which said first and second surfaces of said sample support structure are imaged at the same time.

15. A method for fluorescence microscopy, the method comprising:
producing an excitation beam by a light source of an optical system;
receiving, at an objective lens of the optical system, emission light from a sample on a sample support structure in response to the excitation beam;
receiving at least a portion of the emission light by at least one detection channel comprising optics and a photodetector array; and
capturing an image of at least one fluorescing sample said on said sample support structure;
wherein said optical system is capable of capturing with said photodetector array in-focus images of fluorescence emitting sample sites on first and second surfaces on said sample support structure, said optical system configured to form in-focus images of said first and second surfaces on said photodetector array at the same time.

### Part X

1. An optical system comprising:
at least one light source configured to produce an excitation beam;
an objective lens configured to receive emission light from within a specified field of view (FOV) of a sample on a sample support structure in response to the excitation beam, said objective lens having a numerical aperture of less than 0.6 and a working distance of at least 700 μm, said FOV having an area of at least 1 mm²; and
at least one detection channel comprising optics and a photodetector array configured to receive at least a portion of the emission light and capture an image of at least one fluorescing sample site on said sample support structure.

2. The optical system of claim 1, wherein said sample support structure comprises a low binding surface having a water contact angle of less than 50 degrees, said sample support structure comprising amplified DNA colonies with a density of greater than 10,000 per mm².

3. The optical system of claim 1, wherein said objective lens has a numerical aperture of 0.5 or less.

4. The optical system of claim 3, wherein said objective lens has a numerical aperture of at least 0.3 and not greater than 0.4.

5. The optical system of claim 1, wherein said FOV has an area of at least 1.5 mm².

6. The optical system of claim 1, wherein said FOV has an area of at least 2 mm².

7. The optical system of claim 1, wherein said first surface is between said objective lens and said second surface, said first and second surfaces separated from each other by at least 0.05 mm and not more than 0.7 mm.

8. The optical system of claim 8, wherein said first and second surfaces are separated from each other by at least 0.075 mm and not more than 0.7 mm.

9. The optical system of claim 1, wherein said optical system provides diffraction limited imaging of both said first and second surfaces.

10. The optical system of claim 1, wherein said optical system is capable of capturing with said photodetector array in-focus images of fluorescence emitting sample sites on first and second surfaces on said sample support structure.

11. The optical system of claim 10, further comprising said sample support structure having said first and second surfaces.

12. The optical system of claim 11, wherein said sample support structure comprises a flow cell having a flow channel and said first and second surfaces comprise interior surfaces of said flow cell configured to be in contact with a sample flowing through said flow cell.

13. The optical system of claim 11, wherein said first and second surfaces on said sample support structure each comprise amplified DNA colonies with a density of greater than 10,000 per mm².

14. The optical system of claim 13, wherein said amplified DNA colonies are labeled with a fluorescent tag through a mono or oligo nucleotide such that a contrast-to-noise ratio (CNR) is greater than 4.

15. The optical system of claim 13, wherein said amplified DNA colonies include individual colonies classified from a fluorescent signal uniquely identifying A, G, C, or T through an incorporation, avidity, or binding event.

16. The optical system of claim 1, further comprising a means for sequencing a nucleic acid, wherein sequencing a nucleic acid comprises carrying out a sequencing by binding or sequencing by synthesis reaction on one or more surfaces, and detecting a bound or incorporated base using the optical system of claim 1.

17. The optical system of claim 1, further comprising a means for determining a genotype of a sample comprising a nucleic acid molecule, wherein determining the genotype of a sample comprises preparing said nucleic acid molecule for sequencing, and then sequencing said nucleic acid molecule using the system of claim 1.

18. The optical system of any of the Examples above, wherein said optical system comprises a sequencer.

19. The optical system of any of the Examples above, wherein said optical system comprises a nucleic acid sequencing apparatus.

20. The optical system of any of the Examples above, further comprising a fluid flow controller configured to control the flow of fluid through a flow cell or sample support structure.

21. The optical system of any of the Examples above, further comprising conduits configured to flow fluid through a flow cell or sample support structure.

22. The optical system of any of the Examples above, further comprising tubing that provides fluid communication with a fluid flow control system to provide fluid to a flow cell or sample support structure.

23. The optical system of any of the Examples above, further comprising a fluid flow controller configured to provide programmable control of fluid flow velocity, volumetric fluid flow rate, the timing of reagent or buffer introduction, or any combination thereof.

24. The optical system of any of the Examples above, further comprising a temperature controller configured to control the temperature of a flow cell or sample support structure.

25. The optical system of any of the Examples above, further comprising a reagent reservoir configured to provide reagent to the flow cell or sample support structure.

26. The optical system of any of the Examples above, further comprising a replaceable reagent configured to provide reagent to the flow cell or sample support structure.

27. The optical system of any of the Examples above, further comprising motorized translation stage configured to move the flow cell or sample support structure.

28. The optical system of any of the Examples above, further comprising motor controllers to control movement of a translation stage configured to move the flow cell or sample support structure.

29. The optical system of any of the Examples above, wherein said optical system comprises a nucleic sequencing apparatus.

### Part XI

1. An optical system comprising:
at least one light source configured to produce an excitation beam;
an objective lens configured to receive emission light from within a specified field of view (FOV) of a sample on a sample support structure in response to the excitation beam, said FOV having an area of at least 1 mm²; and
at least one detection channel comprising optics and a photodetector array configured to receive at least a portion of the emission light and capture an image of at least one fluorescing sample site on said sample support structure;
wherein said optical system is capable of capturing with said photodetector array in-focus images of fluorescence emitting sample sites on first and second surfaces on said sample support structure separated from each other by at least 0.05 mm without the insertion of a compensator optical element in order to achieve imaging of any one of said surfaces.

2. The optical system of claim 1, wherein said optical system provides diffraction limited imaging of both said first and second surfaces.

3. The optical system of claim 1, wherein said objective lens has a numerical aperture of less than 0.6.

4. The optical system of claim 1, wherein said objective lens has a numerical aperture of 0.5 or less.

5. The optical system of claim 4, wherein said objective lens has a numerical aperture of at least 0.3 and not greater than 0.4.

6. The optical system of claim 1, wherein said FOV has an area of at least 1.5 mm².

7. The optical system of claim 1, wherein said FOV has an area of at least 2 mm².

8. The optical system of claim 1, wherein said first surface is between said objective lens and said second surface, said first and second surfaces separated from each other by at least 0.05 mm.

9. The optical system of claim 8, wherein said first and second surfaces are separated from each other by at least 0.075 mm.

10. The optical system of claim 1, further comprising said sample support structure having said first and second surfaces.

11. The optical system of claim 10, wherein said sample support structure comprises a low binding surface having a water contact angle of less than 50 degrees, said sample support structure comprising amplified DNA colonies with a density of greater than 10,000 per mm².

12. The optical system of claim 11, wherein said amplified DNA colonies are labeled with a fluorescent tag such that an image of said first surface or said second surface has a contrast-to-noise ratio (CNR) greater than 4.

13. The optical system of claim 12, wherein the CNR is at least 10.

14. The optical system of claim 10, wherein said sample support structure comprises a flow cell having a flow channel and said first and second surfaces comprise interior surfaces of said flow cell configured to be in contact with a sample flowing through said flow cell.

15. The optical system of claim 1, further comprising a means for sequencing a nucleic acid, wherein sequencing a nucleic acid comprises carrying out a sequencing by binding or sequencing by synthesis reaction on one or more surfaces, and detecting a bound or incorporated base using the optical system of claim 1.

16. The optical system of claim 1, further comprising a means for determining a genotype of a sample comprising a nucleic acid molecule, wherein determining the genotype of a sample comprises preparing said nucleic acid molecule for sequencing, and then sequencing said nucleic acid molecule using the system of claim 1.

17. The optical system of any of the Examples above, wherein said optical system comprises a sequencer.

18. The optical system of any of the Examples above, wherein said optical system comprises a nucleic acid sequencing apparatus.

19. The optical system of any of the Examples above, further comprising a fluid flow controller configured to control the flow of fluid through a flow cell or sample support structure.

20. The optical system of any of the Examples above, further comprising conduits configured to flow fluid through a flow cell or sample support structure.

21. The optical system of any of the Examples above, further comprising tubing that provides fluid communication with a fluid flow control system to provide fluid to a flow cell or sample support structure.

22. The optical system of any of the Examples above, further comprising a fluid flow controller configured to provide programmable control of fluid flow velocity, volumetric fluid flow rate, the timing of reagent or buffer introduction, or any combination thereof.

23. The optical system of any of the Examples above, further comprising a temperature controller configured to control the temperature of a flow cell or sample support structure.

24. The optical system of any of the Examples above, further comprising a reagent reservoir configured to provide reagent to the flow cell or sample support structure.

25. The optical system of any of the Examples above, further comprising a replaceable reagent configured to provide reagent to the flow cell or sample support structure.

26. The optical system of any of the Examples above, further comprising motorized translation stage configured to move the flow cell or sample support structure.

27. The optical system of any of the Examples above, further comprising motor controllers to control movement of a translation stage configured to move the flow cell or sample support structure.

28. The optical system of any of the Examples above, wherein said optical system comprises a nucleic sequencing apparatus.

### BRIEF DESCRIPTION OF THE DRAWINGS

FIGS. 1A and 1B illustrate an illumination and imaging module of an example multi-channel fluorescence microscope comprising a dichroic beamsplitter for transmitting an excitation beam to a sample and for receiving and redirecting by reflection resultant fluorescence emission to four detection channels for detecting fluorescence emission of four different respective wavelengths or wavelength bands.

FIGS. 2A and 2B illustrate optical paths of the imaging module of FIGS. 1A and 1B comprising a dichroic beamsplitter for transmitting an excitation beam to a sample and for receiving and redirecting by reflection resultant fluorescence emission to four detection channels for detecting fluorescence emission of four different respective wavelengths or wavelength bands.

FIG. 3 is a graph illustrating a relationship between dichroic filter performance and beam angle of incidence.

FIG. 4 is a graph illustrating a relationship between beam footprint size and beam angle of incidence on a dichroic filter.

FIGS. 5A and 5B schematically illustrate an example configuration of dichroic filters and detection channels of a multi-channel fluorescence microscope wherein the dichroic filters have reflective surface tilted such that the angle between the incident beam (e.g., the central angle) and the reflective surface of the dichroic filter is less than 45.

FIGS. 6 and 7 are graphs illustrating improved dichroic filter performance corresponding to the configuration of FIGS. 5A and 5B.

FIGS. 8A and 8B are graphs illustrating reduced surface deformation resulting from the configuration of FIGS. 5A and 5B.

FIGS. 9A and 9B are graphs illustrating improved excitation filter performance (e.g. sharper transition between pass bands and surrounding stop bands) resulting from use of s-polarization of the excitation beam.

FIGS. 10A and 10B schematically illustrate example dual surface support structures for containing sample sites.

FIGS. 11A and 11B illustrate the modulation transfer function (MTF) of an example dual surface imaging system disclosed herein having a numerical aperture (NA) of 0.3.

FIGS. 12A and 12B illustrate the MTF of an example dual surface imaging system disclosed herein having an NA of 0.4.

FIGS. 13A and 13B illustrate the MTF of an example dual surface imaging system disclosed herein having an NA of 0.5.

FIGS. 14A and 14B illustrate the MTF of an example dual surface imaging system disclosed herein having an NA of 0.6.

FIGS. 15A and 15B illustrate the MTF of an example dual surface imaging system disclosed herein having an NA of 0.7.

FIGS. 16A and 16B illustrate the MTF of an example dual surface imaging system disclosed herein having an NA of 0.8.

FIG. 17A shows a plot of the Strehl ratios for different numerical apertures for different thicknesses.

FIG. 17B shows a plot of the Strehl ratio as a function of numerical aperture for illustrating the decreasing depth of field with NA that results in reduced resolution of imaging a plane through water having a thickness of 0.1 mm.

FIG. 18 provides an optical ray tracing diagram for an objective lens design configured for imaging a surface on the opposite side of a 0.17 mm thick coverslip.

FIG. 19 provides a plot of the modulation transfer function for the objective lens illustrated in FIG. 18 as a function of spatial frequency when used to image a surface on the opposite side of a 0.17 mm thick coverslip.

FIG. 20 provides a plot of the modulation transfer function for the objective lens illustrated in FIG. 18 as a function of spatial frequency when used to image a surface on the opposite side of a 0.3 mm thick coverslip.

FIG. 21 provides a plot of the modulation transfer function for the objective lens illustrated in FIG. 18 as a function of spatial frequency when used to image a surface that is separated from that on the opposite side of a 0.3 mm thick coverslip by a 0.1 mm thick layer of aqueous fluid.

FIG. 22 provides a plot of the modulation transfer function for the objective lens illustrated in FIG. 18 as a function of spatial frequency when used to image a surface on the opposite side of a 1.0 mm thick coverslip.

FIG. 23 provides a plot of the modulation transfer function for the objective lens illustrated in FIG. 18 as a function of spatial frequency when used to image a surface that is separated from that on the opposite side of a 1.0 mm thick coverslip by a 0.1 mm thick layer of aqueous fluid.

FIG. 24 provides a ray tracing diagram for a tube lens design which, if used in conjunction with the objective lens illustrated in FIG. 18, provides for improved dual-side imaging through a 1 mm thick coverslip.

FIG. 25 provides a plot of the modulation transfer function for the combination of objective lens and tube lens illustrated in FIG. 24 as a function of spatial frequency when used to image a surface on the opposite side of a 1.0 mm thick coverslip.

FIG. 26 provides a plot of the modulation transfer function for the combination of objective lens and tube lens illustrated in FIG. 24 as a function of spatial frequency when used to image a surface that is separated from that on the opposite side of a 1.0 mm thick coverslip by a 0.1 mm thick layer of aqueous fluid.

FIG. 27 provides ray tracing diagrams for tube lens design (left) of the present disclosure that is configured to provide high-quality, dual-side imaging performance. Because the tube lens is no longer infinity-corrected, an appropriately designed null lens (right) may be used in combination with the tube lens to compensate for the non-infinity-corrected tube lens for manufacturing and testing purposes.

FIG. 28 provides a schematic illustration of a dual-wavelength excitation/four channel emission fluorescence imaging system of the present disclosure.

FIG. 29 illustrates one embodiment of a single capillary flow cell having 2 fluidic adaptors.

FIG. 30 illustrates one embodiment of a flow cell cartridge comprising a chassis, fluidic adapters, and two capillaries.

FIG. 31 illustrates one embodiment of a system comprising a single capillary flow cell connected to various fluid flow control components, where the single capillary is compatible with mounting on a microscope stage or in a custom imaging instrument for use in various imaging applications.

FIG. 32 illustrates one embodiment of a system that comprises a capillary flow cell cartridge having integrated diaphragm valves to reduce or minimize dead volume and conserve certain key reagents.

FIG. 33 illustrates one embodiment of a system that comprises a capillary flow cell, a microscope setup, and a temperature control mechanism.

FIG. 34 illustrates one non-limiting example for temperature control of the capillary flow cells through the use of a metal plate that is placed in contact with the flow cell cartridge.

FIG. 35 illustrates one non-limiting approach for temperature control of the capillary flow cells that comprises a non-contact thermal control mechanism.

FIG. 36 illustrates visualization of cluster amplification in a capillary lumen.

FIGS. 37A-37C illustrates non-limiting examples of flow cell device preparation: FIG. 37A shows the preparation of one-piece glass flow cell; FIG. 37B shows the preparation of two-piece glass flow cell; and FIG. 37C shows the preparation of three-piece glass flow cell.

FIGS. 38A-38C illustrates non-limiting examples of glass flow cell designs: FIG. 38A shows a one-piece glass flow cell design; FIG. 38B shows a two-piece glass flow cell design; and FIG. 38C shows a three-piece glass flow cell design.

### DETAILED DESCRIPTION

Disclosed herein are optical system design guidelines and high-performance fluorescence imaging methods and systems that may provide any one or more of improved and/or high performance, optical resolution, image quality, and throughput for imaging in fluorescence microscopy-based applications such as genomics applications. Disclosed optical illumination and imaging system designs may possibly provide any one or more of the following advantages: improved image quality, improved dichroic filter performance, increased uniformity of dichroic filter frequency response, improved excitation beam filtering, larger fields-of-view, increased spatial resolution, improved modulation transfer, contrast-to-noise ratio, and image quality, higher spatial sampling frequency, faster transitions between image capture when repositioning the sample plane to capture a series of images (e.g., of different fields-of-view), improved imaging system duty cycle, or higher throughput image acquisition and analysis for one or more configurations although obtaining any such advantages is not required. Various implementations of optical systems such as optical imaging systems are configured to image sample sites, for example, on a sample support structures such as a flow cell. In some implementations, the optical systems may be configured to form images using light emitted from the sample such as fluorescence emission from the sample sites. In many implementations such sample sites are small. Accordingly, the optical system or optical imaging systems may be referred to herein as a microscope such as a fluorescence microscope. Such microscopes, e.g., fluorescence microscope, need not include one or more oculars or eyepieces for viewing through by the eyes of a person as is the case on certain laboratory microscope such as certain binocular and monocular laboratory microscopes. Rather, such microscopes may form images of samples or sample sites on a detector array such as a CCD or CMOS optical sensor array to collect images. Such images may be viewed by viewers on a display, computer screen, monitor, or television. Additionally, such microscopes or optical systems may be computer driven, electrically driven and/or be automated or semi-automated. Once again, such microscopes need not include one or more ocular or eyepieces for viewing through by a person who places their eyes in proximity to the ocular or eyepiece to look through the ocular or eyepiece or to receive light from the ocular or eyepiece to form an image in the person's eye. Likewise, throughout the present disclosure, the terms “optical system,” “optical imaging system,” “imaging system,” and “fluorescence microscope” may be used interchangeably to describe systems and devices such as for imaging samples possibly using fluorescence emission. Similarly, the optical systems, optical imaging systems, imaging systems, etc. described herein may comprises, may be, or may include one or more fluorescence microscopes, and any of the fluorescence microscopes described herein may comprise, may be or may include one or more optical systems. In various embodiments, any of the optical systems and/or fluorescence microscopes described herein may include one or more components for capturing images of samples such as sample sites on a sample support structure such as a flow cell possibly automatically or semi-automatically. Such images may be formed using emission from the sample such as fluorescence in some cases. Moreover, in various embodiments, any of the optical systems and/or fluorescence microscopes described herein may include one or more components for capturing an image of two spaced surfaces of a support structure sequentially, simultaneously, and/or automatically.

Various optical system designs and/or multi-channel fluorescence microscope designs may include illumination and imaging modules comprising folded optics (e.g., one or more beam splitters or combiners such as dichroic beamsplitters or combiners) that direct an excitation beam to an objective lens and direct emission light transmitted through the objective lens to a plurality of detection channels. Some particularly advantageous features of the fluorescence microscopes described herein include dichroic filter incidence angles that result in sharper and/or more uniform transitions between passband and stopband wavelength regions of the dichroic filters. Such filters may be included within the folded optics and may comprise dichroic beamsplitters or combiners. Further advantageous features of the microscope designs disclosed herein may include the positions and orientations of excitation light sources and detection optics with respect to the microscope objective and to a dichroic filter that received the excitation beam. The excitation beam may also be linearly polarized and the orientation of the linear polarization may be such that s-polarized light is incident on the dichroic reflective surface of the dichroic filter. Such features may potentially improve excitation beam filtering and/or reduce wavefront error introduced into the emission light beam due to surface deformation of dichroic filters. The fluorescence microscope described herein may or may not include any of these features and may or may not include any of these advantages. A wide range of systems and methods are disclosed herein.

In some instances, improvements in imaging performance, e.g., for dual-side (flow cell) imaging applications, may be achieved by using an electro-optical phase plate in combination with an objective lens to compensate for the optical aberrations induced by the layer of fluid separating the upper (near) and lower (far) interior surfaces of a flow cell. In some instances, this design approach may also compensate for vibrations introduced by, e.g., a motion-actuated compensator that is moved in or out of the optical path depending on which surface of the flow cell is being imaged.

In some instances, improvements in imaging performance, e.g., for dual-side (flow cell) imaging applications comprising the use of thick flow cell walls (e.g., wall (or coverslip) thickness >700 μm) and fluid channels (e.g., fluid channel height or thickness of 50-200 μm) may be achieved even when using commercially-available, off-the-shelf objectives by using a tube lens design that corrects for the optical aberrations induced by the thick flow cell walls and/or intervening fluid layer in combination with the objective.

In some instances, improvements in imaging performance, e.g., for multichannel (e.g., two-color or four-color) imaging applications, may be achieved by using multiple tube lenses, one for each imaging channel, where each tube lens design has been improved or optimized for the specific wavelength range used in that imaging channel.

It shall be understood that different aspects of the disclosed methods, devices, and systems can be appreciated individually, collectively, or in combination with each other. Although discussed herein primarily in the context of fluorescence imaging, it will be understood by those of skill in the art that many of the disclosed design approaches and features are applicable to other imaging modes, e.g., bright-field imaging, dark-field imaging, phase contrast imaging, and the like.

Fluorescence imaging viewed as an information pipeline: A useful abstraction of the role that fluorescence imaging systems plays in typical genomic assay techniques (including nucleic acid sequencing applications) is as an information pipeline, where the photon signal enters at one end of the pipeline, e.g., the objective lens used for imaging, and location specific information regarding the fluorescence signal emerges at the other end of the pipeline, e.g., at the position of the image sensor. When more information is pumped through this pipeline, some content, inevitably, will be lost during this transfer process and never recovered. An example of this case is when too many labeled molecules (or clonally-amplified clusters of molecules) are present within a small region of a substrate surface to be clearly resolved in the image; at the position of the image sensor, it becomes difficult to differentiate photon signals arising from adjacent clusters of molecules, thus increasing the probability of attributing the signal to the wrong cluster and leading to detection errors.

Design of optical imaging modules: One goal of designing an optical imaging module can thus be to increase or maximize the flow of information content through this detection pipeline and/or to reduce or minimize detection errors. Several design elements may potentially be addressed in the design process, including any one or more of the following:
1) Matching the physical feature density on the substrate surface to be imaged with the overall image quality of the optical imaging system and the pixel sampling frequency of the image sensor used. A mismatch of these parameters may result in loss of information or sometimes even the generation of false information, e.g., spatial aliasing may arise when pixel sampling frequency is lower than twice the optical resolution limit.
2) Matching the size of the area to be imaged with the overall image quality of the optical imaging system and/or focus quality across the entire field of view.
3) Matching the optical collection efficiency, modulation transfer function, and image sensor performance characteristics of the optical system design with the fluorescence photon flux expected for the input excitation photon flux, dye efficiency (related to dye extinction coefficient and fluorescence quantum yield), while accounting for background signal and system noise characteristics.
4) Improving, increasing or maximizing the separation of spectral content to reduce cross talk between fluorescence imaging channels.
5) Effective synchronization of image acquisition steps with repositioning of the sample or optics between image capture of different fields-of-view to reduce or minimize the down time (or improve or maximize the duty cycle) of the imaging system and thus improve or maximize the overall throughput of the image capture process.

This disclosure describes systematic ways to address each of the design elements outlined above and to create component level specifications for the imaging system.

Improved optical resolution and image quality to improve or maximize information transfer and throughput: One non-limiting design practice may be to start with the optical resolution required to distinguish two adjacent features as specified in terms of a number, X, of line pairs per mm (lp/mm) and translate it to a corresponding numerical aperture (NA) requirement. The numerical aperture requirement can then be used to assess the resulting impact on modulation transfer function and image contrast.

The standard modulation transfer function (MTF) describes the spatial frequency response for image contrast (modulation) transferred through an optical system; image contrast decreases as a function of spatial frequency and increases with increasing NA. This function limits the contrast/modulation that can be achieved for a given NA. Furthermore, wave front error can negatively impact the MTF, thus making it desirable to improve or optimize the optical system design using the true system MTF instead of that predicted by diffraction-limited optics. Note that, as used herein, MTF will refer to the total system MTF (including the complete optical path from coverslip to image sensor) although design practice may primarily consider the MTF of the objective lens. In genomic testing applications, where the target to be imaged is an array of high density “spots” on a surface (either randomly distributed or patterned), one can determine the minimum modulation transfer value required by downstream analysis to resolve two adjacent spots and discriminate between four possible states (e.g., ON-OFF, ON-ON, OFF-ON and OFF-OFF). For example, assume that the spots are small enough to be approximated as point sources of light. Assuming that the detection task is to determine if the two adjacent spots separated by a distance, d, are ON or OFF (in other words, bright or dark), and that the contrast-to-noise ratio (CNR) for the fluorescence signals arising from the spots at the sample plane (or object plane) is Cₛₐₘₚₗₑ, then under ideal conditions the CNR of the readout signal for the two adjacent spots at the image sensor plane, C_{image}, can be closely approximated as C_{image}=Cₛₐₘₚₗₑ*MTF(1/d), where MTF(1/d) is the MTF value at spatial frequency=(1/d).

In a typical design, the value of C may need to be at least 4 so that a simple threshold method can be used to avoid misclassification of fluorescence signals. Assuming a Gaussian distribution of fluorescence signal intensities around a mean value, at C_{image}>4, the expected error in correctly classifying fluorescence signals (e.g., as being ON or OFF) is <0.035%. The use of proprietary high CNR sequencing and surface chemistry, such as that described in U.S. patent application Ser. No. 16/363,842, allows one to achieve sample plane CNR (Cₛₐₘₚₗₑ) values for clusters of clonally-amplified, labeled oligonucleotide molecules tethered to a substrate surface of greater than 12 (or even much higher) when measured for a sparse field (i.e., at a low surface density of clusters or spots) where the MTF has a value of close to 100%. Assuming a sample plane CNR value of Cₛₐₘₚₗₑ>12 and targeting a classification error rate of <0.1% (thus, C_{image}>4), in some implementations, the minimum value for M(1/d) can be determined as M(1/d=4/12˜33%. Thus, a modulation transfer function threshold of at least 33% may be used to retain the information content of the transferred image.

Design practice can relate the minimum separation distance of two features or spots, d, to the optical resolution requirement (specified as noted above in terms of X (lp/mm)) as d=(1 mm)/X, i.e., d is the minimum separation distance between two features or spots which can be fully resolved by the optical system. In some designs disclosed in the present disclosure, where the objective of the design analysis is to increase or maximize relevant information transfer, this design criterion can be relaxed to d=(1 mm)/X/A, where 2>A>1. For the same optical resolution of X lp/mm, the value of d, the minimum resolvable spot separation distance at the sample plane, is reduced, thereby enabling the use of higher feature densities.

Design practice determines the minimum spatial sampling frequency at the sample plane using the Nyquist criteria, where spatial sampling frequency S≥2*X (and where X is the optical resolution of the imaging system specified in terms of X lp/mm as noted above). When the system spatial sampling frequency is close to the Nyquist criteria, as is often the case, imaging system resolution of greater than S results in aliasing as the higher frequency information resolved by the optical system cannot be sufficiently sampled by the image sensor.

In some designs in the present disclosure, an oversampling scheme based on the relationship S=B*Y (where B≥2 and Y is the true optical system MTF limit) may be used to further improve the information transfer capacity of the imaging system. As indicated above, X (lp/mm) corresponds to a practical, non-zero (>33%) minimum modulation transfer value, whereas Y (lp/mm) is the limit of optical resolution so modulation at Y (lp/mm) is 0. Thus, in the disclosed designs, Y (lp/mm) may advantageously be significantly greater than X. For values of B≥2, the disclosed designs are oversampling for the sample object frequency X, i.e., S≥B*Y>2*X.

The above relationship can be used to determine the system magnification and may provide an upper bound for image sensor pixel size. The choice of image sensor pixel size is matched to the system optical quality as well to the spatial sampling frequency required to reduce aliasing. The lower bound of image sensor pixel size can be determined based on photon throughput, as relative noise contributions increase with smaller pixels.

Other design approaches however are possible. For example, reducing the NA, for example, to less to less than 6 (e.g., 5 or less) may provide increased depth of field. Such increased depth of field may enable dual surface imaging wherein two surfaces at different depths can be imaged at the same time. As discussed above, reducing NA may reduce resolution. In some implementations, higher excitation beam power such as greater than 0.8 W, e.g., 1 W or more, may be employed to produce strong signal. A high CNR, for example, of >20 may also be used to facilitate imaging. In some designs, support structures such as flow cells having hydrophilic surface are used to reduce background noise.

In various implementations, large field-of-view (FOV) is provided by the optical system. For example, a FOV equal to or greater than 1, 2, or 3 mm may be provided with some optical imaging systems comprising, e.g., an objective lens and a tube lens. In some cases, the optical imaging system provides a reduced magnification, for example, of less than 10×, for example, of 8× or 5× or less. Such reduced magnification may in some implementations facilitate large FOV designs. Despite reduced magnification, resolution can be sufficient, as detector arrays having small pixel size or pitch may be used. In some implementations, the pixel size is smaller than (or at least as small as) twice the optical resolution provided by the optical imaging system (e.g., objective and tube lens) to satisfy Nyquist theorem. Other designs and methods, however, are possible. In some designs configured to provide for dual surface imaging wherein two surfaces at different depths can be imaged at the same time, the optical imaging system (e.g., the objective lens and/or tube lens) are configured to reduce aberration for imaging said two surfaces (e.g., two planes) at those two respective depths more than other locations (e.g., other planes) at other depths. Additionally, the optical imaging system may be configured to reduce aberration for imaging said two surfaces (e.g., two planes) at those two respective depths through a transmissive layer on said support structure such as a layer of glass (e.g., cover slip) and solution (e.g., an aqueous solution) comprising the sample.

Described herein are systems and devices to analyze a large number of different nucleic acid sequences from e.g., amplified nucleic acid arrays in flow cells or from an array of immobilized nucleic acids. The systems and devices described herein can also be useful in, e.g., sequencing for comparative genomics, tracking gene expression, micro RNA sequence analysis, epigenomics, and aptamer and phage display library characterization, and other sequencing applications. The systems and devices herein comprise various combinations of optical, mechanical, fluidic, thermal, electrical, and computing devices/aspects. The advantages conferred by the disclosed flow cell devices, cartridges, and systems include, but are not limited to: (i) reduced device and system manufacturing complexity and cost, (ii) significantly lower consumable costs (e.g., as compared to those for currently available nucleic acid sequencing systems), (iii) compatibility with typical flow cell surface functionalization methods, (iv) flexible flow control when combined with microfluidic components, e.g., syringe pumps and diaphragm valves, etc., and (v) flexible system throughput.

Described herein are capillary flow-cell devices and capillary flow cell cartridges that are constructed from off-the-shelf, disposable, single lumen (e.g., single fluid flow channel) capillaries that may also comprise fluidic adaptors, cartridge chassis, one or more integrated fluid flow control components, or any combination thereof. Also disclosed herein are capillary flow cell-based systems that may comprise one or more capillary flow cell devices, one or more capillary flow cell cartridges, fluid flow controller modules, temperature control modules, imaging modules, or any combination thereof.

The design features of some disclosed capillary flow cell devices, cartridges, and systems include, but are not limited to, (i) unitary flow channel construction, (ii) sealed, reliable, and repetitive switching between reagent flows that can be implemented with a simple load/unload mechanism such that fluidic interfaces between the system and capillaries are reliably sealed, facilitating capillary replacement and system reuse, and enabling precise control of reaction conditions such as temperature and pH, (iii) replaceable single fluid flow channel devices or capillary flow cell cartridges comprising multiple flow channels that can be used interchangeably to provide flexible system throughput, and (iv) compatibility with a wide variety of detection methods such as fluorescence imaging.

Although the disclosed single flow cell devices and systems, capillary flow cell cartridges, capillary flow cell-based systems, microfluidic chip flow cell device, and microfluidic chip flow cell systems, are described primarily in the context of their use for nucleic acid sequencing applications, various aspects of the disclosed devices and systems may be applied not only to nucleic acid sequencing but also to any other type of chemical analysis, biochemical analysis, nucleic acid analysis, cell analysis, or tissue analysis application. It shall be understood that different aspects of the disclosed devices and systems can be appreciated individually, collectively, or in combination with each other.

### Example Fluorescence Microscope Illumination and Imaging Modules

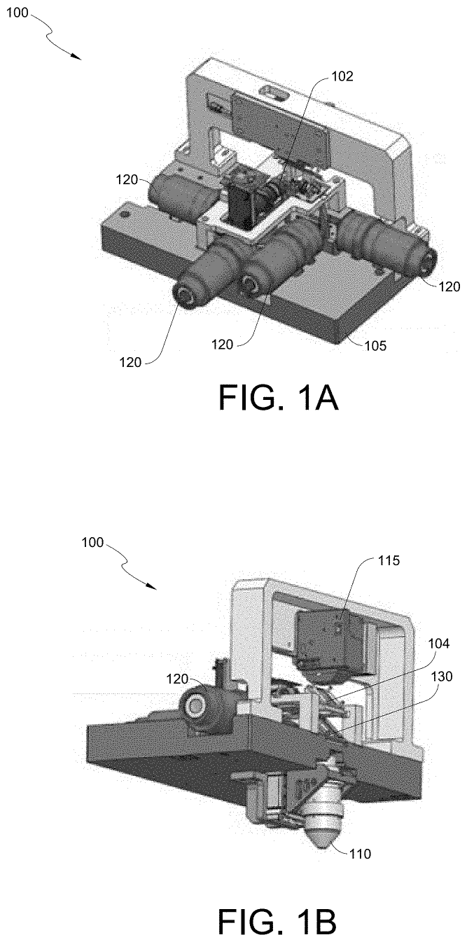

FIGS. 1A and 1B illustrate an illumination and imaging module 100 of an example multi-channel fluorescence microscope. The illumination and imaging module 100 includes an objective lens 110, an illumination source 115, a plurality of detection channels 120, and a first dichroic filter 130, which may comprise a dichroic reflector or beamsplitter. An autofocus system, which may include an autofocus laser 102, for example, that projects a spot the size of which is monitored to determine when the imaging system is in-focus may be included in some designs. Some or all components of the illumination and imaging module 100 may be coupled to a baseplate 105.

The illumination or light source 115 may include any suitable light source configured to produce light of at least a desired excitation wavelength. The light source may be a broadband source that emits light of one or more broad band of wavelengths, or the light source may be a narrowband source that emits light one or more narrower band or even a single isolated wavelength or line corresponding to the desired excitation wavelength or multiple isolated wavelengths or lines. Of course, lines may have some bandwidth in various cases. Example light sources that may be suitable for use in the illumination source 115 include, but are not limited to, an incandescent filament, xenon arc lamp, mercury-vapor lamp, a light-emitting diode, a laser source such as a laser diode or a solid state laser, or other types of light sources. As discussed below, in some designs, the light source comprises a polarized light source such as a linearly polarized light source. In some implementations, the orientation of the light source is such that s-polarized light is incident on one or more surfaces of one or more optical components such as the dichroic reflective surface of one or more dichroic filters.

In some implementations, the light source 115 outputs a sufficiently large amount of light to produce sufficiently strong fluorescence emission. Stronger fluorescence emission can increase the signal-to-noise ratio (SNR) and the contrast-to-noise ratio (CNR) of the image. In some implementations, the light source outputs at least 0.8 W and may output 1 W or more. Depending on the design, application, and/or configuration, the light source may be configured, for example, to output at least 0.5 W, at least 0.6 W, at least 0.7 W, at least 0.8 W, at least 1 W, at least 1.1 W, at least 1.2 W, at least 1.3 W, at least 1.4 W, at least 1.5 W, at least 1.6 W, at least 1.8 W, at least 2.0 W, or more possibly as much as 2.5 W, as much as 3.0 W or more or any amount of power in any range formed by any of these values. In some implementations, multiple light sources are included in the illumination and imaging module 100. In some such implementation, different light sources may each produce sufficiently high output power. For example, two light sources may be included that both output at least 0.8 W or at least 1 W or any of the values and ranges between any of the values recited above. Similarly, in some implementations, three, four, five or more light sources may be included and these light sources may each output at least 0.8 W or at least 1 W or any of the values and ranges between any of the values recited above depending on the design. In some implementations, the output power of the light source is sufficient to provide for a CNR ratio of images obtained by the illumination and imaging system of 20 or more, 21 or more, 22 or more, 23 or more, 24 or more, 25 or more, 30 or more, 35 or more, 40 or more, 50 or more, or any CNR ratio in any range formed by any of these values.

In some implementations, the light source or light sources comprise lasers such as laser diodes. In some implementations, the light source or light sources comprise visible color light sources such as green and/or red light sources.

The illumination source 115 may further include one or more additional optical components such as lenses, filters, optical fibers, or any other suitable transmissive or reflective optics as appropriate to output an excitation beam having suitable characteristics toward a first dichroic filter 130. For example, beam shaping optics may be included, for example, to receive light from a light emitter in the light source and produce a beam and/or provide a desired beam characteristic. Such optics may, for example, comprises a collimating lens configured to reduce the divergence of light and/or increase collimation and/or to collimate the light.

In some implementations, the excitation beam is sufficiently large to produce strong fluorescence emission. The excitation beam may, for example, be at least 0.8 W and may be 1 W or more. Depending on the design, application, and/or configuration, the excitation beam may be at least 0.5 W, at least 0.6 W, at least 0.7 W, at least 0.8 W, at least 1 W, at least 1.1 W, at least 1.2 W, at least 1.3 W, at least 1.4 W, at least 1.5 W, at least 1.6 W, at least 1.8 W, at least 2.0 W, or more possibly as much as 2.5 W, as much as 3.0 W or more or any amount of power in any range formed by any of these values. As discussed above, in some implementations, multiple light sources are included in the illumination and imaging module 100. In some such implementation, different light sources produce light having different spectral characteristics, for example, so as to produce different fluorescence, e.g., to excite different fluorescence dyes. Light from the different light source may overlap and form an aggregate excitation beam. This composite excitation beam may be composed of excitation beams from each of the light sources. The composite excitation beam will have more optical power than the individual beams that overlap to form the composite beam. For example, in some implementations that include two light sources outputting excitation beams, both beams may be at least 0.8 W or at least 1 W or any of the values and ranges between any of the values recited above. Likewise, this composite beam may have optical power that is the sum of the optical power of the individual beams that form the composite beam. Similarly, in some implementations, three, four, five or more light sources may be included and these light sources may each output excitation beams having at least 0.8 W or at least 1 W or any of the values and ranges between any of the values recited above depending on the design. Likewise, this composite beam will have optical power that is the sum of the optical power of the individual beams that form the composite beam. In some implementations, the composite excitation beam and/or the individual excitation beams that form the composite beam have sufficient power to provide for a CNR ratio of images obtained by the illumination and imaging system of 20 or more, 21 or more, 22 or more, 23 or more, 24 or more, 25 or more, 30 or more, 35 or more, 40 or more, 50 or more, or any CNR ratio in any range formed by any of these values.

As referenced above, the first dichroic filter 130 is disposed with respect to the light source to receive light therefrom. The first dichroic filter may comprise a dichroic mirror or reflector or dichroic beamsplitter or beam combiner such as a dichroic beamsplitter or beam combiner configured to transmit light in a first spectral region and reflect light having a second spectral region. The first spectral region may include one or more spectral bands such as a band of wavelengths in the ultraviolet and blue. Similarly, a second spectral region may include one or more band such as a band of wavelengths extending from the green to red and infrared. Of course, other regions are possible.

In some implementations, the first dichroic filter may be configured to transmit light from the light source to sample support structure such as to a flow cell or microfluidic chip or other substrate or support structure. The sample support structure may also comprise a capillary tube in some cases. The sample support structure supports sample with respect to the illumination and imaging module 100. Accordingly, a first optical path extends from the light source to the sample via the first dichroic filter.

In various implementations, the sample support structure includes at least one surface to which sample binds. The sample may, for example, bind to different localized regions or sites on the at least one surface of the sample support structure. This sample may in some implementations fluoresce when illuminated with the excitation beams. Accordingly, a plurality of fluorescing sample sites may be included on at least one surface of the sample support structure that are illuminated and imaged by the illumination and imaging module. In some designs, the support structure includes two surfaces located at different depths to which sample binds and thus include fluorescing sample sites. As discussed below, for example, a flow cell may comprise a channel formed at least in part by first and second (e.g., upper and lower) interior surfaces. The sample may flow along these surfaces and some sample may adhere to localized sites on these surfaces that are appropriately treated to bind with the sample. The first and second surface may be separated by the region corresponding to the channel through which the solution flow and thus be at different distances or depth with respect to the illumination and imaging module 100, for example, with respect to the object lens 110.

The objective lens 110 may be included in the first optical path between the first dichroic filter and the sample. This objective lens may be configured, for example, to have a focal length, work distance, and/or be positioned to direct and/or focus light from the light source onto the sample, e.g., onto the flow cell or microfluidic chip or other substrate or capillary tube or support structure. In some implementations, the objective need not bring the light to an extremely focused spot but may direct the beam onto the sample, possibly converging the beam and/or reducing the beam size that is directed onto the sample. Similarly, the objective lens may be configured to have suitable focal length, work distance, and/or be positioned to collect light from the sample and to form an image of the sample.

This objective lens may comprise a microscope objective such as an off-the-shelf objective or may comprise a custom objective. An example objective lens is show below and in U.S. Provisional Application No. 62/962,723 filed Jan. 17, 2020, which is incorporated herein by reference in its entirety. This objective lens may have a numerical aperture of 0.6 or more. For example, the objective lens may have a numerical aperture of 0.6 or more, or 0.7 or more, or 0.8 or more or 0.9 or more or may have a numerical aperture or any numerical aperture in any range between any of these values.

The objective lens may be designed to reduce or minimize aberration at two locations such as two planes corresponding to two surfaces on a flow cell or other sample support structure, for example, where fluorescing sample sites are located. The objective may be designed to reduce the aberration at the selected locations or planes relative to other locations or planes such as first and second surfaces containing fluorescing sample sites on a dual surface flow cell. For example, the objective may be designed to reduce the aberration at two depths or planes located at different distances from the objective lens as compared to the aberrations associate with other depths or planes at other distances from the objective. For example, optical aberration may be less for imaging the first and second surfaces than elsewhere in a region from 1 to 10 mm from the objective lens. Additionally, a custom objective may in some embodiments be configured to compensate for aberration induced by transmission of emission light through one or more portions of the sample support structure such as a layer that includes one of the surfaces on which sample adheres as well as possibly a solution corresponding to the sample. This layer may comprise, e.g., glass, quartz, plastic, or other transparent material having a refractive index and introduce aberration. A custom objective, for example, may in some embodiments be configured to compensate for aberration induced by a sample support structure coverslip or other components as well as possibly a solution corresponding to the sample.

Although numerical apertures of at least 0.6 were discussed above, in various implementations, this objective lens may have a numerical aperture of 0.6 or less. Accordingly, this objective lens may have a numerical aperture of 0.6, less than 0.6, 0.5 or less, 0.4 or less, 0.3 or less, 0.2 or less, 0.1 or less, or any numerical aperture in any range between any of these values. Such a numerical aperture may provide for increased depth of focus and/or depth of field. Such increased depth of focus and/or depth of field may increase the ability to image planes separated by a distance such as first and second surfaces on a dual surface flow cell. Such planes may therefore correspond to object planes or locations that can be imaged and where such first and second surfaces might be located when the flow cell or other sample support structure is in place for evaluation. As discussed above, a flow cell may comprise, for example, first and second layers separated by a channel through which an analyte can flow. The objective lens may be configured to provide a depth for field sufficiently large to image the first and second interior surfaces of the flow cell where sample may bind and fluoresce (or to image object planes, surfaces, or locations where such first and second surfaces might be located when the flow cell or other sample support structure is in place for evaluation). The depth of field may be at least as large or larger than the distance separating the first and second surfaces of the flow cell to be imaged such as the first and second interior surfaces of the flow cell (or to image object planes, surfaces, or locations where such first and second surfaces might be located when the flow cell or other sample support structure is in place for evaluation). The first and second surfaces of the dual surface flow cell (or the object planes, surfacdes, or locations where such first and second surfaces might be located when the flow cell or other sample support structure is in place for evaluation) may be separated, for example, by a distance of at least 0.075 mm. The first and second surfaces to be imaged (or the object planes, surfaces, or locations where such first and second surfaces might be located when the flow cell or other sample support structure is in place for evaluation) may, for example, be separated by 0.05 mm or more, 0.075 mm or more, 0.1 mm or more, 0.125 mm or more, 0.150 mm or more, 0.175 mm or more, 0.2 mm or more, 0.250 mm or more, 0.3 mm or more, 0.4 mm or more, 0.5 mm or more, 0.6 mm or more, 0.7 mm or more, or any distance in any range between any of these values. For example, the first and second surfaces to be imaged may (or the object planes, surfaces, or locations where such first and second surfaces might be located when the flow cell or other sample support structure is in place for evaluation), for example, be separated by 0.05 mm to 0.250 mm, or 0.05 mm to 0.2 mm, or 0.05 mm to 0.15 mm or 0.05 mm to 0.125 mm or 0.05 to 0.100 mm. For example, the first and second surfaces to be imaged may (or the object planes, surfaces, or locations where such first and second surfaces might be located when the flow cell or other sample support structure is in place for evaluation), for example, be separated by 0.075 mm to 0.250 mm, or 0.075 mm to 0.2 mm, or 0.075 mm to 0.15 mm or 0.075 mm to 0.125 mm or 0.075 to 0.100 mm or 0.075 to 0.150 mm or 0.075 to 0.200 mm or from 0.100 to 0.200 mm or from 0.200 to 0.300 mm or from 0.300 to 0.400 mm or from 0.400 mm to 0.500 mm. Other ranges formed by any of the value listed above are possible. Likewise, the objective lens may be configured to provide, for example, a depth of field and/or depth of focus of 0.05 mm or more, 0.075 mm or more, 0.1 mm or more, 0.125 mm or more, 0.150 mm or more, 0.175 mm or more, 0.2 mm or more, 0.250 mm or more, 0.3 mm or more, 0.4 mm or more, 0.5 mm or more, 0.6 mm or more, 0.7 mm or more, or any value in any range between any of these values. For example, the depth of field and/or depth of focus of the objective and/or fluorescent microscope can, for example, be in a range from 0.05 mm to 0.250 mm, or 0.05 mm to 0.2 mm, or 0.05 mm to 0.15 mm or 0.05 mm to 0.125 mm or 0.05 to 0.100 mm or 0.05 to 0.150 mm or 0.05 to 0.200 mm. Alternatively, the depth of field and/or depth of focus of the objective and/or fluorescent microscope can, for example, be from 0.075 mm to 0.250 mm, or 0.075 mm to 0.2 mm, or 0.075 mm to 0.15 mm or 0.075 mm to 0.125 mm or 0.075 to 0.100 mm or 0.075 to 0.150 mm or 0.075 to 0.200 mm or from 0.100 to 0.200 mm or from 0.200 to 0.300 mm or from 0.300 to 0.400 mm or from 0.400 mm to 0.500 mm. Other ranges formed by any of the value listed above are possible.

In some designs, compensation optics may move into or out of an optical path in the imaging module, for example, in an optical path of light collected by the objective lens 110 to enable the imaging module to image the first and second surfaces of the dual surface flow cell. The imaging module, for example, may be configured to image the first surface when the compensation optics is included in the optical path between the objective lens and a photodetector array or sensor configured to capture an image of the first surface. In such a design, the imaging module may be configured to image the second surface when the compensation optics is removed from or not included in the optical path between the objective lens 110 and the photodetector array or sensor configured to capture an image of the second surface. In some implementations, the optical compensation system comprises a refractive optical element such as a lens or a plate of transparent material such as glass. Other configurations may be employed to enable the first and second surfaces to be imaged at different times. For example, one or more lenses or optical elements may be configured to be translated along an optical path between the objective lens 110 and the photodetector.

In certain designs, however, the objective lens 110 is configured to provide sufficiently large depth of focus and/or depth of field to enable the first and surfaces to be imaged without such compensation optics moving into and out of an optical path in the imaging module such as an optical path between the objective lens and the photodetector array. Similarly, in various designs, the objective lens is configured to provide sufficiently large depth of focus and/or depth of field to enable the first and surfaces to be imaged without optics being moved, such as one or more lenses or other optical components being translated along an optical path in the imaging module such as an optical path between the objective lens and the photodetector array.

In some implementations, the objective lens (or microscope objective) 110 is configured to have reduced magnification. The objective lens 110 may be configured, for example, such that the fluorescence microscope has a magnification of less than 10 (10×). The objective lens 110 may be configured, for example, such that the fluorescence microscope has a magnification of 9× or less, 8× or less, 7× or less, 6× or less, 5× or less, 4× or less, 3× or less, 2× or less or a range between any of these values. Such reduced magnification may alter design constraints such that other design parameters can be achieved. For example, the objective lens 110 may also be configured such that the fluorescence microscope has a large field-of-view (FOV), for example, a field-of-view of at least 3.0 mm or at least 3.2 mm (e.g., in width or diameter). The objective lens 110, may also be configured such that the fluorescence microscope has an FOV of at least 1 mm, 1.5 mm, at least 2.0 mm, at least 2.5 mm, at least 3.0 mm or at least 3.2 mm, at least 3.5 mm, at least 4.0 mm, at least 4.5 mm, at least 5.0 mm (e.g., in width or diameter) or any FOV in a range between any of these values. The objective lens 110 may be configured to provide the fluorescence microscope with such a field-of-view such that the FOV has less than 0.1 waves of aberration over at least 80% of field. Similarly, the objective lens 110 may be configured such that the fluorescence microscope has such a FOV and is diffraction limited or is diffraction limited over such an FOV.

As discussed above, the first dichroic beamsplitter or combiner is disposed in the first optical path between the light source and the sample so as to illuminate the sample with one or more excitation beams. This first dichroic beamsplitter or combiner is also in one or more second optical path from the sample to the different optical channels used to detect the fluorescent emission. Accordingly, the first dichroic filter 130 couples the first optical path of the excitation beam emitted by the illumination source 115 and second optical path of the emission light emitted by a specimen to the various optical channels where the light continues onto respective photodetector arrays for capturing images of the sample.

In various implementations, the first dichroic filter 130, e.g., first dichroic reflector or beamsplitter or combiner, has a passband selected so as to transmit light from the illumination source 115 at only a band of wavelengths or possibly a plurality of wavelength bands including the desired excitation wavelength or wavelengths. For example, the first dichroic beamsplitter 130 includes a reflective surface comprising a dichroic reflector that has spectral transmissivity response that is configured to transmit light having at least some of the wavelengths output by the light source that form part of the emission beam. The spectral transmissivity response may be configured not to transmit (e.g., instead to reflect) light of one or more other wavelengths, for example, of one or more other fluorescent emission wavelengths. In some implementations, the spectral transmissivity response may also be configured not to transmit (e.g., instead to reflect) light of one or more other wavelengths output by the light source. Accordingly, the first dichroic filter 130 may be utilized to select which wavelength or wavelengths output by the light source that reaches the sample. Conversely, the dichroic reflector in the first dichroic beam splitter 130 has a spectral reflectivity response that reflects light having one or more wavelengths corresponding to the desired fluorescent emission from the sample and possible reflects light having one or more wavelengths output from the light source that is not intended to reach the sample. Accordingly, in some implementations, the dichroic reflector has a spectral transmissivity that includes one or more pass bands to transmit the light to be incident on the sample and one or more stop bands that reflects light outside the pass bands, for example, one or more emission wavelengths and possible one or more wavelengths output by the light source that are not intended to reach the sample. Likewise, in some implementations the dichroic reflector has a spectral reflectivity that includes one or more spectral regions configured to reflect one or more emission wavelengths and possible one or more wavelengths output by the light source that are not intended to reach the sample and includes one or more regions that transmits light outside these reflection regions. The dichroic reflector included in the first dichroic filter 130 may comprise a reflective filter such as an interference filter (e.g., a quarter-wave stack) configured to provide the appropriate spectral transmission and reflection distributions. (FIGS. 1A and 1B also show a dichroic filter 105, which may comprise for example a dichroic beamsplitter or combiner, that may be used to direct light from the autofocus laser 102 through the objective and to the sample support structure.)

Although the imaging module 100 shown in FIGS. 1A and 1B and discussed above is configured such that the excitation beam is transmitted by the first dichroic filter 130 to the objective lens 110, in some designs the illumination source 115 may be disposed with respect to the first dichroic filter 130 and/or the first dichroic filter is configured (e.g., oriented) such that the excitation beam is reflected by the first dichroic filter 130 to the objective lens 110. Similarly, in some such designs, the first dichroic filter 130 is configured to transmit fluorescent emission from the sample and possibly transmit light having one or more wavelengths output from the light source that is not intended to reach the sample. As will be discussed below, a design where the fluorescent emission is transmitted instead of reflected may potentially reduce wavefront error in the detected emission and/or possibly have other advantages. In either case, various implementations the first dichroic reflector 130 is disposed in the second optical path so as to receive fluorescent emission from the sample, at least some of which continues onto the detection channels 120.

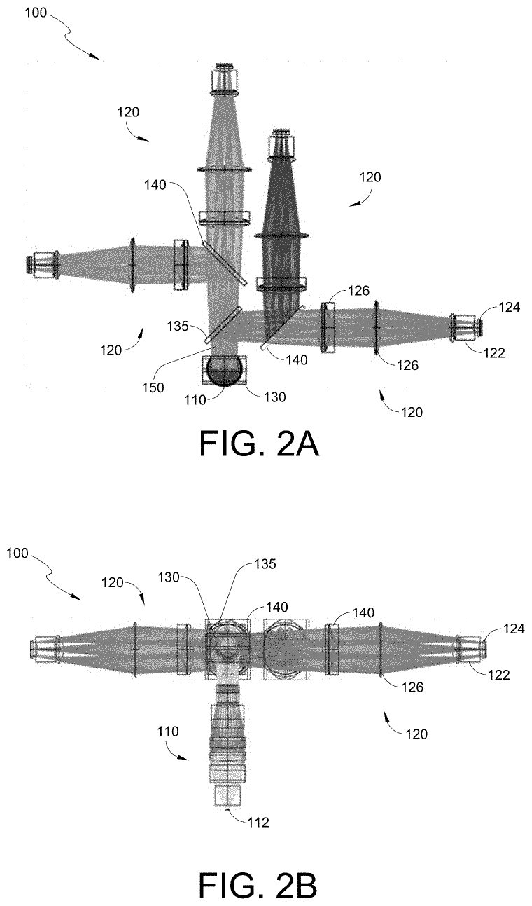

In the example show in FIGS. 1A and 2A, the detection channels 120 are disposed to receive fluorescent emission from a specimen that is transmitted by the objective lens 110 and reflected by the first dichroic filter 130. (As referred to above and described more below, in some designs the detection channels 120 may be disposed to receive the portion of the emission light that is transmitted, rather than reflected, by the first dichroic filter.) In either case, the detection channels 120 may include optics for receiving at least a portion of the emission light. For example, the detection channels 120 may include one or more lenses, such as tube lenses, and may include one or more sensors or detectors such as photodetector arrays (e.g., CCD or CMOS sensor arrays) for imaging or otherwise producing a signal based on the received light. The tube lenses may, for example, comprise one or more lens elements configured to form an image of the sample onto the sensor or photodetector array to capture an image thereof. Additional discussion of detection channels is included below and in U.S. Provisional Application No. 62/962,723 filed Jan. 17, 2020, which is incorporated herein by reference in its entirety. In some embodiments, improved optical resolution may be achieved using a sensor having relatively high sensitivity, small pixels, and high pixel count, in conjunction with a suitable sampling scheme, which may include oversampling or undersampling.

FIGS. 2A and 2B are ray tracing diagrams illustrating optical paths of the illumination and imaging module 100 of FIGS. 1A and 1B. FIG. 2A corresponds to a top view of the illumination and imaging module 100. FIG. 2B corresponds to a side view of the illumination and imaging module 100. The illumination and imaging module 100 includes four detection channels 120. However, it will be understood that the present technology may equally be implemented in systems including more or fewer than four detection channels 120. For example, the multi-channel systems disclosed herein may be implemented with as few as one detection channel 120, two detection channels, two detection channels, or up to five, six, seven, eight, or more detection channels, without departing from the spirit or scope of the present disclosure.

The example imaging module 100 of FIGS. 2A and 2B includes four detection channels 120, a first dichroic filter 130 that reflects a beam 150 of emission light, a second dichroic filter (e.g., dichroic beamsplitter) 135 that splits the beam 150 into a transmitted portion and a reflected portion, and two channel-specific dichroic filters (e.g., dichroic beamsplitters) 140 that further split the transmitted and reflected portions of the beam 150 among individual detection channels 120. The dichroic reflecting surface in the dichroic beam splitters 135, 140 for splitting the beam 150 among detection channels are shown disposed at 45 degrees relative to a central beam axis of the beam 150 or an optical axis of the imaging module. However, as discussed below, an angle smaller than 45 degrees may be employed and may offer advantages such as sharper transition from pass band to stop band.

The different detection channels 120 includes imaging devices 124, which may include a sensor or photodetector array (CCD or CMOS detector array). In various implementations, the different detection channels 120 further includes optics 126 such as lenses (e.g., one or more tube lenses comprising one or more lens elements) disposed to direct and/or focus the portion of the emission light entering the detection channel 120 at a focal plane coincident with a plane of the photodetector array 124. The optics 126 (e.g., tube lens) combined with the objective lens 110 are configured to form an image of the sample onto the photodetector array 124 to capture an image of the sample, for example, an image of a surface on the flow cell or other sample support structure after the sample has bound to that surface. As discussed herein, in some implementations the sample is not necessarily in sharp focus on the photodetector array 124. Sample sites that are unfocused or out of focus may nevertheless be imaged by the photodetector array 124. The photodetector array 124 may be able to produce images of the sample wherein the fluorescing sample sites are resolvable even if blurry or not in focus. Accordingly, such an image of the sample may comprise a plurality of fluorescent emitting spots or regions across a spatial extent of the sample support structure where the sample is emitting fluorescent light. The objective lens together with the optics 126 (e.g., tube lens) may provide a field of view (FOV) that includes a portion or the entire sample. Similarly, the photodetector array 124 of the different detection channels 120 may be configured to capture images of a full field of view (FOV) provided by the objective lens and the tube lens or a portion thereof. In some implementations, the photodetector array 124 of some or all detection channels 120 can detect the emission light emitted by a sample bound to the sample support structure, e.g., the flow cell, or a portion thereof and record electronic data representing an image thereof. In some implementations, the photodetector array 124 of some or all detection channels 120 can detect features in the emission light emitted by a specimen without capturing and/or storing an image of the sample bound to the flow cell and/or of the full FOV provided by the objective lens and optics 126 (e.g., tube lens). In various embodiments, the FOV of the systems disclosed herein may be, for example, between 1 mm and 5 mm, between 1.5 mm and 4.5 mm, between 2 mm and 4 mm, between 2.5 mm and 3.5 mm, between 3.0 mm and 4.0 mm or between 3.0 mm and 5.0 mm, within any other suitable range such as any range formed by any of these values. In one example, the FOV is approximately 3.2 mm. The FOV may be selected, for example, to provide a balance between magnification and resolution of the system and/or based on one or more characteristics of the photodetectors and/or objective lenses. For example, a relatively smaller FOV may be provided in conjunction with a smaller and faster imaging sensor to achieve high throughput.

In some implementations, the optics 126 in the detection channel (e.g., the tube lens) may be configured to reduce optical aberration in imaging. In some implementations having multiple channels for imaging at different wavelengths, the optics 126 in the detection channel (e.g., tube lenses) in the different channels have different designs to reduce aberration for the respective wavelengths at which that particular channel is configured to image. In some implementations, the optics 126 in the detection channel (e.g., the tube lens) may be configured to reduce aberrations when imaging the surface (e.g., plane, object plane, etc.) on the sample support structure having the fluorescing sample sites as compared to other locations (e.g., other planes in object space). Similarly, in some implementations, the optics 126 in the detection channel (e.g., the tube lens) may be configured to reduce aberrations when imaging first and second surfaces (e.g., plane, object plane, etc.) on a dual surface sample support structure (e.g., dual surface flow cell) having the fluorescing sample sites as compared to other locations (e.g., other planes in object space). For example, the optics 126 in the detection channel (e.g., tube lens) may be designed to reduce the aberration at two depths or planes located at different distances from the objective lens as compared to the aberrations associate with other depths or planes at other distances from the objective. For example, optical aberration may be less for imaging the first and second surfaces than elsewhere in a region from 1 to 10 mm from the objective lens. Additionally, custom optic 126 in the detection channel (e.g., tube lens) may in some embodiments be configured to compensate for aberration induced by transmission of emission light through one or more portions of the sample support structure such as a layer that includes one of the surfaces on which sample adheres as well as possibly a solution corresponding to the sample. This layer may comprise, e.g., glass, quartz, plastic, or other transparent material having a refractive index and introduce aberration. Custom optic 126 in the detection channel (e.g., the tube lens), for example, may in some implementations be configured to compensate for aberration induced by a sample support structure coverslip or other components as well as possibly a solution corresponding to the sample.

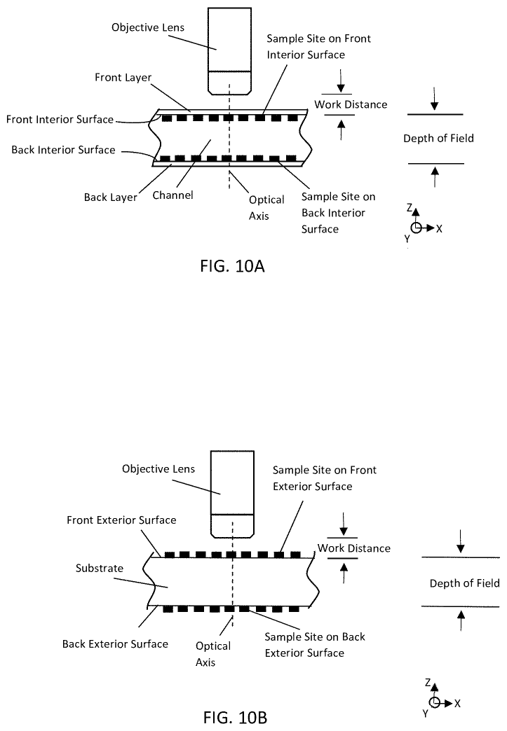

In some implementations, the optics 126 in the detection channel (e.g. tube lens) are configured to have reduced magnification. The optics 126 in the detection channel (e.g. tube lens) may be configured, for example, such that the fluorescence microscope has a magnification of less than 10 (10×). The optics 126 in the detection channel (e.g. tube lens) may be configured, for example, such that the fluorescence microscope has a magnification of 9× or less, 8× or less, 7× or less, 6× or less, 5× or less, 4× or less, 3× or less, 2× or less or a range between any of these values. Such reduced magnification may alter design constraints such that other design parameters can be achieved. For example, the optics 126 in the detection channel (e.g. tube lens) may also be configured such that the fluorescence microscope has a large field-of-view (FOV), for example, a field-of-view of at least 3.0 mm or at least 3.2 mm (e.g., in width or diameter). The optics 126 in the detection channel (e.g. tube lens) may also be configured such that the fluorescence microscope has an FOV of at least 1.0 mm, at least 1.5 mm, at least 2.0 mm, at least 2.5 mm, at least 3.0 mm or at least 3.2 mm, at least 3.5 mm, at least 4.0 mm, at least 4.5 mm, at least 5.0 mm (e.g., in width or diameter) or any FOV in a range between any of these values. The optics 126 in the detection channel (e.g. tube lens) may be configured to provide the fluorescence microscope with such a field-of-view such that the FOV has less than 0.1 waves of aberration over at least 80% of field. Similarly, the optics 126 in the detection channel (e.g. tube lens) may be configured such that the fluorescence microscope has such a FOV and is diffraction limited or is diffraction limited over such an FOV. In various implementations, a sample is located at or near a focal position 112 of the objective lens 110. As described above with reference to FIGS. 1A and 1B, a light source such as a laser source provides an excitation beam to the sample to induce fluorescence. At least a portion of fluorescent emission is collected by the objective lens 110 as emission light. The objective lens 110 transmits the emission light toward the first dichroic filter 130, which reflects some or all of the emission light as the beam 150 incident upon the second dichroic filter 135 and to the different detection channels where optics 126 forms an image of the sample (e.g., a plurality of fluorescing sample sites on a surface of a sample support structure) onto the photodetector array 124. As discussed above, in some implementations, the sample support structure comprises a flow cell such as a dual surface flow cell having two surfaces (e.g., two interior surfaces, a first surface and a second surface) containing sample sites that emit fluorescent emission. These two surfaces may be separated by a distance from each other in the longitudinal (Z) direction (see FIGS. 10A and 10B) along the direction of the central axis of the excitation beam, the central axis of the objective lens, and/or the optical axis of the objective lens. This direction may be orthogonal to the scan direction which may, for example, be in X and/or Y (e.g., see FIGS. 10A and 10B) and parallel to the direction of adjustment to provide focus. This separation may correspond, for example, to a flow channel within the flow cell. Analyte may be flowed through the flow channel and contact the first and second interior surfaces of the flow cell which may be treated with a binding element such that fluorescent emission is radiated from a plurality of sites on these surfaces. The imaging optics (e.g., objective lens) may be positioned at a suitable distance (e.g., a distance corresponding to the work distance) from the sample to form in-focus or possibly not precisely in focus (e.g., possibly blurred or out of focus) images of the sample on the detector array 124. As discussed above, one or more of the sample sites, e.g., the fluorescing sample sites, may not be precisely in focus and may possibly be blurred or out of focus. The image processing electronics and/or processor may be configured resolve such sample sites such as fluorescing sample sites even if not in-focus but possibly blurred or out of focus. As discussed above, however, in various designs, the objective lens (possibly together with the optics 126) have a depth of field and/or depth of focus that is at least as large as the longitudinal separation between the first and second surfaces. The objective lens and the optics 126 can thus simultaneously form in focus images of both the first and the second surface on the photodetector array 124 and these images of the first and second surfaces are in focus. In certain implementations, the objective lens and the optics 126 can simultaneously form images of both the first and the second surface on the photodetector array 124 wherein one or both of the first and second surfaces are not completely or fully in focus, or are blurred or not in focus. In various implementations, compensation optics need not be moved into or out of an optical path of the imaging module (e.g., into or out of the first and/or second optical paths) to form images such as in focus images or possibly blurred or not in-focus of the first and second surfaces. Similarly, in various implementations, one or more optical elements (e.g., lens elements) in the imaging module (e.g., in the object lens or optics 126) need not be moved, for example, in the longitudinal direction along the first and/or second optical paths to form images such as in focus or not in-focus images of the first surface in comparison to the location of said one or more optical element when used to image the second surface. In some implementations, however, the imaging module includes an autofocus system configured to provide both the first and second surface in focus at the same time or possibly to bring one of the surfaces in focus. In some implementations, however, neither the first nor second surfaces are fully or completely in focus or are blurred or not in focus. In various implementations, the sample is in focus to sufficiently resolve the sample sites, which are closely spaced together in lateral directions (e.g., X and Y directions).

However as discussed above, in various implementations, the sample need not be in focus or fully or completely in focus to sufficiently resolve the sample sites, for example, in the lateral directions (e.g., X and Y directions). Rather, sample sites may be out of focus or blurred or not fully in focus. Processing of the images may be configured to resolve the sample sites even if the sample sites are not completely or fully in focus or may be blurred or out of focus. Likewise, the sample sites on one or both the first and second surfaces may be not in-focus or not fully in focus and may be blurred or out of focus yet the photodetector array 124 may obtain images that are processed and/or used.

As discussed above, the dichroic filters may comprise interference filters that selectively transmit and reflect light of different wavelengths based on the principle of thin-film interference, using layers of optical coatings having different refractive indices and particular thickness. Accordingly, the spectral response (e.g., transmission and/or reflection spectrums) of the dichroic filters implemented within multi-channel fluorescence microscopes may be at least partially dependent upon the angle of incidence, or range of angles of incidence, at which the light of the excitation and/or emission beams are incident upon the dichroic filters. Such effects may be especially significant with respect to the dichroic filters of the detection optics (e.g., the dichroic filters 135, 140 of FIGS. 2A and 2B).

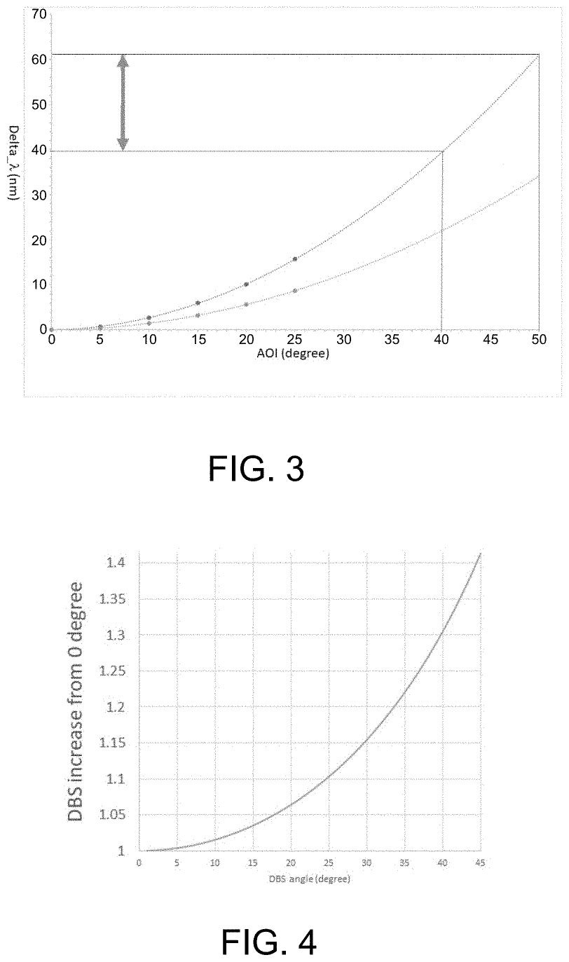

FIG. 3 is a graph illustrating a relationship between dichroic filter performance and beam angle of incidence. Specifically, the graph of FIG. 3 illustrates the effect of angle of incidence on the transition width or spectral span of a dichroic filter, which correspond to the range of wavelengths where the spectral response (e.g., transmission spectrum and/or reflection spectrum) transitions between the passband and stopband regions of a dichroic filter. Thus, a transmission edge (or reflection edge) having a relatively small spectral span (e.g., a small delta λ value in the graph of FIG. 3) corresponds to a sharper transition between passband and stopband regions or the transmission and reflection regions (or conversely between reflection and transmission regions), while a transmission edge (or reflection edge) having a relatively large spectral span (e.g., a large delta_λ value in the graph of FIG. 3) corresponds to a less sharp transition between passband and stopband regions. In various implementations, sharper transitions between passband and stopband regions are generally desirable. Moreover, it may also be desirable to have increased consistency or a relatively consistent transition width across most or all of the field of view and/or beam area.

Fluorescence microscopes, in which the dichroic mirrors are disposed at 45 degrees relative to a central beam axis of the emission light or the optical axis of the optical paths (e.g., of the objective lens and/or tube lens), accordingly can have a transition width of roughly 50 nm for an example dichroic filter, as shown in FIG. 3. Because the emission light beam is not collimated and has some degree of divergence, fluorescence microscopes may have a range of angles of incidence of approximately 5 degrees between opposing sides of the beam. Thus, as shown in FIG. 3, different portions of the beam of emission light may be incident upon a channel splitting dichroic filter at various angles of incidence between 40 degrees and 50 degrees. This range of relatively large angles of incidence corresponds to a range of transition widths between about 40 nm and about 62 nm. This range of relatively large angles of incidence thereby leads to an increase in transition width of the dichroic filter in the imaging module. Performance of multi-channel fluorescence microscopes could thus be improved by providing smaller angles of incidence across the full beam thereby making the transmission edge sharper.

FIG. 4 is a graph illustrating a relationship between beam footprint size and beam angle of incidence on a dichroic filter. For a number of reasons, a relatively smaller beam footprint may be desirable. For example, a small beam footprint allows smaller dichroic filters to be used to split a beam. The suitability of smaller dichroic filters in turn reduces manufacturing costs and improves ease of manufacturing suitably flat dichroic filters. As shown in FIG. 4, any angle of incidence greater than 0 degrees (e.g., perpendicular to the surface of the dichroic filter) results in an elliptical beam footprint having an area larger than the cross-sectional area of the beam. A 45 degree angle of incidence results in a large footprint greater than 1.4 times the cross-sectional area of the beam.

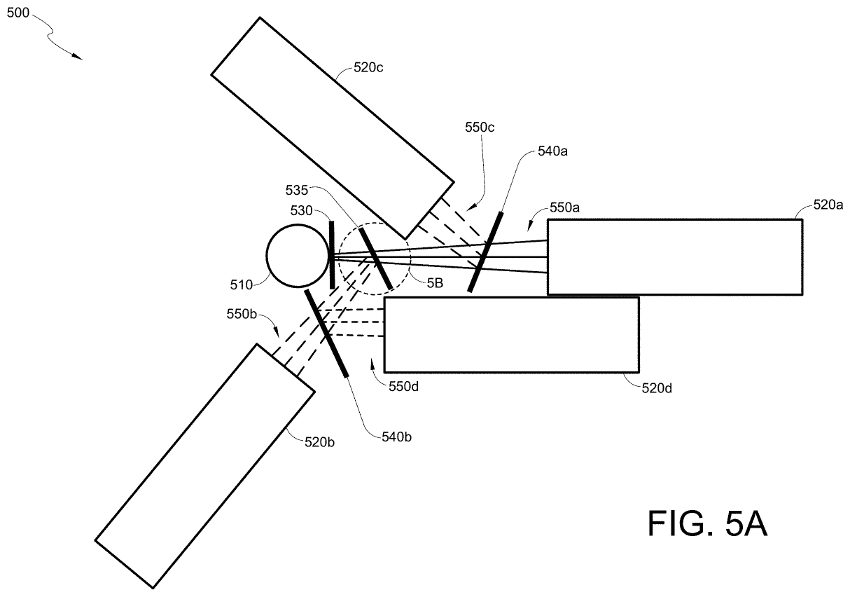

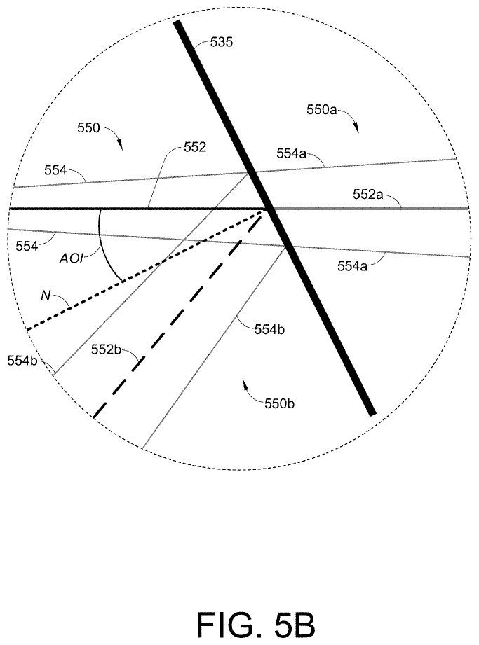

FIGS. 5A and 5B schematically illustrate an example configuration of dichroic filters and detection channels of a multi-channel fluorescence microscope wherein the dichroic mirrors are disposed at an angle less than 45 degrees relative to a central beam axis of the emission light or the optical axis of the optical paths (e.g., of the objective lens and/or tube lens). In particular, FIG. 5A depicts an imaging module 500 including a plurality of detection channels 520a, 520b, 520c, 520d. FIG. 5B is a detailed view of the portion of the imaging module 500 within the circle 5B as shown in FIG. 5A. As will be described in greater detail, the configuration illustrated in FIGS. 5A and 5B includes a number of aspects that may result in significant improvements over conventional multi-channel fluorescence microscope designs. Systems and devices, however, may be implemented with one or a subset of the features described with respect to FIGS. 5A and 5B without departing from the spirit or scope of the present disclosure.

The imaging module 500 includes an objective lens 510 and four detection channels 520a, 520b, 520c, and 520d disposed to receive and/or image emission light transmitted by the objective lens 510. A first dichroic filter 530 is provided to couple the excitation and detection optical paths. In contrast to the design shown in FIGS. 1A and 1B as well as 2A and 2B, the first dichroic filter (e.g., dichroic beamsplitter or combiner) 530, is configured to reflect light from the light source to the objective lens 510 and sample and transmit fluorescent emission from the sample to the detection channels 520a, 520b, 520c, and 520d. A second dichroic filter 535 splits a beam 550 of emission light among at least two detection channels 520a, 520b by transmitting a first portion 550a and reflecting a second portion 550b. Additional dichroic filters 540a, 540b are provided to further split the emission light. Dichroic filter 540a transmits at least a portion of the first portion 550a of the emission light and reflects a portion 550c to a third detection channel 520c. Dichroic filter 540b transmits at least a portion of the second portion 550b of the emission light, and reflects a portion 550d to a fourth detection channel 520d. Although the imaging module 500 is depicted with four detection channels, in various embodiments the imaging module 500 may include more or fewer detection channels, with a correspondingly larger or smaller number of dichroic filters as appropriate to provide a portion of the emission light to each detection channel. For example, in some embodiments, the features of the imaging module 500 may be implemented with similar advantageous effects in a simplified imaging module including only two detection channels 520a, 520b, and omitting additional dichroic filters 540a, 540b. In some implementations, only one detection channel may be included. Alternatively, three or more detection channels may be employed.

The detection channel 520a, 520b, 520c, 520d may include some or all of the same or similar components to those of the detection channels 120 illustrated in FIGS. 1A-2B. For example, different detection channel 520a, 520b, 520c, 520d may include one or more photodetectors arrays and may include transmissive and/or reflective optics such as one or more lenses (e.g., tube lenses) that direct and/or focus the light received by the detection channel onto its respective photodetector array.

The objective lens 510 is disposed to receive emission light emitted by fluorescence from a specimen. In particular, the first dichroic filter 530 is disposed to receive the emission light transmitted by the objective lens 510. As discussed above and shown in FIG. 5A, in some designs, an illumination source (e.g., the illumination source 115 of FIGS. 1A and 1B) such as a laser source or the like is disposed to provide an excitation beam which is incident on the first dichroic filter 530 such that the first dichroic filter 530 reflects the excitation beam into the same objective lens 510 that transmits the emission light, for example, in an epifluorescence configuration. (In some other designs, the illumination source may be directed to the specimen by other optical components along a different optical path that does not include the same objective lens 510. In such configurations, the first dichroic filter 530 may be omitted.)

Similarly, as discussed above and shown in FIG. 5A, the detection optics (e.g., including the detection channels 520a, 520b, 520c, 520d and any optical components such as dichroic filters 535, 540a, 540b along the optical path between the objective lens 510 and the detection channels 520a, 520b, 520c, 520d) may be disposed on the transmission path of the first dichroic filter 530, rather than on the reflected path of the first dichroic filter 530. In one example implementation, the objective lens 510 and detection optics are disposed such that the objective lens 510 transmits the beam 550 of emission light directly toward the second dichroic filter 535. The wavefront quality of the emission light is degraded somewhat by the presence of the first dichroic filter 530 along the path of the beam 550 of emission light (e.g., by imparting some wavefront error to the beam 550). However, the wavefront error introduced by a beam transmitted through a dichroic reflector of a dichroic beamsplitter is generally significantly smaller than the wavefront error of a beam reflected from the dichroic reflecting surface of a dichroic beamsplitter (e.g., an order of magnitude smaller). Thus, the wavefront quality and subsequent imaging quality of the emission light in a multi-channel fluorescence microscope may be substantially improved by placing the detection optics along the transmitted beam path of the first dichroic filter 530 rather than along the reflected beam path.

Within the detection optics of the imaging module 500, dichroic filters 535, 540a, and 540b are provided to split the beam 550 of emission light among the detection channels 520a, 520b, 520c, 520d. For example, the dichroic filters 535, 540a, and 540b split the beam 550 on the basis of wavelength, such that a first wavelength or wavelength band of the emission light can be received by the first detection channel 520a, a second wavelength or wavelength band of the emission light can be received by the second detection channel 520b, a third wavelength or wavelength band of the emission light can be received by the third detection channel 520c, and a fourth wavelength or wavelength band of the emission light can be received by the fourth detection channel 520d. In some implementations, multiple separated wavelengths or wavelength bands can be received by the detection channel.

In contrast to the multi-channel fluorescence microscope design shown in FIGS. 1A and 1B as well as 2A and 2B, the imaging module 500 has dichroic filters 535, 540a, and 540b disposed at angles of incidence of less than 45 degrees with respect to the central beam axis of the incident beams. As shown in FIG. 5B, the different beams 550, 550a, 550b have respective central beam axes 552, 552a, 552b. In various implementations, the central beam axes 552, 552a, 552b is at the center of a cross-section of the beam orthogonal to the propagation direction of the beam. These central beam axes 552, 552a, 552b may correspond to the optical axis of the objective lens and/or the optics within the separate channels, for example, the optical axes of the respective tube lenses. Additional rays 554, 554a, 554b of each beam 550, 550a, 550b are illustrated in FIG. 5B to indicate the diameter of each beam 550, 550a, 550b. Beam diameter may be defined, for example, as a full width at half maximum diameter, a D4τ or second-moment width, or any other suitable definition of beam diameter.

The central beam axis 552 of the beam 550 of emission light may serve as a reference point for defining the angle of incidence of the beam 550 on the second dichroic filter 535. Accordingly, the “angle of incidence” (AOI) of a beam 550 may be the angle between the central beam axis 552 of the incident beam 550 and a line N normal to the surface the beam is incident on, for example, the dichroic reflective surface. When the beam 550 of emission light is incident upon the dichroic reflective surface of the second dichroic filter 535 at an angle of incidence AOI, the second dichroic filter 535 transmits a first portion 550a of the emission light (e.g., the portion having wavelengths within the passband region of the second dichroic filter 535) and reflects a second portion 550b of the emission light (e.g., the portion having wavelengths within the stopband region of the second dichroic filter 535). The first portion 550a and the second portion 550b may each be similarly described in terms of a central beam axis 552a, 552b. As referred to above, the optical axis may alternatively or additionally be used.

In the example configuration of FIGS. 5A and 5B, the second dichroic filter 535 is disposed such that the central beam axis 552 of the beam 550 is incident at an angle of incidence of 30 degrees. Similarly, the additional dichroic filters 540a, 540b are disposed such that the central beam axes 552a, 552b of the first and second portions 550a, 550b of the beam 550 are also incident at angles of incidence of 30 degrees. However, in various implementations these angles of incidence may be other angles smaller than 45 degrees, such as, for example, angles between 20 degrees and 40 degrees, between 25 degrees and 35 degrees, between 27.5 degrees and 32.5 degrees, or any other suitable angle of incidence. Moreover, the angles of incidence on each of the dichroic filters 535, 540a, 540b need not necessarily be the same. In some embodiments, some or all of the dichroic filters 535, 540a, 540b may be disposed such that their incident beams 550, 550a, 550b have different angles of incidence. As described above, the angle of incidence may be with respect to the optical axis of the optics within the imaging module, for example, the objective lens and/or the optics in the detection channels (e.g., the tube lenses) and the dichroic reflective surface in the respective dichroic beamsplitter. The same ranges and values for the angle of incidence apply to the case when the optical axis is used to specify the AOI.

The beams 550, 550a, 550b of emission light in a fluorescence microscopy system are typically diverging beams. As noted above, the beams of emission light can have a beam divergence large enough that regions of the beam within the beam diameter are incident upon the dichroic filters at angles of incidence that differ by up to 5 degrees or more relative to the angle of incidence of the central beam axis and/or optical axis of the optics. In some designs, the objective lens 510 may be configured, for example, have an f-number or numerical aperture selected to produce a smaller beam diameter for a given field of view of the microscope. In one example, the f-number or numerical aperture of the objective lens 510 may be selected such that the full diameter of the beams 550, 550a, 550b are incident upon dichroic filters 535, 540a, 540b at angles of incidence within, for example, 2 degrees, 2.5 degrees, 3 degrees, 3.5 degrees, 4 degrees, or 4.5 degrees of the angle of incidence of the central beam axes 552, 552a, 552b. In some implementations, example objective lens focal lengths suitable for producing such a narrow beam diameter may be longer than those typically employed in fluorescence microscopes, such as between 30 mm and 40 mm, between 35 mm and 37 mm, or other suitable range. In one example, an objective lens 510 having a focal length of 36 mm may produce a beam 550 characterized by a divergence small enough that light across the full diameter of the beam 550 is incident upon the second dichroic filter 535 at angles within 2.5 degrees of the angle of incidence of the central beam axis.

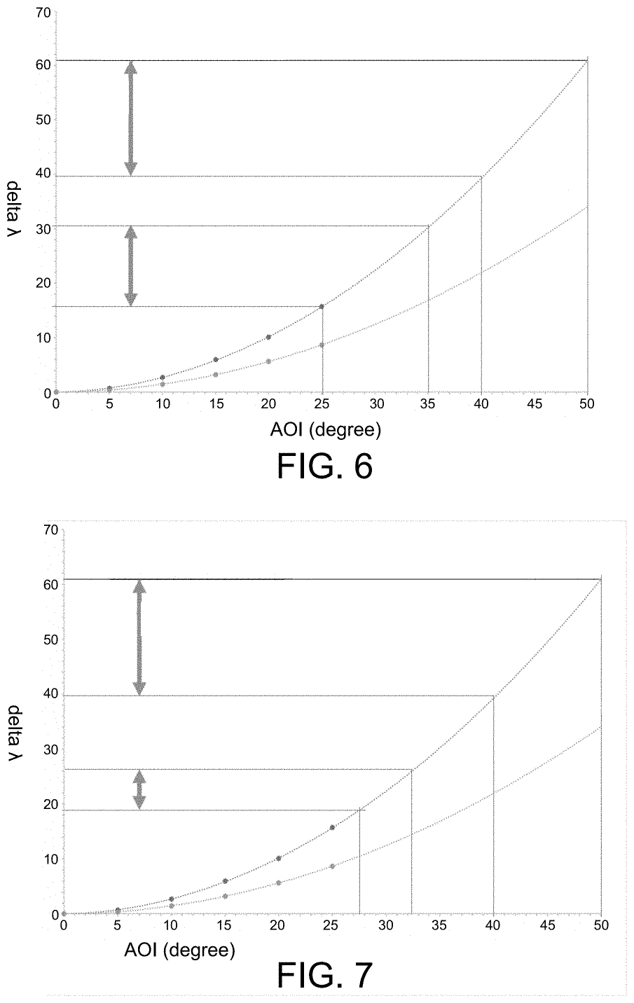

FIGS. 6 and 7 are graphs illustrating improved dichroic filter performance due to aspects of the configuration of FIGS. 5A and 5B. FIG. 6 is a graph similar to that of FIG. 3, illustrating the effect of angle of incidence on the transition width (e.g., the spectral span of the transmission edge) of a dichroic filter. FIG. 6 shows an example where the orientation of a dichroic filter (e.g., dichroic filters 535, 540a, 540b) and the dichroic reflective surface therein is such that its incident beam has an angle of incidence of 30 degrees, rather than 45 degrees. FIG. 6 shows how this reduced angle of incidence significantly improves the sharpness and the uniformity of the transition width across the full beam diameter. For example, while an angle of incidence of 45 degrees at the central beam axis results in a range of transition widths between about 40 nm and about 62 nm, an angle of incidence of 30 degrees at the central beam axis results in a range of transition widths between about 16 nm and about 30 nm. In this example, the average transition width is reduced from about 51 nm to about 23 nm, indicating a sharper transition between passband and stopband. Moreover, the variation in transition widths across the beam diameter is reduced by nearly 40% from a 22 nm range to a 14 nm range, indicating a more uniform sharpness of the transition over the area of the beam.

FIG. 7 illustrates additional advantages that may be realized by selecting the appropriate f-number or numerical aperture to reduce beam divergence. In some implementations a longer focal length is used. In the example of FIG. 7, the objective lens 510 has a focal length of 36 mm, which with the appropriate numerical aperture (e.g., less than 5), reduces the range of angles of incidence within the beam 550 from 30 degrees±5 degrees to 30 degrees±2.5 degrees. With this design, the range of transition widths may be reduced to between about 19 nm and about 26 nm. When compared to the improved system of FIG. 6, although the average transition width is substantially the same (e.g., a spectral span of roughly 23 nm), the variation in transition widths across the beam diameter is further reduced to a 7 nm range, representing a reduction of nearly 70% relative to the range of transition widths illustrated in FIG. 3.

Referring back to FIG. 4, the reduction in angle of incidence from 45 degrees to 30 degrees at the central beam axis is further advantageous because it reduces the beam spot size on the dichroic filter. As shown in FIG. 4, an angle of incidence of 45 degrees results in a beam footprint on the dichroic filter having an area greater than 1.4 times the cross-sectional area of the beam. However, an angle of incidence of 30 degrees results in a beam footprint on the dichroic filter having an area only about 1.15 times the cross-sectional area of the beam. Thus, reducing the angle of incidence at the dichroic filters 535, 540a, 540b from 45 degrees to 30 degrees results in a reduction of about 18% in the area of the beam footprint on the dichroic filters 535, 540a, 540b. This reduction in beam footprint area allows smaller dichroic filters to be used.

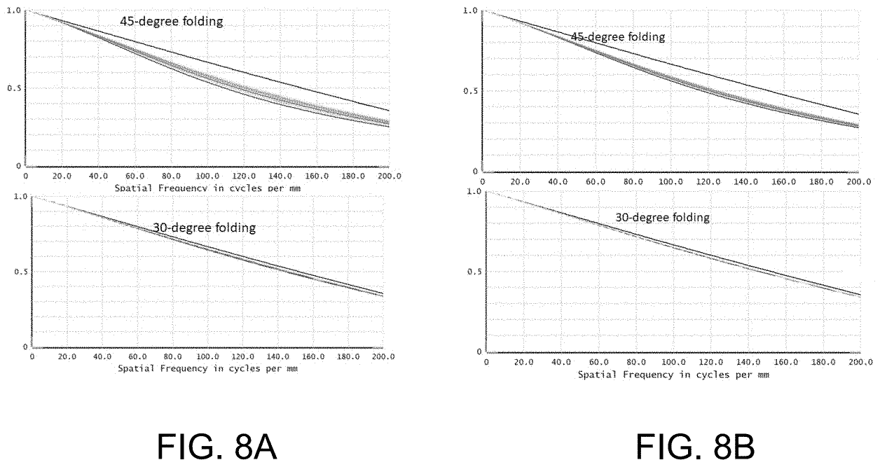

Referring now jointly to FIGS. 8A and 8B, the reduction in angle of incidence from 45 degrees to 30 degrees may also provide improved performance with regard to surface deformation caused by the dichroic filters. In general, the amount of surface deformation increase with larger area elements. If a larger areas on the dichroic filter is employed, a larger amount of surface deformation is encountered, introducing more wavefront error into the beam. As shown in FIGS. 8A and 8B, the reduction in angle of incidence to 30 degrees significantly reduces surface deformation to achieve close to diffraction-limited performance of the detection optics.

In some implementations, the polarization state of the excitation beam may be utilized to further improve the performance of the multi-channel fluorescence microscopes disclosed herein. Referring back to FIGS. 1A, 1B, and 5A, many implementations of the multi-channel fluorescence microscope have an epifluorescence configuration in which a first dichroic filter 130 or 530 merges the optical paths of the excitation beam and the beam of emission light such that both the excitation and emission light are transmitted through that objective lens 110, 510. As discussed above, the illumination source 115 may include a light source such as a laser or other source which provides the light that forms the excitation beam. In some designs, the light source comprises a linearly polarized light source and the excitation beam may be linearly polarized. In some designs, polarization optics are included to polarize the light and/or rotation the polarization. For example, a polarizer such as a linear polarizer may be included in an optical path of the excitation beam to polarize the excitation beam.

Retarders such as half wave retarders or a plurality of quarter wave retarders or retarders having other amounts of retardance may be included to rotate the linear polarization in some designs.

The linearly polarized excitation beam, when it is incident upon any dichroic filter or other planar interface, may be p-polarized (e.g., having an electric field component parallel to the plane of incidence), s-polarized (e.g., having an electric field component normal to the plane of incidence), or may have a combination of p-polarization and s-polarization states within the beam. The p- or s-polarization state of the excitation beam may be selected and/or changed by selecting the orientation of the illumination source 115 and/or one or more components thereof with respect to the first dichroic filter 130, 530 and/or with respect to any other surfaces with which the excitation beam will interact. In some implementations where the light source output linearly polarized light, the light source can be configured to provide s-polarized light. For example, the light source may comprise an emitter such as a solid state laser or a laser diode that may be rotated about its optical axis or the central axis of the beam to orient the linearly polarized light output therefrom. Alternatively or in addition, retarders may be employed to rotate the linear polarization about the optical axis or the central axis of the beam. As discussed above, in some implementations, for example when the light source does not output polarized light, a polarizer disposed in the optical path of the excitation beam can polarize the excitation beam. In some designs, for example, a linear polarizer is disposed in the optical path of the excitation beam. This polarizer may be rotated to provide the proper orientation of the linear polarization to provide s-polarized light.

In some designs, the linear polarization is rotated about the optical axis or the central axis of the beam such that s-polarization is incident on the dichroic reflector of the dichroic beamsplitter. When s-polarized light is incident on the dichroic reflector of the dichroic beamsplitter the transition between the pass band and the stop band is sharper as opposed to when p-polarized light is incident on the dichroic reflector of the dichroic beamsplitter.

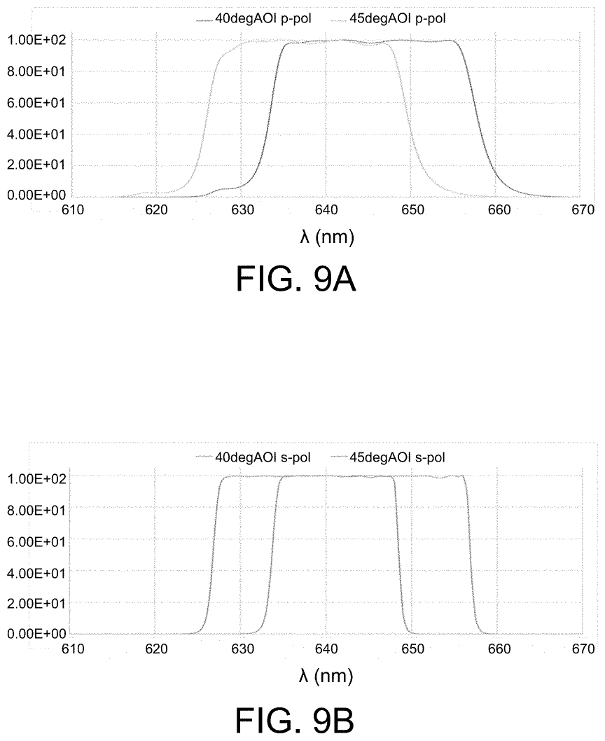

As shown in FIGS. 9A and 9B, use of the p- or s-polarization state of the excitation beam may significantly affect the narrowband performance of any excitation filters such as the first dichroic filter 130, 530. FIG. 9A illustrates a transmission spectrum between 610 nm and 670 nm for an example bandpass dichroic filter at angles of incidence of 40 degrees and 45 degrees, where the incident beam is linearly polarized and is p-polarized with respect to the plane of the dichroic filter. As shown in FIG. 9B, changing the orientation of the light source with respect to the dichroic filter, such that the incident beam is s-polarized with respect to the plane of the dichroic filter, results in a substantially sharper edge between the passband and the stopband of the dichroic filter. Thus, the illumination and imaging modules 100, 500 disclosed herein may advantageously have an illumination source 115 oriented relative to the first dichroic filter 130, 530 such that the excitation beam is s-polarized with respect to the plane of the first dichroic filter 130, 530. As discussed above, in some implementation, a polarizer such as a linear polarizer may be used to polarize the excitation beam. This polarizer may be rotated so as to provide an orientation of the linearly polarized light corresponding to s-polarized light. Also as discussed above, in some implementations, other approaches to rotating the linearly polarized light may be used. For example, optical retarders such as half wave retarders or multiple quarter wave retarders may be used to rotate the polarization direction. Other arrangements are possible.

As discussed above, in some implementations, the sample support structure may comprise a flow cell such as a dual surface flow cell having two surfaces containing sample sites that emit fluorescent light. FIGS. 10A and 10B schematically illustrate two such dual surface support structures. FIG. 10A shows a dual surface support structure such as a flow cell including an internal flow channel through which an analyte can be flowed. The flow channel may be formed between first and second, top and bottom, and/or front and back layers such as first and second, top and bottom, and/or front and back plates as shown. One or more of the plates may include a glass plate, such as a coverslip, or the like. In some implementations, the layer comprises borosilicate glass, quartz, or plastic. Interior surfaces of these top and bottom layers provide walls of the flow channel that assist in confining the flow of analyte through the flow channel of the flow cell. In some designs, these interior surfaces are planar. Similarly, the top and bottom layers may be planar. In some designs, at least one additional layer (not shown) is disposed between the top and bottom layers. This additional layer may have one or more pathways cut therein that assist in defining the flow channel and controlling the flow the flow of the analyte within the flow channel. Additional discussion of sample support structures can be found below.

FIG. 10A schematically illustrates a plurality of fluorescing sample sites on the first and second, top and bottom, and/or front and back interior surfaces of the flow cell.

In some implementations, reactions may occur at these at these sites to bind sample such that fluorescence is emitted from these sites. (Note that FIG. 10A is schematic and not drawn to scale. For example, the size and spacing of the fluorescing sample sites may be smaller than shown.)

FIG. 10B shows another dual surface support structure having two surfaces containing fluorescing sample sites to be imaged. The sample support structure comprises a substrate having first and second, top and bottom, and/or front and back exterior surfaces. In some designs, these exterior surfaces are planar. In various implementations, the analyte is flowed across these first and second exterior surfaces. FIG. 10B schematically illustrates a plurality of fluorescing sample sites on the first and second, top and bottom, and/or front and back exterior surfaces of the sample support structure. In some implementations, reactions may occur at these at these sites to bind sample such that fluorescence is emitted from these sites. (Note that FIG. 10B is schematic and not drawn to scale. For example, the size and spacing of the fluorescing sample sites may be smaller than shown.)

The fluorescence microscope described herein is configured to image these fluorescing sample sites. In some designs, only one of these first and second surfaces is in focus at one time. Accordingly, in such designs, one of the first or second surfaces is imaged at a first time and the other surface is imaged at a second time. The focus of the fluorescence microscope may be change after imaging one of the surfaces in order to image the other surface as the images are not simultaneously in focus. In some such designs, an optical compensation element is introduced into the optical path from the sample support structure and a photodetector array that captures and image of the surface. The depth of field of such fluorescence microscopes may not be sufficiently large to include both the first and second surfaces or include both first and second surfaces in focus.

In certain implementations described herein, both the first and second surfaces can be imaged at the same time. For example, the fluorescence microscope may have a depth of field that include both surfaces. This increased depth of field may be provided by reducing the numerical aperture of the objective lens (or microscope objective). For example, as discussed above, in various implementations the numerical aperture is less than 0.6, possibly 0.55 or less, 0.5 or less, 0.45 or less, 0.4 or less, 0.35 or less, 0.3 or less, 0.25 or less, 0.2 or less, 0.15 or less, 0.1 or less, 0.05 or less, or in any range formed by any of these values. The depth of field and/or depth of focus of the objective lens and/or fluorescent microscope can be 0.05 mm or more, 0.075 mm or more, 0.1 mm or more, 0.125 mm or more, 0.15 mm or more, 0.175 mm or more, 0.2 mm or more, 0.25 mm or more, 0.3 mm or more, 0.35 mm or more, 0.4 mm or more, 0.45 mm or more, 0.5 mm or more, or in any range between any of these values. For example, the depth of field and/or depth of focus of the objective and/or fluorescent microscope can, for example, be in a range from 0.05 mm to 0.250 mm, or 0.05 mm to 0.2 mm, or 0.05 mm to 0.15 mm or 0.05 mm to 0.125 mm or 0.05 to 0.100 mm or 0.05 to 0.150 mm or 0.05 to 0.200 mm. Alternatively, the depth of field and/or depth of focus of the objective and/or fluorescent microscope can be, for example, in a range from 0.075 mm to 0.250 mm, or 0.075 mm to 0.2 mm, or 0.075 mm to 0.15 mm or 0.075 mm to 0.125 mm or 0.075 to 0.100 mm or 0.075 to 0.150 mm or 0.075 to 0.200 mm or from 0.100 to 0.200 mm or from 0.200 to 0.300 mm or from 0.300 to 0.400 mm or from 0.400 mm to 0.500 mm. Other ranges formed by any of the values listed above are possible.

In some implementations, the first and second surfaces can be separated by 0.075 mm or more. For example, the first and second surfaces can be separated by 0.05 mm or more, 0.075 mm or more, 0.1 mm or more, 0.125 mm or more, 0.15 mm or more, 0.175 mm or more, 0.2 mm or more, 0.25 mm or more, 0.3 mm or more, 0.35 mm or more, 0.4 mm or more, 0.45 mm or more, 0.5 mm or more, or in any range between any of these values. For example, the separation of the first and second surfaces may be, for example, in a range from 0.05 mm to 0.250 mm, or 0.05 mm to 0.2 mm, or 0.05 mm to 0.15 mm or 0.05 mm to 0.125 mm or 0.05 to 0.100 mm. For example, the separation of the first and second surfaces may be, for example, in a range from 0.075 mm to 0.250 mm, or 0.075 mm to 0.2 mm, or 0.075 mm to 0.15 mm or 0.075 mm to 0.125 mm or 0.075 to 0.100 mm or from 0.075 mm to 0.250 mm, from 0.100 mm to 0.200 mm or from 0.200 to 0.300 mm or from 0.300 to 0.400 mm or from 0.400 mm to 0.500 mm. Other ranges formed by any of the values listed above are possible.

As shown in FIGS. 10A and 10B, the imaging optics (e.g., objective lens) may be positioned at a suitable distance (e.g., a distance corresponding to the work distance) from the first and second surfaces to form an image of the first and second surfaces on the detector array 124. In some implementations, both the first and second surfaces are in focus in the image formed on the detector array 124. In some implementations, neither of the first and second surfaces are fully or completely in focus in the image formed on the detector array 124. In some implementations, the first and second surfaces are blurred or not in focus in the image formed on the detector array 124. In some implementations, one of the first or second surfaces or both are not fully or completely in focus in the image formed on the detector array 124. In some implementations, one of the first or second surfaces or both are blurred or not in focus in the image formed on the detector array 124. In some implementations, sample sites such as fluorescing sample sites in both the first and second surfaces are in focus in the image formed on the detector array 124. In some implementations, sample sites such as fluorescing sample sites one of the first and second surfaces are not fully or completely in focus in the image formed on the detector array 124. In some implementations, sample sites such as fluorescing sample sites on both the first and second surfaces are not fully or completely in focus in the image formed on the detector array 124. In some implementations, sample sites such as fluorescing sample sites one of the first and second surfaces are blurred or not in focus in the image formed on the detector array 124. In some implementations, sample sites such as fluorescing sample sites on both the first and second surfaces are blurred or not in focus in the image formed on the detector array 124. Nevertheless, the sample sites such as the fluorescing sample sites may be resolvable, for example, in the lateral (e.g., X and/or Y directions) by the processing electronics, processor or other device(s) or at least be able to be processed.

As shown in the example of FIGS. 10A and 10B, the first surface is between said objective lens and said second surface. For example, as illustrated, the microscope objective is disposed above both the first and second surfaces and the first surface is disposed over the second surface. The first and second surfaces, for example, are at different depths. The first and second surfaces are at different distances from any one or more of the fluorescence microscope, the illumination and imaging module, imaging optics, the objective lens. The first and second surfaces are separated from each other with the first surface spaced apart above the second surface. In the example shown, the first and second surfaces are planar surfaces and said first is separated from each other along a direction normal to said first and second planar surfaces. Also, in the example shown, said objective lens has an optical axis and said first and second surfaces are separated from each other along the direction of said optical axis. Similarly, the separation between the first and second surfaces may correspond to the longitudinal distance such as along the optical path of the excitation beam and/or along an optical axis through the fluorescence microscope and/or the objective lens. Accordingly, these two surfaces may be separated by a distance from each other in the longitudinal (Z) direction (see FIGS. 10A and 10B), which may be along the direction of the central axis of the excitation beam, the central axis of the objective lens, and/or the optical axis of the objective lens and/or the fluorescence microscope. This direction may be orthogonal to the scan direction which may, for example, be in X and/or Y (e.g., see FIGS. 10A and 10B) and parallel to the direction (e.g., Z) of adjustment to provide focus. This separation may correspond, for example, to a flow channel within the flow cell in some implementations.

As discussed above, in various designs, the objective lens (possibly together with the optics 126) have a depth of field and/or depth of focus that is at least as large as the longitudinal separation (in Z direction) between the first and second surfaces (or between two object planes, surfaces or locations where the first and second surface would be located). The objective lens and the optics 126 can thus simultaneously form images of both the first and the second surface (or of two object planes or surfaces where the first and second surface would be located) on the photodetector array 124 and these images of the first and second surfaces may be in focus. In various implementations, compensation optics need not be moved into or out of an optical path of the imaging module to form an image or images such as an in-focus image or images of the first and second surfaces. Similarly, in various implementations, one or more optical elements (e.g., lens elements) in the imaging module (e.g., in the objective lens or optics 126) need not be moved, for example, in the longitudinal direction along the first and/or second optical paths (e.g., along the optical axis of the imaging optics—tube lens and/or microscope objective, etc.) to form an image or images such as an in-focus image or images of the first surface in comparison to the location of said one or more optical element when used to image the second surface. In some implementations, however, the imaging module includes an autofocus system configured to provide both the first and second surface in focus at the same time. In various implementations, the sample is in focus to sufficiently resolve the sample sites, which are closely spaced together in lateral directions (e.g., X and Y directions). Accordingly, in various implementations, no optical element enters an optical path between the sample support structure (e.g., between a translation stage that supports the sample support structure) and a photodetector array in said at least one detection channel in order to form an image such as an in-focus image of fluorescing sample sites on a first surface of the sample support structure onto the photodetector array and exits the optical path to form an image such as an in-focus images of fluorescing sample sites on a second surface of said sample support structure onto the photodetector array. Similarly, in various implementations, no optical compensation is used to form an image such as an in-focus image of fluorescing sample sites on a first surface of the sample support structure onto the photodetector array that is not identical to optical compensation used to form an image such as an in-focus image of fluorescing sample sites on a second surface of the sample support structure onto the photodetector array. Additionally, in certain implementations, no optical element in an optical path between the sample support structure (e.g., between a translation stage that supports the sample support structure) and a photodetector array in the at least one detection channels is adjusted differently to form an image such as an in-focus image of fluorescing sample sites on a first surface of the sample support structure onto the photodetector array than to form an image such as an in-focus image of fluorescing sample sites on a second surface of the sample support structure onto the photodetector array. Similarly, in some various implementations, no optical element in an optical path between the sample support structure (e.g., between a translation stage that supports the sample support structure) and a photodetector array in the at least one detection channels is moved a different amount or a different direction to form an images such as an in-focus image of fluorescing sample sites on the a first surface of the sample support structure onto the photodetector array than to form an image such as an in-focus image of fluorescing sample sites on a second surface of said sample support structure onto the photodetector array. Any combination of the features are possible. For example, in some implementations, an image or images such as an in-focus image or images of the upper interior surface and the lower interior surface of the flow cell can be obtained without moving an optical compensator into or out of an optical path between the flow cell and the at least one image sensor and without moving one or more optical elements of the imaging system (e.g., the objective and/or tube lens) along the optical path (e.g., optical axis) therebetween. For example, an image or images such as an in-focus image or images of the upper interior surface and the lower interior surface of the flow cell can be obtained without moving one or more optical elements of the tube lens into or out of the optical path or without moving one or more optical elements of the tube lens along the optical path (e.g., optical axis) therebetween.

Any one or more of the fluorescence microscope, illumination and imaging module 100, the imaging optics (e.g., optics 126), the objective lens or the tube lens may be designed to reduce or minimize aberration at two locations such as two planes corresponding to two surfaces on a flow cell or other sample support structure, for example, where fluorescing sample sites are located. Any one or more of the fluorescence microscope, illumination and imaging module 100, the imaging optics (e.g., optics 126), the objective lens or the tube lens may be designed to reduce the aberration at the selected locations or planes relative to other locations or planes such as first and second surfaces containing fluorescing sample sites on a dual surface flow cell. For example, any one or more of the fluorescence microscope, illumination and imaging module 100, the imaging optics (e.g., optics 126), the objective lens or the tube lens may be designed to reduce the aberration at two depths or planes located at different distances from the objective lens as compared to the aberrations associated with other depths or planes at other distances from the objective. For example, optical aberration may be less for imaging the first and second surfaces than elsewhere in a region from 1 to 10 mm from the objective lens. Additionally, any one or more of the fluorescence microscope, illumination and imaging module 100, the imaging optics (e.g., optics 126), the objective lens or the tube lens may in some embodiments be configured to compensate for aberration induced by transmission of emission light through one or more portions of the sample support structure such as a layer that includes one of the surfaces on which sample adheres as well as possibly a solution corresponding to the sample. This layer may comprise, e.g., glass, quartz, plastic, or other transparent material having a refractive index and introduce aberration. Any one or more of the fluorescence microscope, illumination and imaging module 100, the imaging optics (e.g., optics 126), the objective lens or the tube lens may in some embodiments be configured to compensate for aberration induced by a sample support structure coverslip or other components as well as possibly a solution corresponding to the sample.

Accordingly, the imaging performance may be substantially the same when imaging the first and second surface. The optical transfer functions (OTF) and/or modulation transfer functions (MTF) may be the same for imaging of the first and second surfaces. Either or both of these transfer functions may, for example, be within 20% of each other, be within 15% of each other, be within 10% of each other, within 5% of each other, within 2.5% over each other, within 1% of each other or in any range formed by any of these values. Accordingly, an imaging performance metric may be substantially the same for imaging the upper interior surface or the lower interior surface of the flow cell without moving an optical compensator into or out of an optical path between the flow cell and the at least one image sensor, and without moving one or more optical elements of the imaging system (e.g., the objective and/or tube lens) along the optical path (e.g., optical axis) therebetween. For example, an imaging performance metric may be substantially the same for imaging the upper interior surface or the lower interior surface of the flow cell without moving one or more optical elements of the tube lens into or out of the optical path or without moving one or more optical elements of the tube lens along the optical path (e.g., optical axis) therebetween. Discussion of MTF is included below and in U.S. Provisional Application No. 62/962,723 filed Jan. 17, 2020, which is incorporated herein by reference in its entirety.

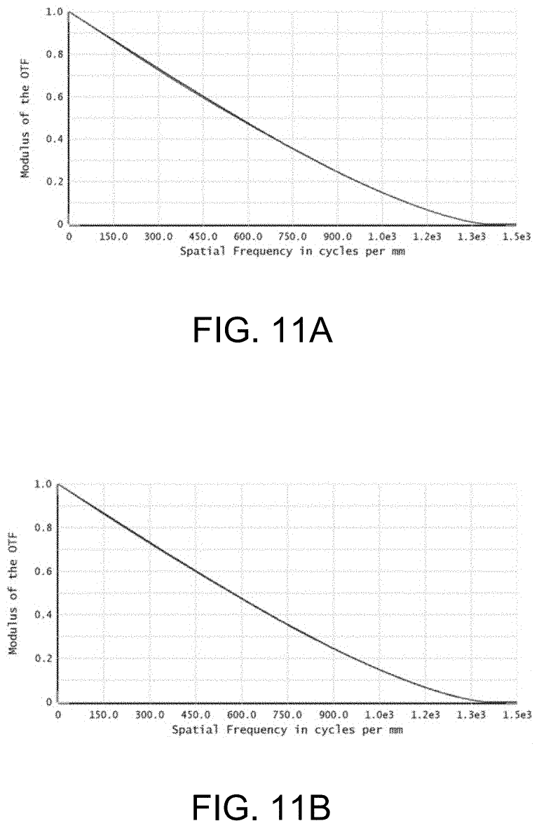

As discussed above, reducing the numerical aperture (NA) of the fluorescence microscope and/or of the objective lens, may increase the field-of-view to enable the comparable imaging of the two surfaces. FIGS. 11A-16B, show how the MTF is more similar at first and second surfaces separated by 1 mm of glass for lower numerical apertures than for larger numerical apertures.

FIGS. 11A and 11B show the MTF at first and second surfaces for an NA of 0.3.

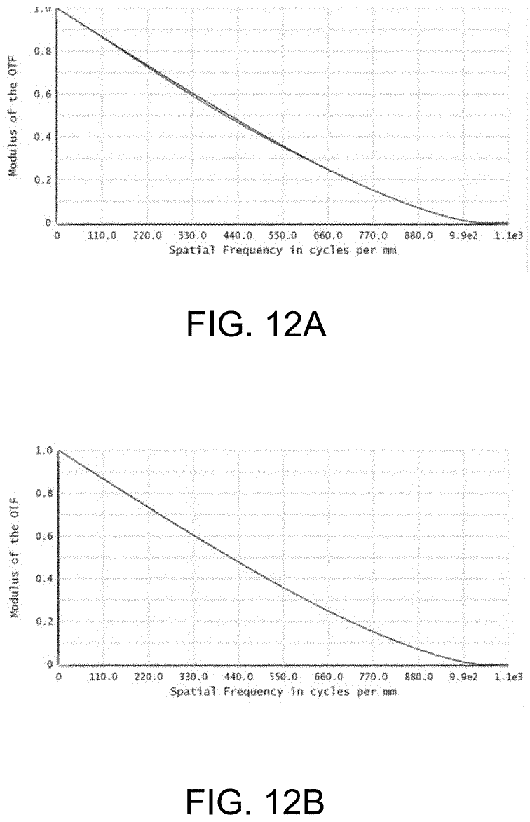

FIGS. 12A and 12B show the MTF at first and second surfaces for an NA of 0.4.

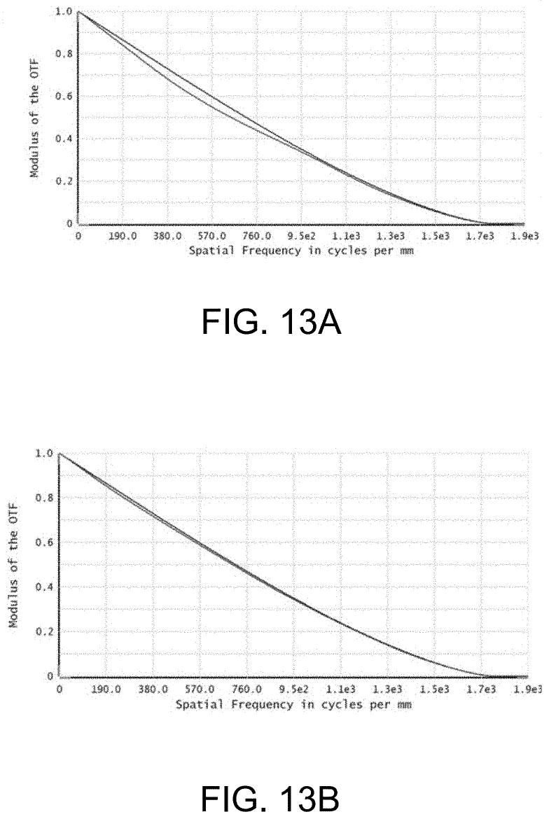

FIGS. 13A and 13B show the MTF at first and second surfaces for an NA of 0.5.

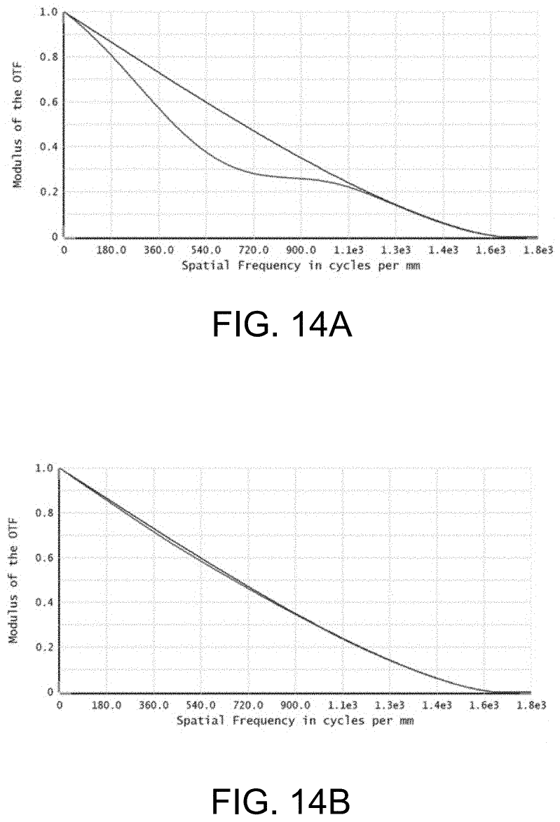

FIGS. 14A and 14B show the MTF at first and second surfaces for an NA of 0.6.

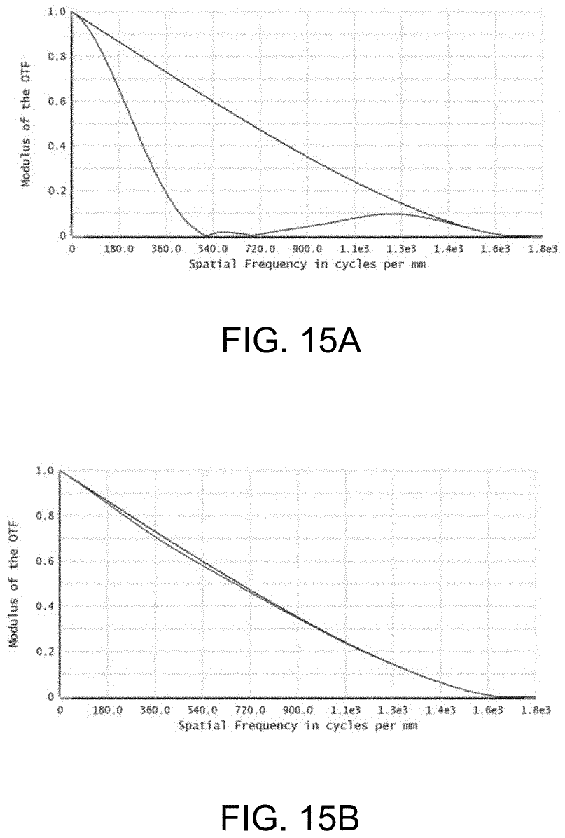

FIGS. 15A and 15B show the MTF at first and second surfaces for an NA of 0.7.

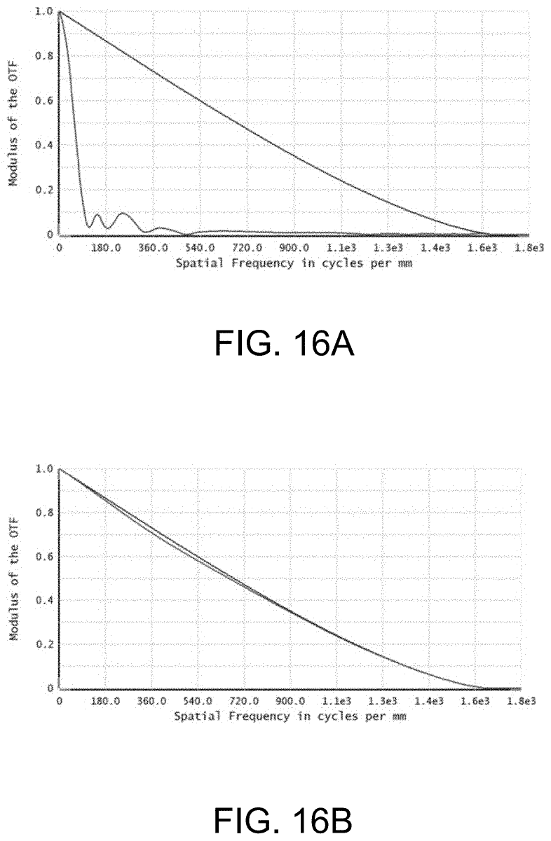

FIGS. 16A and 16B show the MTF at first and second surfaces for an NA of 0.8. The first and second images correspond to top and bottom surfaces.

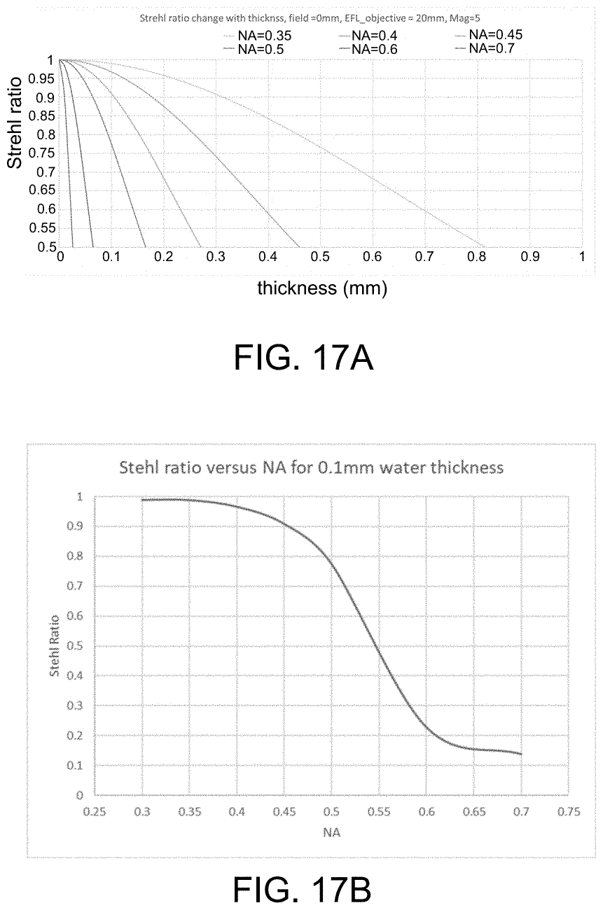

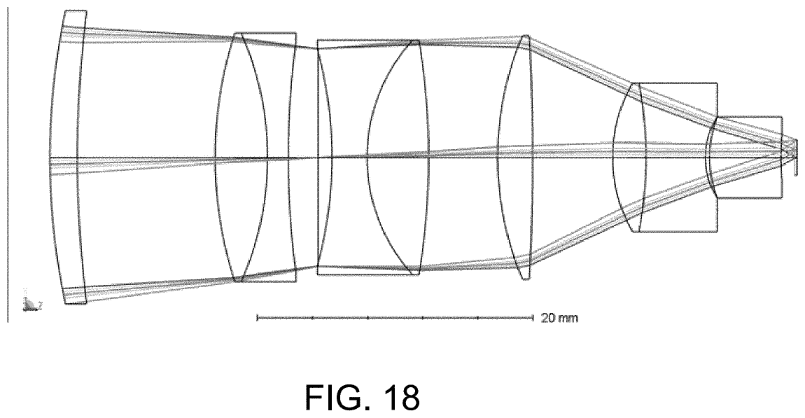

FIG. 17A shows a plot of the Strehl ratios for different numerical apertures for different thicknesses. The Strehl ratio is shown to decrease with separation, for example between the first and second surfaces. One of the surfaces would thus have deteriorated image quality with increasing separation between the two surfaces. However, this fall off in performance with separation is reduced for smaller numeral apertures imaging systems as compared to larger numerical aperture imaging systems.

FIG. 17B also shows a plot of the Strehl ratio as a function of numerical aperture. This plot illustrates the decreasing depth of field with NA. With increasing NA, the depth of field decreases such that the second surface is not in focus as much and not resolved as well. In this example, to obtain an indication of the effect of the separation, for example, between the first and second surfaces of the flow cell on imaging, imaging a plane through water having a thickness of 0.1 mm is simulated.

In general, however, reducing the numeral aperture reduces achievable resolution. This image quality can be at least partially offset by providing an increase contrast-to-noise ratio for images obtained. For example, the chemistry can be such that the fluorescence emission is stronger and/or background emission is weaker. Sample support structures comprising hydrophilic coating and/or hydrophilic substrates may be employed. In some cases, such hydrophilic coatings and/or hydrophilic substrates may reduce background noise. Additional discussions of sample support structures, hydrophilic surfaces and contrast-to-noise can be found below.

In some implementations, any one or more of the fluorescence microscope, the illumination and imaging module 100, the imaging optics (e.g., optics 126), the objective lens or the tube lens is configured to have reduced magnification. Any one or more of the fluorescence microscope, illumination and imaging module 100, the imaging optics (e.g., optics 126), the objective lens or the tube lens may be configured, for example, such that the fluorescence microscope has a magnification of less than 10 (10×). Any one or more of the fluorescence microscope, illumination and imaging module 100, the imaging optics (e.g., optics 126), the objective lens or the tube lens may be configured, for example, such that the fluorescence microscope has a magnification of 9× or less, 8× or less, 7× or less, 6× or less, 5× or less, 4× or less, 3× or less, 2× or less or a range between any of these values. Such reduced magnification adjust design constraints such that other design parameters can be achieved. For example, any one or more of the fluorescence microscope, illumination and imaging module 100, the imaging optics (e.g., optics 126), the objective lens or the tube lens may also be configured such that the fluorescence microscope has a large field-of-view (FOV), for example, a field-of-view of at least 3.0 mm or at least 3.2 mm (e.g., in width or diameter).

Any one or more of the fluorescence microscope, illumination and imaging module 100, the imaging optics (e.g., optics 126), the objective lens or the tube lens may also be configured such that the fluorescence microscope has an FOV at least 1.0 mm, at least 1.5 mm, at least 2.0 mm, at least 2.5 mm, at least 3.0 mm or at least 3.2 mm, at least 3.5 mm, at least 4.0 mm, at least 4.5 mm, at least 5.0 mm (e.g., in width or diameter) or any FOV in a range between any of these values. Any one or more of the fluorescence microscope, illumination and imaging module 100, the imaging optics (e.g., optics 126), the objective lens or the tube lens may be configured to provide the fluorescence microscope with such a field-of-view such that the FOV has less than 0.1 waves of aberration over at least 80% of field. Similarly, any one or more of the fluorescence microscope, illumination and imaging module 100, the imaging optics (e.g., optics 126), the objective lens or the tube lens may be configured such that the fluorescence microscope has such a FOV and is diffraction limited or is diffraction limited over such an FOV.

As discussed above, in various implementations, large field-of-view (FOV) is provided by the optical system. In some implementations, obtaining an increased FOV is facilitated in part by the use of larger photodetector arrays. The photodetector array, for example, may have an active area with a diagonal of at least 15 mm. The photodetector array, for example, may have an active area with a diagonal 10 mm or more, 11 mm or more, 12 mm or more, 13 mm or more, 14 mm or more, 15 mm or more, 16 mm or more, 17 mm or more, 18 mm or more, 19 mm or more, 20 mm or more, or any size (e.g., across the diagonal) in a range between any of these values. As discussed above, in some implementations the optical imaging system provides a reduced magnification, for example, of less than 10×, for example, of 8× or 5× or less, which may facilitate large FOV designs. Despite reduced magnification, resolution can be sufficient, as detector arrays having small pixel size or pitch may be used. The pixel size or pitch may, for example, be 5 mm or less, 4 mm or less, 3 mm or less, 2.5 mm or less, 2 mm or less, 1 mm or less, or any pixel size or pitch in a range between any of these values. In some implementations, the pixel size is smaller than twice the optical resolution provided by the optical imaging system (e.g., objective and tube lens) to satisfy Nyquist theorem. Accordingly, the pixel dimension or pitch for the image sensor may be such that a spatial sampling frequency for the fluorescence imaging system is at least twice an optical resolution of the fluorescence imaging system. For example, the spatial sampling frequency for the photodetector array may be is at least 2 times, at least 2.5 times, at least 3 times, at least 4 times, at least 5 times the optical resolution of the fluorescence imaging system (e.g., the illumination and imaging module, the objective and tube lens, the object lens and optics 126 in the detection channel, the imaging optics between the sample support structure or stage configured to support the sample support stage and the photodetector array) or any spatial sampling frequency in a range between any of these values. Other designs are possible. Some additional designs and design considerations for the fluorescence microscope, sample support structure (e.g., flow cell), and associated methods are discussed below. Although a wide range of features are discussed herein with respect to fluorescence microscopes, any of the features and methods describe herein may be applied to other types of optical systems such as other types of optical imaging systems including without limitation bright-field and dark-field imaging and may apply to luminescence or phosphorescence imaging.

Improved or optimized objective and/or tube lens for use with thicker coverslips: Existing design practice includes the design of objective lenses and/or use of commonly available off-the-shelf microscope objectives to optimize image quality when images are acquired through thin (e.g., <200 μm thick) microscope coverslips. When used to image on both sides of a fluidic channel or flow cell, the extra height of the gap between the two surfaces (i.e., the height of the fluid channel; typically, about 50 μm to 200 μm) introduces optical aberration in images captured for the non-optimal side of the fluidic channel, thereby causing lower optical resolution. This is primarily because the additional gap height is significant compared to the optimal coverslip thickness (typical fluid channel or gap heights of 50-200 μm vs. coverslip thicknesses of <200 μm). Another design practice is to utilize an additional “compensator” lens in the optical path when imaging is to be performed on the non-optimal side of the fluid channel or flow cell. This “compensator” lens and the mechanism required to move it in or out of the optical path so that either side of the flow cell may be imaged further increases system complexity and imaging system down time, and potentially degrades image quality due to vibration, etc.

In the present disclosure, the imaging system can be designed for compatibility with flow cell consumables that comprise a thicker coverslip or flow cell wall (thickness ≥700 μm). The objective lens design may be improved or optimized for a coverslip that is equal to the true cover slip thickness plus half of the effective gap thickness (e.g., 700 μm+½ *fluid channel (gap) height). This design significantly reduces the effect of gap height on image quality for the two surfaces of the fluid channel and balances the optical quality for images of the two surfaces, as the gap height is small relative to the total coverslip thickness and thus its impact on optical quality is reduced.

Additional advantages of using a thicker coverslip include improved control of thickness tolerance error during manufacturing, and a reduced likelihood that the coverslip undergoes deformation due to thermal and mounting-induced stress. Coverslip thickness error and deformation adversely impact imaging quality for both the top surface and the bottom surface of a flow cell.

To further improve the dual surface imaging quality for sequencing applications, our optical system design places a strong emphasis on improving or optimizing MTF (e.g., through improving or optimizing the objective lens and/or tube lens design) in the mid- to high-spatial frequency range that is most suitable for imaging and resolving small spots or clusters.

Improved or optimized tube lens design for use in combination with commercially-available, off-the-shelf objectives: For low-cost sequencer design, the use of a commercially-available, off-the-shelf objective lens may be preferred due to its relatively low price. However, as noted above, low-cost, off-the-shelf objectives are mostly optimized for use with thin coverslips of about 170 μm in thickness. In some instances, the disclosed optical systems may utilize a tube lens design that compensates for a thicker flow cell coverslip while enabling high image quality for both interior surfaces of a flow cell in dual-surface imaging applications. In some instances, the tube lens designs disclosed herein enable high quality imaging for both interior surfaces of a flow cell without moving an optical compensator into or out of the optical path between the flow cell and an image sensor, without moving one or more optical elements or components of the tube lens along the optical path, and without moving one or more optical elements or components of the tube lens into or out of the optical path.

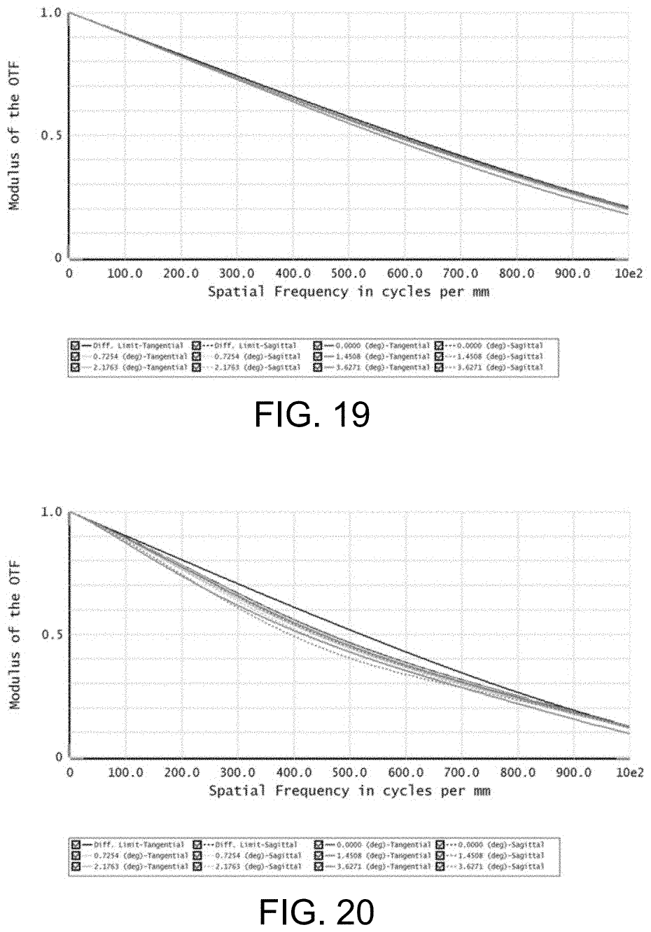

FIG. 18 provides an optical ray tracing diagram for a low light objective lens design that has been improved or optimized for imaging a surface on the opposite side of a 0.17 mm thick coverslip. The plot of modulation transfer function for this objective, shown in FIG. 19, indicates near-diffraction limited imaging performance when used with the designed-for 0.17 mm thick coverslip.

FIG. 20 provides a plot of the modulation transfer function for the same objective lens illustrated in FIG. 18 as a function of spatial frequency when used to image a surface on the opposite side of a 0.3 mm thick coverslip. The relatively minor deviations of MTF value over the spatial frequency range of about 100 to about 800 lines/mm (or cycles/mm) indicates that the image quality obtained even when using a 0.3 mm thick coverslip is still reasonable.

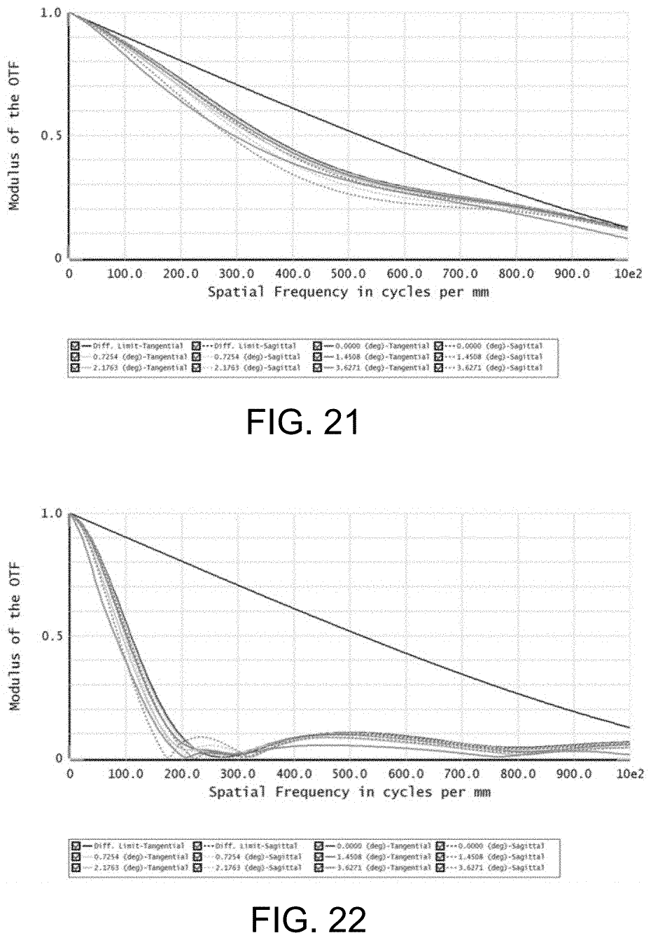

FIG. 21 provides a plot of the modulation transfer function for the same objective lens illustrated in FIG. 18 as a function of spatial frequency when used to image a surface that is separated from that on the opposite side of a 0.3 mm thick coverslip by a 0.1 mm thick layer of aqueous fluid (i.e., under the kind of conditions encountered for dual-side imaging of a flow cell when imaging the far surface). As can be seen in the plot of FIG. 21, imaging performance is degraded, as indicated by the deviations of the MTF curves from those for the an ideal, diffraction-limited case over the spatial frequency range of about 50 lp/mm to about 900 lp/mm.

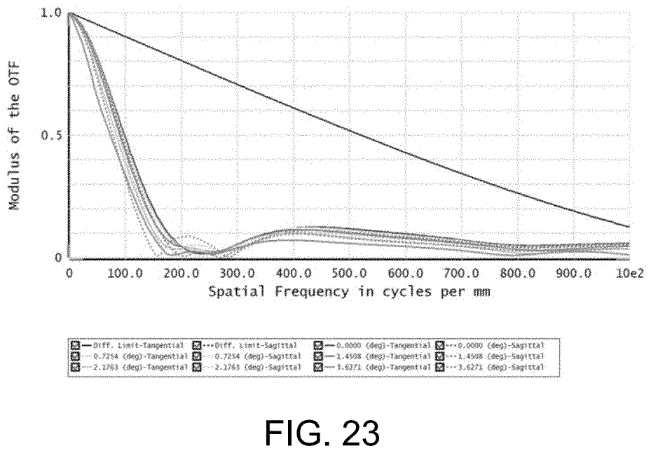

FIG. 22 and FIG. 23 provide plots of the modulation transfer function as a function of spatial frequency for the upper (or near) interior surface (FIG. 22) and lower (or far) interior surface (FIG. 23) of a flow cell when imaged using the objective lens illustrated in FIG. 18 through a 1.0 mm thick coverslip, and when the upper and lower interior surfaces are separated by a 0.1 mm thick layer of aqueous fluid. As can be seen, imaging performance is significantly degraded for both surfaces.

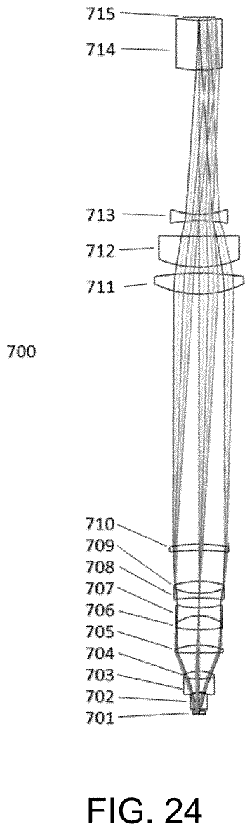

FIG. 24 provides a ray tracing diagram for a tube lens design which, if used in conjunction with the objective lens illustrated in FIG. 18, provides for improved dual-side imaging through a 1 mm thick coverslip. The optical design 700 comprising a compound objective (lens elements 702, 703, 704, 705, 706, 707, 708, 709, and 710) and a tube lens (lens elements 711, 712, 713, and 714) is improved or optimized for use with flow cells comprising a thick coverslip (or wall), e.g., greater than 700 μm thick, and a fluid channel thickness of at least 50 μm, and transfers the image of an interior surface from the flow cell 701 to the image sensor 715 with dramatically improved optical image quality and higher CNR.

In some instances, the tube lens (or tube lens assembly) may comprise at least two optical lens elements, at least three optical lens elements, at least four optical lens elements, at least five optical lens elements, at least six optical lens elements, at least seven optical lens elements, at least eight optical lens elements, at least nine optical lens elements, at least ten optical lens elements, or more, where the number of optical lens elements, the surface geometry of each element, and the order in which they are placed in the assembly is improved or optimized to correct for optical aberrations induced by the thick wall of the flow cell, and in some instances, allows one to use a commercially-available, off-the-shelf objective while still maintaining high-quality, dual-side imaging capability.

In some instances, as illustrated in FIG. 24, the tube lens assembly may comprise, in order, a first asymmetric convex-convex lens 711, a second convex-plano lens 712, a third asymmetric concave-concave lens 713, and a fourth asymmetric convex-concave lens 714.

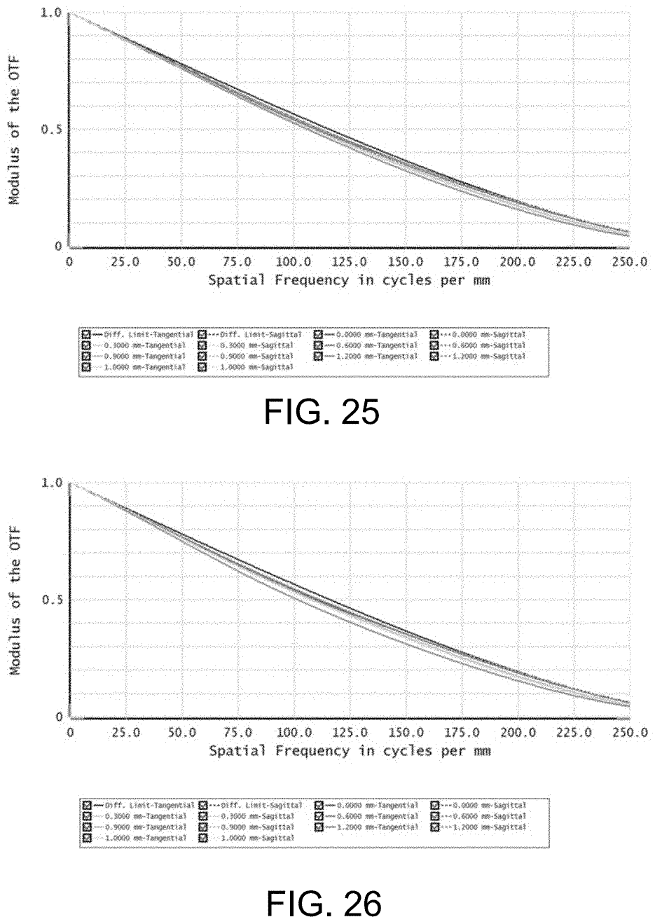

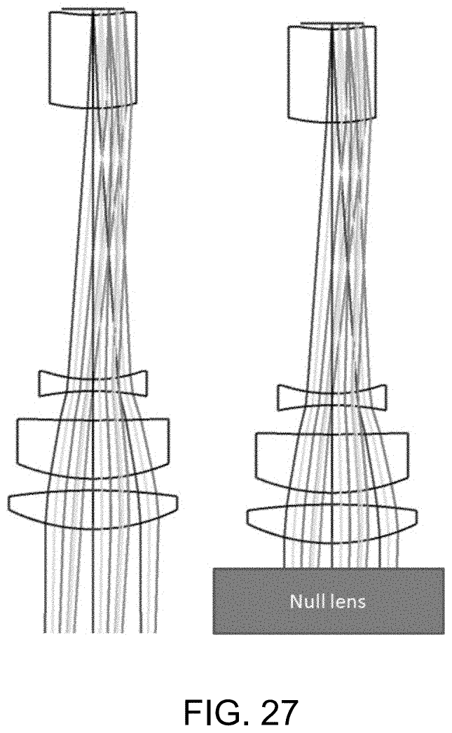

FIG. 25 and FIG. 26 provide plots of the modulation transfer function as a function of spatial frequency for the upper (or near) interior surface (FIG. 25) and lower (or far) interior surface (FIG. 26) of a flow cell when imaged using the objective lens (corrected for a 0.17 mm coverslip) and tube lens combination illustrated in FIG. 24 through a 1.0 mm thick coverslip, and when the upper and lower interior surfaces are separated by a 0.1 mm thick layer of aqueous fluid. As can be seen, the imaging performance achieved is nearly that expected for a diffraction-limited optical design.

FIG. 27 provides ray tracing diagrams for tube lens design (left) of the present disclosure that has been improved or optimized to provide high-quality, dual-side imaging performance. Because the tube lens is no longer infinity-corrected, an appropriately designed null lens (right) may be used in combination with the tube lens to compensate for the non-infinity-corrected tube lens for manufacturing and testing purposes.

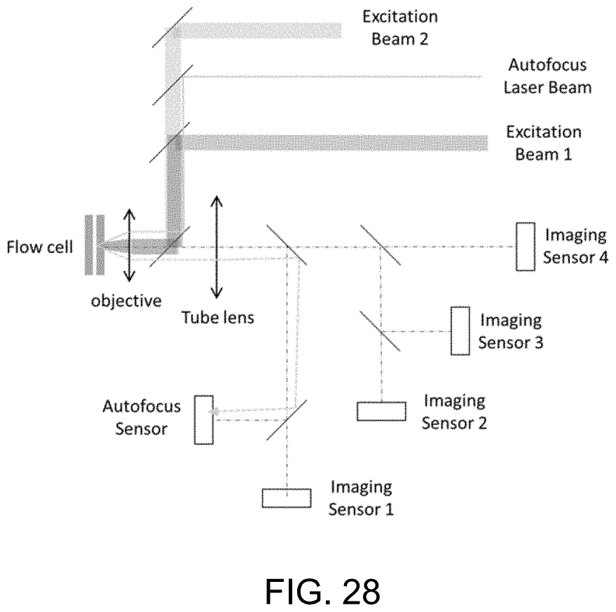

Dual wavelength excitation/four channel imaging system: FIG. 28 illustrates a dual excitation wavelength/four channel imaging system for dual-side imaging applications that includes an objective and tube lens combination that is scanned in a direction perpendicular to the optical axis to provide for large area imaging, e.g., by tiling several images to create a composite image having a total field-of-view (FOV) that is much larger than that for each individual image. The system comprises two excitation light sources, e.g., lasers or laser diodes, operating at different wavelengths and an autofocus laser. The two excitation light beams and autofocus laser beam are combined using a series of mirrors and/or dichroic reflectors and delivered to an upper or lower interior surface of the flow cell through the objective. Fluorescence that is emitted by labeled oligonucleotides (or other biomolecules) tethered to one of the flow cell surfaces is collected by the objective, transmitted through the tube lens, and directed to one of four imaging sensors according to the wavelength of the emitted light by a series of intermediate dichroic reflectors. Autofocus laser light that has been reflected from the flow cell surface is collected by the objective, transmitted through the tube lens, and directed to an autofocus sensor by a series of intermediate dichroic reflectors. The system allows accurate focus to be maintained (e.g., by adjusting the relative distance between the flow cell surface and the objective using a precision linear actuator, translation stage, or microscope turret-mounted focus adjustment mechanism, to reduce or minimize the reflected light spot size on the autofocus image sensor) while the objective/tube lens combination is scanned in a direction perpendicular to the optical axis of the objective. Dual wavelength excitation used in combination with four channel (i.e. four wavelength) imaging capability provides for high-throughput imaging of the upper (near) and lower (far) interior surfaces of the flow cell.

Imaging channel-specific tube lens adaptation or optimization: In imaging system design, it is possible to improve or optimize both the objective lens and the tube lens in the same wavelength region for all imaging channels. Typically, the same objective lens is shared by all imaging channels (see, for example FIG. 28), and each imaging channel either uses the same tube lens or has a tube lens that shares the same design.

In some instances, the imaging systems disclosed herein may further comprise a tube lens for each imaging channel where the tube lens has been independently tailored, improved or optimized for the specific imaging channel to improve image quality, e.g., to reduce or minimize distortion and field curvature, and improve depth-of-field (DOF) performance for each channel. Because the wavelength range (or bandpass) for each specific imaging channel is much narrower than the combined wavelength range for all channels, the wavelength- or channel-specific adaptation or optimization of the tube lens used in the disclosed systems results in significant improvements in imaging quality and performance.

This channel-specific adaptation or optimization results in improved image quality for both the top and bottom surfaces of the flow cell in dual-side imaging applications.

Dual-side imaging w/o fluid present in flow cell: For optimal imaging performance of both top and bottom interior surfaces of a flow cell, a motion-actuated compensator can be used to correct for optical aberrations induced by the fluid in the flow cell (typically comprising a fluid layer thickness of about 50-200 μm). In some instances of the disclosed optical system designs, the top interior surface of the flow cell may be imaged with fluid present in the flow cell. Once the sequencing chemistry cycle has been completed, the fluid may be extracted from the flow cell for imaging of the bottom interior surface. Thus, in some instances, even without the use of a compensator, the image quality for the bottom surface is maintained.

Compensation for optical aberration and/or vibration using electro-optical phase plates: In some instances, dual-surface image quality may be improved without requiring the removal of the fluid from the flow cell by using an electro-optical phase plate (or other corrective lens) in combination with the objective to cancel the optical aberrations induced by the presence of the fluid. In some instances, the use of an electro-optical phase plate (or lens) may be used to remove the effects of vibration arising from the mechanical motion of a motion-actuated compensator and may provide faster image acquisition times and sequencing cycle times for genomic sequencing applications.

Fluorescence imaging module specifications: In some instances, the numerical aperture of the disclosed optical system designs may range from about 0.1 to about 1.4. In some instances, the numerical aperture may be at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, or at least 1.4. In some instances, the numerical aperture may be at most 1.4, at most 1.3, at most 1.2, at most 1.1, at most 1.0, at most 0.9, at most 0.8, at most 0.7, at most 0.6, at most 0.5, at most 0.4, at most 0.3, at most 0.2 or at most 0.1. Any of the lower and upper values described in this paragraph may be combined to form a range included within the present disclosure, for example, in some instances the numerical aperture may range from about 0.1 to about 0.6. Those of skill in the art will recognize that the numerical aperture may have any value within this range, e.g., about 0.55.

In some instances, depending on the numerical aperture of the optical system, the minimum resolvable spot (or feature) separation distance at the sample plane achieved by the disclosed optical system designs may range from about 0.5 μm to about 2 μm. In some instances, the minimum resolvable spot separation distance at the sample plane may be at least 0.5 μm, at least 0.6 μm, at least 0.7 μm, at least 0.8 μm, at least 0.9 μm, at least 1.0 μm, at least 1.2 μm, at least 1.4 μm, at least 1.6 μm, at least 1.8 μm, or at least 1.0 μm. In some instances, the minimum resolvable spot separation distance may be at most 2.0 μm, at most 1.8 μm, at most 1.6 μm, at most 1.4 μm, at most 1.2 μm, at most 1.0 μm, at most 0.9 μm, at most 0.8 μm, at most 0.7 μm, at most 0.6 μm, or at most 0.5 μm. Any of the lower and upper values described in this paragraph may be combined to form a range included within the present disclosure, for example, in some instances the minimum resolvable spot separation distance may range from about 0.8 μm to about 1.6 μm. Those of skill in the art will recognize that the minimum resolvable spot separation distance may have any value within this range, e.g., about 0.95 μm.

In some instances of the disclosed optical designs, a spatial oversampling scheme is utilized wherein the spatial sampling frequency is at least 2×, 2.5×, 3×, 3.5×, 4×, 4.5×, 5×, 6×, 7×, 8×, 9×, or 10× the optical resolution X (lp/mm). The spatial sampling frequency may also be with one or more ranges formed by any of these foregoing values. For example, in some instances the spatial sampling frequency may range from about 2× to about 3× or from 2× to 4×, etc.

In some instances, the optical system magnification may range from about 2× to about 20×. In some instances, the optical system magnification may be at least 2×, at least 3×, at least 4×, at least 5×, at least 10×, at least 15×, or at least 20×. In some instances, the optical system magnification may be at most 20×, at most 15×, at most 10×, at most 5×, at most 4×, at most 3×, or at most 2×. Any of the lower and upper values described in this paragraph may be combined to form a range included within the present disclosure, for example, in some instances the optical system magnification may range from about 3× to about 10×. Those of skill in the art will recognize that the optical system magnification may have any value within this range, e.g., about 12.5×.

In some instances, the pixel size selected for the image sensor used in the disclosed optical system designs may range in at least one dimension from about 1 μm to about 10 μm. In some instances, the pixel size may be at least 1 μm, at least 2 μm, at least 3 μm, at least 4 μm, at least 5 μm, at least 6 μm, at least 7 μm, at least 8 μm, at least 9 μm, or at least 10 μm. In some instances, the pixel size may be at most 10 μm, at most 9 μm, at most 8 μm, at most 7 μm, at most 6 μm, at most 5 μm, at most 4 μm, at most 3 μm, at most 2 μm, or at most 1 μm. Any of the lower and upper values described in this paragraph may be combined to form a range included within the present disclosure, for example, in some instances the pixel size may range from about 3 μm to about 9 μm. Those of skill in the art will recognize that the pixel size may have any value within this range, e.g., about 1.4 μm.

In some instances of the disclosed optical designs, the design of the objective lens may be improved or optimized for a different coverslip of flow cell thickness. For example, in some instances the objective lens may be designed for optimal optical performance for a coverslip that is from about 200 μm to about 1,000 μm thick. In some instances, the objective lens may be designed for optimal performance with a coverslip that is at least 200 μm, at least 300 μm, at least 400 μm, at least 500 μm, at least 600 μm, at least 700 μm, at least 800 μm, at least 900 μm, or at least 1,000 μm thick. In some instances, the objective lens may be designed for optimal performance with a coverslip that is at most 1,000 μm, at most 900 μm, at most 800 μm, at most 700 μm, at most 600 μm, at most 500 μm, at most 400 μm, at most 300 μm, or at most 200 μm thick. Any of the lower and upper values described in this paragraph may be combined to form a range included within the present disclosure, for example, in some instances the objective lens may be designed for optimal optical performance for a coverslip that may range from about 300 μm to about 900 μm. Those of skill in the art will recognize that the objective lens may be designed for optimal optical performance for a coverslip that may have any value within this range, e.g., about 725 μm.

Improved contrast-to-noise ratio (CNR), field-of-view (FOV), spectral separation, and timing design to increase or maximize information transfer and throughput: Another way to increase or maximize information transfer in imaging systems designed for genomics applications is to increase the size of the field-of-view (FOV) and reduce the time required to image a specific FOV. With typical large NA optical imaging systems, it may be common to acquire images for fields-of-view that are on the order of 1 mm² in area, where in the presently disclosed imaging system designs large FOV objectives with long working distances are specified to enable imaging of areas of 1 mm² or larger.

In some cases, the disclosed imaging systems are designed for use in combination with proprietary low-binding substrate surfaces and DNA amplification processes that reduce fluorescence background arising from a variety of confounding signals including, but are not limited to, nonspecific adsorption of fluorescent dyes to substrate surfaces, nonspecific nucleic acid amplification products, e.g., nucleic acid amplification products that arise on the substrate surface in areas between the spots or features corresponding to clonally-amplified clusters of nucleic acid molecules (i.e., specifically amplified colonies), nonspecific nucleic acid amplification products that may arise within the amplified colonies, phased and pre-phased nucleic acid strands, etc. The use of low-binding substrate surfaces and DNA amplification processes that reduce fluorescence background in combination with the disclosed optical imaging systems may significantly cut down on the time required to image each FOV.

The presently disclosed system designs may further reduce the required imaging time through imaging sequence improvement or optimization where multiple channels of fluorescence images are acquired simultaneously or with overlapping timing, and where spectral separation of the fluorescence signals is designed to reduce cross-talks between fluorescence detection channels and between the excitation light and the fluorescence signal(s).

The presently disclosed system designs may further reduce the required imaging time through improvement or optimization of scanning motion sequence. In the typical approach, an X-Y translation stage is used to move the target FOV into position underneath the objective, an autofocus step is performed where optimal focal position is determined and the objective is moved in the Z direction to the determined focal position, and an image is acquired. A sequence of fluorescence images is acquired by cycling through a series of target FOV positions. From an information transfer duty cycle perspective, information is only transferred during the fluorescence image acquisition portion of the cycle. In the presently disclosed imaging system designs, a single-step motion in which all axes (X-Y-Z) are repositioned simultaneously is performed, and the autofocus step is used to check focal position error. The additional Z motion is only commanded if the focal position error (i.e., the difference between the focal plane position and the sample plane position) exceeds a certain limit (e.g., a specified error threshold). Coupled with high speed X-Y motion, this approach increases the duty cycle of the system, and thus increases the imaging throughput per unit time.

Furthermore, by matching the optical collection efficiency, modulation transfer function, and image sensor performance characteristics of the design with the fluorescence photon flux expected for the input excitation photon flux, dye efficiency (related to dye extinction coefficient and fluorescence quantum yield), while accounting for background signal and system noise characteristics, the time required to acquire high quality (high contrast-to-noise ratio (CNR) images) may be reduced or minimized.

The combination of efficient image acquisition and improved or optimized translation stage step and settle times leads to fast imaging times (i.e., the overall time required per field-of-view) and higher throughput imaging system performance.

Along with the large FOV and fast image acquisition duty cycle, the disclosed designs may comprise also specifying image plane flatness, chromatic focus performance between fluorescence detection channels, sensor flatness, image distortion, and focus quality specifications.

Chromatic focus performance is further improved by individually aligning the image sensors for different fluorescence detection channels such that the best focal plane for each detection channel overlaps. The design goal is to ensure that images across more than 90 percent of the field-of-view are acquired within ±100 nm (or less) relative to the best focal plane for each channel, thus increasing or maximizing the transfer of individual spot intensity signals. In some instances, the disclosed designs further ensure that images across 99 percent of the field-of-view are acquired within ±150 nm (or less) relative to the best focal plane for each channel, and that images across more, e.g., the entire field-of-view, are acquired within ±200 nm (or less) relative to the best focal plane for each imaging channel.

Fluorescence imaging module specifications (continued): In some instances of the disclosed optical system designs, the area of the field-of-view may range from about 1 mm² to about 5 mm². In some instances, the field-of-view may be at least 1 mm², at least 2 mm², at least 3 mm², at least 4 mm², or at least 5 mm² in area. In some instances, the field-of-view may be at most 5 mm², at most 4 mm², at most 3 mm², or at most 2 mm² in area. Any of the lower and upper values described in this paragraph may be combined to form a range included within the present disclosure, for example, in some instances the field-of-view may range from about 3 mm² to about 4 mm² in area. Those of skill in the art will recognize that the area of the field-of-view may have any value within this range, e.g., 2.75 mm².

In some instances of the disclosed optical imaging modules, the maximum translation stage velocity on any one axis may range from about 1 mm/sec to about 5 mm/sec. In some instances, the maximum translation stage velocity may be at least 1 mm/sec, at least 2 mm/sec, at least 3 mm/sec, at least 4 mm/sec, or at least 5 mm/sec. In some instances, the maximum translation stage velocity may be at most 5 mm/sec, at most 4 mm/sec, at most 3 mm/sec, at most 2 mm/sec, or at most 1 mm/sec. Any of the lower and upper values described in this paragraph may be combined to form a range included within the present disclosure, for example, in some instances the maximum translation stage velocity may range from about 2 mm/sec to about 4 mm/sec. Those of skill in the art will recognize that the maximum translation stage velocity may have any value within this range, e.g., about 2.6 mm/sec.

In some instances of the disclosed optical imaging modules, the maximum acceleration on any one axis of motion may range from about 2 mm/sec² to about 10 mm/sec². In some instances, the maximum acceleration may be at least 2 mm/sec², at least 3 mm/sec², at least 4 mm/sec², at least 5 mm/sec², at least 6 mm/sec², at least 7 mm/sec², at least 8 mm/sec², at least 9 mm/sec², or at least 10 mm/sec². In some instances, the maximum acceleration may be at most 10 mm/sec², at most 9 mm/sec², at most 8 mm/sec², at most 7 mm/sec², at most 6 mm/sec², at most 5 mm/sec², at most 4 mm/sec², at most 3 mm/sec², or at most 2 mm/sec². Any of the lower and upper values described in this paragraph may be combined to form a range included within the present disclosure, for example, in some instances the maximum acceleration may range from about 2 mm/sec² to about 8 mm/sec². Those of skill in the art will recognize that the maximum acceleration may have any value within this range, e.g., about 3.7 mm/sec².

In some instances of the disclosed optical imaging modules, the repeatability of positioning for any one axis may range from about 0.1 μm to about 2 μm. In some instances, the repeatability of positioning may be at least 0.1 μm, at least 0.2 μm, at least 0.3 μm, at least 0.4 μm, at least 0.5 μm, at least 0.6 μm, at least 0.7 μm, at least 0.8 μm, at least 0.9 μm, at least 1.0 μm, at least 1.2 μm, at least 1.4 μm, at least 1.6 μm, at least 1.8 μm, or at least 2.0 μm. In some instances, the repeatability of positioning may be at most 2.0 μm, at most 1.8 μm, at most 1.6 μm, at most 1.4 μm, at most 1.2 μm, at most 1.0 μm, at most 0.9 μm, at most 0.8 μm, at most 0.7 μm, at most 0.6 μm, at most 0.5 μm, at most 0.4 μm, at most 0.3 μm, at most 0.2 μm, or at most 0.1 μm. Any of the lower and upper values described in this paragraph may be combined to form a range included within the present disclosure, for example, in some instances the repeatability of positioning may range from about 0.3 μm to about 1.2 μm. Those of skill in the art will recognize that the repeatability of positioning may have any value within this range, e.g., about 0.47 μm.

In some instances of the disclosed optical imaging modules, the maximum time required to reposition the sample plane (field-of-view) relative to the optics, or vice versa, may range from about 0.1 sec to about 0.5 sec. In some instances, the maximum repositioning time (i.e., the scan stage step and settle time) may be at least 0.1 sec, at least 0.2 sec, at least 0.3 sec, at least 0.4 sec, or at least 0.5 sec. In some instances, the maximum repositioning time may be at most 0.5 sec, at most 0.4 sec, at most 0.3 sec, at most 0.2 sec, or at most 0.1 sec. Any of the lower and upper values described in this paragraph may be combined to form a range included within the present disclosure, for example, in some instances the maximum repositioning time may range from about 0.2 sec to about 0.4 sec. Those of skill in the art will recognize that the maximum repositioning time may have any value within this range, e.g., about 0.45 sec.

In some instances of the disclosed optical imaging modules, the specified error threshold for triggering an autofocus correction may range from about 50 nm to about 200 nm. In some instances, the error threshold may be at least 50 nm, at least 75 nm, at least 100 nm, at least 125 nm, at least 150 nm, at least 175 nm, or at least 200 nm. In some instances, the error threshold may be at most 200 nm, at most 175 nm, at most 150 nm, at most 125 nm, at most 100 nm, at most 75 nm, or at most 50 nm. Any of the lower and upper values described in this paragraph may be combined to form a range included within the present disclosure, for example, in some instances the error threshold may range from about 75 nm to about 150 nm. Those of skill in the art will recognize that the error threshold may have any value within this range, e.g., about 105 nm.

In some instances of the disclosed optical imaging modules, the image acquisition time may range from about 0.001 sec to about 1 sec. In some instances, the image acquisition time may be at least 0.001 sec, at least 0.01 sec, at least 0.1 sec, or at least 1 sec. in some instances, the image acquisition time may be at most 1 sec, at most 0.1 sec, at most 0.01 sec, or at most 0.001 sec. Any of the lower and upper values described in this paragraph may be combined to form a range included within the present disclosure, for example, in some instances the image acquisition time may range from about 0.01 sec to about 0.1 sec. Those of skill in the art will recognize that the image acquisition time may have any value within this range, e.g., about 0.250 seconds.

In some instances, the imaging times may range from about 0.5 seconds to about 3 seconds per field-of-view. In some instances, the imaging time may be at least 0.5 seconds, at least 1 second, at least 1.5 seconds, at least 2 seconds, at least 2.5 seconds, or at least 3 seconds per FOV. In some instances, the imaging time may be at most 3 seconds, at most 2.5 seconds, at most 2 seconds, at most 1.5 seconds, at most 1 second, or at most 0.5 seconds per FOV. Any of the lower and upper values described in this paragraph may be combined to form a range included within the present disclosure, for example, in some instances the imaging time may range from about 1 second to about 2.5 seconds. Those of skill in the art will recognize that the imaging time may have any value within this range, e.g., about 1.85 seconds.

In some instances, images across 80%, 90%, 95%, 98%, 99%, or 100% percent of the field-of-view are acquired within ±200 nm, ±175 nm, ±150 nm, ±125 nm, ±100 nm, ±75 nm, or ±50 nm relative to the best focal plane for each fluorescence (or other imaging mode) detection channel. Any of these the values described in this paragraph may be combined to form a range included within the present disclosure, for example, in some instances images across from 80% to 99% of the field-of-view are acquired within ±200 nm to ±50 nm relative to the best focal plane for each fluorescence (or other imaging mode) detection channel.

Illumination optical path design: Another factor for improving signal-to-noise ratio (SNR), contrast-to-noise ratio (CNR), and/or increasing throughput is to increase illumination power density to the sample. In some instances, the disclosed imaging systems may comprise an illumination path design that utilizes a high-power laser or laser diode coupled with a liquid light guide. The liquid light guide removes optical speckle that is intrinsic to coherent light sources such as lasers and laser diodes. Furthermore, the coupling optics are designed in such a way as to underfill the entrance aperture of the liquid light guide. The underfilling of the liquid light guide entrance aperture reduces the effective numerical aperture of the illumination beam entering the objective lens, and thus improves light delivery efficiency through the objective onto the sample plane. With this design innovation, one can achieve illumination power densities up to 3× that for conventional designs over a large field-of-view (FOV).

By utilizing the angle-dependent discrimination of s- and p-polarization, in some instances, the illumination beam polarization may be orientated to reduce the amount of back-scattered and back-reflected illumination light that reaches the imaging sensors.

Assessing image quality: For any of the embodiments of the optical imaging designs disclosed herein, imaging performance or imaging quality may be assessed using any of a number of performance metrics known to those of skill in the art. Examples include, but are not limited to, measurements of modulation transfer function (MTF) at one or more specified spatial frequencies, defocus, spherical aberration, chromatic aberration, coma, astigmatism, field curvature, image distortion, contrast-to-noise ratio (CNR), or any combination thereof.

In some instances, the disclosed optical designs for dual-side imaging (e.g., the disclosed tube lens designs, the use of an electro-optical phase plate in combination with an objective, etc.) may yield significant improvements for image quality for both the upper (near) and lower (far) interior surfaces of a flow cell, such that the difference in an imaging performance metric for imaging the upper interior surface and the lower interior surface of the flow cell is less than 20%, less than 15%, less than 10%, less than 5%, less than 4%, less than 3%, less than 2%, or less than 1% for any of the imaging performance metrics listed above, either individually or in combination. Any of these the values described in this paragraph may be combined to form a range included within the present disclosure, for example, in some instances the difference in the imaging performance metric is in the range from 1%-5%.

In some instances, the disclosed optical designs for dual-side imaging (e.g., comprising the disclosed tube lens designs, the use of an electro-optical phase plate in combination with an objective, etc.) may yield significant improvements for image quality such that an image quality performance metric for dual-side imaging provides for an at least 1%, at least 2%, at least 3%, at least 4%, at least 5%, at least 10%, at least 15%, at least 20%, at least 25%, or at least 30% improvement in the imaging performance metric for dual-side imaging compared to that for a conventional system comprising, e.g., an objective lens, a motion-actuated compensator (that is moved out of or into the optical path when imaging the near or far interior surfaces of a flow cell), and an image sensor for any of the imaging performance metrics listed above, either individually or in combination. In some instances, fluorescence imaging systems comprising one or more of the disclosed tube lens designs provides for an at least equivalent or better improvement in an imaging performance metric for dual-side imaging compared to that for a conventional system comprising an objective lens, a motion-actuated compensator, and an image sensor. In some instances, fluorescence imaging systems comprising one or more of the disclosed tube lens designs provides for an at least 5%, 10%, 15%, 20%, 25%, 30%, 35%, 40%, 45%, or 50% improvement in an imaging performance metric for dual-side imaging compared to that for a conventional system comprising an objective lens, a motion-actuated compensator, and an image sensor. Any of these the values described in this paragraph may be combined to form a range included within the present disclosure, for example, in some instances the improvement is in the range from 5-10% or 10%-20%.

Imaging modules and systems: It will be understood by those of skill in the art that the disclosed imaging systems or modules may, in some instances, be stand-alone optical systems designed for imaging a sample or substrate surface. In some instances, they may comprise one or more processors or computers. In some instances, they may comprise one or more software packages that provide instrument control functionality and/or image processing functionality. In some instances, in addition to optical components such as light sources (e.g., solid-state lasers, dye lasers, diode lasers, arc lamps, tungsten-halogen lamps, etc.), lenses, prisms, mirrors, dichroic reflectors, optical filters, optical bandpass filters, apertures, and image sensors (e.g., complementary metal oxide semiconductor (CMOS) image sensors and cameras, charge-coupled device (CCD) image sensors and cameras, etc.), they may also include mechanical and/or optomechanical components, such as an X-Y translation stage, an X-Y-Z translation stage, a piezoelectic focusing mechanism, and the like. In some instances, they may function as modules, components, sub-assemblies, or sub-systems of larger systems designed for genomics applications (e.g., genetic testing and/or nucleic acid sequencing applications). For example, in some instances, they may function as modules, components, sub-assemblies, or sub-systems of larger systems that further comprise light-tight and/or other environmental control housings, temperature control modules, fluidics control modules, fluid dispensing robotics, pick-and-place robotics, one or more processors or computers, one or more local and/or cloud-based software packages (e.g., instrument/system control software packages, image processing software packages, data analysis software packages), data storage modules, data communication modules (e.g., Bluetooth, WiFi, intranet, or internet communication hardware and associated software), display modules, or any combination thereof.

### Example Flow Cell Embodiments

Definitions: As used herein, fluorescence is ‘specific’ if it arises from fluorophores that are annealed or otherwise tethered to the surface, such as through a nucleic acid having a region of reverse complementarity to a corresponding segment of an oligo on the surface and annealed to said corresponding segment. This fluorescence is contrasted with fluorescence arising from fluorophores not tethered to the surface through such an annealing process, or in some cases to background florescence of the surface.

Nucleic acids: As used herein, a “nucleic acid” (also referred to as a “polynucleotide”, “oligonucleotide”, ribonucleic acid (RNA), or deoxyribonucleic acid (DNA)) is a linear polymer of two or more nucleotides joined by covalent internucleosidic linkages, or variants or functional fragments thereof. In naturally occurring examples of nucleic acids, the internucleoside linkage is typically a phosphodiester bond. However, other examples optionally comprise other internucleoside linkages, such as phosphorothiolate linkages and may or may not comprise a phosphate group. Nucleic acids include double- and single-stranded DNA, as well as double- and single-stranded RNA, DNA/RNA hybrids, peptide-nucleic acids (PNAs), hybrids between PNAs and DNA or RNA, and may also include other types of nucleic acid modifications.

As used herein, a “nucleotide” refers to a nucleotide, nucleoside, or analog thereof. In some cases, the nucleotide is an N- or C-glycoside of a purine or pyrimidine base (e.g., a deoxyribonucleoside containing 2-deoxy-D-ribose or ribonucleoside containing D-ribose). Examples of other nucleotide analogs include, but are not limited to, phosphorothioates, phosphoramidates, methyl phosphonates, chiral-methyl phosphonates, 2-O-methyl ribonucleotides, and the like.

Nucleic acids may optionally be attached to one or more non-nucleotide moieties such as labels and other small molecules, large molecules (such as proteins, lipids, sugars, etc.), and solid or semi-solid supports, for example through covalent or non-covalent linkages with either the 5′ or 3′ end of the nucleic acid. Labels include any moiety that is detectable using any of a variety of detection methods known to those of skill in the art, and thus renders the attached oligonucleotide or nucleic acid similarly detectable. Some labels emit electromagnetic radiation that is optically detectable or visible. Alternately or in combination, some labels comprise a mass tag that renders the labeled oligonucleotide or nucleic acid visible in mass spectral data, or a redox tag that renders the labeled oligonucleotide or nucleic acid detectable by amperometry or voltametry. Some labels comprise a magnetic tag that facilitates separation and/or purification of the labeled oligonucleotide or nucleic acid. The nucleotide or polynucleotide is often not attached to a label, and the presence of the oligonucleotide or nucleic acid is directly detected.

Flow Cell Devices: Disclosed herein are flow devices that include a first reservoir housing a first solution and having an inlet end and an outlet end, wherein the first agent flows from the inlet end to the outlet end in the first reservoir; a second reservoir housing a second solution and having an inlet end and an outlet end, wherein the second agent flows from the inlet end to the outlet end in the second reservoir; a central region having an inlet end fluidically coupled to the outlet end of the first reservoir and the outlet end of the second reservoir through at least one valve. In the flow cell device, the volume of the first solution flowing from the outlet of the first reservoir to the inlet of the central region is less than the volume of the second solution flowing from the outlet of the second reservoir to the inlet of the central region.

The reservoirs described in the device can be used to house different reagents. In some aspects, the first solution housed in the first reservoir is different from the second solution that is housed in the second reservoir. The second solution comprises at least one reagent common to a plurality of reactions occurring in the central region. In some aspects, the second solution comprises at least one reagent selected from the list consisting of a solvent, a polymerase, and a dNTP. In some aspects, the second solution comprise low cost reagents. In some aspects, the first reservoir is fluidically coupled to the central region through a first valve and the second reservoir is fluidically coupled to the central region through a second valve. The valve can be a diaphragm valve or other suitable valves.

The design of the flow cell device can achieve a more efficient use of the reaction reagents than other sequencing device, particularly for costly reagents used in a variety of sequencing steps. In some aspects, the first solution comprises a reagent and the second solution comprises a reagent and the reagent in the first solution is more expensive than the reagent in the second solution. In some aspects, the first solution comprises a reaction-specific reagent and the second solution comprises nonspecific reagent common to all reaction occurring in the central region, and wherein the reaction specific reagent is more expensive than the nonspecific reagent. In some aspects, the first reservoir is positioned in close proximity to the inlet of the central region to reduce dead volume for delivery of the first solutions. In some aspects, the first reservoir is places closer to the inlet of the central region than the second reservoir. In some aspects, the reaction-specific reagent is configured in close proximity to the second diaphragm valve so as to reduce dead volume relative to delivery of the plurality of nonspecific reagents from the plurality of reservoirs to the first diaphragm valve.

Central Region: The central region can include a capillary tube or microfluidic chip having one or more microfluidic channels. In some embodiments, the capillary tube is an off-shelf product. The capillary tube or the microfluidic chip can also be removable from the device. In some embodiments, the capillary tube or microfluidic channel comprises an oligonucleotide population directed to sequence a eukaryotic genome. In some embodiments, the capillary tube or microfluidic channel in the central region can be removable.

Capillary flow cell devices: Disclosed herein are single capillary flow cell devices that comprise a single capillary and one or two fluidic adapters affixed to one or both ends of the capillary, where the capillary provides a fluid flow channel of specified cross-sectional area and length, and where the fluidic adapters are configured to mate with standard tubing to provide for convenient, interchangeable fluid connections with an external fluid flow control system.

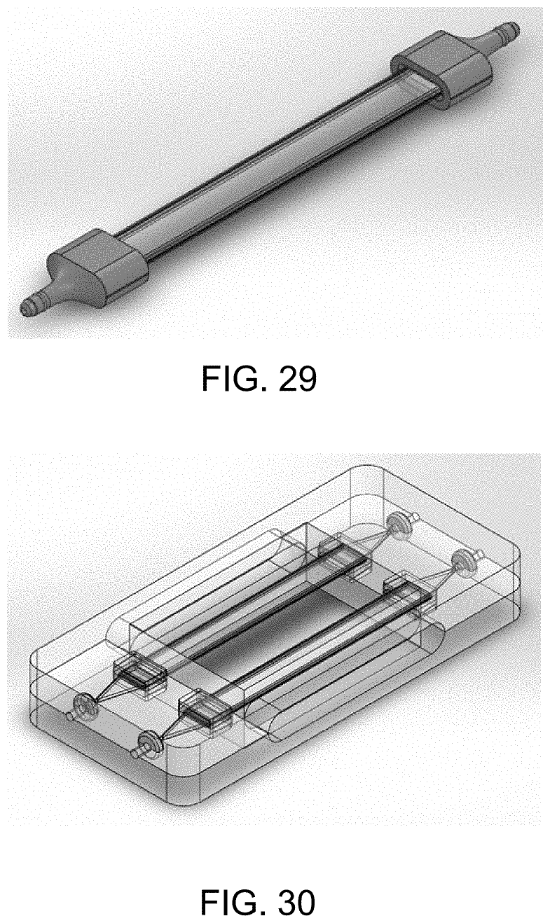

FIG. 29 illustrates one non-limiting example of a single glass capillary flow cell device that comprises two fluidic adaptors—one affixed to each end of the piece of glass capillary—that are designed to mate with standard OD fluidic tubing. The fluidic adaptors can be attached to the capillary using any of a variety of techniques known to those of skill in the art including, but not limited to, press fit, adhesive bonding, solvent bonding, laser welding, etc., or any combination thereof.

In general, the capillary used in the disclosed flow cell devices (and flow cell cartridges to be described below) will have at least one internal, axially-aligned fluid flow channel (or “lumen”) that runs the full length of the capillary. In some aspects, the capillary may have two, three, four, five, or more than five internal, axially-aligned fluid flow channels (or “lumen”).

A number specified cross-sectional geometries for a single capillary (or lumen thereof) are consistent with the disclosure herein, including, but not limited to, circular, elliptical, square, rectangular, triangular, rounded square, rounded rectangular, or rounded triangular cross-sectional geometries. In some aspects, the single capillary (or lumen thereof) may have any specified cross-sectional dimension or set of dimensions. For example, in some aspects the largest cross-sectional dimension of the capillary lumen (e.g. the diameter if the lumen is circular in shape or the diagonal if the lumen is square or rectangular in shape) may range from about 10 μm to about 10 mm. In some aspects, the largest cross-sectional dimension of the capillary lumen may be at least 10 μm, at least 25 μm, at least 50 μm, at least 75 μm, at least 100 μm, at least 200 μm, at least 300 μm, at least 400 μm, at least 500 μm, at least 600 μm, at least 700 μm, at least 800 μm, at least 900 μm, at least 1 mm, at least 2 mm, at least 3 mm, at least 4 mm, at least 5 mm, at least 6 mm, at least 7 mm, at least 8 mm, at least 9 mm, or at least 10 mm. In some aspects, the largest cross-sectional dimension of the capillary lumen may be at most 10 mm, at most 9 mm, at most 8 mm, at most 7 mm, at most 6 mm, at most 5 mm, at most 4 mm, at most 3 mm, at most 2 mm, at most 1 mm, at most 900 μm, at most 800 μm, at most 700 μm, at most 600 μm, at most 500 μm, at most 400 μm, at most 300 μm, at most 200 μm, at most 100 μm, at most 75 μm, at most 50 μm, at most 25 μm, or at most 10 μm. Any of the lower and upper values described in this paragraph may be combined to form a range included within the present disclosure, for example, in some aspects the largest cross-sectional dimension of the capillary lumen may range from about 100 μm to about 500 μm. Those of skill in the art will recognize that the largest cross-sectional dimension of the capillary lumen may have any value within this range, e.g., about 124 μm.

The length of the one or more capillaries used to fabricate the disclosed single capillary flow cell devices or flow cell cartridges may range from about 5 mm to about 5 cm or greater. In some instances, the length of the one or more capillaries may be less than 5 mm, at least 5 mm, at least 1 cm, at least 1.5 cm, at least 2 cm, at least 2.5 cm, at least 3 cm, at least 3.5 cm, at least 4 cm, at least 4.5 cm, or at least 5 cm. In some instances, the length of the one or more capillaries may be at most 5 cm, at most 4.5 cm, at most 4 cm, at most 3.5 cm, at most 3 cm, at most 2.5 cm, at most 2 cm, at most 1.5 cm, at most 1 cm, or at most 5 mm. Any of the lower and upper values described in this paragraph may be combined to form a range included within the present disclosure, for example, in some instances the length of the one or more capillaries may range from about 1.5 cm to about 2.5 cm. Those of skill in the art will recognize that the length of the one or more capillaries may have any value within this range, e.g., about 1.85 cm. In some instances, devices or cartridges may comprise a plurality of two or more capillaries that are the same length. In some instances, devices or cartridges may comprise a plurality of two or more capillaries that are of different lengths.

Capillaries in some cases have a gap height of about or exactly 50, 75, 100, 125, 150, 175, 200, 225, 250, 275, 300, 350, 400, or 500 um, or any value falling within the range defined thereby. Some preferred embodiments have gap heights of about 50 um-200 um, 50 um to 150 um, or comparable gap heights. The capillaries used for constructing the disclosed single capillary flow cell devices or capillary flow cell cartridges may be fabricated from any of a variety of materials known to those of skill in the art including, but not limited to, glass (e.g., borosilicate glass, soda lime glass, etc.), fused silica (quartz), polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), polydimethylsiloxane (PDMS), etc.), polyetherimide (PEI) and perfluoroelastomer (FFKM) as more chemically inert alternatives. PEI is somewhere between polycarbonate and PEEK in terms of both cost and compatibility. FFKM is also known as Kalrez or any combination thereof.

The capillaries used for constructing the disclosed single capillary flow cell devices or capillary flow cell cartridges may be fabricated using any of a variety of techniques known to those of skill in the art, where the choice of fabrication technique is often dependent on the choice of material used, and vice versa. Examples of suitable capillary fabrication techniques include, but are not limited to, extrusion, drawing, precision computer numerical control (CNC) machining and boring, laser photoablation, and the like. Devices can be pour molded or injection molded to fabricate any three dimension structure for adapting to single piece flow cell.

Examples of commercial vendors that provide precision capillary tubing include Accu-Glass (St. Louis, Mo.; precision glass capillary tubing), Polymicro Technologies (Phoenix, Ariz.; precision glass and fused-silica capillary tubing), Friedrich & Dimmock, Inc. (Millville, N.J.; custom precision glass capillary tubing), and Drummond Scientific (Broomall, Pa.; OEM glass and plastic capillary tubing).

Microfluidic chip flow cell devices: Disclosed herein also include flow cell devices that comprise one or more microfluidic chips and one or two fluidic adapters affixed to one or both ends of the microfluidic chips, where the microfluidic chip provides one or more fluid flow channels of specified cross-sectional area and length, and where the fluidic adapters are configured to mate with the microfluidic chip to provide for convenient, interchangeable fluid connections with an external fluid flow control system.

A non-limiting example of a microfluidic chip flow cell device that comprises two fluidic adaptors—one affixed to each end of the microfluidic chip (e.g., the inlet of the microfluidic channels). The fluidic adaptors can be attached to the chip or channel using any of a variety of techniques known to those of skill in the art including, but not limited to, press fit, adhesive bonding, solvent bonding, laser welding, etc., or any combination thereof. In some instances, the inlet and/or outlet of the microfluidic channels on the chip are apertures on the top surface of the chip, and the fluidic adaptors can be attached or coupled to the inlet and outlet of the microfluidic chips.

When the central region comprises a microfluidic chip, the chip microfluidic chip used in the disclosed flow cell deices will have at least a single layer having one or more channels. In some aspects, the microfluidic chip has two layers bonded together to form one or more channels. In some aspects, the microfluidic chip can include three layers bonded together to form one or more channels. In some embodiments, the microfluidic channel has an open top. In some embodiments, the microfluidic channel is positioned between a top layer and a bottom layer.

In general, the microfluidic chip used in the disclosed flow cell devices (and flow cell cartridges to be described below) will have at least one internal, axially-aligned fluid flow channel (or “lumen”) that runs the full length or a partial length of the chip. In some aspects, the microfluidic chip may have two, three, four, five, or more than five internal, axially-aligned microfluidic channels (or “lumen”). The microfluidic channel can be divided into a plurality of frames.

A number specified cross-sectional geometries for a single channels are consistent with the disclosure herein, including, but not limited to, circular, elliptical, square, rectangular, triangular, rounded square, rounded rectangular, or rounded triangular cross-sectional geometries. In some aspects, the channel may have any specified cross-sectional dimension or set of dimensions.

The microfluidic chip used for constructing the disclosed flow cell devices or flow cell cartridges may be fabricated from any of a variety of materials known to those of skill in the art including, but not limited to, glass (e.g., borosilicate glass, soda lime glass, etc.), quartz, polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), polydimethylsiloxane (PDMS), etc.), polyetherimide (PEI) and perfluoroelastomer (FFKM) as more chemically inert alternatives. In some embodiments, the microfluidic chip comprises quartz. In some embodiments, the microfluidic chip comprises borosilicate glass.

The microfluidic chips used for constructing the described flow cell devices or flow cell cartridges may be fabricated using any of a variety of techniques known to those of skill in the art, where the choice of fabrication technique is often dependent on the choice of material used, and vice versa. The microfluidic channels on the chip can be constructed using techniques suitable for forming micro-structure or micro-pattern on the surface. In some aspects, the channel is formed by laser irradiation. In some aspects, the microfluidic channel is formed by focused femtosecond laser radiation. In some aspects, the microfluidic channel is formed by etching, including but not limited to chemical or laser etching.

When the microfluidic channels are formed on the microfluidic chip through etching, the microfluidic chip will comprise at least one etched layer. In some aspects, the microfluidic chip can include comprise one non-etched layer, and one non-etched layer, with the etched layer being bonded to the non-etched layer such that the non-etched layer forms a bottom layer or a cover layer for the channels. In some aspects, the microfluidic chip can include comprise one non-etched layer, and two non-etched layers, and wherein the etched layer is positioned between the two non-etched layers.

The chip described herein includes one or more microfluidic channels etched on the surface of the chip. The microfluidic channels are defined as fluid conduits with at least one minimum dimension from <1 nm to 1000 μm. The microfluidic channels can be fabricated through several different methods, such as laser radiation (e.g., femtosecond laser radiation), lithography, chemical etching, and any other suitable methods. Channels on the chip surface can be created by selective patterning and plasma or chemical etching. The channels can be open, or they can be sealed by a conformal deposited film or layer on top to create subsurface or buried channels in the chip. In some embodiments, the channels are created from the removal of a sacrificial layer on the chip. This method does not require the bulk wafer to be etched away. Instead, the channel is located on the surface of the wafer. Examples of direct lithography include electron beam direct-write and focused ion beam milling.

The microfluidic channel system is coupled with an imaging system to capture or detect signals of DNA bases. The microfluidic channel system, fabricated on either a glass or silicon substrate, has channel heights and widths on the order of <1 nm to 1000 μm. For example, in some embodiments a channel may have a depth of 1-50 μm, 1-100 μm, 1-150 μm, 1-200 μm, 1-250 μm, 1-300 μm, 50-100 μm, 50-200 μm, or 50-300 μm, or greater than 300 μm, or a range defined by any two of these values. In some embodiments, a channel may have a depth of 3 mm or more. In some embodiments, a channel may have a depth of 30 mm or more. In some embodiments, a channel may have a length of less than 0.1 mm, between 0.1 mm and 0.5 mm, between 0.1 mm and 1 mm, between 0.1 mm and 5 mm, between 0.1 mm and 10 mm, between 0.1 mm and 25 mm, between 0.1 mm and 50 mm, between 0.1 mm and 100 mm, between 0.1 mm and 150 mm, between 0.1 mm and 200 mm, between 0.1 mm and 250 mm, between 1 mm and 5 mm, between 1 mm and 10 mm, between 1 mm and 25 mm, between 1 mm and 50 mm, between 1 mm and 100 mm, between 1 mm and 150 mm, between 1 mm and 200 mm, between 1 mm and 250 mm, between 5 mm and 10 mm, between 5 mm and 25 mm, between 5 mm and 50 mm, between 5 mm and 100 mm, between 5 mm and 150 mm, between 5 mm and 200 mm, between 1 mm and 250 mm, or greater than 250 mm, or a range defined by any two of these values. In some embodiments, a channel may have a length of 2 m or more. In some embodiments, a channel may have a length of 20 m or more. In some embodiments, a channel may have a width of less than 0.1 mm, between 0.1 mm and 0.5 mm, between 0.1 mm and 1 mm, between 0.1 mm and 5 mm, between 0.1 mm and 10 mm, between 0.1 mm and 15 mm, between 0.1 mm and 20 mm, between 0.1 mm and 25 mm, between 0.1 mm and 30 mm, between 0.1 mm and 50 mm, or greater than 50 mm, or a range defined by any two of these values. In some embodiments, a channel may have a width of 500 mm or more. In some embodiments, a channel may have a width of 5 m or more. The channel length can be in the micrometer range.

The one or more materials used to fabricate the capillaries or microfluidic chips for the disclosed devices are often optically transparent to facilitate use with spectroscopic or imaging-based detection techniques. The entire capillary will be optically transparent. Alternately, only a portion of the capillary (e.g., an optically transparent “window”) will be optically transparent. In some instances, the entire microfluidic chip will be optically transparent. In some instances, only a portion of the microfluidic chip (e.g., an optically transparent “window”) will be optically transparent.

As noted above, the fluidic adapters that are attached to the capillaries or microfluidic channels of the flow cell devices and cartridges disclosed herein are designed to mate with standard OD polymer or glass fluidic tubing or microfluidic channel. As illustrated in FIG. 29, one end of the fluidic adapter may be designed to mate to capillary having specific dimensions and cross-sectional geometry, while the other end may be designed to mate with fluidic tubing having the same or different dimensions and cross-sectional geometry. The adapters may be fabricated using any of a variety of suitable techniques (e.g., extrusion molding, injection molding, compression molding, precision CNC machining, etc.) and materials (e.g., glass, fused-silica, ceramic, metal, polydimethylsiloxane, polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), etc.), where the choice of fabrication technique is often dependent on the choice of material used, and vice versa.

Surface coatings: An interior surface (or surface of a capillary lumen) of one or more capillaries or the channel on the microfluidic chip is often coated using any of a variety of surface modification techniques or polymer coatings known to those of skill in the art.

Examples of suitable surface modification or coating techniques include, but are not limited to, the use of silane chemistries (e.g., aminopropyltrimethoxysilane (APTMS), aminopropyltriethoxysilane (APTES), triethoxysilane, diethoxydimethylsilane, and other linear, branched, or cyclic silanes) for covalent attachment of functional groups or molecules to capillary lumen surfaces, covalently or non-covalently attached polymer layers (e.g., layers of streptavidin, polyacrilamide, polyester, dextran, poly-lysine, polyacrylamide/poly-lysine copolymers, polyethylene glycol (PEG), poly (n-isopropylacrylamide) (PNIPAM), poly(2-hydroxyethyl methacrylate), (PHEMA), poly(oligo(ethylene glycol) methyl ether methacrylate (POEGMA), polyacrylic acid (PAA), poly(vinylpyridine), poly(vinylimidazole) and poly-lysine copolymers), or any combination thereof.

Examples of conjugation chemistries that may be used to graft one or more layers of material (e.g. polymer layers) to the support surface and/or to cross-link the layers to each other include, but are not limited to, biotin-streptavidin interactions (or variations thereof), his tag -Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane chemistries.

The number of layers of polymer or other chemical layers on the interior or lumen surface may range from 1 to about 10 or greater than 10. In some instances, the number of layers is at least 1, at least 2, at least 3, at least 4, at least 5, at least 6, at least 7, at least 8, at least 9, or at least 10. In some instances, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some instances the number of layers may range from about 2 to about 4. In some instances, all of the layers may comprise the same material. In some instances, each layer may comprise a different material. In some instances, the plurality of layers may comprise a plurality of materials.

In a preferred aspect, one or more layers of a coating material may be applied to the capillary lumen surface or the interior surface of the channel on the microfluidic chip, where the number of layers and/or the material composition of each layer is chosen to adjust one or more surface properties of the capillary or channel lumen, as noted in U.S. patent application Ser. No. 16/363,842.

Examples of surface properties that may be adjusted include, but are not limited to, surface hydrophilicity/hydrophobicity, overall coating thickness, the surface density of chemically-reactive functional groups, the surface density of grafted linker molecules or oligonucleotide primers, etc. In some preferred applications, one or more surface properties of the capillary or channel lumen are adjusted to, for example, (i) provide for very low non-specific binding of proteins, oligonucleotides, fluorophores, and other molecular components of chemical or biological analysis applications, including solid-phase nucleic acid amplification and/or sequencing applications, (ii) provide for improved solid-phase nucleic acid hybridization specificity and efficiency, and (iii) provide for improved solid-phase nucleic acid amplification rate, specificity, and efficiency.

One or more surface modification and/or polymer layers may be applied by flowing one or more appropriate chemical coupling or coating reagents through the capillaries or channel prior to use for their intended application. One or more coating reagents may be added to a buffer used, e.g., a nucleic acid hybridization, amplification reaction, and/or sequencing reaction to provide for dynamic coating of the capillary lumen surface.

Low non-specific binding surface: The interior surface of the channel and capillary tube described herein can be grafted or coated with a composition comprising low non-specific binding surface compositions that enable improved nucleic acid hybridization and amplification performance.

In some instances, fluorescence images of the disclosed low non-specific binding surfaces when used in nucleic acid hybridization or amplification applications to create clusters of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

In order to scale primer surface density and add additional dimensionality to hydrophilic or amphoteric surfaces, substrates comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the surface significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some instances, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some instances, high primer density materials may be constructed in solution and subsequently layered onto the surface in multiple steps.

30 degrees.40 degrees. Those of skill in the art will realize that a given hydrophilic, low-binding support surface of the present disclosure may exhibit a water contact angle having a value of anywhere within this range.

The disclosed interior surface of the channel and capillary may comprise a substrate (or support structure), one or more layers of a covalently or non-covalently attached low-binding, chemical modification layers, e.g., silane layers, polymer films, and one or more covalently or non-covalently attached primer sequences that may be used for tethering single-stranded template oligonucleotides to the support surface. In some instances, the formulation of the surface, e.g., the chemical composition of one or more layers, the coupling chemistry used to cross-link the one or more layers to the support surface and/or to each other, and the total number of layers, may be varied such that non-specific binding of proteins, nucleic acid molecules, and other hybridization and amplification reaction components to the support surface is minimized or reduced relative to a comparable monolayer. Often, the formulation of the surface may be varied such that non-specific hybridization on the support surface is minimized or reduced relative to a comparable monolayer. The formulation of the surface may be varied such that non-specific amplification on the support surface is minimized or reduced relative to a comparable monolayer. The formulation of the surface may be varied such that specific amplification rates and/or yields on the support surface are increased or maximized. Amplification levels suitable for detection are achieved in no more than 2, 3, 4, 5, 6, 7, 8, 9, 10, 15, 20, 25, 30, or more than 30 amplification cycles in some cases disclosed herein.

Examples of materials from which the substrate or support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

The substrate or support structure may be rendered in any of a variety of geometries and dimensions known to those of skill in the art, and may comprise any of a variety of materials known to those of skill in the art. For example, in some instances the substrate or support structure may be locally planar (e.g., comprising a microscope slide or the surface of a microscope slide). Globally, the substrate or support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some instances, the surface of the substrate or support structure used for nucleic acid hybridization and amplification may be a solid, non-porous surface. In some instances, the surface of the substrate or support structure used for nucleic acid hybridization and amplification may be porous, such that the coatings described herein penetrate the porous surface, and nucleic acid hybridization and amplification reactions performed thereon may occur within the pores.

The substrate or support structure that comprises the one or more chemically-modified layers, e.g., layers of a low non-specific binding polymer, may be independent or integrated into another structure or assembly. For example, in some instances, the substrate or support structure may comprise one or more surfaces within an integrated or assembled microfluidic flow cell. The substrate or support structure may comprise one or more surfaces within a microplate format, e.g., the bottom surface of the wells in a microplate. As noted above, in some preferred embodiments, the substrate or support structure comprises the interior surface (such as the lumen surface) of a capillary. In alternate preferred embodiments the substrate or support structure comprises the interior surface (such as the lumen surface) of a capillary etched into a planar chip.

The chemical modification layers may be applied uniformly across the surface of the substrate or support structure. Alternately, the surface of the substrate or support structure may be non-uniformly distributed or patterned, such that the chemical modification layers are confined to one or more discrete regions of the substrate. For example, the substrate surface may be patterned using photolithographic techniques to create an ordered array or random pattern of chemically-modified regions on the surface. Alternately or in combination, the substrate surface may be patterned using, e.g., contact printing and/or ink-jet printing techniques. In some instances, an ordered array or random patter of chemically-modified discrete regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions, or any intermediate number spanned by the range herein.

In order to achieve low nonspecific binding surfaces (also referred to herein as “low binding” or “passivated” surfaces), hydrophilic polymers may be nonspecifically adsorbed or covalently grafted to the substrate or support surface. Typically, passivation is performed utilizing poly(ethylene glycol) (PEG, also known as polyethylene oxide (PEO) or polyoxyethylene), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, dextran, or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some instances, two or more layers of a hydrophilic polymer, e.g., a linear polymer, branched polymer, or multi-branched polymer, may be deposited on the surface. In some instances, two or more layers may be covalently coupled to each other or internally cross-linked to improve the stability of the resulting surface. In some instances, oligonucleotide primers with different base sequences and base modifications (or other biomolecules, e.g., enzymes or antibodies) may be tethered to the resulting surface layer at various surface densities. In some instances, for example, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the final primer density. Primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence Reading then compared with that for a dye solution of known concentration.

In some embodiments, the hydrophilic polymer can be a cross linked polymer. In some embodiments, the cross-linked polymer can include one type of polymer cross linked with another type of polymer. Examples of the crossed-linked polymer can include poly(ethylene glycol) cross-linked with another polymer selected from polyethylene oxide (PEO) or polyoxyethylene), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, dextran, or other hydrophilic polymers. In some embodiments, the cross-linked polymer can be a poly(ethylene glycol) cross-linked with polyacrylamide.

The interior surface of one or more capillaries or the channels on the microfluidic chip or wall of the capillary can exhibit low non-specific binding of proteins and other amplification reaction reagents or components, and improved stability to repetitive exposure to different solvents, changes in temperature, chemical affronts such as low pH, or long term storage.

The disclosed low non-specific binding supports comprising one or more polymer coatings, e.g., PEG polymer films, that reduce or minimize non-specific binding of protein and labeled nucleotides to the solid support. The subsequent demonstration of improved nucleic acid hybridization and amplification rates and specificity may be achieved through one or more of the following additional aspects of the present disclosure: (i) primer design (sequence and/or modifications), (ii) control of tethered primer density on the solid support, (iii) the surface composition of the solid support, (iv) the surface polymer density of the solid support, (v) the use of improved hybridization conditions before and during amplification, and/or (vi) the use of improved amplification formulations that decrease non-specific primer amplification or increase template amplification efficiency.

The advantages of the disclosed low non-specific binding supports and associated hybridization and amplification methods confer one or more of the following additional advantages for any sequencing system: (i) decreased fluidic wash times (due to reduced non-specific binding, and thus faster sequencing cycle times), (ii) decreased imaging times (and thus faster turnaround times for assay readout and sequencing cycles), (iii) decreased overall work flow time requirements (due to decreased cycle times), (iv) decreased detection instrumentation costs (due to the improvements in CNR), (v) improved readout (base-calling) accuracy (due to improvements in CNR), (vi) improved reagent stability and decreased reagent usage requirements (and thus reduced reagents costs), and (vii) fewer run-time failures due to nucleic acid amplification failures.

The low binding hydrophilic surfaces (multilayer and/or monolayer) for surface bioassays, e.g., genotyping and sequencing assays, are created by using any combination of the following.

Polar protic, polar aprotic and/or nonpolar solvents for depositing and/or coupling linear or multi-branched hydrophilic polymer subunits on a substrate surface. Some multi-branched hydriphilic polymer subunits may contain functional end groups to promote covalent coupling or non-covalent binding interactions with other polymer subunites. Examples of suitable functional end groups include biotin, methoxy ether, carboxylate, amine, ester compounds, azide, alkyne, maleimide, thiol, and silane groups.

Any combination of linear, branched, or multi-branched polymer subunits coupled through subsequent layered addition via modified coupling chemistry/solvent/buffering systems that may include individual subunits with orthogonal end coupling chemistries or any of the respective combinations, such that resultant surface is hydrophilic and exhibits low nonspecific binding of proteins and other molecular assay components. In some instances, the hydrophilic, functionalized substrate surfaces of the present disclosure exhibit contact angle measurements that do not exceed 35 degrees.

Subsequent biomolecule attachment (e.g., of proteins, peptides, nucleic acids, oligonucleotides, or cells) on the low binding/hydrophilic substrates via any of a variety of individual conjugation chemistries to be described below, or any combination thereof. Layer deposition and/or conjugation reactions may be performed using solvent mixtures which may contain any ratio of the following components: ethanol, methanol, acetonitrile, acetone, DMSO, DMF, H2O, and the like. In addition, compatible buffering systems in the desirable pH range of 5-10 may be used for controlling the rate and efficiency of deposition and coupling, whereby coupling rates is excess of >5× of those for conventional aqueous buffer-based methods may be achieved.

The disclosed low non-specific binding supports and associated nucleic acid hybridization and amplification methods may be used for the analysis of nucleic acid molecules derived from any of a variety of different cell, tissue, or sample types known to those of skill in the art. For example, nucleic acids may be extracted from cells, or tissue samples comprising one or more types of cells, derived from eukaryotes (such as animals, plants, fungi, protista), archaebacteria, or eubacteria. In some cases, nucleic acids may be extracted from prokaryotic or eukaryotic cells, such as adherent or non-adherent eukaryotic cells. Nucleic acids are variously extracted from, for example, primary or immortalized rodent, porcine, feline, canine, bovine, equine, primate, or human cell lines. Nucleic acids may be extracted from any of a variety of different cell, organ, or tissue types (e.g., white blood cells, red blood cells, platelets, epithelial cells, endothelial cells, neurons, glial cells, astrocytes, fibroblasts, skeletal muscle cells, smooth muscle cells, gametes, or cells from the heart, lungs, brain, liver, kidney, spleen, pancreas, thymus, bladder, stomach, colon, or small intestine). Nucleic acids may be extracted from normal or healthy cells. Alternately or in combination, acids are extracted from diseased cells, such as cancerous cells, or from pathogenic cells that are infecting a host. Some nucleic acids may be extracted from a distinct subset of cell types, e.g., immune cells (such as T cells, cytotoxic (killer) T cells, helper T cells, alpha beta T cells, gamma delta T cells, T cell progenitors, B cells, B-cell progenitors, lymphoid stem cells, myeloid progenitor cells, lymphocytes, granulocytes, Natural Killer cells, plasma cells, memory cells, neutrophils, eosinophils, basophils, mast cells, monocytes, dendritic cells, and/or macrophages, or any combination thereof), undifferentiated human stem cells, human stem cells that have been induced to differentiate, rare cells (e.g., circulating tumor cells (CTCs), circulating epithelial cells, circulating endothelial cells, circulating endometrial cells, bone marrow cells, progenitor cells, foam cells, mesenchymal cells, or trophoblasts). Other cells are contemplated and consistent with the disclosure herein.

As a result of the surface passivation techniques disclosed herein, proteins, nucleic acids, and other biomolecules do not “stick” to the substrates, that is, they exhibit low nonspecific binding (NSB). Examples are shown below using standard monolayer surface preparations with varying glass preparation conditions. Hydrophilic surface that have been passivated to achieve ultra-low NSB for proteins and nucleic acids require novel reaction conditions to improve primer deposition reaction efficiencies, hybridization performance, and induce effective amplification. All of these processes require oligonucleotide attachment and subsequent protein binding and delivery to a low binding surface. As described below, the combination of a new primer surface conjugation formulation (Cy3 oligonucleotide graft titration) and resulting ultra-low non-specific background (NSB functional tests performed using red and green fluorescent dyes) yielded results that demonstrate the viability of the disclosed approaches. Some surfaces disclosed herein exhibit a ratio of specific (e.g., hybridization to a tethered primer or probe) to nonspecific binding (e.g., Binter) of a fluorophore such as Cy3 of at least 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 50:1, 75:1, 100:1, or greater than 100:1, or any intermediate value spanned by the range herein. Some surfaces disclosed herein exhibit a ratio of specific to nonspecific fluorescence signal (e.g., for specifically-hybridized to nonspecifically bound labeled oligonucleotides, or for specifically-amplified to nonspecifically-bound (Binter) or non-specifically amplified (Bintra) labeled oligonucleotides or a combination thereof (Binter+Bintra)) for a fluorophore such as Cy3 of at least 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 50:1, 75:1, 100:1, or greater than 100:1, or any intermediate value spanned by the range herein.

Grafting low non-specific binding layer: The attachment chemistry used to graft a first chemically-modified layer to an interior surface of the flow cell (capillary or channel) will generally be dependent on both the material from which the support is fabricated and the chemical nature of the layer. In some instances, the first layer may be covalently attached to the support surface. In some instances, the first layer may be non-covalently attached, e.g., adsorbed to the surface through non-covalent interactions such as electrostatic interactions, hydrogen bonding, or van der Waals interactions between the surface and the molecular components of the first layer. In either case, the substrate surface may be treated prior to attachment or deposition of the first layer. Any of a variety of surface preparation techniques known to those of skill in the art may be used to clean or treat the support surface. For example, glass or silicon surfaces may be acid-washed using a Piranha solution (a mixture of sulfuric acid (H2SO4) and hydrogen peroxide (H2O2)) and/or cleaned using an oxygen plasma treatment method.

Silane chemistries constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding support surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support surface, where the choice of components used may be varied to alter one or more properties of the support surface, e.g., the surface density of functional groups and/or tethered oligonucleotide primers, the hydrophilicity/hydrophobicity of the support surface, or the three three-dimensional nature (i.e., “thickness”) of the support surface. Examples of preferred polymers that may be used to create one or more layers of low non-specific binding material in any of the disclosed support surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the support surface and/or to cross-link the layers to each other include, but are not limited to, biotin-streptavidin interactions (or variations thereof), his tag -Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

One or more layers of a multi-layered surface may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo(ethylene glycol) methyl ether methacrylate) (branched POEGMA), branched polyglutamic acid (branched PGA), branched poly-lysine, branched poly-glucoside, and dextran.

In some instances, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches. Molecules often exhibit a ‘power of 2’ number of branches, such as 2, 4, 8, 16, 32, 64, or 128 branches.

Exemplary PEG multilayers include PEG (8 arm, 16 arm, 8 arm) on PEG-amine-APTES. Similar concentrations were observed for 3-layer multi-arm PEG (8 arm, 16 arm, 8 arm) and (8 arm, 64 arm, 8 arm) on PEG-amine-APTES exposed to 8 uM primer, and 3-layer multi-arm PEG (8 arm, 8 arm, 8 arm) using star-shape PEG-amine to replace 16 arm and 64 arm. PEG multilayers having comparable first, second and third PEG layers are also contemplated.

Linear, branched, or multi-branched polymers used to create one or more layers of any of the multi-layered surfaces disclosed herein may have a molecular weight of at least 500, at least 1,000, at least 1,500, at least 2,000, at least 2,500, at least 3,000, at least 3,500, at least 4,000, at least 4,500, at least 5,000, at least 7,500, at least 10,000, at least 12,500, at least 15,000, at least 17,500, at least 20,000, at least 25,000, at least 30,000, at least 35,000, at least 40,000, at least 45,000, or at least 50,000 Daltons. In some instances, the linear, branched, or multi-branched polymers used to create one or more layers of any of the multi-layered surfaces disclosed herein may have a molecular weight of at most 50,000, at most 45,000, at most 40,000, at most 35,000, at most 30,000, at most 25,000, at most 20,000, at most 17,500, at most 15,000, at most 12,500, at most 10,000, at most 7,500, at most 5,000, at most 4,500, at most 4,000, at most 3,500, at most 3,000, at most 2,500, at most 2,000, at most 1,500, at most 1,000, or at most 500 Daltons. Any of the lower and upper values described in this paragraph may be combined to form a range included within the present disclosure, for example, in some instances the molecular weight of linear, branched, or multi-branched polymers used to create one or more layers of any of the multi-layered surfaces disclosed herein may range from about 1,500 to about 20,000 Daltons. Those of skill in the art will recognize that the molecular weight of linear, branched, or multi-branched polymers used to create one or more layers of any of the multi-layered surfaces disclosed herein may have any value within this range, e.g., about 1,260 Daltons.

In some instances, e.g., wherein at least one layer of a multi-layered surface comprises a branched polymer, the number of covalent bonds between a branched polymer molecule of the layer being deposited and molecules of the previous layer may range from about one covalent linkages per molecule and about 32 covalent linkages per molecule. In some instances, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32, or more than 32 covalent linkages per molecule. In some instances, the number of covalent bonds between a branched polymer molecule of the new layer and molecules of the previous layer may be at most 32, at most 30, at most 28, at most 26, at most 24, at most 22, at most 20, at most 18, at most 16, at most 14, at most 12, at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some instances the number of covalent bonds between a branched polymer molecule of the new layer and molecules of the previous layer may range from about 4 to about 16. Those of skill in the art will recognize that the number of covalent bonds between a branched polymer molecule of the new layer and molecules of the previous layer may have any value within this range, e.g., about 11 in some instances, or an average number of about 4.6 in other instances.

Any reactive functional groups that remain following the coupling of a material layer to the support surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface of the disclosed low binding supports may range from 1 to about 10. In some instances, the number of layers is at least 1, at least 2, at least 3, at least 4, at least 5, at least 6, at least 7, at least 8, at least 9, or at least 10. In some instances, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some instances the number of layers may range from about 2 to about 4. In some instances, all of the layers may comprise the same material. In some instances, each layer may comprise a different material. In some instances, the plurality of layers may comprise a plurality of materials. In some instances at least one layer may comprise a branched polymer. In some instance, all of the layers may comprise a branched polymer.

One or more layers of low non-specific binding material may in some cases be deposited on and/or conjugated to the substrate surface using a polar protic solvent, a polar aprotic solvent, a nonpolar solvent, or any combination thereof. In some instances the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some instances, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, or any percentage spanned or adjacent to the range herein, with the balance made up of water or an aqueous buffer solution. In some instances, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, or any percentage spanned or adjacent to the range herein, with the balance made up of an organic solvent. The pH of the solvent mixture used may be less than 5, 5, 5, 5, 6, 6, 6.5, 7, 7.5, 8, 8.5, 9, 9.5, 10, or greater than 10, or any value spanned or adjacent to the range described herein.

In some instances, one or more layers of low non-specific binding material may be deposited on and/or conjugated to the substrate surface using a mixture of organic solvents, wherein the dielectric constant of at least once component is less than 40 and constitutes at least 50% of the total mixture by volume. In some instances, the dielectric constant of the at least one component may be less than 10, less than 20, less than 30, less than 40. In some instances, the at least one component constitutes at least 20%, at least 30%, at least 40%, at least 50%, at least 50%, at least 60%, at least 70%, or at least 80% of the total mixture by volume.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, in some instances, exposure of the surface to fluorescent dyes (e.g., Cy3, Cy5, etc.), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some instances, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations—provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some instances, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

As noted, in some instances, the degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some instances, the label may comprise a fluorescent label. In some instances, the label may comprise a radioisotope. In some instances, the label may comprise any other detectable label known to one of skill in the art. In some instances, the degree of non-specific binding exhibited by a given support surface formulation may thus be assessed in terms of the number of non-specifically bound protein molecules (or other molecules) per unit area. In some instances, the low-binding supports of the present disclosure may exhibit non-specific protein binding (or non-specific binding of other specified molecules, e.g., Cy3 dye) of less than 0.001 molecule per μm2, less than 0.01 molecule per μm2, less than 0.1 molecule per μm2, less than 0.25 molecule per μm2, less than 0.5 molecule per μm2, less than 1 molecule per μm2, less than 10 molecules per μm2, less than 100 molecules per μm2, or less than 1,000 molecules per μm2. Those of skill in the art will realize that a given support surface of the present disclosure may exhibit non-specific binding falling anywhere within this range, for example, of less than 86 molecules per μm2. For example, some modified surfaces disclosed herein exhibit nonspecific protein binding of less than 0.5 molecule/um2 following contact with a 1 uM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per um2. In independent nonspecific binding assays, 1 uM labeled Cy3 SA (ThermoFisher), 1 uM Cy5 SA dye (ThermoFisher), 10 uM Aminoallyl-dUTP-ATTO-647N (Jena Biosciences), 10 uM Aminoallyl-dUTP-ATTO-Rho11 (Jena Biosciences), 10 uM Aminoallyl-dUTP-ATTO-Rho11 (Jena Biosciences), 10 uM 7-Propargylamino-7-deaza-dGTP-Cy5 (Jena Biosciences, and 10 uM 7-Propargylamino-7-deaza-dGTP-Cy3 (Jena Biosciences) were incubated on the low binding substrates at 37° C. for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon (GE Healthcare Lifesciences, Pittsburgh, Pa.) instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (Olympus Corp., Center Valley, Pa.) with a total internal reflectance fluorescence (TIRF) objective (20×, 0.75 NA or 100×, 1.5 NA, Olympus), an sCMOS Andor camera (Zyla 4.2. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm2.

In some instances, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some instances, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 3:1, 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 3:1, 4:1, 5:1, 6:1, 7:1, 8:1, 9:1, 10:1, 15:1, 20:1, 30:1, 40:1, 50:1, or more than 50:1.

In some instances, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some instances, a static contact angle may be determined. In some instances, an advancing or receding contact angle may be determined. In some instances, the water contact angle for the hydrophilic, low-binding support surfaced disclosed herein may range from about 0 degrees to about 50 degrees. In some instances, the water contact angle for the hydrophilic, low-binding support surfaced disclosed herein may no more than 50 degrees, 45 degrees, 40 degrees, 35 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than any value within this range, e.g., no more than 40 degrees. Those of skill in the art will realize that a given hydrophilic, low-binding support surface of the present disclosure may exhibit a water contact angle having a value of anywhere within this range, e.g., about 27 degrees.

In some instances, the hydrophilic surfaces disclosed herein facilitate reduced wash times for bioassays, often due to reduced nonspecific binding of biomolecules to the low-binding surfaces. In some instances, adequate wash steps may be performed in less than 60, 50, 40, 30, 20, 15, 10, or less than 10 seconds. For example, in some instances adequate wash steps may be performed in less than 30 seconds.

Oligonucleotide primers and adapter sequences: In general, at least one layer of the one or more layers of low non-specific binding material may comprise functional groups for covalently or non-covalently attaching oligonucleotide molecules, e.g., adapter or primer sequences, or the at least one layer may already comprise covalently or non-covalently attached oligonucleotide adapter or primer sequences at the time that it is deposited on the support surface. In some instances, the oligonucleotides tethered to the polymer molecules of at least one third layer may be distributed at a plurality of depths throughout the layer.

In some instances, the oligonucleotide adapter or primer molecules are covalently coupled to the polymer in solution, i.e., prior to coupling or depositing the polymer on the surface. In some instances, the oligonucleotide adapter or primer molecules are covalently coupled to the polymer after it has been coupled to or deposited on the surface. In some instances, at least one hydrophilic polymer layer comprises a plurality of covalently-attached oligonucleotide adapter or primer molecules. In some instances, at least two, at least three, at least four, or at least five layers of hydrophilic polymer comprise a plurality of covalently-attached adapter or primer molecules.

In some instances, the oligonucleotide adapter or primer molecules may be coupled to the one or more layers of hydrophilic polymer using any of a variety of suitable conjugation chemistries known to those of skill in the art. For example, the oligonucleotide adapter or primer sequences may comprise moieties that are reactive with amine groups, carboxyl groups, thiol groups, and the like. Examples of suitable amine-reactive conjugation chemistries that may be used include, but are not limited to, reactions involving isothiocyanate, isocyanate, acyl azide, NHS ester, sulfonyl chloride, aldehyde, glyoxal, epoxide, oxirane, carbonate, aryl halide, imidoester, carbodiimide, anhydride, and fluorophenyl ester groups. Examples of suitable carboxyl-reactive conjugation chemistries include, but are not limited to, reactions involving carbodiimide compounds, e.g., water soluble EDC (1-ethyl-3-(3-dimethylaminopropyl)carbodiimide.HCL). Examples of suitable sulfydryl-reactive conjugation chemistries include maleimides, haloacetyls and pyridyl disulfides.

One or more types of oligonucleotide molecules may be attached or tethered to the support surface. In some instances, the one or more types of oligonucleotide adapters or primers may comprise spacer sequences, adapter sequences for hybridization to adapter-ligated template library nucleic acid sequences, forward amplification primers, reverse amplification primers, sequencing primers, and/or molecular barcoding sequences, or any combination thereof. In some instances, 1 primer or adapter sequence may be tethered to at least one layer of the surface. In some instances, at least 2, 3, 4, 5, 6, 7, 8, 9, 10, or more than 10 different primer or adapter sequences may be tethered to at least one layer of the surface.

In some instances, the tethered oligonucleotide adapter and/or primer sequences may range in length from about 10 nucleotides to about 100 nucleotides. In some instances, the tethered oligonucleotide adapter and/or primer sequences may be at least 10, at least 20, at least 30, at least 40, at least 50, at least 60, at least 70, at least 80, at least 90, or at least 100 nucleotides in length. In some instances, the tethered oligonucleotide adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some instances the length of the tethered oligonucleotide adapter and/or primer sequences may range from about 20 nucleotides to about 80 nucleotides. Those of skill in the art will recognize that the length of the tethered oligonucleotide adapter and/or primer sequences may have any value within this range, e.g., about 24 nucleotides.

In some instances, the tetheredadapter or primer sequences may comprise modifications designed to facilitate the specificity and efficiency of nucleic acid amplification as performed on the low-binding supports. For example, in some instances the primer may comprise polymerase stop points such that the stretch of primer sequence between the surface conjugation point and the modification site is always in single-stranded form and functions as a loading site for 5′ to 3′ helicases in some helicase-dependent isothermal amplification methods. Other examples of primer modifications that may be used to create polymerase stop points include, but are not limited to, an insertion of a PEG chain into the backbone of the primer between two nucleotides towards the 5′ end, insertion of an abasic nucleotide (i.e., a nucleotide that has neither a purine nor a pyrimidine base), or a lesion site which can be bypassed by the helicase.

As will be discussed further in the examples below, it may be desirable to vary the surface density of tethered oligonucleotide adapters or primers on the support surface and/or the spacing of the tetheredadapter or primers away from the support surface (e.g., by varying the length of a linker molecule used to tether the adapter or primers to the surface) in order to “tune” the support for optimal performance when using a given amplification method. As noted below, adjusting the surface density of tethered oligonucleotide adapters or primers may impact the level of specific and/or non-specific amplification observed on the support in a manner that varies according to the amplification method selected. In some instances, the surface density of tethered oligonucleotideadapters or primers may be varied by adjusting the ratio of molecular components used to create the support surface. For example, in the case that an oligonucleotide primer—PEG conjugate is used to create the final layer of a low-binding support, the ratio of the oligonucleotide primer—PEG conjugate to a non-conjugated PEG molecule may be varied. The resulting surface density of tethered primer molecules may then be estimated or measured using any of a variety of techniques known to those of skill in the art. Examples include, but are not limited to, the use of radioisotope labeling and counting methods, covalent coupling of a cleavable molecule that comprises an optically-detectable tag (e.g., a fluorescent tag) that may be cleaved from a support surface of defined area, collected in a fixed volume of an appropriate solvent, and then quantified by comparison of fluorescence signals to that for a calibration solution of known optical tag concentration, or using fluorescence imaging techniques provided that care has been taken with the labeling reaction conditions and image acquisition settings to ensure that the fluorescence signals are linearly related to the number of fluorophores on the surface (e.g., that there is no significant self-quenching of the fluorophores on the surface).

In some instances, the resultant surface density of oligonucleotide adapters or primers on the low binding support surfaces of the present disclosure may range from about 100 primer molecules per μm2 to about 1,000,000 primer molecules per μm2. In some instances, the surface density of oligonucleotide adapters or primers may be at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 5,500, at least 6,000, at least 6,500, at least 7,000, at least 7,500, at least 8,000, at least 8,500, at least 9,000, at least 9,500, at least 10,000, at least 15,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, at least 800,000, at least 850,000, at least 900,000, at least 950,000, or at least 1,000,000 molecules per μm2. In some instances, the surface density of oligonucleotide adapters or primers may be at most 1,000,000, at most 950,000, at most 900,000, at most 850,000, at most 800,000, at most 750,000, at most 700,000, at most 650,000, at most 600,000, at most 550,000, at most 500,000, at most 450,000, at most 400,000, at most 350,000, at most 300,000, at most 250,000, at most 200,000, at most 150,000, at most 100,000, at most 95,000, at most 90,000, at most 85,000, at most 80,000, at most 75,000, at most 70,000, at most 65,000, at most 60,000, at most 55,000, at most 50,000, at most 45,000, at most 40,000, at most 35,000, at most 30,000, at most 25,000, at most 20,000, at most 15,000, at most 10,000, at most 9,500, at most 9,000, at most 8,500, at most 8,000, at most 7,500, at most 7,000, at most 6,500, at most 6,000, at most 5,500, at most 5,000, at most 4,500, at most 4,000, at most 3,500, at most 3,000, at most 2,500, at most 2,000, at most 1,500, at most 1,000, at most 900, at most 800, at most 700, at most 600, at most 500, at most 400, at most 300, at most 200, or at most 100 molecules per μm2. Any of the lower and upper values described in this paragraph may be combined to form a range included within the present disclosure, for example, in some instances the surface density of adapters or primers may range from about 10,000 molecules per μm2 to about 100,000 molecules per μm2. Those of skill in the art will recognize that the surface density of adapter or primer molecules may have any value within this range, e.g., about 3,800 molecules per μm2 in some instances, or about 455,000 molecules per μm2 in other instances. In some instances, as will be discussed further below, the surface density of template library nucleic acid sequences (e.g., sample DNA molecules) initially hybridized to adapter or primer sequences on the support surface may be less than or equal to that indicated for the surface density of tethered oligonucleotide primers. In some instances, as will also be discussed further below, the surface density of clonally-amplified template library nucleic acid sequences hybridized to adapter or primer sequences on the support surface may span the same range or a different range as that indicated for the surface density of tethered oligonucleotide adapters or primers.

Local surface densities of adapter or primer molecules as listed above do not preclude variation in density across a surface, such that a surface may comprise a region having an oligo density of, for example, 500,000/um2, while also comprising at least a second region having a substantially different local density.

Hybridization of nucleic acid molecules to low-binding supports: In some aspects of the present disclosure, hybridization buffer formulations are described which, in combination with the disclosed low-binding supports, provide for improved hybridization rates, hybridization specificity (or stringency), and hybridization efficiency (or yield). As used herein, hybridization specificity is a measure of the ability of tethered adapter sequences, primer sequences, or oligonucleotide sequences in general to correctly hybridize only to completely complementary sequences, while hybridization efficiency is a measure of the percentage of total available tethered adapter sequences, primer sequences, or oligonucleotide sequences in general that are hybridized to complementary sequences.

Improved hybridization specificity and/or efficiency may be achieved through improvement or optimization of the hybridization buffer formulation used with the disclosed low-binding surfaces, and will be discussed in more detail in the examples below. Examples of hybridization buffer components that may be adjusted to achieve improved performance include, but are not limited to, buffer type, organic solvent mixtures, buffer pH, buffer viscosity, detergents and zwitterionic components, ionic strength (including adjustment of both monovalent and divalent ion concentrations), antioxidants and reducing agents, carbohydrates, BSA, polyethylene glycol, dextran sulfate, betaine, other additives, and the like.

By way of non-limiting example, suitable buffers for use in formulating a hybridization buffer may include, but are not limited to, phosphate buffered saline (PBS), succinate, citrate, histidine, acetate, Tris, TAPS, MOPS, PIPES, HEPES, MES, and the like. The choice of appropriate buffer will generally be dependent on the target pH of the hybridization buffer solution. In general, the desired pH of the buffer solution will range from about pH 4 to about pH 8.4. In some embodiments, the buffer pH may be at least 4.0, at least 4.5, at least 5.0, at least 5.5, at least 6.0, at least 6.2, at least 6.4, at least 6.6, at least 6.8, at least 7.0, at least 7.2, at least 7.4, at least 7.6, at least 7.8, at least 8.0, at least 8.2, or at least 8.4. In some embodiments, the buffer pH may be at most 8.4, at most 8.2, at most 8.0, at most 7.8, at most 7.6, at most 7.4, at most 7.2, at most 7.0, at most 6.8, at most 6.6, at most 6.4, at most 6.2, at most 6.0, at most 5.5, at most 5.0, at most 4.5, or at most 4.0. Any of the lower and upper values described in this paragraph may be combined to form a range included within the present disclosure, for example, in some instances, the desired pH may range from about 6.4 to about 7.2. Those of skill in the art will recognize that the buffer pH may have any value within this range, for example, about 7.25.

Suitable detergents for use in hybridization buffer formulation include, but are not limited to, zitterionic detergents (e.g., 1-Dodecanoyl-sn-glycero-3-phosphocholine, 3-(4-tert-Butyl-1-pyridinio)-1-propanesulfonate, 3-(N,N-Dimethylmyristylammonio)propanesulfonate, 3-(N,NDimethylmyristylammonio) propanesulfonate, ASB-C80, C7BzO, CHAPS, CHAPS hydrate, CHAPSO, DDMAB, Dimethylethylammoniumpropane sulfonate, N,N-Dimethyldodecylamine Noxide, N-Dodecyl-N,N-dimethyl-3-ammonio-1-propanesulfonate, or N-Dodecyl-N,N-dimethyl-3-ammonio-1-propanesulfonate) and anionic, cationic, and non-ionic detergents. Examples of nonionic detergents include poly(oxyethylene) ethers and related polymers (e.g. Brij®, TWEEN®, TRITON®, TRITON X-100 and IGEPAL® CA-630), bile salts, and glycosidic detergents.

The use of the disclosed low-binding supports either alone or in combination with improved or optimized buffer formulations may yield relative hybridization rates that range from about 2× to about 20× faster than that for a conventional hybridization protocol. In some instances, the relative hybridization rate may be at least 2×, at least 3×, at least 4×, at least 5×, at least 6×, at least 7×, at least 8×, at least 9×, at least 10×, at least 12×, at least 14×, at least 16×, at least 18×, at least 20×, at least 25×, at least 30×, or at least 40× that for a conventional hybridization protocol.

In some instances, the use of the disclosed low-binding supports alone or in combination with improved or optimized buffer formulations may yield total hybridization reaction times (i.e., the time required to reach 90%, 95%, 98%, or 99% completion of the hybridization reaction) of less than 60 minutes, 50 minutes, 40 minutes, 30 minutes, 20 minutes, 15 minutes, 10 minutes, or 5 minutes for any of these completion metrics.

In some instances, the use of the disclosed low-binding supports alone or in combination with improved or optimized buffer formulations may yield improved hybridization specificity compared to that for a conventional hybridization protocol. In some instances, the hybridization specificity that may be achieved is better than 1 base mismatch in 10 hybridization events, 1 base mismatch in 20 hybridization events, 1 base mismatch in 30 hybridization events, 1 base mismatch in 40 hybridization events, 1 base mismatch in 50 hybridization events, 1 base mismatch in 75 hybridization events, 1 base mismatch in 100 hybridization events, 1 base mismatch in 200 hybridization events, 1 base mismatch in 300 hybridization events, 1 base mismatch in 400 hybridization events, 1 base mismatch in 500 hybridization events, 1 base mismatch in 600 hybridization events, 1 base mismatch in 700 hybridization events, 1 base mismatch in 800 hybridization events, 1 base mismatch in 900 hybridization events, 1 base mismatch in 1,000 hybridization events, 1 base mismatch in 2,000 hybridization events, 1 base mismatch in 3,000 hybridization events, 1 base mismatch in 4,000 hybridization events, 1 base mismatch in 5,000 hybridization events, 1 base mismatch in 6,000 hybridization events, 1 base mismatch in 7,000 hybridization events, 1 base mismatch in 8,000 hybridization events, 1 base mismatch in 9,000 hybridization events, or 1 base mismatch in 10,000 hybridization events.

In some instances, the use of the disclosed low-binding supports alone or in combination with improved or optimized buffer formulations may yield improved hybridization efficiency (e.g., the fraction of available oligonucleotide primers on the support surface that are successfully hybridized with target oligonucleotide sequences) compared to that for a conventional hybridization protocol. In some instances, the hybridization efficiency that may be achieved is better than 50%, 60%, 70%, 80%, 85%, 90%, 95%, 98%, or 99% for any of the input target oligonucleotide concentrations specified below and in any of the hybridization reaction times specified above. In some instances, e.g., wherein the hybridization efficiency is less than 100%, the resulting surface density of target nucleic acid sequences hybridized to the support surface may be less than the surface density of oligonucleotide adapter or primer sequences on the surface.

In some instances, use of the disclosed low-binding supports for nucleic acid hybridization (or amplification) applications using conventional hybridization (or amplification) protocols, or improved or optimized hybridization (or amplification) protocols may lead to a reduced requirement for the input concentration of target (or sample) nucleic acid molecules contacted with the support surface. For example, in some instances, the target (or sample) nucleic acid molecules may be contacted with the support surface at a concentration ranging from about 10 pM to about 1 μM (i.e., prior to annealing or amplification). In some instances, the target (or sample) nucleic acid molecules may be administered at a concentration of at least 10 pM, at least 20 pM, at least 30 pM, at least 40 pM, at least 50 pM, at least 100 pM, at least 200 pM, at least 300 pM, at least 400 pM, at least 500 pM, at least 600 pM, at least 700 pM, at least 800 pM, at least 900 pM, at least 1 nM, at least 10 nM, at least 20 nM, at least 30 nM, at least 40 nM, at least 50 nM, at least 60 nM, at least 70 nM, at least 80 nM, at least 90 nM, at least 100 nM, at least 200 nM, at least 300 nM, at least 400 nM, at least 500 nM, at least 600 nM, at least 700 nM, at least 800 nM, at least 900 nM, or at least 1 μM. In some instances, the target (or sample) nucleic acid molecules may be administered at a concentration of at most 1 μM, at most 900 nM, at most 800 nm, at most 700 nM, at most 600 nM, at most 500 nM, at most 400 nM, at most 300 nM, at most 200 nM, at most 100 nM, at most 90 nM, at most 80 nM, at most 70 nM, at most 60 nM, at most 50 nM, at most 40 nM, at most 30 nM, at most 20 nM, at most 10 nM, at most 1 nM, at most 900 pM, at most 800 pM, at most 700 pM, at most 600 pM, at most 500 pM, at most 400 pM, at most 300 pM, at most 200 pM, at most 100 pM, at most 90 pM, at most 80 pM, at most 70 pM, at most 60 pM, at most 50 pM, at most 40 pM, at most 30 pM, at most 20 pM, or at most 10 pM. Any of the lower and upper values described in this paragraph may be combined to form a range included within the present disclosure, for example, in some instances the target (or sample) nucleic acid molecules may be administered at a concentration ranging from about 90 pM to about 200 nM. Those of skill in the art will recognize that the target (or sample) nucleic acid molecules may be administered at a concentration having any value within this range, e.g., about 855 nM.

In some instances, the use of the disclosed low-binding supports alone or in combination with improved or optimized hybridization buffer formulations may result in a surface density of hybridized target (or sample) oligonucleotide molecules (i.e., prior to performing any subsequent solid-phase or clonal amplification reaction) ranging from about from about 0.0001 target oligonucleotide molecules per μm2 to about 1,000,000 target oligonucleotide molecules per μm2. In some instances, the surface density of hybridized target oligonucleotide molecules may be at least 0.0001, at least 0.0005, at least 0.001, at least 0.005, at least 0.01, at least 0.05, at least 0.1, at least 0.5, at least 1, at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 5,500, at least 6,000, at least 6,500, at least 7,000, at least 7,500, at least 8,000, at least 8,500, at least 9,000, at least 9,500, at least 10,000, at least 15,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, at least 800,000, at least 850,000, at least 900,000, at least 950,000, or at least 1,000,000 molecules per μm2. In some instances, the surface density of hybridized target oligonucleotide molecules may be at most 1,000,000, at most 950,000, at most 900,000, at most 850,000, at most 800,000, at most 750,000, at most 700,000, at most 650,000, at most 600,000, at most 550,000, at most 500,000, at most 450,000, at most 400,000, at most 350,000, at most 300,000, at most 250,000, at most 200,000, at most 150,000, at most 100,000, at most 95,000, at most 90,000, at most 85,000, at most 80,000, at most 75,000, at most 70,000, at most 65,000, at most 60,000, at most 55,000, at most 50,000, at most 45,000, at most 40,000, at most 35,000, at most 30,000, at most 25,000, at most 20,000, at most 15,000, at most 10,000, at most 9,500, at most 9,000, at most 8,500, at most 8,000, at most 7,500, at most 7,000, at most 6,500, at most 6,000, at most 5,500, at most 5,000, at most 4,500, at most 4,000, at most 3,500, at most 3,000, at most 2,500, at most 2,000, at most 1,500, at most 1,000, at most 900, at most 800, at most 700, at most 600, at most 500, at most 400, at most 300, at most 200, at most 100, at most 90, at most 80, at most 70, at most 60, at most 50, at most 40, at most 30, at most 20, at most 10, at most 5, at most 1, at most 0.5, at most 0.1, at most 0.05, at most 0.01, at most 0.005, at most 0.001, at most 0.0005, or at most 0.0001 molecules per μm2. Any of the lower and upper values described in this paragraph may be combined to form a range included within the present disclosure, for example, in some instances the surface density of hybridized target oligonucleotide molecules may range from about 3,000 molecules per μm2 to about 20,000 molecules per μm2. Those of skill in the art will recognize that the surface density of hybridized target oligonucleotide molecules may have any value within this range, e.g., about 2,700 molecules per μm2.

Stated differently, in some instances the use of the disclosed low-binding supports alone or in combination with improved or optimized hybridization buffer formulations may result in a surface density of hybridized target (or sample) oligonucleotide molecules (i.e., prior to performing any subsequent solid-phase or clonal amplification reaction) ranging from about 100 hybridized target oligonucleotide molecules per mm2 to about 1×107 oligonucleotide molecules per mm2 or from about 100 hybridized target oligonucleotide molecules per mm2 to about 1×1012 hybridized target oligonucleotide molecules per mm2. In some instances, the surface density of hybridized target oligonucleotide molecules may be at least 100, at least 500, at least 1,000, at least 4,000, at least 5,000, at least 6,000, at least 10,000, at least 15,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, at least 800,000, at least 850,000, at least 900,000, at least 950,000, at least 1,000,000, at least 5,000,000, at least 1×107, at least 5×107, at least 1×108, at least 5×108, at least 1×109, at least 5×109, at least 1×1010, at least 5×1010, at least 1×1011, at least 5×1011, or at least 1×1012 molecules per mm2. In some instances, the surface density of hybridized target oligonucleotide molecules may be at most 1×1012, at most 5×1011, at most 1×1011, at most 5×1010, at most 1×1010, at most 5×109, at most 1×109, at most 5×108, at most 1×108, at most 5×107, at most 1×107, at most 5,000,000, at most 1,000,000, at most 950,000, at most 900,000, at most 850,000, at most 800,000, at most 750,000, at most 700,000, at most 650,000, at most 600,000, at most 550,000, at most 500,000, at most 450,000, at most 400,000, at most 350,000, at most 300,000, at most 250,000, at most 200,000, at most 150,000, at most 100,000, at most 95,000, at most 90,000, at most 85,000, at most 80,000, at most 75,000, at most 70,000, at most 65,000, at most 60,000, at most 55,000, at most 50,000, at most 45,000, at most 40,000, at most 35,000, at most 30,000, at most 25,000, at most 20,000, at most 15,000, at most 10,000, at most 5,000, at most 1,000, at most 500, or at most 100 molecules per mm2. Any of the lower and upper values described in this paragraph may be combined to form a range included within the present disclosure, for example, in some instances the surface density of hybridized target oligonucleotide molecules may range from about 5,000 molecules per mm2 to about 50,000 molecules per mm2. Those of skill in the art will recognize that the surface density of hybridized target oligonucleotide molecules may have any value within this range, e.g., about 50,700 molecules per mm2.

In some instances, the target (or sample) oligonucleotide molecules (or nucleic acid molecules) hybridized to the oligonucleotide adapter or primer molecules attached to the low-binding support surface may range in length from about 0.02 kilobases (kb) to about 20 kb or from about 0.1 kilobases (kb) to about 20 kb. In some instances, the target oligonucleotide molecules may be at least 0.001 kb, at least 0.005 kb, at least 0.01 kb, at least 0.02 kb, at least 0.05 kb, at least 0.1 kb in length, at least 0.2 kb in length, at least 0.3 kb in length, at least 0.4 kb in length, at least 0.5 kb in length, at least 0.6 kb in length, at least 0.7 kb in length, at least 0.8 kb in length, at least 0.9 kb in length, at least 1 kb in length, at least 2 kb in length, at least 3 kb in length, at least 4 kb in length, at least 5 kb in length, at least 6 kb in length, at least 7 kb in length, at least 8 kb in length, at least 9 kb in length, at least 10 kb in length, at least 15 kb in length, at least 20 kb in length, at least 30 kb in length, or at least 40 kb in length, or any intermediate value spanned by the range described herein, e.g., at least 0.85 kb in length.

In some instances, the target (or sample) oligonucleotide molecules (or nucleic acid molecules) may comprise single-stranded or double-stranded, multimeric nucleic acid molecules further comprising repeats of a regularly occurring monomer unit. In some instances, the single-stranded or double-stranded, multimeric nucleic acid molecules may be at least 0.001 kb, at least 0.005 kb, at least 0.01 kb, at least 0.02 kb, at least 0.05 kb, at least 0.1 kb in length, at least 0.2 kb in length, at least 0.3 kb in length, at least 0.4 kb in length, at least 0.5 kb in length, at least 1 kb in length, at least 2 kb in length, at least 3 kb in length, at least 4 kb in length, at least 5 kb in length, at least 6 kb in length, at least 7 kb in length, at least 8 kb in length, at least 9 kb in length, at least 10 kb in length, at least 15 kb in length, or at least 20 kb in length, at least 30 kb in length, or at least 40 kb in length, or any intermediate value spanned by the range described herein, e.g., about 2.45 kb in length.

In some instances, the target (or sample) oligonucleotide molecules (or nucleic acid molecules) may comprise single-stranded or double-stranded multimeric nucleic acid molecules comprising from about 2 to about 100 copies of a regularly repeating monomer unit. In some instances, the number of copies of the regularly repeating monomer unit may be at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, and at least 100. In some instances, the number of copies of the regularly repeating monomer unit may be at most 100, at most 95, at most 90, at most 85, at most 80, at most 75, at most 70, at most 65, at most 60, at most 55, at most 50, at most 45, at most 40, at most 35, at most 30, at most 25, at most 20, at most 15, at most 10, at most 5, at most 4, at most 3, or at most 2. Any of the lower and upper values described in this paragraph may be combined to form a range included within the present disclosure, for example, in some instances the number of copies of the regularly repeating monomer unit may range from about 4 to about 60. Those of skill in the art will recognize that the number of copies of the regularly repeating monomer unit may have any value within this range, e.g., about 17. Thus, in some instances, the surface density of hybridized target sequences in terms of the number of copies of a target sequence per unit area of the support surface may exceed the surface density of oligonucleotide primers even if the hybridization efficiency is less than 100%.

Nucleic acid surface amplification (NASA): As used herein, the phrase “nucleic acid surface amplification” (NASA) is used interchangeably with the phrase “solid-phase nucleic acid amplification” (or simply “solid-phase amplification”). In some aspects of the present disclosure, nucleic acid amplification formulations are described which, in combination with the disclosed low-binding supports, provide for improved amplification rates, amplification specificity, and amplification efficiency. As used herein, specific amplification refers to amplification of template library oligonucleotide strands that have been tethered to the solid support either covalently or non-covalently. As used herein, non-specific amplification refers to amplification of primer-dimers or other non-template nucleic acids. As used herein, amplification efficiency is a measure of the percentage of tethered oligonucleotides on the support surface that are successfully amplified during a given amplification cycle or amplification reaction. Nucleic acid amplification performed on surfaces disclosed herein may obtain amplification efficiencies of at least 50%, 60%, 70%, 80%, 90%, 95%, or greater than 95%, such as 98% or 99%.

Any of a variety of thermal cycling or isothermal nucleic acid amplification schemes may be used with the disclosed low-binding supports. Examples of nucleic acid amplification methods that may be utilized with the disclosed low-binding supports include, but are not limited to, polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification, circle-to-circle amplification, helicase-dependent amplification, recombinase-dependent amplification, or single-stranded binding (SSB) protein-dependent amplification.

Often, improvements in amplification rate, amplification specificity, and amplification efficiency may be achieved using the disclosed low-binding supports alone or in combination with formulations of the amplification reaction components. In addition to inclusion of nucleotides, one or more polymerases, helicases, single-stranded binding proteins, etc. (or any combination thereof), the amplification reaction mixture may be adjusted in a variety of ways to achieve improved performance including, but are not limited to, choice of buffer type, buffer pH, organic solvent mixtures, buffer viscosity, detergents and zwitterionic components, ionic strength (including adjustment of both monovalent and divalent ion concentrations), antioxidants and reducing agents, carbohydrates, BSA, polyethylene glycol, dextran sulfate, betaine, other additives, and the like.

The use of the disclosed low-binding supports alone or in combination with improved or optimized amplification reaction formulations may yield increased amplification rates compared to those obtained using conventional supports and amplification protocols. In some instances, the relative amplification rates that may be achieved may be at least 2×, at least 3×, at least 4×, at least 5×, at least 6×, at least 7×, at least 8×, at least 9×, at least 10×, at least 12×, at least 14×, at least 16×, at least 18×, or at least 20× that for use of conventional supports and amplification protocols for any of the amplification methods described above.

In some instances, the use of the disclosed low-binding supports alone or in combination with improved or optimized buffer formulations may yield total amplification reaction times (i.e., the time required to reach 90%, 95%, 98%, or 99% completion of the amplification reaction) of less than 180 mins, 120 mins, 90 min, 60 minutes, 50 minutes, 40 minutes, 30 minutes, 20 minutes, 15 minutes, 10 minutes, 5 minutes, 3 minutes, 1 minute, 50 s, 40 s, 30 s, 20 s, or 10 s for any of these completion metrics.

Some low-binding support surfaces disclosed herein exhibit a ratio of specific binding to nonspecific binding of a fluorophore such as Cy3 of at least 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 50:1, 75:1, 100:1, or greater than 100:1, or any intermediate value spanned by the range herein. Some surfaces disclosed herein exhibit a ratio of specific to nonspecific fluorescence signal for a fluorophore such as Cy3 of at least 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 50:1, 75:1, 100:1, or greater than 100:1, or any intermediate value spanned by the range herein.

In some instances, the use of the disclosed low-binding supports alone or in combination with improved or optimized amplification buffer formulations may enable faster amplification reaction times (i.e., the times required to reach 90%, 95%, 98%, or 99% completion of the amplification reaction) of no more than 60 minutes, 50 minutes, 40 minutes, 30 minutes, 20 minutes, or 10 minutes. Similarly, use of the disclosed low-binding supports alone or in combination with improved or optimized buffer formulations may enable amplification reactions to be completed in some cases in no more than 2, 3, 4, 5, 6, 7, 8, 9, 10, 15, or no more than 30 cycles.

In some instances, the use of the disclosed low-binding supports alone or in combination with improved or optimized amplification reaction formulations may yield increased specific amplification and/or decreased non-specific amplification compared to that obtained using conventional supports and amplification protocols. In some instances, the resulting ratio of specific amplification-to-non-specific amplification that may be achieved is at least 4:1 5:1, 6:1, 7:1, 8:1, 9:1, 10:1, 20:1, 30:1, 40:1, 50:1, 60:1, 70:1, 80:1, 90:1, 100:1, 200:1, 300:1, 400:1, 500:1, 600:1, 700:1, 800:1, 900:1, or 1,000:1.

In some instances, the use of the low-binding supports alone or in combination with improved or optimized amplification reaction formulations may yield increased amplification efficiency compared to that obtained using conventional supports and amplification protocols. In some instances, the amplification efficiency that may be achieved is better than 50%, 60%, 70% 80%, 85%, 90%, 95%, 98%, or 99% in any of the amplification reaction times specified above.

In some instances, the clonally-amplified target (or sample) oligonucleotide molecules (or nucleic acid molecules) hybridized to the oligonucleotide adapter or primer molecules attached to the low-binding support surface may range in length from about 0.02 kilobases (kb) to about 20 kb or from about 0.1 kilobases (kb) to about 20 kb. In some instances, the clonally-amplified target oligonucleotide molecules may be at least 0.001 kb, at least 0.005 kb, at least 0.01 kb, at least 0.02 kb, at least 0.05 kb, at least 0.1 kb in length, at least 0.2 kb in length, at least 0.3 kb in length, at least 0.4 kb in length, at least 0.5 kb in length, at least 1 kb in length, at least 2 kb in length, at least 3 kb in length, at least 4 kb in length, at least 5 kb in length, at least 6 kb in length, at least 7 kb in length, at least 8 kb in length, at least 9 kb in length, at least 10 kb in length, at least 15 kb in length, or at least 20 kb in length, or any intermediate value spanned by the range described herein, e.g., at least 0.85 kb in length.

In some instances, the clonally-amplified target (or sample) oligonucleotide molecules (or nucleic acid molecules) may comprise single-stranded or double-stranded, multimeric nucleic acid molecules further comprising repeats of a regularly occurring monomer unit. In some instances, the clonally-amplified single-stranded or double-stranded, multimeric nucleic acid molecules may be at least 0.1 kb in length, at least 0.2 kb in length, at least 0.3 kb in length, at least 0.4 kb in length, at least 0.5 kb in length, at least 1 kb in length, at least 2 kb in length, at least 3 kb in length, at least 4 kb in length, at least 5 kb in length, at least 6 kb in length, at least 7 kb in length, at least 8 kb in length, at least 9 kb in length, at least 10 kb in length, at least 15 kb in length, or at least 20 kb in length, or any intermediate value spanned by the range described herein, e.g., about 2.45 kb in length.

In some instances, the clonally-amplified target (or sample) oligonucleotide molecules (or nucleic acid molecules) may comprise single-stranded or double-stranded multimeric nucleic acid molecules comprising from about 2 to about 100 copies of a regularly repeating monomer unit. In some instances, the number of copies of the regularly repeating monomer unit may be at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, and at least 100. In some instances, the number of copies of the regularly repeating monomer unit may be at most 100, at most 95, at most 90, at most 85, at most 80, at most 75, at most 70, at most 65, at most 60, at most 55, at most 50, at most 45, at most 40, at most 35, at most 30, at most 25, at most 20, at most 15, at most 10, at most 5, at most 4, at most 3, or at most 2. Any of the lower and upper values described in this paragraph may be combined to form a range included within the present disclosure, for example, in some instances the number of copies of the regularly repeating monomer unit may range from about 4 to about 60. Those of skill in the art will recognize that the number of copies of the regularly repeating monomer unit may have any value within this range, e.g., about 12. Thus, in some instances, the surface density of clonally-amplified target sequences in terms of the number of copies of a target sequence per unit area of the support surface may exceed the surface density of oligonucleotide primers even if the hybridization and/or amplification efficiencies are less than 100%.

In some instances, the use of the disclosed low-binding supports alone or in combination with improved or optimized amplification reaction formulations may yield increased clonal copy number compared to that obtained using conventional supports and amplification protocols. In some instances, e.g., wherein the clonally-amplified target (or sample) oligonucleotide molecules comprise concatenated, multimeric repeats of a monomeric target sequence, the clonal copy number may be substantially smaller than compared to that obtained using conventional supports and amplification protocols. Thus, in some instances, the clonal copy number may range from about 1 molecule to about 100,000 molecules (e.g., target sequence molecules) per amplified colony. In some instances, the clonal copy number may be at least 1, at least 5, at least 10, at least 50, at least 100, at least 500, at least 1,000, at least 2,000, at least 3,000, at least 4,000, at least 5,000, at least 6,000, at least 7,000, at least 8,000, at least 9,000, at least 10,000, at least 15,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, or at least 100,000 molecules per amplified colony. In some instances, the clonal copy number may be at most 100,000, at most 95,000, at most 90,000, at most 85,000, at most 80,000, at most 75,000, at most 70,000, at most 65,000, at most 60,000, at most 55,000, at most 50,000, at most 45,000, at most 40,000, at most 35,000, at most 30,000, at most 25,000, at most 20,000, at most 15,000, at most 10,000, at most 9,000, at most 8,000, at most 7,000, at most 6,000, at most 5,000, at most 4,000, at most 3,000, at most 2,000, at most 1,000, at most 500, at most 100, at most 50, at most 10, at most 5, or at most 1 molecule per amplified colony. Any of the lower and upper values described in this paragraph may be combined to form a range included within the present disclosure, for example, in some instances the clonal copy number may range from about 2,000 molecules to about 9,000 molecules. Those of skill in the art will recognize that the clonal copy number may have any value within this range, e.g., about 2,220 molecules in some instances, or about 2 molecules in others.

As noted above, in some instances the amplified target (or sample) oligonucleotide molecules (or nucleic acid molecules) may comprise concatenated, multimeric repeats of a monomeric target sequence. In some instances, the amplified target (or sample) oligonucleotide molecules (or nucleic acid molecules) may comprise a plurality of molecules each of which comprises a single monomeric target sequence. Thus, the use of the disclosed low-binding supports alone or in combination with improved or optimized amplification reaction formulations may result in a surface density of target sequence copies that ranges from about 100 target sequence copies per mm2 to about 1×1012 target sequence copies per mm2. In some instances, the surface density of target sequence copies may be at least 100, at least 500, at least 1,000, at least 5,000, at least 10,000, at least 15,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, at least 800,000, at least 850,000, at least 900,000, at least 950,000, at least 1,000,000, at least 5,000,000, at least 1×107, at least 5×107, at least 1×108, at least 5×108, at least 1×109, at least 5×109, at least 1×1010, at least 5×1010, at least 1×1011, at least 5×1011, or at least 1×1012 of clonally amplified target sequence molecules per mm2. In some instances, the surface density of target sequence copies may be at most 1×1012, at most 5×1011, at most 1×1011, at most 5×1010, at most 1×1010, at most 5×109, at most 1×109, at most 5×108, at most 1×108, at most 5×107, at most 1×107, at most 5,000,000, at most 1,000,000, at most 950,000, at most 900,000, at most 850,000, at most 800,000, at most 750,000, at most 700,000, at most 650,000, at most 600,000, at most 550,000, at most 500,000, at most 450,000, at most 400,000, at most 350,000, at most 300,000, at most 250,000, at most 200,000, at most 150,000, at most 100,000, at most 95,000, at most 90,000, at most 85,000, at most 80,000, at most 75,000, at most 70,000, at most 65,000, at most 60,000, at most 55,000, at most 50,000, at most 45,000, at most 40,000, at most 35,000, at most 30,000, at most 25,000, at most 20,000, at most 15,000, at most 10,000, at most 5,000, at most 1,000, at most 500, or at most 100 target sequence copies per mm2. Any of the lower and upper values described in this paragraph may be combined to form a range included within the present disclosure, for example, in some instances the surface density of target sequence copies may range from about 1,000 target sequence copies per mm2 to about 65,000 target sequence copies mm2. Those of skill in the art will recognize that the surface density of target sequence copies may have any value within this range, e.g., about 49,600 target sequence copies per mm2.

In some instances, the use of the disclosed low-binding supports alone or in combination with improved or optimized amplification buffer formulations may result in a surface density of clonally-amplified target (or sample) oligonucleotide molecules (or clusters) ranging from about from about 100 molecules per mm2 to about 1×1012 colonies per mm2. In some instances, the surface density of clonally-amplified molecules may be at least 100, at least 500, at least 1,000, at least 5,000, at least 10,000, at least 15,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, at least 800,000, at least 850,000, at least 900,000, at least 950,000, at least 1,000,000, at least 5,000,000, at least 1×107, at least 5×107, at least 1×108, at least 5×108, at least 1×109, at least 5×109, at least 1×1010, at least 5×1010, at least 1×1011, at least 5×1011, or at least 1×1012 molecules per mm2. In some instances, the surface density of clonally-amplified molecules may be at most 1×1012, at most 5×1011, at most 1×1011, at most 5×1010, at most 1×1010, at most 5×109, at most 1×109, at most 5×108, at most 1×108, at most 5×107, at most 1×107, at most 5,000,000, at most 1,000,000, at most 950,000, at most 900,000, at most 850,000, at most 800,000, at most 750,000, at most 700,000, at most 650,000, at most 600,000, at most 550,000, at most 500,000, at most 450,000, at most 400,000, at most 350,000, at most 300,000, at most 250,000, at most 200,000, at most 150,000, at most 100,000, at most 95,000, at most 90,000, at most 85,000, at most 80,000, at most 75,000, at most 70,000, at most 65,000, at most 60,000, at most 55,000, at most 50,000, at most 45,000, at most 40,000, at most 35,000, at most 30,000, at most 25,000, at most 20,000, at most 15,000, at most 10,000, at most 5,000, at most 1,000, at most 500, or at most 100 molecules per mm2. Any of the lower and upper values described in this paragraph may be combined to form a range included within the present disclosure, for example, in some instances the surface density of clonally-amplified molecules may range from about 5,000 molecules per mm2 to about 50,000 molecules per mm2. Those of skill in the art will recognize that the surface density of clonally-amplified colonies may have any value within this range, e.g., about 48,800 molecules per mm2.

In some instances, the use of the disclosed low-binding supports alone or in combination with improved or optimized amplification buffer formulations may result in a surface density of clonally-amplified target (or sample) oligonucleotide molecules (or clusters) ranging from about from about 100 molecules per mm2 to about 1×1012 colonies per mm2. In some instances, the surface density of clonally-amplified molecules may be at least 100, at least 500, at least 1,000, at least 5,000, at least 10,000, at least 15,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, at least 800,000, at least 850,000, at least 900,000, at least 950,000, at least 1,000,000, at least 5,000,000, at least 1×107, at least 5×107, at least 1×108, at least 5×108, at least 1×109, at least 5×109, at least 1×1010, at least 5×1010, at least 1×1011, at least 5×1011, or at least 1×1012 molecules per mm2. In some instances, the surface density of clonally-amplified molecules may be at most 1×1012, at most 5×1011, at most 1×1011, at most 5×1010, at most 1×1010, at most 5×109, at most 1×109, at most 5×108, at most 1×108, at most 5×107, at most 1×107, at most 5,000,000, at most 1,000,000, at most 950,000, at most 900,000, at most 850,000, at most 800,000, at most 750,000, at most 700,000, at most 650,000, at most 600,000, at most 550,000, at most 500,000, at most 450,000, at most 400,000, at most 350,000, at most 300,000, at most 250,000, at most 200,000, at most 150,000, at most 100,000, at most 95,000, at most 90,000, at most 85,000, at most 80,000, at most 75,000, at most 70,000, at most 65,000, at most 60,000, at most 55,000, at most 50,000, at most 45,000, at most 40,000, at most 35,000, at most 30,000, at most 25,000, at most 20,000, at most 15,000, at most 10,000, at most 5,000, at most 1,000, at most 500, or at most 100 molecules per mm2. Any of the lower and upper values described in this paragraph may be combined to form a range included within the present disclosure, for example, in some instances the surface density of clonally-amplified molecules may range from about 5,000 molecules per mm2 to about 50,000 molecules per mm2. Those of skill in the art will recognize that the surface density of clonally-amplified colonies may have any value within this range, e.g., about 48,800 molecules per mm2.

In some instances, the use of the disclosed low-binding supports alone or in combination with improved or optimized amplification buffer formulations may result in a surface density of clonally-amplified target (or sample) oligonucleotide colonies (or clusters) ranging from about from about 100 colonies per mm2 to about 1×1012 colonies per mm2. In some instances, the surface density of clonally-amplified colonies may be at least 100, at least 500, at least 1,000, at least 5,000, at least 10,000, at least 15,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, at least 800,000, at least 850,000, at least 900,000, at least 950,000, at least 1,000,000, at least 5,000,000, at least 1×107, at least 5×107, at least 1×108, at least 5×108, at least 1×109, at least 5×109, at least 1×1010, at least 5×1010, at least 1×1011, at least 5×1011, or at least 1×1012 colonies per mm2. In some instances, the surface density of clonally-amplified colonies may be at most 1×1012, at most 5×1011, at most 1×1011, at most 5×1010, at most 1×1010, at most 5×109, at most 1×109, at most 5×108, at most 1×108, at most 5×107, at most 1×107, at most 5,000,000, at most 1,000,000, at most 950,000, at most 900,000, at most 850,000, at most 800,000, at most 750,000, at most 700,000, at most 650,000, at most 600,000, at most 550,000, at most 500,000, at most 450,000, at most 400,000, at most 350,000, at most 300,000, at most 250,000, at most 200,000, at most 150,000, at most 100,000, at most 95,000, at most 90,000, at most 85,000, at most 80,000, at most 75,000, at most 70,000, at most 65,000, at most 60,000, at most 55,000, at most 50,000, at most 45,000, at most 40,000, at most 35,000, at most 30,000, at most 25,000, at most 20,000, at most 15,000, at most 10,000, at most 5,000, at most 1,000, at most 500, or at most 100 colonies per mm2. Any of the lower and upper values described in this paragraph may be combined to form a range included within the present disclosure, for example, in some instances the surface density of clonally-amplified colonies may range from about 5,000 colonies per mm2 to about 50,000 colonies per mm2. Those of skill in the art will recognize that the surface density of clonally-amplified colonies may have any value within this range, e.g., about 48,800 colonies per mm2.

In some cases the use of the disclosed low-binding supports alone or in combination with improved or optimized amplification reaction formulations may yield signal from the amplified and labeled nucleic acid populations (e.g., a fluorescence signal) that has a coefficient of variance of no greater than 50%, such as 50%, 40%, 30%, 20%, 15%, 10%, 5%, or less than 5%.

Similarly, in some cases the use of improved or optimized amplification reaction formulations in combination with the disclosed low-binding supports yield signal from the nucleic acid populations that has a coefficient of variance of no greater than 50%, such as 50%, 40%, 30%, 20%, 10% or less than 10%.

In some cases, the support surfaces and methods as disclosed herein allow amplification at elevated extension temperatures, such as at 15 C, 20 C, 25 C, 30 C, 40 C, or greater, or for example at about 21 C or 23 C.

In some cases, the use of the support surfaces and methods as disclosed herein enable simplified amplification reactions. For example, in some cases amplification reactions are performed using no more than 1, 2, 3, 4, or 5 discrete reagents.

In some cases, the use of the support surfaces and methods as disclosed herein enable the use of simplified temperature profiles during amplification, such that reactions are executed at temperatures ranging from a low temperature of 15 C, 20 C, 25 C, 30 C, or 40 C, to a high temperature of 40 C, 45 C, 50 C, 60 C, 65 C, 70 C, 75 C, 80 C, or greater than 80 C, for example, such as a range of 20 C to 65 C.

Amplification reactions are also improved such that lower amounts of template (e.g., target or sample molecules) are sufficient to lead to discernable signals on a surface, such as 1 pM, 2 pM, 5 pM, 10 pM, 15 pM, 20 pM, 30 pM, 40 pM, 50 pM, 60 pM, 70 pM, 80 pM, 90 pM, 100 pM, 200 pM, 300 pM, 400 pM, 500 pM, 600 pM, 700 pM, 800 pM, 900 pM, 1,000 pM, 2,000 pM, 3,000 pM, 4,000 pM, 5,000 pM, 6,000 pM, 7,000 pM, 8,000 pM, 9,000 pM, 10,000 pM or greater than 10,000 pM of a sample, such as 500 nM. In exemplary embodiments, inputs of about 100 pM are sufficient to generate signals for reliable signal determination.

Fluorescence imaging of support surfaces: The disclosed solid-phase nucleic acid amplification reaction formulations and low-binding supports may be used in any of a variety of nucleic acid analysis applications, e.g., nucleic acid base discrimination, nucleic acid base classification, nucleic acid base calling, nucleic acid detection applications, nucleic acid sequencing applications, and nucleic acid-based (genetic and genomic) diagnostic applications. In many of these applications, fluorescence imaging techniques may be used to monitor hybridization, amplification, and/or sequencing reactions performed on the low-binding supports.

Fluorescence imaging may be performed using any of a variety of fluorophores, fluorescence imaging techniques, and fluorescence imaging instruments known to those of skill in the art. Examples of suitable fluorescence dyes that may be used (e.g., by conjugation to nucleotides, oligonucleotides, or proteins) include, but are not limited to, fluorescein, rhodamine, coumarin, cyanine, and derivatives thereof, including the cyanine derivatives Cyanine dye-3 (Cy3), Cyanine dye-5 (Cy5), Cyanine dye-7 (Cy7), etc. Examples of fluorescence imaging techniques that may be used include, but are not limited to, fluorescence microscopy imaging, fluorescence confocal imaging, two-photon fluorescence, and the like. Examples of fluorescence imaging instruments that may be used include, but are not limited to, fluorescence microscopes equipped with an image sensor or camera, confocal fluorescence microscopes, two-photon fluorescence microscopes, or custom instruments that comprise a suitable selection of light sources, lenses, mirrors, prisms, dichroic reflectors, apertures, and image sensors or cameras, etc. A non-limiting example of a fluorescence microscope equipped for acquiring images of the disclosed low-binding support surfaces and clonally-amplified colonies (or clusters) of target nucleic acid sequences hybridized thereon is the Olympus IX83 inverted fluorescence microscope equipped with) 20×, 0.75 NA, a 532 nm light source, a bandpass and dichroic mirror filter set adapted or optimized for 532 nm long-pass excitation and Cy3 fluorescence emission filter, a Semrock 532 nm dichroic reflector, and a camera (Andor sCMOS, Zyla 4.2) where the excitation light intensity is adjusted to avoid signal saturation. Often, the support surface may be immersed in a buffer (e.g., 25 mM ACES, pH 7.4 buffer) while the image is acquired.

In some instances, the performance of nucleic acid hybridization and/or amplification reactions using the disclosed reaction formulations and low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal−Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times should be reduced or minimized), as shown in the example below. At high CNR the imaging time required to reach accurate discrimination (and thus accurate base-calling in the case of sequencing applications) can be drastically reduced even with moderate improvements in CNR.

In most ensemble-based sequencing approaches, the background term is typically measured as the signal associated with ‘interstitial’ regions. In addition to “interstitial” background (Binter), “intrastitial” background (Bintra) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Binter background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (S)—Binter in the FOV) yields a discernable feature that can be classified. In some instances, the intrastitial background (Bintra) can contribute a confounding fluorescence signal that is not specific to the target of interest, but is present in the same ROI thus making it far more difficult to average and subtract.

As will be demonstrated in the examples below, the implementation of nucleic acid amplification on the low-binding substrates of the present disclosure may decrease the Binter background signal by reducing non-specific binding, may lead to improvements in specific nucleic acid amplification, and may lead to a decrease in non-specific amplification that can impact the background signal arising from both the interstitial and intrastitial regions. In some instances, the disclosed low-binding support surfaces, optionally used in combination with the disclosed hybridization and/or amplification reaction formulations, may lead to improvements in CNR by a factor of 2, 5, 10, 100, or 1000-fold over those achieved using conventional supports and hybridization, amplification, and/or sequencing protocols. Although described here in the context of using fluorescence imaging as the read-out or detection mode, the same principles apply to the use of the disclosed low-binding supports and nucleic acid hybridization and amplification formulations for other detection modes as well, including both optical and non-optical detection modes.

The disclosed low-binding supports, optionally used in combination with the disclosed hybridization and/or amplification protocols, yield solid-phase reactions that exhibit: (i) negligible non-specific binding of protein and other reaction components (thus reducing or minimizing substrate background), (ii) negligible non-specific nucleic acid amplification product, and (iii) provide tunable nucleic acid amplification reactions. Although described herein primarily in the context of nucleic acid hybridization, amplification, and sequencing assays, it will be understood by those of skill in the art that the disclosed low-binding supports may be used in any of a variety of other bioassay formats including, but not limited to, sandwich immunoassays, enzyme-linked immunosorbent assays (ELISAs), etc.

Plastic surface: Examples of materials from which the substrate or support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), or any combination thereof. Various compositions of both glass and plastic substrates are contemplated.

Modification of a surface for the purposes disclosed herein involves making surfaces reactive against many chemical groups (—R), including amines. When prepared on an appropriate substrate, these reactive surfaces can be stored long term at room temperature for example for at least 3 months or more. Such surfaces can be further grafted with R-PEG and R-primer oligomer for on-surface amplification of nucleic acids, as described elsewhere herein. Plastic surfaces, such as cyclic olefin polymer (COP), may be modified using any of a large number of methods known in the art. For example, they can be treated with Ti:Sapphire laser ablation, UV-mediated ethylene glycol methacrylate photografting, plasma treatment, or mechanical agitation (e.g., sand blasting, or polishing, etc.) to create hydrophilic surfaces that can stay reactive for months against many chemical groups, such as amines. These groups may then allow conjugation of passivation polymers such as PEG, or biomolecules such as DNA or proteins, without loss of biochemical activity. For example, attachment of DNA primer oligomers allows DNA amplification on a passivated plastic surface while reducing or minimizing the non-specific adsorption of proteins, fluorophore molecules, or other hydrophobic molecules.

Additionally, surface modification can be combined with, e.g., laser printing or UV masking, to create patterned surfaces. This allows patterned attachment of DNA oligomers, proteins, or other moieties, providing for surface-based enzymatic activity, binding, detection, or processing. For example, DNA oligomers may be used to amplify DNA only within patterned features, or to capture amplified long DNA concatemers in a patterned fashion. In some embodiments, enzyme islands may be generated in the patterned areas that are capable of reacting with solution-based substrates. Because plastic surfaces are especially amenable to these processing modes, in some embodiments as contemplated herein, plastic surfaces may be recognized as being particularly advantageous.

Furthermore, plastic can be injection molded, embossed, or 3D printed to form any shape, including microfluidic devices, much more easily than glass substrates, and thus can be used to create surfaces for the binding and analysis of biological samples in multiple configurations, e.g., sample-to-result microfluidic chips for biomarker detection or DNA sequencing.

Specific localized DNA amplification on modified plastic surfaces can be prepared and can produce spots with an ultra-high contrast to noise ratio and very low background when probed with fluorescent labels.

Hydrophilized and amine reactive cyclic olefin polymer surface with amine-primer and amine-PEG can be prepared and it supports rolling circle amplification. When probed with fluorophore labeled primers, or when labeled dNTPs added to the hybridized primers by a polymerase, bright spots of DNA amplicons were observed that exhibited signal to noise ratios greater than 100 with backgrounds that are extremely low, indicating highly specific amplification, and ultra-low levels of protein and hydrophobic fluorophore binding which are hallmarks of the high accuracy detection systems such as fluorescence-based DNA sequencers.

Oligonucleotide primers and adapter sequences: In general, at least one layer of the one or more surface modification or polymer layers applied to the capillary or channel lumen surface may comprise functional groups for covalently or non-covalently attaching oligonucleotide adapter or primer sequences, or the at least one layer may already comprise covalently or non-covalently attached oligonucleotide adapter or primer sequences at the time that it is grafted to or deposited on the support surface. In some aspects, the capillary or the microfluidic channel comprises an oligonucleotide population directed to sequence a prokaryotic genome. In some aspects, the capillary or the microfluidic channel comprises an oligonucleotide population directed to sequence a transcriptome.

The central region of the flow cell devices or systems can include a surface having at least one oligonucleotide tethered thereto. In some embodiments, the surface can be an interior surface of a microfluidic channel or capillary tube. In some aspects, the surface is a locally planar surface. In some embodiments, the oligonucleotide is directly tethered to the surface. In some embodiments, the oligonucleotide is tethered to the surface through an intermediate molecule.

The oligonucleotide tethered to the interior surface of the central region can include segments that bind to different targets. In some instance, the oligonucleotide exhibits a segment that specifically hybridizes to a eukaryotic genomic nucleic acid segment. In some instance, the oligonucleotide exhibits a segment that specifically hybridizes to a prokaryotic genomic nucleic acid segment. In some instance, the oligonucleotide exhibits a segment that specifically hybridizes to a viral nucleic acid segment. In some instance, the oligonucleotide exhibits a segment that specifically hybridizes to a transcriptome nucleic acid segment.

When the central region comprises a surface having one or more oligonucleotide tethered thereto, the interior volume of the central region can be adjusted based on the types of sequencing performed. In some embodiments, the central region comprises an interior volume suitable for sequencing a eukaryotic genome. In some embodiments, the central region comprises an interior volume suitable for sequencing a prokaryotic genome. In some embodiments, the central region comprises an interior volume suitable for sequencing a transcriptome. For example, in some embodiments, the interior volume of the central region may comprise a volume of less than 0.050, between 0.05 μl and 0.1 μl, between 0.05 μl and 0.2 μl, between 0.05 μl and 0.5 μl, between 0.05 μl and 0.8 μl, between 0.05 μl and 1 μl, between 0.05 μl and 1.2 μl, between 0.05 μl and 1.5 μl, between 0.1 μl and 1.5 μl, between 0.2 μl and 1.5 μl, between 0.5 μl and 1.5 μl, between 0.8 μl and 1.5 μl, between 1 μl and 1.5 μl, between 1.2 μl and 1.5 μl, or greater than 1.5 μl, or a range defined by any two of the foregoing. In some embodiments, the interior volume of the central region may comprise a volume of less than 0.50, between 0.5 μl and 1 μl, between 0.5 μl and 2 μl, between 0.5 μl and 5 μl, between 0.5 μl and 8 μl, between 0.5 μl and 10 μl, between 0.5 μl and 12 μl, between 0.5 μl and 15 μl, between 1 μl and 15 μl, between 2 μl and 15 μl, between 5 μl and 15 μl, between 8 μl and 15 μl, between 10 μl and 15 μl, between 12 μl and 15 μl, or greater than 15 μl, or a range defined by any two of the foregoing. In some embodiments, the interior volume of the central region may comprise a volume of less than 50, between 5 μl and 10 μl, between 5 μl and 20 μl, between 5 μl and 500 μl, between 5 μl and 80 μl, between 5 μl and 100 μl, between 5 μl and 120 μl, between 5 μl and 150 μl, between 10 μl and 150 μl, between 20 μl and 150 μl, between 50 μl and 150 μl, between 80 μl and 150 μl, between 100 μl and 150 μl, between 120 μl and 150 μl, or greater than 150 μl, or a range defined by any two of the foregoing. In some embodiments, the interior volume of the central region may comprise a volume of less than 500, between 50 μl and 100 μl, between 50 μl and 200 μl, between 50 μl and 500 μl, between 50 μl and 800 μl, between 50 μl and 1000 μl, between 50 μl and 1200 μl, between 50 μl and 1500 μl, between 100 μl and 1500 μl, between 200 μl and 1500 μl, between 500 μl and 1500 μl, between 800 μl and 1500 μl, between 1000 μl and 1500 μl, between 1200 μl and 1500 μl, or greater than 1500 μl, or a range defined by any two of the foregoing. In some embodiments, the interior volume of the central region may comprise a volume of less than 500 μl, between 500 μl and 1000 μl, between 500 μl and 2000 μl, between 500 μl and 5 ml, between 500 μl and 8 ml, between 500 μl and 10 ml, between 500 μl and 12 ml, between 500 μl and 15 ml, between 1 ml and 15 ml, between 2 ml and 15 ml, between 5 ml and 15 ml, between 8 ml and 15 ml, between 10 ml and 15 ml, between 12 ml and 15 ml, or greater than 15 ml, or a range defined by any two of the foregoing. In some embodiments, the interior volume of the central region may comprise a volume of less than 5 ml, between 5 ml and 10 ml, between 5 ml and 20 ml, between 5 ml and 50 ml, between 5 ml and 80 ml, between 5 ml and 100 ml, between 5 ml and 120 ml, between 5 ml and 150 ml, between 10 ml and 150 ml, between 20 ml and 150 ml, between 50 ml and 150 ml, between 80 ml and 150 ml, between 100 ml and 150 ml, between 120 ml and 150 ml, or greater than 150 ml, or a range defined by any two of the foregoing. In some embodiments, the methods and systems described herein comprise an array or collection of flow cell devices or systems comprising multiple discrete capillaries, microfluidic channels, fluidic channels, chambers, or lumenal regions, wherein the combined interior volume is, comprises, or includes one or more of the values within a range disclosed herein.

One or more types of oligonucleotide primer may be attached or tethered to the support surface. In some instances, the one or more types of oligonucleotide adapters or primers may comprise spacer sequences, adapter sequences for hybridization to adapter-ligated template library nucleic acid sequences, forward amplification primers, reverse amplification primers, sequencing primers, and/or molecular barcoding sequences, or any combination thereof.

The tethered oligonucleotide adapter and/or primer sequences may range in length from about 10 nucleotides to about 100 nucleotides. In some instances, the tethered oligonucleotide adapter and/or primer sequences may be no more than 10, at least 10, at least 20, at least 30, at least 40, at least 50, at least 60, at least 70, at least 80, at least 90, or at least 100 nucleotides in length. In some instances, the tethered oligonucleotide adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some instances the length of the tethered oligonucleotide adapter and/or primer sequences may range from about 20 nucleotides to about 80 nucleotides. Those of skill in the art will recognize that the length of the tethered oligonucleotide adapter and/or primer sequences may have any value within this range, e.g., about 24 nucleotides.

The number of coating layers and/or the material composition of each layer is chosen so as to adjust the resultant surface density of oligonucleotide primers (or other attached molecules) on the coated capillary lumen surface. In some instances, the surface density of oligonucleotide primers may range from about 1,000 primer molecules per μm2 to about 1,000,000 primer molecules per μm2. In some instances, the surface density of oligonucleotide primers may be at least 1,000, at least 10,000, at least 100,000, or at least 1,000,000 molecules per μm2. In some instances, the surface density of oligonucleotide primers may be at most 1,000,000, at most 100,000, at most 10,000, or at most 1,000 molecules per μm2. Any of the lower and upper values described in this paragraph may be combined to form a range included within the present disclosure, for example, in some instances the surface density of primers may range from about 10,000 molecules per μm2 to about 100,000 molecules per μm2. Those of skill in the art will recognize that the surface density of primer molecules may have any value within this range, e.g., about 455,000 molecules per μm2. In some instances, the surface properties of the capillary or channel lumen coating, including the surface density of tethered oligonucleotide primers, may be adjusted so as to improve or optimize, e.g., solid-phase nucleic acid hybridization specificity and efficiency, and/or solid-phase nucleic acid amplification rate, specificity, and efficiency.

Capillary flow cell cartridges: Also disclosed herein are capillary flow cell cartridges that may comprise one, two, or more capillaries to create independent flow channels. FIG. 30 provides a non-limiting example of capillary flow cell cartridge that comprises two glass capillaries, fluidic adaptors (two per capillary in this example), and a cartridge chassis that mates with the capillaries and/or fluidic adapters such that the capillaries are held in a fixed orientation relative to the cartridge. In some instances, the fluidic adaptors may be integrated with the cartridge chassis. In some instances, the cartridge may comprise additional adapters that mate with the capillaries and/or capillary fluidic adapters. In some instances, the capillaries are permanently mounted in the cartridge. In some instances, the cartridge chassis is designed to allow one or more capillaries of the flow cell cartridge to be interchangeable removed and replaced. For example, in some instances, the cartridge chassis may comprise a hinged “clamshell” configuration which allows it to be opened so that one or more capillaries may be removed and replaces. In some instances, the cartridge chassis is configured to mount on, for example, the stage of a microscope system or within a cartridge holder of an instrument system.

The capillary flow cell cartridges of the present disclosure may comprise a single capillary. In some instances, the capillary flow cell cartridges of the present disclosure may comprise 2, 3, 4, 5, 6, 7, 8, 9, 10, 11, 12, 13, 14, 15, 16, 17, 18, 19, 20, or more than 20 capillaries. The one or more capillaries of the flow cell cartridge may have any of the geometries, dimensions, material compositions, and/or coatings as described above for the single capillary flow cell devices. Similarly, the fluidic adapters for the individual capillaries in the cartridge (typically two fluidic adapters per capillary) may have any of the geometries, dimensions, and material compositions as described above for the single capillary flow cell devices, except that in some instances the fluidic adapters may be integrated directly with the cartridge chassis as illustrated in FIG. 30. In some instances, the cartridge may comprise additional adapters (i.e., in addition to the fluidic adapters) that mate with the capillaries and/or fluidic adapters and help to position the capillaries within the cartridge. These adapters may be constructed using the same fabrication techniques and materials as those outlined above for the fluidic adapters.

In some embodiments, one or more devices according to the present disclosure may comprise a first surface in an orientation generally facing the interior of the flow channel, wherein said surface may further comprise a polymer coating as disclosed elsewhere herein, and wherein said surface may further comprise one or more oligonucleotides such as a capture oligonucleotide, an adapter oligonucleotide, or any other oligonucleotide as disclosed herein. In some embodiments, said devices may further comprise a second surface in an orientation generally facing the interior of the flow channel and further generally facing or parallel to the first surface, wherein said surface may further comprise a polymer coating as disclosed elsewhere herein, and wherein said surface may further comprise one or more oligonucleotides such as a capture oligonucleotide, an adapter oligonucleotide, or any other oligonucleotide as disclosed herein. In some embodiments, a device of the present disclosure may comprise a first surface in an orientation generally facing the interior of the flow channel, a second surface in an orientation generally facing the interior of the flow channel and further generally facing or parallel to the first surface, a third surface generally facing the interior of a second flow channel, and a fourth surface, generally facing the interior of the second flow channel and generally opposed to or parallel to the third surface; wherein said second and third surfaces may be located on or attached to opposite sides of a generally planar substrate which may be a reflective, transparent, or translucent substrate. In some embodiments, an imaging surface or imaging surfaces within a flowcell may be located within the center of a flowcell or within or as part of a division between two subunits or subdivisions of a flowcell, wherein said flowcell may comprise a top surface and a bottom surface, one or both of which may be transparent to such detection mode as may be utilized; and wherein a surface comprising oligonucleotides or polynucleotides and/or one or more polymer coatings, may be placed or interposed within the lumen of the flowcell. In some embodiments, the top and/or bottom surfaces do not include attached oligonucleotides or polynucleotides. In some embodiments, said top and/or bottom surfaces do comprise attached oligonucleotides and/or polynucleotides. In some embodiments, either said top or said bottom surface may comprise attached oligonucleotides and/or polynucleotides. A surface or surfaces placed or interposed within the lumen of a flowcell may be located on or attached one side, an opposite side, or both sides of a generally planar substrate which may be a reflective, transparent, or translucent substrate. In some embodiments, an optical apparatus as provided elsewhere herein or as otherwise known in the art is utilized to provide images of a first surface, a second surface, a third surface, a fourth surface, a surface interposed within the lumen of a flowcell, or any other surface provided herein which may contain one or more oligonucleotides or polynucleotides attached thereto.

Microfluidic chip flow cell cartridges: Also disclosed herein are microfluidic channel flow cell cartridges that may a plurality of independent flow channels. A non-limiting example of microfluidic chip flow cell cartridge that comprises a chip having two or more parallel glass channels formed on the chip, fluidic adaptors coupled to the chip, and a cartridge chassis that mates with the chip and/or fluidic adapters such that the chip is posited in a fixed orientation relative to the cartridge. In some instances, the fluidic adaptors may be integrated with the cartridge chassis. In some instances, the cartridge may comprise additional adapters that mate with the chip and/or fluidic adapters. In some instances, the chip is permanently mounted in the cartridge. In some instances, the cartridge chassis is designed to allow one or more chips of the flow cell cartridge to be interchangeable removed and replaced. For example, in some instances, the cartridge chassis may comprise a hinged “clamshell” configuration which allows it to be opened so that one or more capillaries may be removed and replaces. In some instances, the cartridge chassis is configured to mount on, for example, the stage of a microscope system or within a cartridge holder of an instrument system. Even through only one chip is described in the non-limiting example, it is understood that more than one chip can be used in the microfluidic channel flow cell cartridge

The flow cell cartridges of the present disclosure may comprise a single microfluidic chip or a plurality of microfluidic chips. In some instances, the flow cell cartridges of the present disclosure may comprise 2, 3, 4, 5, 6, 7, 8, 9, 10, 11, 12, 13, 14, 15, 16, 17, 18, 19, 20, or more than 20 microfluidic chips. In some instances, the microfluidic chip can have one channel. In some instances, the microfluidic chip can have 2, 3, 4, 5, 6, 7, 8, 9, 10, 11, 12, 13, 14, 15, 16, 17, 18, 19, 20, or more than 20 channels. The one or more chips of the flow cell cartridge may have any of the geometries, dimensions, material compositions, and/or coatings as described above for the single microfluidic chip flow cell devices. Similarly, the fluidic adapters for the individual chip in the cartridge (typically two fluidic adapters per capillary) may have any of the geometries, dimensions, and material compositions as described above for the single microfluidic chip flow cell devices, except that in some instances the fluidic adapters may be integrated directly with the cartridge chassis. In some instances, the cartridge may comprise additional adapters (i.e., in addition to the fluidic adapters) that mate with the chip and/or fluidic adapters and help to position the chip within the cartridge. These adapters may be constructed using the same fabrication techniques and materials as those outlined above for the fluidic adapters.

The cartridge chassis (or “housing”) may be fabricated from metal and/or polymer materials such as aluminum, anodized aluminum, polycarbonate (PC), acrylic (PMMA), or Ultem (PEI), while ohter materials are also consistent with the disclosure. A housing may be fabricated using CNC machining and/or molding techniques, and designed so that one, two, or more than two capillaries are constrained by the chassis in a fixed orientation to create independent flow channels. The capillaries may be mounted in the chassis using, e.g., a compression fit design, or by mating with compressible adapters made of silicone or a fluoroelastomer. In some instance, two or more components of the cartridge chassis (e.g., an upper half and a lower half) are assembled using, e.g., screws, clips, clamps, or other fasteners so that the two halves are separable. In some instances, two or more components of the cartridge chassis are assembled using, e.g., adhesives, solvent bonding, or laser welding so that the two or more components are permanently attached.

Some flow cell cartridges of the present disclosure further comprise additional components that are integrated with the cartridge to provide enhanced performance for specific applications. Examples of additional components that may be integrated into the cartridge include, but are not limited to, fluid flow control components (e.g., miniature valves, miniature pumps, mixing manifolds, etc.), temperature control components (e.g., resistive heating elements, metal plates that serve as heat sources or sinks, piezoelectric (Peltier) devices for heating or cooling, temperature sensors), or optical components (e.g., optical lenses, windows, filters, mirrors, prisms, fiber optics, and/or light-emitting diodes (LEDs) or other miniature light sources that may collectively be used to facilitate spectroscopic measurements and/or imaging of one or more capillary flow channels).

Systems and system components: The flow cell devices and flow cell cartridges disclosed herein may be used as components of systems designed for a variety of chemical analysis, biochemical analysis, nucleic acid analysis, cell analysis, or tissue analysis application. In general, such systems may comprise one or more fluid flow control modules, temperature control modules, spectroscopic measurement and/or imaging modules, and processors or computers, as well as one or more of the single capillary flow cell devices and capillary flow cell cartridges or the microfluidic chip flow cell devices and flow cell cartridges described herein.

The systems disclosed herein may comprise 1, 2, 3, 4, 5, 6, 7, 8, 9, 10, or more than 10 single capillary flow cell devices or capillary flow cell cartridges. In some instances the single capillary flow cell devices or capillary flow cell cartridges may be removable, exchangeable components of the disclosed systems. In some instances, the single capillary flow cell devices or capillary flow cell cartridges may be disposable or consumable components of the disclosed systems. The systems disclosed herein may comprise 1, 2, 3, 4, 5, 6, 7, 8, 9, 10, or more than 10 single microfluidic channel flow cell devices or microfluidic channel flow cell cartridges. In some instances the single microfluidic channel flow cell devices or microfluidic channel flow cell cartridges may be removable, exchangeable components of the disclosed systems. In some instances, the flow cell devices or flow cell cartridges may be disposable or consumable components of the disclosed systems.

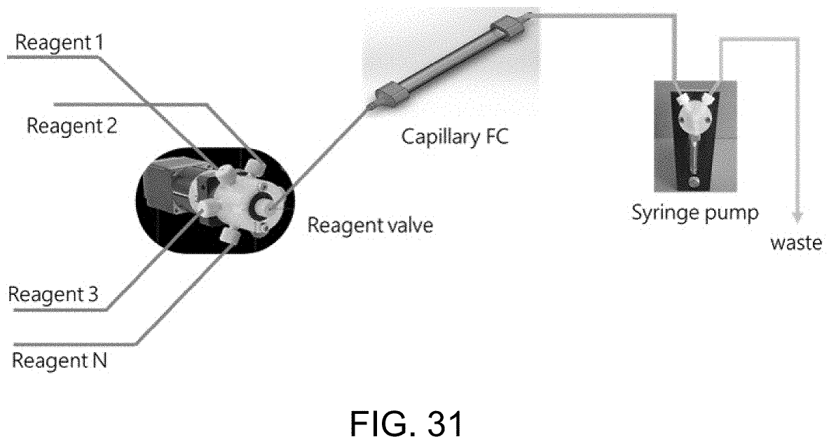

FIG. 31 illustrates one embodiment of a simple system comprising a single capillary flow cell connected to various fluid flow control components, where the single capillary is optically accessible and compatible with mounting on a microscope stage or in a custom imaging instrument for use in various imaging applications. A plurality of reagent reservoirs are fluidically-coupled with the inlet end of the single capillary flow cell device, where the reagent flowing through the capillary at any given point in time is controlled by means of a programmable rotary valve that allows the user to control the timing and duration of reagent flow. In this non-limiting example, fluid flow is controlled by means of a programmable syringe pump that provides precise control and timing of volumetric fluid flow and fluid flow velocity.

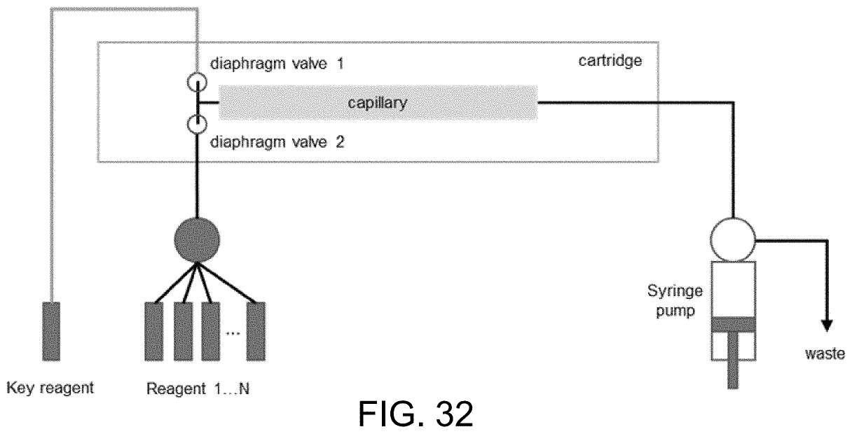

FIG. 32 illustrates one embodiment of a system that comprises a capillary flow cell cartridge having integrated diaphragm valves to reduce or minimize dead volume and conserve certain key reagents. The integration of miniature diaphragm valves into the cartridge allows the valve to be positioned in close proximity to the inlet of the capillary, thereby reducing or minimizing dead volume within the device and reducing the consumption of costly reagents. The integration of valves and other fluid control components within the capillary flow cell cartridge also allows greater fluid flow control functionality to be incorporated into the cartridge design.

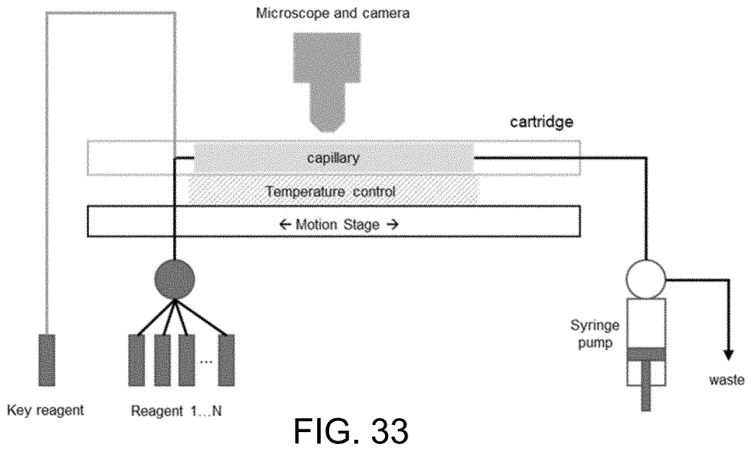

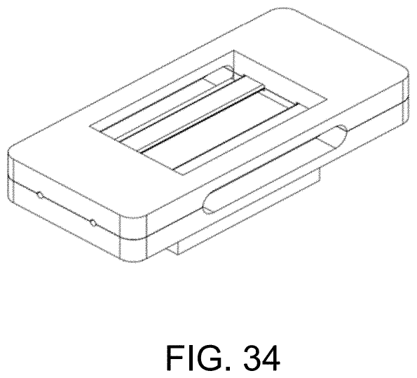

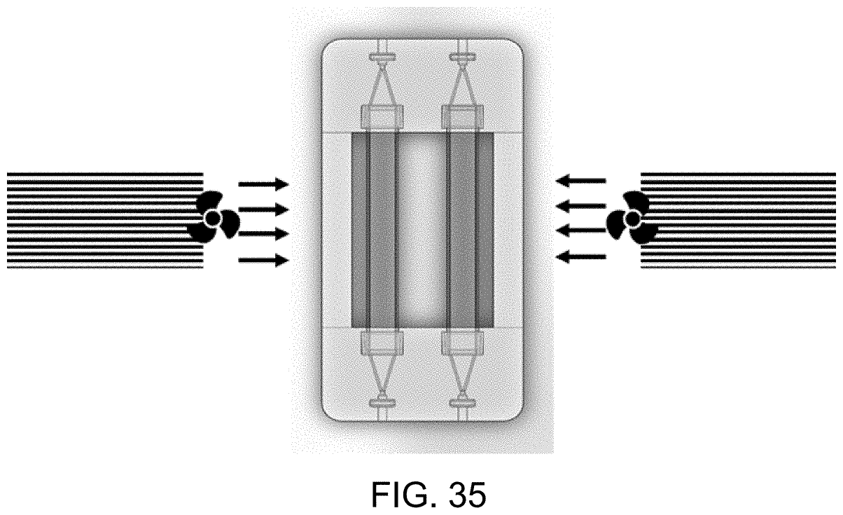

FIG. 33 shows an example of a capillary flow cell cartridge-based fluidics system used in combination with a microscope setup, where the cartridge incorporates or mates with a temperature control component such as a metal plate that makes contact with the capillaries within the cartridge and serves as a heat source/sink. The microscope setup consists of an illumination system (e.g., including a laser, LED, or halogen lamp, etc., as a light source), an objective lens, an imaging system (e.g., a CMOS or CCD camera), and a translation stage to move the cartridge relative to the optical system, which allows, e.g., fluorescence and/or bright field images to be acquired for different regions of the capillary flow cells as the stage is moved.

FIG. 34 illustrates one non-limiting example for temperature control of the flow cells (e.g., capillary or microfluidic channel flow cells) through the use of a metal plate that is placed in contact with the flow cell cartridge. In some instances, the metal plate may be integrated with the cartridge chassis. In some instances, the metal plate may be temperature controlled using a Peltier or resistive heater.

FIG. 35 illustrates one non-limiting approach for temperature control of the flow cells (e.g., capillary or microfluidic channel flow cells) that comprises a non-contact thermal control mechanism. In this approach, a stream of temperature-controlled air is directed through the flow cell cartridge (e.g., towards a single capillary flow cell device or a microfluidic channel flow cell device) using an air temperature control system. The air temperature control system comprises a heat exchanger, e.g., a resistive heater coil, fins attached to a Peltier device, etc., that is capable of heating and/or cooling the air and holding it at a constant, user-specified temperature. The air temperature control system also comprises an air delivery device, such as a fan, that directs the stream of heated or cooled air to the capillary flow cell cartridge. In some instances, the air temperature control system may be set to a constant temperature T1 so that the air stream, and consequently the flow cell or cartridge (e.g., capillary flow cell or microfluidic channel flow cell) is kept at a constant temperature T2, which in some cases may differ from the set temperature T1 depending on the environment temperature, air flow rate, etc. In some instances, two or more such air temperature control systems may be installed around the capillary flow cell device or flow cell cartridge so that the capillary or cartridge may be rapidly cycled between several different temperatures by controlling which one of the air temperature control systems is active at a given time. In another approach, the temperature setting of the air temperature control system may be varied so the temperature of the capillary flow cell or cartridge may be changed accordingly.

Fluid flow control module: In general, the disclosed instrument systems will provide fluid flow control capability for delivering samples or reagents to the one or more flow cell devices or flow cell cartridges (e.g., single capillary flow cell device or microfluidic channel flow cell device) connected to the system. Reagents and buffers may be stored in bottles, reagent and buffer cartridges, or other suitable containers that are connected to the flow cell inlets by means of tubing and valve manifolds. The disclosed systems may also include processed sample and waste reservoirs in the form of bottles, cartridges, or other suitable containers for collecting fluids downstream of the capillary flow cell devices or capillary flow cell cartridges. In some embodiments, the fluid flow control (or “fluidics”) module may provide programmable switching of flow between different sources, e.g. sample or reagent reservoirs or bottles located in the instrument, and the central region (e.g., capillary or microfluidic channel) inlet(s). In some embodiments, the fluid flow control module may provide programmable switching of flow between the central region (e.g., capillary or microfluidic channel) outlet(s) and different collection points, e.g., processed sample reservoirs, waste reservoirs, etc., connected to the system. In some instances, samples, reagents, and/or buffers may be stored within reservoirs that are integrated into the flow cell cartridge itself. In some instances, processed samples, spent reagents, and/or used buffers may be stored within reservoirs that are integrated into the flow cell cartridge itself.

Control of fluid flow through the disclosed systems will typically be performed through the use of pumps (or other fluid actuation mechanisms) and valves (e.g., programmable pumps and valves). Examples of suitable pumps include, but are not limited to, syringe pumps, programmable syringe pumps, peristaltic pumps, diaphragm pumps, and the like. Examples of suitable valves include, but are not limited to, check valves, electromechanical two-way or three-way valves, pneumatic two-way and three-way valves, and the like. In some embodiments, fluid flow through the system may be controlled by means of applying positive pneumatic pressure to one or more inlets of the reagent and buffer containers, or to inlets incorporated into flow cell cartridge(s) (e.g., capillary or microfluidic channel flow cell cartridges). In some embodiments, fluid flow through the system may be controlled by means of drawing a vacuum at one or more outlets of waste reservoir(s), or at one or more outlets incorporated into flow cell cartridge(s) (e.g., capillary or microfluidic channel flow cell cartridges).

In some instances, different modes of fluid flow control are utilized at different points in an assay or analysis procedure, e.g. forward flow (relative to the inlet and outlet for a given capillary flow cell device), reverse flow, oscillating or pulsatile flow, or combinations thereof. In some applications, oscillating or pulsatile flow may be applied, for example, during assay wash/rinse steps to facilitate complete and efficient exchange of fluids within the one or more flow cell devices or flow cell cartridges (e.g., single capillary flow cell devices or cartridges and microfluidic chip flow cell devices or cartridges).

Similarly, in some cases different fluid flow rates may be utilized at different points in the assay or analysis process workflow, for example, in some instances, the volumetric flow rate may vary from −100 ml/sec to +100 ml/sec. In some embodiment, the absolute value of the volumetric flow rate may be at least 0.001 ml/sec, at least 0.01 ml/sec, at least 0.1 ml/sec, at least 1 ml/sec, at least 10 ml/sec, or at least 100 ml/sec. In some embodiments, the absolute value of the volumetric flow rate may be at most 100 ml/sec, at most 10 ml/sec, at most 1 ml/sec, at most 0.1 ml/sec, at most 0.01 ml/sec, or at most 0.001 ml/sec. The volumetric flow rate at a given point in time may have any value within this range, e.g. a forward flow rate of 2.5 ml/sec, a reverse flow rate of −0.05 ml/sec, or a value of 0 ml/sec (i.e., stopped flow).

Temperature control module: As noted above, in some instances the disclosed systems will include temperature control functionality for the purpose of facilitating the accuracy and reproducibility of assay or analysis results. Examples of temperature control components that may be incorporated into the instrument system (or capillary flow cell cartridge) design include, but are not limited to, resistive heating elements, infrared light sources, Peltier heating or cooling devices, heat sinks, thermistors, thermocouples, and the like. In some instances, the temperature control module (or “temperature controller”) may provide for a programmable temperature change at a specified, adjustable time prior to performing specific assay or analysis steps. In some instances, the temperature controller may provide for programmable changes in temperature over specified time intervals. In some embodiments, the temperature controller may further provide for cycling of temperatures between two or more set temperatures with specified frequency and ramp rates so that thermal cycling for amplification reactions may be performed.

Spectroscopy or imaging modules: As indicated above, in some instances the disclosed systems will include optical imaging or other spectroscopic measurement capabilities. For example, any of a variety of imaging modes known to those of skill in the art may be implemented including, but not limited to, bright-field, dark-field, fluorescence, luminescence, or phosphorescence imaging. In some embodiments, the central region comprises a window that allows at least a part of the central region to be illuminated and imaged. In some embodiments, the capillary tube comprises a window that allows at least a part of the capillary tube to be illuminated and imaged. In some embodiments, the microfluidic chip comprises a window that allows at least a part of the chip channel to be illuminated and imaged.

In some embodiments, single wavelength excitation and emission fluorescence imaging may be performed. In some embodiments, dual wavelength excitation and emission (or multi-wavelength excitation or emission) fluorescence imaging may be performed. In some instances, the imaging module is configured to acquire video images. The choice of imaging mode may impact the design of the flow cells devices or flow cell cartridges in that all or a portion of the capillaries or cartridge will necessarily need to be optically transparent over the spectral range of interest. In some instances, a plurality of capillaries within a capillary flow cell cartridge may be imaged in their entirety within a single image. In some embodiments, only a single capillary or a subset of capillaries within a capillary flow cell cartridge, or portions thereof, may be imaged within a single image. In some embodiments, a series of images may be “tiled” to create a single high resolution image of one, two, several, or the entire plurality of capillaries within a cartridge. In some instances, a plurality of channels within a microfluidic chip may be imaged in their entirety within a single image. In some embodiments, only a single channel or a subset of channels within a microfluidic chip, or portions thereof, may be imaged within a single image. In some embodiments, a series of images may be “tiled” to create a single high resolution image of one, two, several, or the entire plurality of capillaries or microfluidic channels within a cartridge.

A spectroscopy or imaging module may comprise, e.g., a microscope equipped with a CMOS of CCD camera. In some instances, the spectroscopy or imaging module may comprise, e.g., a custom instrument configured to perform a specific spectroscopic or imaging technique of interest. In general, the hardware associated with the imaging module may include light sources, detectors, and other optical components, as well as processors or computers.

Light sources: Any of a variety of light sources may be used to provide the imaging or excitation light, including but not limited to, tungsten lamps, tungsten-halogen lamps, arc lamps, lasers, light emitting diodes (LEDs), or laser diodes. In some instances, a combination of one or more light sources, and additional optical components, e.g. lenses, filters, apertures, diaphragms, mirrors, and the like, may be configured as an illumination system (or sub-system).

Detectors: Any of a variety of image sensors may be used for imaging purposes, including but not limited to, photodiode arrays, charge-coupled device (CCD) cameras, or complementary metal-oxide-semiconductor (CMOS) image sensors. As used herein, “imaging sensors” may be one-dimensional (linear) or two-dimensional array sensors. In many instances, a combination of one or more image sensors, and additional optical components, e.g. lenses, filters, apertures, diaphragms, mirrors, and the like, may be configured as an imaging system (or sub-system). In some instances, e.g., where spectroscopic measurements are performed by the system rather than imaging, suitable detectors may include, but are not limited to, photodiodes, avalanche photodiodes, and photomultipliers.

Other optical components: The hardware components of the spectroscopic measurement or imaging module may also include a variety of optical components for steering, shaping, filtering, or focusing light beams through the system. Examples of suitable optical components include, but are not limited to, lenses, mirrors, prisms, apertures, diffraction gratings, colored glass filters, long-pass filters, short-pass filters, bandpass filters, narrowband interference filters, broadband interference filters, dichroic reflectors, optical fibers, optical waveguides, and the like. In some instances, the spectroscopic measurement or imaging module may further comprise one or more translation stages or other motion control mechanisms for the purpose of moving capillary flow cell devices and cartridges relative to the illumination and/or detection/imaging sub-systems, or vice versa.

Total internal reflection: In some instances, the optical module or sub-system may be designed to use all or a portion of an optically transparent wall of the capillaries or microfluidic channels in flow cell devices and cartridges as a waveguide for delivering excitation light to the capillary or channel lumen(s) via total internal reflection. When incident excitation light strikes the surface of the capillary or channel lumen at an angle with respect to a normal to the surface that is larger than the critical angle (determined by the relative refractive indices of the capillary or channel wall material and the aqueous buffer within the capillary or channel), total internal reflection occurs at the surface and the light propagates through the capillary or channel wall along the length of the capillary or channel. Total internal reflection generates an evanescent wave at the lumen surface which penetrates the lumen interior for extremely short distances, and which may be used to selectively excite fluorophores at the surface, e.g., labeled nucleotides that have been incorporated by a polymerase into a growing oligonucleotide through a solid-phase primer extension reaction.

Imaging processing software: In some instances, the system may further comprise a computer (or processor) and computer-readable medium that includes code for providing image processing and analysis capability. Examples of image processing and analysis capability that may be provided by the software include, but are not limited to, manual, semi-automated, or fully-automated image exposure adjustment (e.g. white balance, contrast adjustment, signal-averaging and other noise reduction capability, etc.), automated edge detection and object identification (e.g., for identifying clonally-amplified clusters of fluorescently-labeled oligonucleotides on the lumen surface of capillary flow cell devices), automated statistical analysis (e.g., for determining the number of clonally-amplified clusters of oligonucleotides identified per unit area of the capillary lumen surface, or for automated nucleotide base-calling in nucleic acid sequencing applications), and manual measurement capabilities (e.g. for measuring distances between clusters or other objects, etc.). Optionally, instrument control and image processing/analysis software may be written as separate software modules. In some embodiments, instrument control and image processing/analysis software may be incorporated into an integrated package.

System control software: In some instances, the system may comprise a computer (or processor) and a computer-readable medium that includes code for providing a user interface as well as manual, semi-automated, or fully-automated control of all system functions, e.g., control of the fluidics module, the temperature control module, and/or the spectroscopy or imaging module, as well as other data analysis and display options. The system computer or processor may be an integrated component of the system (e.g. a microprocessor or mother board embedded within the instrument) or may be a stand-alone module, for example, a main frame computer, a personal computer, or a laptop computer. Examples of fluid control functions provided by the system control software include, but are not limited to, volumetric fluid flow rates, fluid flow velocities, the timing and duration for sample and reagent addition, buffer addition, and rinse steps. Examples of temperature control functions provided by the system control software include, but are not limited to, specifying temperature set point(s) and control of the timing, duration, and ramp rates for temperature changes. Examples of spectroscopic measurement or imaging control functions provided by the system control software include, but are not limited to, autofocus capability, control of illumination or excitation light exposure times and intensities, control of image acquisition rate, exposure time, and data storage options.

Processors and computers: In some instances, the disclosed systems may comprise one or more processors or computers. The processor may be a hardware processor such as a central processing unit (CPU), a graphic processing unit (GPU), a general-purpose processing unit, or a computing platform. The processor may be comprised of any of a variety of suitable integrated circuits, microprocessors, logic devices, field-programmable gate arrays (FPGAs) and the like. In some instances, the processor may be a single core or multi core processor, or a plurality of processors may be configured for parallel processing. Although the disclosure is described with reference to a processor, other types of integrated circuits and logic devices are also applicable. The processor may have any suitable data operation capability. For example, the processor may perform 512 bit, 256 bit, 128 bit, 64 bit, 32 bit, or 16 bit data operations.

The processor or CPU can execute a sequence of machine-readable instructions, which can be embodied in a program or software. The instructions may be stored in a memory location. The instructions can be directed to the CPU, which can subsequently program or otherwise configure the CPU to implement, e.g., the system control methods of the present disclosure. Examples of operations performed by the CPU can include fetch, decode, execute, and write back.

Some processors are a processing unit of a computer system. The computer system may enable cloud-based data storage and/or computing. In some instances, the computer system may be operatively coupled to a computer network (“network”) with the aid of a communication interface. The network may be the internet, an intranet and/or extranet, an intranet and/or extranet that is in communication with the internet, or a local area network (LAN). The network in some cases is a telecommunication and/or data network. The network may include one or more computer servers, which may enable distributed computing, such as cloud-based computing.

The computer system may also include computer memory or memory locations (e.g., random-access memory, read-only memory, flash memory), electronic storage units (e.g., hard disk), communication interfaces (e.g., network adapters) for communicating with one or more other systems, and peripheral devices, such as cache, other memory units, data storage units and/or electronic display adapters. In some instances, the communication interface may allow the computer to be in communication with one or more additional devices. The computer may be able to receive input data from the coupled devices for analysis. Memory units, storage units, communication interfaces, and peripheral devices may be in communication with the processor or CPU through a communication bus (solid lines), such as may be incorporated into a motherboard. A memory or storage unit may be a data storage unit (or data repository) for storing data. The memory or storage units may store files, such as drivers, libraries and saved programs. The memory or storage units may store user data, e.g., user preferences and user programs.

The system control, image processing, and/or data analysis methods as described herein can be implemented by way of machine-executable code stored in an electronic storage location of the computer system, such as, for example, in the memory or electronic storage unit. The machine-executable or machine-readable code can be provided in the form of software. During use, the code can be executed by the processor. In some cases, the code can be retrieved from the storage unit and stored in memory for ready access by the processor. In some situations, the electronic storage unit can be precluded, and machine-executable instructions are stored in memory.

In some instances, the code may be pre-compiled and configured for use with a machine having a processer adapted to execute the code. In some instances, the code may be compiled during runtime. The code can be supplied in a programming language that can be selected to enable the code to execute in a pre-compiled or as-compiled fashion.

Some aspects of the systems and methods provided herein can be embodied in software. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

In some instances, the system control, image processing, and/or data analysis methods of the present disclosure may be implemented by way of one or more algorithms. An algorithm may be implemented by way of software upon execution by the central processing unit.

Nucleic acid sequencing applications: Nucleic acid sequencing provides one non-limiting example of an application for the disclosed flow cell devices and cartridges (e.g., capillary flow cell or microfluidic chip flow cell devices and cartridges). Many “second generation” and “third generation” sequencing technologies utilize a massively parallel, cyclic array approach to sequencing-by-synthesis (SBS), in which accurate decoding of a single-stranded template oligonucleotide sequence tethered to a solid support relies on successfully classifying signals that arise from the stepwise addition of A, G, C, and T nucleotides by a polymerase to a complementary oligonucleotide strand. These methods typically require the oligonucleotide template to be modified with a known adapter sequence of fixed length, affixed to a solid support (e.g., the lumen surface(s) of the disclosed capillary or microfluidic chip flow cell devices and cartridges) in a random or patterned array by hybridization to surface-tethered probes of known sequence that is complementary to that of the adapter sequence, and then probed through a cyclic series of single base addition primer extension reactions that use, e.g., fluorescently-labeled nucleotides to identify the sequence of bases in the template oligonucleotides. These processes thus require the use of miniaturized fluidics systems that offer precise, reproducible control of the timing of reagent introduction to the flow cell in which the sequencing reactions are performed, and small volumes to reduce or minimize the consumption of costly reagents.

Existing commercially-available NGS flow cells are constructed from layers of glass that have been etched, lapped, and/or processed by other methods to meet the tight dimensional tolerances required for imaging, cooling, and/or other requirements. When flow cells are used as consumables, the costly manufacturing processes required for their fabrication result in costs per sequencing run that are too high to make sequencing routinely accessible to scientists and medical professionals in the research and clinical spaces.

This disclosure provides a low-cost flow cell architecture that includes low cost glass or polymer capillaries or microfluidic channels, fluidics adapters, and cartridge chassis. Utilizing glass or polymer capillaries that are extruded in their final cross-sectional geometry eliminates the need for multiple high-precision and costly glass manufacturing processes. Robustly constraining the orientation of the capillaries or channels and providing convenient fluidic connections using molded plastic and/or elastomeric components further reduces cost. Laser bonding the components of the polymer cartridge chassis provides a fast and efficient means of sealing the capillary or the microfluidic channels and structurally-stabilizing the capillaries or channels and flow cell cartridge without requiring the use of fasteners or adhesives.

Applications of flow cell devices and systems: The flow cell devices and systems described herein can be used in a variety of applications such as sequencing analysis to improve the efficient use of the costly reagents. For examples, a method of sequencing a nucleic acid sample and a second nucleic acid sample can include delivering a plurality of oligonucleotides to an interior surface of an at least partially transparent chamber; delivering a first nucleic acid sample to the interior surface; delivering a plurality of nonspecific reagents through a first channel to the interior surface; delivering a specific reagent through a second channel to the interior surface, wherein the second channel has a lower volume than the first channel; visualizing a sequencing reaction on the interior surface of the at least partially transparent chamber; and replacing the at least partially transparent chamber prior to a second sequencing reaction. In some aspects, flowing an air current past an exterior surface of the at least partially transparent surface. In some aspects, the described method can include selecting the plurality of oligonucleotides to sequence a eukaryotic genome. In some aspects, the described method can include selecting a prefabricated tube as the at least partially transparent chamber. In some aspects, the described method can include selecting the plurality of oligonucleotides to sequence a prokaryotic genome. In some aspects, the described method can include selecting the plurality of oligonucleotides to sequence a transcriptome. In some aspects, the described method can include selecting a capillary tube as the at least partially transparent chamber. In some aspects, the described method can include selecting a microfluidic chip as the at least partially transparent chamber.

The described devices and systems can also be used in a method of reducing a reagent used in a sequencing reaction, comprising providing a first reagent in a first reservoir; providing a second reagent in a first second reservoir, wherein each of the first reservoir and the second reservoir are fluidicaly coupled to a central region, and wherein the central region comprises a surface for the sequencing reaction; and sequentially introducing the first reagent and the second reagent into a central region of the flow cell device, wherein the volume of the first reagent flowing from the first reservoir to the inlet of the central region is less than the volume of the second reagent flowing from the second reservoir to the central region.

An additional use of the described devices and systems is a method of increasing the efficient use of a regent in a sequencing reaction, comprising: providing a first reagent in a first reservoir; providing a second reagent in a first second reservoir, wherein each of the first reservoir and the second reservoir are fluidicaly coupled to a central region, and wherein the central region comprises a surface for the sequencing reaction; and maintaining the volume of the first reagent flowing from the first reservoir to the inlet of the central region to be less than the volume of the second reagent flowing from the second reservoir to the central region.

In general, the first reagent is more expensive than the second agent. In some aspects, the first reagent is selected from the group consisting of a polymerase, a nucleotide, and a nucleotide analog.

Method of fabricating the microfluidic chip: The microfluidic chip can be manufactured by a combination of microfabrication process. The method of manufacturing the microfluidic chip described herein includes providing a surface; and forming at least one channel on the surface. The method of manufacturing can also include providing a first substrate which has at least a first planar surface, wherein the first surface has a plurality of channels; providing a second substrate having at least a second planar surface; and binding the first planar surface of the first substrate to the second planar surface of the second substrate. In some instances, the channels on the first surface have an open top side and closed bottom side, and the second surface is bond to the first surface through the bottom side of the channels and therefore leaving the open top side of the channels unaffected. In some instances, the method described herein further includes providing a third substrate having a third planar surface, and bonding the third surface to the first surface through the open top side of the channels. The bonding conditions can include, e.g., heating the substrates, or applying an adhesive to one of the planar surfaces of the first or second substrate.

Typically, because the devices are microfabricated, substrate materials will be selected based upon their compatibility with known microfabrication techniques, e.g., photolithography, wet chemical etching, laser ablation, laser irradiation, air abrasion techniques, injection molding, embossing, and other techniques. The substrate materials are also generally selected for their compatibility with the full range of conditions to which the microfluidic devices may be exposed, including extremes of pH, temperature, salt concentration, and application of illumination or electric fields. Accordingly, in some preferred aspects, the substrate material may include silica based substrates, such as borosilicate glass, quartz, as well as other substrate materials.

In additional preferred aspects, the substrate materials will comprise polymeric materials, e.g., plastics, such as polymethylmethacrylate (PMMA), polycarbonate, polytetrafluoroethylene (TEFLON™), polyvinylchloride (PVC), polydimethylsiloxane (PDMS), polysulfone, and the like. Such polymeric substrates are readily manufactured using available microfabrication techniques, as described above, or from microfabricated masters, using well known molding techniques, such as injection molding, embossing or stamping, or by polymerizing the polymeric precursor material within the mold (See U.S. Pat. No. 5,512,131). Such polymeric substrate materials are preferred for their ease of manufacture, low cost and disposability, as well as their general inertness to most extreme reaction conditions. Again, these polymeric materials may include treated surfaces, e.g., derivatized or coated surfaces, to enhance their utility in the microfluidic system, e.g., provide enhanced fluid direction.

The channels and/or chambers of the microfluidic devices are typically fabricated into the upper surface of the first substrate, as microscale channels (e.g., grooves, indentations) using the above described microfabrication techniques. The first substrate comprises a top side having a first planar surface and a bottom side. In the microfluidic devices prepared in accordance with the methods described herein, the plurality of channels (e.g., grooves and/or indentations) are formed on the first planar surface. In some instances, the channels (e.g., grooves and/or indentations) formed in the first planar surface (prior to adding a second substrate) has bottom and side walls with the top remaining open. In some instances, the channels (e.g., grooves and/or indentations) in the first planar surface (prior to adding a second substrate) has bottom and side walls and the top remaining closed. In some instances, the channels (e.g., grooves and/or indentations) in the first planar surfaces (prior to adding a second substrate) has only side walls and no top or bottom surface.

When the first planar surface of the first substrate is placed into contact with, and bonded to the planar surface of the second substrate, the second substrate can cover and/or seal the grooves and/or indentations in the surface of the first substrate, to form the channels and/or chambers (e.g., the interior portion) of the device at the interface of these two components.

After the first substrate is bonded to a second substrate, the structure can further placed into contact with and bonded to a third substrate. The third substrate can be placed into contact with the side of the first substrate that is not in contact with the second substrate. In some embodiments, the first substrate is placed between the second substrate and the third substrate. In some embodiments, the second substrate and the third substrate can cover and/or seal the grooves, indentations, or apertures on the first substrate to form the channels and/or chambers (e.g., the interior portion) of the device at the interface of these components.

The device can have openings that are oriented such that they are in communication with at least one of the channels and/or chambers formed in the interior portion of the device from the grooves or indentations. In some embodiments, the openings are formed on the first substrate. In some embodiments, the openings are formed on the first and the second substrate. In some embodiments, the openings are formed on the first, the second, and the third substrate. In some embodiments, the openings are positioned at the top side of the device. In some embodiments, the openings are positioned at the bottom side of the device. In some embodiments, the openings are positioned at the first and/or the second ends of the device, and the channels run along the direction from the first end to the second end.

Conditions under which substrates may be bonded together are generally widely understood, and such bonding of substrates is generally carried out by any of a number of methods, which may vary depending upon the nature of the substrate materials used. For example, thermal bonding of substrates may be applied to a number of substrate materials, including, e.g., glass or silica based substrates, as well as polymer based substrates. Such thermal bonding typically comprises mating together the substrates that are to be bonded, under conditions of elevated temperature and, in some cases, application of external pressure. The precise temperatures and pressures will generally vary depending upon the nature of the substrate materials used.

For example, for silica-based substrate materials, i.e., glass (borosilicate glass, Pyrex™, soda lime glass, etc.), quartz, and the like, thermal bonding of substrates is typically carried out at temperatures ranging from about 500° C. to about 1400° C., and preferably, from about 500° C. to about 1200° C. For example, soda lime glass is typically bonded at temperatures around 550° C., whereas borosilicate glass typically is thermally bonded at or near 800° C. Quartz substrates, on the other hand, are typically thermally bonded at temperatures at or near 1200° C. These bonding temperatures are typically achieved by placing the substrates to be bonded into high temperature annealing ovens.

Polymeric substrates that are thermally bonded, on the other hand, will typically utilize lower temperatures and/or pressures than silica-based substrates, in order to prevent excessive melting of the substrates and/or distortion, e.g., flattening of the interior portion of the device, i.e., channels or chambers. Generally, such elevated temperatures for bonding polymeric substrates will vary from about 80° C. to about 200° C., depending upon the polymeric material used, and will preferably be between about 90° C. and 150° C. Because of the significantly reduced temperatures required for bonding polymeric substrates, such bonding may typically be carried out without the need for high temperature ovens, as used in the bonding of silica-based substrates. This allows incorporation of a heat source within a single integrated bonding system, as described in greater detail below.

Adhesives may also be used to bond substrates together according to well known methods, which typically comprise applying a layer of adhesive between the substrates that are to be bonded and pressing them together until the adhesive sets. A variety of adhesives may be used in accordance with these methods, including, e.g., UV curable adhesives, that are commercially available. Alternative methods may also be used to bond substrates together in accordance with the present invention, including e.g., acoustic or ultrasonic welding and/or solvent welding of polymeric parts.

Typically, a number of the described microfluidic chips or devices will be manufactured at a time. For example, polymeric substrates may be stamped or molded in large separable sheets which can be mated and bonded together. Individual devices or bonded substrates may then be separated from the larger sheet. Similarly, for silica-based substrates, individual devices can be fabricated from larger substrate wafers or plates, allowing higher throughput of the manufacturing process. Specifically, a number of channel structures can be manufactured into a first substrate wafer or plate which is then overlaid with a second substrate wafer or plate, and optionally further overlaid with a third substrate wafer or plate. The resulting multiple devices are then segmented from the larger substrates using known methods, such as sawing, scribing and breaking, and the like.

As noted above, the top or second substrate is overlaid upon the bottom or first substrate to seal the various channels and chambers. In carrying out the bonding process according to the methods of the present invention, the bonding of the first and second substrates is carried out using vacuum to maintain the two substrate surfaces in optimal contact. In particular, the bottom substrate may be maintained in optimal contact with the top substrate by mating the planar surface of the bottom substrate with the planar surface of the top substrate, and applying a vacuum through the holes that are disposed through the top substrate. Typically, application of a vacuum to the holes in the top substrate is carried out by placing the top substrate on a vacuum chuck, which typically comprises a mounting table or surface, having an integrated vacuum source. In the case of silica-based substrates, the bonded substrates are subjected to elevated temperatures in order to create an initial bond, so that the bonded substrates may then be transferred to the annealing oven, without any shifting relative to each other.

Alternate bonding systems for incorporation with the apparatus described herein include, e.g., adhesive dispensing systems, for applying adhesive layers between the two planar surfaces of the substrates. This may be done by applying the adhesive layer prior to mating the substrates, or by placing an amount of the adhesive at one edge of the adjoining substrates, and allowing the wicking action of the two mated substrates to draw the adhesive across the space between the two substrates.

In certain embodiments, the overall bonding system can include automatable systems for placing the top and bottom substrates on the mounting surface and aligning them for subsequent bonding. Typically, such systems include translation systems for moving either the mounting surface or one or more of the top and bottom substrates relative to each other. For example, robotic systems may be used to lift, translate and place each of the top and bottom substrates upon the mounting table, and within the alignment structures, in turn. Following the bonding process, such systems also can remove the finished product from the mounting surface and transfer these mated substrates to a subsequent operation, e.g., separation operation, annealing oven for silica-based substrates, etc., prior to placing additional substrates thereon for bonding.

In some instances, the manufacturing of the microfluidic chip includes the layering or laminating of two or more layers of substrates, in order to produce the chip. For example, in microfluidic devices, the microfluidic elements of the device are typically produced by laser irradiation, etching or otherwise fabricating features into the surface of a first substrate. A second substrate is then laminated or bonded to the surface of the first to seal these features and provide the fluidic elements of the device, e.g., the fluid channels.

### EXAMPLES

These examples are provided for illustrative purposes only and not to limit the scope of the claims provided herein.

### Design Specifications for a Fluorescence Imaging Module for Genomics Applications

A non-limiting example of design specifications for a fluorescence imaging module of the present disclosure is provided in Table 1.

TABLE 1
Examples of design specifications for a fluorescence
imaging module for genomics applications.

| Design Parameter | Specification | |
|---|---|---|
| Numerical aperture | ≥0.3 | |
| Image quality | Diffraction limited | |
| Field-of-view (FOV) | ≥1.0 | mm² |
| Image plane curvature | Best focal plane within 100 nm for >90% of the | |
| | FOV, within 150 nm for 99% of the FOV, and | |
| | within 200 nm for the entire FOV | |
| Image distortion | <0.5% across the FOV | |
| Magnification | 2x to 20x | |
| Camera pixel size at | ≥2 x optical system modulation transfer | |
| sample plane | function (MTF) limit | |
| Coverslip thickness | >700 | μm |
| Number of fluorescence | ≥3 | |
| imaging channels | | |
| Chromatic focal plane | ≤100 nm equivalent at sample plane | |
| difference at camera | | |
| between all imaging | | |
| channels | | |
| Number of AF channels | 1 | |
| Imaging time | ≤2 seconds per FOV | |
| Autofocus | Single step autofocus with error correction | |
| Autofocus accuracy | <100 | nm |
| Scanning stage step and | <0.4 | seconds |
| settle time | | |

| | |
|---|---|
| Channel-specific | 1 per imaging channel |
| optimized tube lens | |
| Illumination optical path | Liquid light guide with underfilled entrance |
| | aperture |

### Example Flow Cell Designs

### Example 1

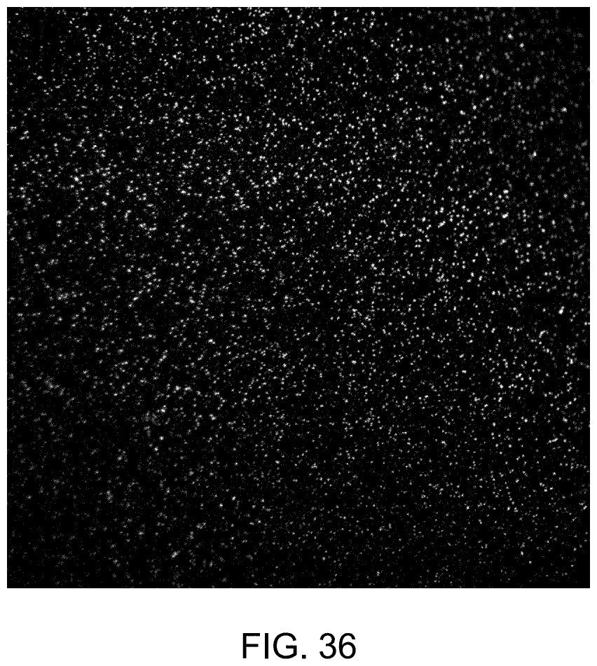

Nucleic acid clusters were established within a capillary and subjected to fluorescence imaging. A flow device having a capillary tube was used for the test. The resulting cluster images were presented in FIG. 36. The figure demonstrated that clusters within the lumen of a capillary system as disclosed herein can be reliably amplified and visualized.

### Example 2

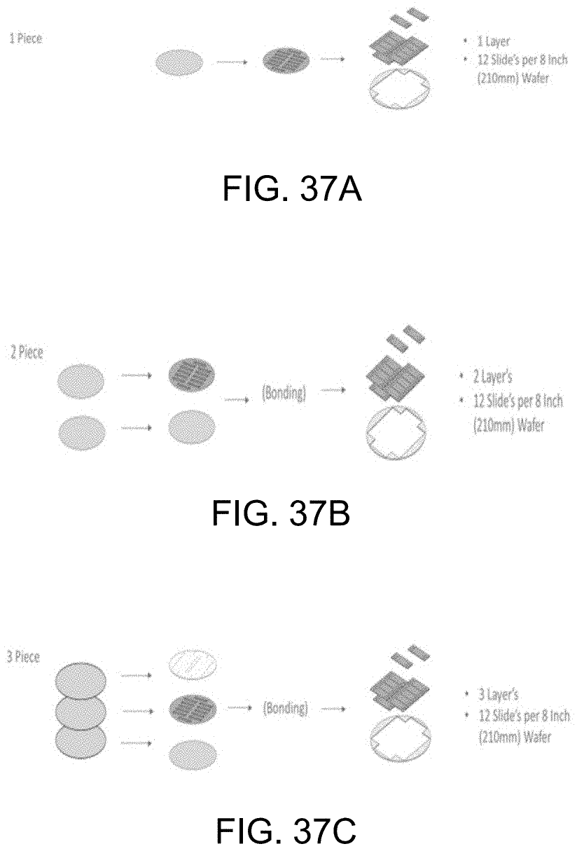

Flow cell device can be constructed from one, two, or three layer of glasses using one of the steps as shown in FIGS. 37A-37C. In FIGS. 37A-37C, the flow cell devices can be made form one, two, or three layers of glasses. The glasses can be either quarts or borosilicate glass. FIGS. 37A-37C show the methods to make such devices at wafer level with technologies such as focused femtosecond laser radiation (1 piece) and/or laser glass bonding (2 or 3 piece construction).

In FIG. 37A, the first layer of wafer is processed with a laser (e.g., femtosecond laser radiation) to ablate the wafer material and provide a patterned surface. The patterned surface can be a plurality of channels on the surface such as 12 channels per wafer. The wafer has a diameter of 210 mm. The processed wafer can be then placed on a support plate to form channels that can be used to direct fluid flow through a particular direction.

In FIG. 37B, the first layer of wafer having a patterned surface can be placed in contact with and bonded to a second layer of wafer. The bonding can be performed using a laser glass bonding technology. The second layer can cover and/or seal the grooves, indentations, or apertures on the wafer having the patterned surface to form the channels and/or chambers (e.g., the interior portion) of the device at the interface of these components. The bonded structure with two layers of wafer can then be placed on a support plate. The patterned surface can be a plurality of channels on the surface such as 12 channels per wafer. The wafer can have a diameter of 210 mm.

In FIG. 37C, the first layer of wafer having a patterned surface can be placed in contact with and bonded to a second layer of wafer on one side, and a third layer of wafer can be bonded to the first wafer layer on the other side so that the first player of wafer is positioned between the second and the third layers of wafer. The bonding can be performed using a laser glass bonding technology. The second layer and the third layer of wafers can cover and/or seal the grooves, indentations, or apertures on the wafer having the patterned surface to form the channels and/or chambers (e.g., the interior portion) of the device. The bonded structure with three layers of wafer can then be placed on a support plate. The patterned surface can be a plurality of channels on the surface such as 12 channels per wafer. The wafer can have a diameter of 210 mm.

### Example 3

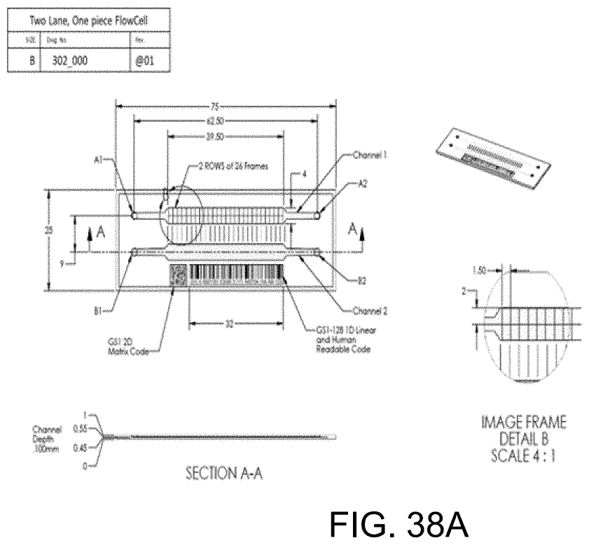

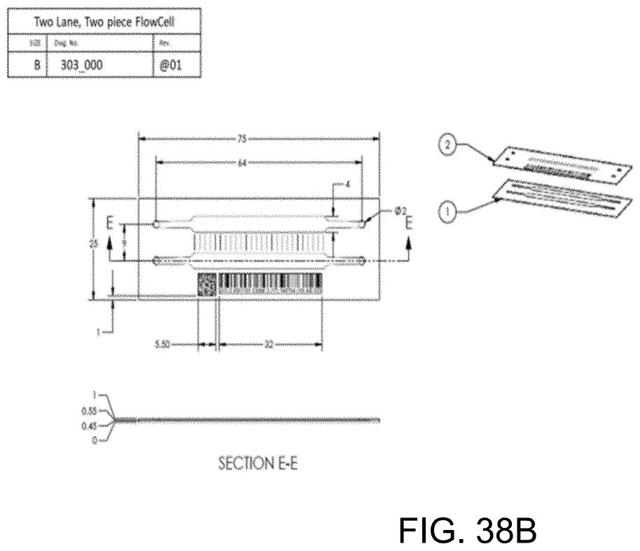

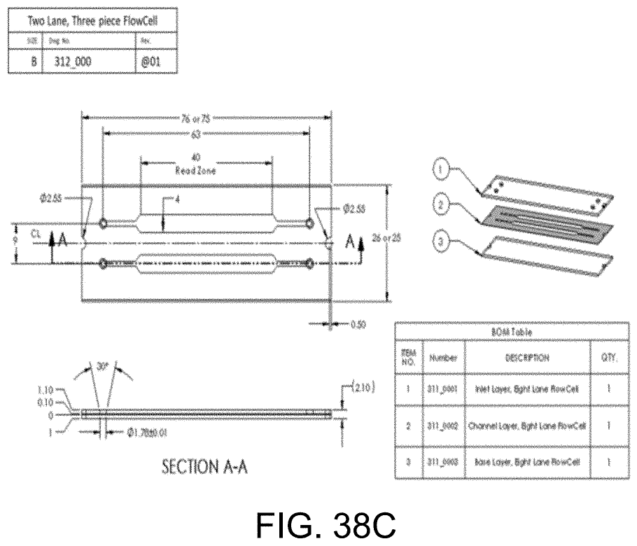

FIG. 38A shows a one-piece glass flow cell design. In this design, flow channels and inlet outlet holes can be fabricated using focused femtosecond laser radiation method. There are two channels/lanes on the flow cell, and each channel has 2 rows with 26 frames in each row. The channel can have a depth of about 100 μm. Chanel 1 has an inlet hole A1 and an outlet hole A2, and channel 2 has an inlet hole B1 and an outlet hole B2. The flow cell can also have a 1D linear and human readable code, and optionally a 2D matrix code.

FIG. 38B shows a two-piece glass flow cell. In this design, flow channels and inlet and outlet holes can be fabricated using focused femtosecond laser radiation or chemical etching technology. The 2 pieces can be bonded together with laser glass bonding technology. The inlet and outlet holes can be positioned on the top layer of the structure and oriented in a way such that they are in communication with at least one of the channels and/or chambers formed in the interior portion of the device. There are two channels on the cell, and each channel has 2 rows with 26 frames in each row. The channel can have a depth of about 100 μm. Chanel 1 has an inlet hole A1 and an outlet hole A2, and channel 2 has an inlet hole B1 and an outlet hole B2. The flow cell can also have a 1D linear and human readable code, and optionally a 2D matrix code.

FIG. 38C shows a three-piece glass flow cell. In this design, flow channels and inlet and outlet holes can be fabricated using focused femtosecond laser radiation or chemical etching technology. The 3 pieces can be bonded together with laser glass bonding technology. The first layer of wafer having a patterned surface can be bonded to a second layer of wafer on one side, and a third layer of wafer can be bonded to the first wafer layer on the other side so that the first player of wafer is positioned between the second and the third layers of wafer. The inlet and outlet holes can be positioned on the top layer of the structure and oriented in a way such that they are in communication with at least one of the channels and/or chambers formed in the interior portion of the device. There are two channels on the cell, and each channel has 2 rows with 26 frames in each row. The channel can have a depth of about 100 μm. Chanel 1 has an inlet hole A1 and an outlet hole A2, and channel 2 has an inlet hole B1 and an outlet hole B2. The flow cell can also have a 1D linear and human readable code, and optionally a 2D matrix code.

### Example 4

Flow cells were coated by washing prepared glass channels with KOH followed by rinsing with ethanol and silanization for 30 minutes at 65° C. Channel surfaces were activated with EDC-NHS for 30 min. followed by grafting of primers by incubation with 5 μm primer for 20 min., and then passivation with 30 μm PEG-NH2.

Multilayer surfaces are made following the approach of Example 4, where following PEG passivation, a multi-armed PEG-NHS is flowed through the channels following addition of the PEG-NH2, optionally followed by another incubation with PEG-NHS, and optionally another incubation with multi-armed PEG-NH2. For these surfaces, primer may be grafted at any step, especially following the last addition of multi-armed PEG-NH2.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in any combination in practicing the invention. It is intended that the following claims define the scope of the invention and that methods and structures within the scope of these claims and their equivalents be covered thereby.

### Additional Embodiments

Various example embodiments of the present technology are described herein. Reference is made to these examples in a non-limiting sense. They are provided to illustrate more broadly applicable aspects of the present disclosure. Various changes may be made to the technology described and equivalents may be substituted without departing from the spirit and scope of the present disclosure.

In addition, many modifications may be made to adapt a particular situation, material, composition of matter, process, process act, or step(s) to the objective(s), spirit, or scope of the present technology. Further, as will be appreciated by those with skill in the art that each of the individual variations described and illustrated herein has discrete components and features which may be readily separated from or combined with the features of any of the other several embodiments without departing from the scope or spirit of the present disclosure. All such modifications are intended to be within the scope of claims associated with this disclosure.

The present disclosure includes methods that may be performed using the subject devices. The methods may comprise the act of providing such a suitable device. Such provision may be performed by the user. In other words, the “providing” act merely requires the user obtain, access, approach, position, set-up, activate, power-up or otherwise act to provide the requisite device in the subject method. Methods recited herein may be carried out in any order of the recited events that is logically possible, as well as in the recited order of events.

Example aspects of the present technology, together with details regarding material selection and manufacture have been set forth above. As for other details of the present technology, these may be appreciated in connection with the above-referenced patents and publications as well as generally known or appreciated by those with skill in the art. The same may hold true with respect to method-based aspects of the present technology in terms of additional acts as commonly or logically employed.

In addition, though the present technology has been described in reference to several examples optionally incorporating various features, the present disclosure is not to be limited to that which is described or indicated as contemplated with respect to each variation of the present technology. Various changes may be made to the technology described and equivalents (whether recited herein or not included for the sake of some brevity) may be substituted without departing from the spirit and scope of the present disclosure. In addition, where a range of values is provided, it is understood that every intervening value, between the upper and lower limit of that range and any other stated or intervening value in that stated range, is encompassed within the scope of the present disclosure.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

What is claimed is:

1. An optical system comprising:
at least one light source configured to produce an excitation beam;
an objective lens configured to receive emission light from within a specified field of view (FOV) of a sample on a sample support structure in response to the excitation beam, said objective lens having a numerical aperture of less than 0.6 and a working distance of at least 700 μm, said FOV having an area of at least 1 mm²; and
at least one detection channel comprising optics and a photodetector array configured to receive at least a portion of the emission light and capture an image of at least one fluorescing sample site on said sample support structure.

2. The optical system of claim 1, wherein said sample support structure comprises a low binding surface having a water contact angle of less than 50 degrees, said sample support structure comprising amplified DNA colonies with a density of greater than 10,000 per mm².

3. The optical system of claim 1, wherein said objective lens has a numerical aperture of 0.5 or less.

4. The optical system of claim 3, wherein said objective lens has a numerical aperture of at least 0.3 and not greater than 0.4.

5. The optical system of claim 1, wherein said FOV has an area of at least 1.5 mm².

6. The optical system of claim 1, wherein said FOV has an area of at least 2 mm².

7. The optical system of claim 1, said objective lens having an optical axis, said sample support structure comprising a first surface and a second surface, wherein said first surface is between said objective lens and said second surface, said first and second surfaces separated from each other along said optical axis by at least 0.05 mm and not more than 0.7 mm.

8. The optical system of claim 7, wherein said first and second surfaces are separated from each other by at least 0.075 mm and not more than 0.7 mm.

9. The optical system of claim 7, wherein said optical system provides diffraction limited imaging of both said first and second surfaces.

10. The optical system of claim 7, wherein said optical system is capable of capturing with said photodetector array images of fluorescence emitting sample sites on first and second surfaces on said sample support structure.

11. The optical system of claim 10, further comprising said sample support structure having said first and second surfaces.

12. The optical system of claim 11, wherein said sample support structure comprises a flow cell having a flow channel and said first and second surfaces comprise interior surfaces of said flow cell configured to be in contact with a sample flowing through said flow cell.

13. The optical system of claim 11, wherein said first and second surfaces on said sample support structure each comprise amplified DNA colonies with a density of greater than 10,000 per mm².

14. The optical system of claim 13, wherein said amplified DNA colonies are labeled with a fluorescent tag through a mono or oligo nucleotide such that a contrast-to-noise ratio (CNR) is greater than 4.

15. The optical system of claim 13, wherein said amplified DNA colonies include individual colonies classified from a fluorescent signal uniquely identifying A, G, C, or T through an incorporation, avidity, or binding event.

16. The optical system of claim 1, further comprising a means for sequencing a nucleic acid, wherein sequencing a nucleic acid comprises carrying out a sequencing by binding or sequencing by synthesis reaction on one or more surfaces, and detecting a bound or incorporated base using the optical system of claim 1.

17. The optical system of claim 1, further comprising a means for determining a genotype of a sample comprising a nucleic acid molecule, wherein determining the genotype of a sample comprises preparing said nucleic acid molecule for sequencing, and then sequencing said nucleic acid molecule using the system of claim 1.
